SEQUENCE LISTING (41 sequences)

SEQ ID NO: 1 - DNA, 552 bp, Artificial Sequence
  Synthetic Construct
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 2 - DNA, 1698 bp, Influenza A H1N1 virus
  Strain A/California/07/2009
atgaaagcgattctggtggtgctgctgtatacctttgcgaccgcgaacgcggataccctgtgcattggctatcatgcgaacaacagcaccgataccgtggataccgtgctggaaaaaaacgtgaccgtgacccatagcgtgaacctgctggaagataaacataacggcaaactgtgcaaactgcgcggcgtggcgccgctgcatctgggcaaatgcaacattgcgggctggattctgggcaacccggaatgcgaaagcctgagcaccgcgagcagctggagctatattgtggaaaccccgagcagcgataacggcacctgctatccgggcgattttattgattatgaagaactgcgcgaacagctgagcagcgtgagcagctttgaacgctttgaaatttttccgaaaaccagcagctggccgaaccatgatagcaacaaaggcgtgaccgcggcgtgcccgcatgcgggcgcgaaaagcttttataaaaacctgatttggctggtgaaaaaaggcaacagctatccgaaactgagcaaaagctatattaacgataaaggcaaagaagtgctggtgctgtggggcattcatcatccgagcaccagcgcggatcagcagagcctgtatcagaacgcggatgcgtatgtgtttgtgggcagcagccgctatagcaaaaaatttaaaccggaaattgcgattcgcccgaaagtgcgcgatcaggaaggccgcatgaactattattggaccctggtggaaccgggcgataaaattacctttgaagcgaccggcaacctggtggtgccgcgctatgcgtttgcgatggaacgcaacgcgggcagcggcattattattagcgataccccggtgcatgattgcaacaccacctgccagaccccgaaaggcgcgattaacaccagcctgccgtttcagaacattcatccgattaccattggcaaatgcccgaaatatgtgaaaagcaccaaactgcgcctggcgaccggcctgcgcaacattccgagcattcagagccgcggcctgtttggcgcgattgcgggctttattgaaggcggctggaccggcatggtggatggctggtatggctatcatcatcagaacgaacagggcagcggctatgcggcggatctgaaaagcacccagaacgcgattgatgaaattaccaacaaagtgaacagcgtgattgaaaaaatgaacacccagtttaccgcggtgggcaaagaatttaaccatctggaaaaacgcattgaaaacctgaacaaaaaagtggatgatggctttctggatatttggacctataacgcggaactgctggtgctgctggaaaacgaacgcaccctggattatcatgatagcaacgtgaaaaacctgtatgaaaaagtgcgcagccagctgaaaaacaacgcgaaagaaattggcaacggctgctttgaattttatcataaatgcgataacacctgcatggaaagcgtgaaaaacggcacctatgattatccgaaatatagcgaagaagcgaaactgaaccgcgaagaaattgatggcgtgaaactggaaagcacccgcatttatcagattctggcgatttatagcaccgtggcgagcagcctggtgctggtggtgagcctgggcgcgattagcttttggatgtgcagcaacggcagcctgcagtgccgcatttgcatt

SEQ ID NO: 3 - DNA, 1695 bp, Influenza A H1N1 virus
  Strain A/Puerto Rico/8/1934
atgaaagcgaacctgctggtgctgctgtgcgcgctggcggcggcggatgcggataccatttgcattggctatcatgcgaacaacagcaccgataccgtggataccgtgctggaaaaaaacgtgaccgtgacccatagcgtgaacctgctggaagatagccataacggcaaactgtgccgcctgaaaggcattgcgccgctgcagctgggcaaatgcaacattgcgggctggctgctgggcaacccggaatgcgatccgctgctgccggtgcgcagctggagctatattgtggaaaccccgaacagcgaaaacggcatttgctatccgggcgattttattgattatgaagaactgcgcgaacagctgagcagcgtgagcagctttgaacgctttgaaatttttccgaaagaaagcagctggccgaaccataacaccaacggcgtgaccgcggcgtgcagccatgaaggcaaaagcagcttttatcgcaacctgctgtggctgaccgaaaaagaaggcagctatccgaaactgaaaaacagctatgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgccgctttaccccggaaattgcggaacgcccgaaagtgcgcgatcaggcgggccgcatgaactattattggaccctgctgaaaccgggcgataccattatttttgaagcgaacggcaacctgattgcgccgatgtatgcgtttgcgctgagccgcggctttggcagcggcattattaccagcaacgcgagcatgcatgaatgcaacaccaaatgccagaccccgctgggcgcgattaacagcagcctgccgtatcagaacattcatccggtgaccattggcgaatgcccgaaatatgtgcgcagcgcgaaactgcgcatggtgaccggcctgcgcaacattccgagcattcagagccgcggcctgtttggcgcgattgcgggctttattgaaggcggctggaccggcatgattgatggctggtatggctatcatcatcagaacgaacagggcagcggctatgcggcggatcagaaaagcacccagaacgcgattaacggcattaccaacaaagtgaacaccgtgattgaaaaaatgaacattcagtttaccgcggtgggcaaagaatttaacaaactggaaaaacgcatggaaaacctgaacaaaaaagtggatgatggctttctggatatttggacctataacgcggaactgctggtgctgctggaaaacgaacgcaccctggattttcatgatagcaacgtgaaaaacctgtatgaaaaagtgaaaagccagctgaaaaacaacgcgaaagaaattggcaacggctgctttgaattttatcataaatgcgataacgaatgcatggaaagcgtgcgcaacggcacctatgattatccgaaatatagcgaagaaagcaaactgaaccgcgaaaaagtggatggcgtgaaactggaaagcatgggcatttatcagattctggcgatttatagcaccgtggcgagcagcctggtgctgctggtgagcctgggcgcgattagcttttggatgtgcagcaacggcagcctgcagtgccgcatttgcatt

SEQ ID NO: 4 - DNA, 84 bp, Artificial Sequence
  Synthetic Construct
aaaaaacaaaaaacaaaacggctattaatagccgaaaaacaaaaaacaaaaaaaacaaaaaaaaaaccaaaaaaacaaaacaca

SEQ ID NO: 5 - RNA, 96 nt, Hepatitis delta virus
ggcucaucucgacaagaggcggcaguccucaguacucuuacucuuuucuguaaagaggagacugcuggacucgccgcccaaguucgagcaugagcc

SEQ ID NO: 6 - RNA, 74 nt, Hepatitis delta virus
ggcuagaggcggcaguccucaguacucuuacucuuuucuguaaagaggagacugcuggacucgccgcccgagcc

SEQ ID NO: 7 - protein, 8 aa, Artificial Sequence
  Synthetic Construct
  misc_feature (2)..(2): Xaa is Val or Ile
  misc_feature (4)..(4): Xaa is any amino acid
DXEXNPGP

SEQ ID NO: 8 - protein, 6 aa, Artificial Sequence
  Synthetic Construct
CHYSEL

SEQ ID NO: 9 - protein, 9 aa, Artificial Sequence
  Synthetic Construct
  misc_feature (1)..(1): Xaa is absent or Gly or His
  misc_feature (2)..(2): Xaa is absent or Asp or Gly
  misc_feature (3)..(3): Xaa is Asp or Val or Ile or Ser or Met
  misc_feature (5)..(5): Xaa is any amino acid
XXXEXNPGP

SEQ ID NO: 10 - protein, 9 aa, Artificial Sequence
  Synthetic Construct
GDVESNPGP

SEQ ID NO: 11 - protein, 9 aa, Artificial Sequence
  Synthetic Construct
GDIEENPGP

SEQ ID NO: 12 - protein, 7 aa, Artificial Sequence
  Synthetic Construct
VEPNPGP

SEQ ID NO: 13 - protein, 7 aa, Artificial Sequence
  Synthetic Construct
IETNPGP

SEQ ID NO: 14 - protein, 9 aa, Artificial Sequence
  Synthetic Construct
GDIESNPGP

SEQ ID NO: 15 - protein, 9 aa, Artificial Sequence
  Synthetic Construct
GDVELNPGP

SEQ ID NO: 16 - protein, 9 aa, Artificial Sequence
  Synthetic Construct
GDIETNPGP

SEQ ID NO: 17 - protein, 8 aa, Artificial Sequence
  Synthetic Construct
GDVENPGP

SEQ ID NO: 18 - protein, 9 aa, Artificial Sequence
  Synthetic Construct
GDVEENPGP

SEQ ID NO: 19 - protein, 9 aa, Artificial Sequence
  Synthetic Construct
GDVEQNPGP

SEQ ID NO: 20 - protein, 7 aa, Artificial Sequence
  Synthetic Construct
IESNPGP

SEQ ID NO: 21 - protein, 9 aa, Artificial Sequence
  Synthetic Construct
GDIELNPGP

SEQ ID NO: 22 - protein, 9 aa, Artificial Sequence
  Synthetic Construct
HDIETNPGP

SEQ ID NO: 23 - protein, 9 aa, Artificial Sequence
  Synthetic Construct
HDVETNPGP

SEQ ID NO: 24 - protein, 9 aa, Artificial Sequence
  Synthetic Construct
HDVEMNPGP

SEQ ID NO: 25 - protein, 9 aa, Artificial Sequence
  Synthetic Construct
GDMESNPGP

SEQ ID NO: 26 - protein, 9 aa, Artificial Sequence
  Synthetic Construct
GDVETNPGP

SEQ ID NO: 27 - protein, 9 aa, Artificial Sequence
  Synthetic Construct
GDIEQNPGP

SEQ ID NO: 28 - protein, 8 aa, Artificial Sequence
  Synthetic Construct
DSEFNPGP

SEQ ID NO: 29 - RNA, 10 nt, Artificial Sequence
  Synthetic Construct
  misc_feature (3)..(3): n is a or g
  misc_feature (4)..(4): n is a or g
ccnnccaugg

SEQ ID NO: 30 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - DNA, 3819 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca

SEQ ID NO: 32 - protein, 241 aa, Severe acute respiratory syndrome coronavirus 2
MGVKVLFALICIAVAEAKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 33 - DNA, 723 bp, Severe acute respiratory syndrome coronavirus 2
atgggagtcaaagttctgtttgccctgatctgcattgctgtggccgaggccaagagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 34 - protein, 566 aa, Influenza A H1N1 virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 35 - DNA, 1698 bp, Influenza A H1N1 virus
atgaaagcaatactagtagttcttctatatacatttgcaaccgctaacgctgatacattgtgtataggatatcacgcgaacaactccaccgatacagtagatacagtactagagaagaacgtaacagtaacacattctgttaatcttctagaagacaagcataacggcaaactgtgcaaactaagaggtgtagccccattgcatctaggaaagtgtaatatagctggctggattttgggaaatccagagtgtgaatcattaagtacagcaagctcctggtcctatatagtggaaacacctagtagtgataacggaacgtgttacccaggagattttatagattacgaggagctaagagagcagctgtcgtcagtatcatcatttgaaaggtttgaaattttcccgaaaacatcctcctggcccaatcacgatagtaacaaaggagtaacagcagcctgtcctcacgctggagcaaaaagcttctataaaaatttaatctggctagtgaagaagggaaattcatatccaaagctaagtaaaagttatataaacgataagggcaaggaagtactcgtactgtggggcattcatcatccatctactagtgctgatcaacaaagtttatatcaaaacgcagacgcatacgtttttgtggggtcaagtagatatagcaagaaatttaaaccagaaatagcaataagacctaaagtaagggatcaagaaggcagaatgaactattattggacactagtagaaccgggagataaaataacttttgaagcaacaggaaatctagtggttcccaggtacgcatttgcaatggaaagaaacgctggatcaggcatcattatatctgatacaccagtccacgattgtaatacaacttgtcaaacacctaaaggagctataaacaccagcttaccatttcaaaatattcatcctatcacaattggaaagtgtccaaaatacgtaaaaagtacaaaattgagattggccacaggattacgaaatattccatcaattcaatctagaggactttttggtgcaattgcaggtttcatagaaggaggctggactgggatggtagacggctggtacggttatcatcatcaaaacgaacagggaagtggatacgcagctgatcttaaaagtacacaaaacgcaattgacgagattactaataaagtaaattctgtaattgaaaaaatgaatactcagtttacagcagtagggaaagagtttaaccacctggaaaaaagaatagaaaatttaaataaaaaagtagacgacggatttcttgacatttggacttataacgccgaactattggtattactagaaaacgaaagaactctagattatcacgattcaaacgtaaaaaatttatacgaaaaagtaagaagccaacttaaaaataacgcaaaagaaataggaaacggctgttttgaattttatcacaagtgtgataatacctgcatggaaagtgttaaaaacgggacatacgattatccaaaatactcagaagaagcaaaattaaatagagaagaaatagacggcgtaaaattagaatcaacaaggatatatcaaatattagcaatatattcaactgtcgcttcttcattggtactggtagtttctctaggtgcaatatcattttggatgtgctctaacggctccctacagtgtagaatttgtata

SEQ ID NO: 36 - protein, 185 aa, Artificial Sequence
  Synthetic Construct
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 37 - DNA, 558 bp, Artificial Sequence
  Synthetic Construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 38 - DNA, 1614 bp, Homo sapiens
atggctccccaaaagctgaccatcagctggttcgccatcgtgctgctcgtgagccccctgatggccatgtgggagctggagaaggacgtgtacgtggtcgaggtggactggacccccgacgcccccggcgaaacagtgaacctgacctgcgacacccccgaggaagacgatatcacctggacaagcgaccagcggcacggcgtgatcggcagcggcaagaccctgaccatcaccgtgaaggagttcctggacgccggccagtacacctgccacaagggcggagaaacactgagccacagccacctgctgttgcacaagaaagagaacggcatctggagcaccgagatcctcaagaatttcaagaacaagaccttcctgaagtgcgaggcccccaactacagcggccggttcacctgcagctggctggtgcagcggaacatggacctgaagttcaacatcaagagctccagctctagccccgacagccgggccgtgacctgcggcatggccagcctgagcgccgagaaggtgaccctggaccagcgggactacgagaagtacagcgtgagctgccaggaggacgtgacctgccctaccgctgaggaaacactgcccatcgagctggccctggaggcccggcagcaaaacaagtacgagaactacagcaccagcttctttatccgggacatcattaagcccgacccccctaagaacctgcagatgaagcccctgaagaacagccaggtggaggtgagctgggagtaccccgacagctggagcaccccccacagctacttcagcctgaagttcttcgtgcggatccagcggaagaaggagaagatgaaggaaacagaggagggctgcaaccagaagggcgccttcctggtggaaaagacaagcacagaggtgcagtgcaagggcggcaacgtgtgcgtgcaggcccaggaccggtactataacagcagctgcagcaagtgggcctgcgtgccctgccgggtgcggagtgtgcctggcgtcggcgtgcccggcgtgggccgggtgatccccgtgagcggccccgcccggtgcctgagccagagccggaacctgctgaagaccacagacgacatggtgaagaccgcccgggagaagctgaagcactacagctgcaccgccgaggacatcgaccacgaggacatcacccgggaccagaccagcaccctgaagacctgcctccccctggagctgcacaagaacgagagctgcctggccacccgggaaacaagctctaccacacggggcagctgcctgcctcctcagaagaccagcctgatgatgaccctgtgcctgggcagcatctacgaggacctgaagatgtaccagaccgagttccaggccatcaacgccgctctgcagaaccacaaccaccagcagatcatcctggacaagggcatgctggtggccatcgacgagctgatgcagagcctgaaccacaacggcgaaacactgcggcagaagcccccagtgggcgaggccgacccctaccgggtgaagatgaagctgtgcatcctgctgcacgccttcagcacccgggtggtgaccatcaaccgggtgatgggctacctgagcagcgcc

SEQ ID NO: 39 - protein, 538 aa, Homo sapiens
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 40 - DNA, 1158 bp, Gallus gallus
atgggcagcatcggcgccgctagcatggagttctgctttgacgtcttcaaagagctgaaggtgcaccacgccaacgagaacatcttctactgccctattgccatcatgagcgccctggccatggtgtacctgggcgccaaggacagcacccggacccagatcaacaaggtggtccggttcgacaagctgcccggcttcggcgacagcatcgaggcccagtgcggcaccagcgtgaacgtgcacagctccctgcgggacatcctgaaccagatcaccaagcccaacgacgtgtacagcttcagcctggccagccggctgtacgccgaggaacggtaccccatcctgcccgagtacctgcagtgcgtgaaggagctgtaccggggcggactggagcccatcaacttccagaccgccgccgaccaggcccgggagctgatcaacagctgggtggagagccagaccaacggcatcattcggaacgtgctgcagcccagcagcgtggacagccagaccgccatggtgctggtgaacgccatcgtgttcaagggcctgtgggagaaggccttcaaggacgaggacacccaggccatgcccttccgggtgaccgagcaggagagcaagcccgtgcagatgatgtaccagatcggcctgttccgggtggccagcatggccagcgagaagatgaagatcctggagctgcccttcgccagcggcaccatgagcatgctggtgctgctccccgacgaggtgagcggcctggagcagctggagagcatcatcaacttcgagaagctgaccgagtggaccagctctaacgtgatggaggagcggaagatcaaggtgtacctgccccggatgaagatggaggagaagtacaacctgaccagcgtgctgatggccatgggcatcaccgacgtgttcagcagcagcgccaacctgagcggcatcagctccgccgagagcctgaagatcagccaggccgtgcacgccgcacacgccgagatcaacgaggccggccgggaggtggtgggcagcgccgaggccggcgtggacgccgccagcgtgagcgaggagttccgggccgaccaccccttcctgttctgcatcaagcatattgccaccaacgccgtgctgttctttggccggtgcgtgagcccc

SEQ ID NO: 41 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

(19) United States
(12) Patent Application Publication
(10) Pub. No.: US 2024/0009298 A1
(43) Pub. Date: Jan. 11, 2024

(54) IMMUNOGENIC COMPOSITIONS AND USES THEREOF

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US); Yann Paul Guy Régis ECHELARD, Jamaica Plain, MA (US); Nicholas McCartney PLUGIS, Duxbury, MA (US); Erica Gabrielle WEINSTEIN, Newton, MA (US); Jennifer A. NELSON, Brookline, MA (US)

(21) Appl. No.: 18/024,542

(22) PCT Filed: Sep. 3, 2021
(86) PCT No.: PCT/US2021/049077

§ 371 Date: Mar. 3, 2023

Related U.S. Application Data
(60) Provisional application No. 63/191,020, filed on May 20, 2021.
(60) Provisional application No. 63/074,428, filed on Sep. 3, 2020.

(51) Int. Cl. A61K 39/215; C12N 15/85; A61P 31/14

(52) U.S. Cl. CPC A61K 39/215; C12N 15/85; A61P 31/14; C12N 2840/203; C12N 2840/206; A61K 2039/53

(57) ABSTRACT

This disclosure provides compositions, pharmaceutical preparations, and uses of polyribonucleotides encoding one or more immunogenic polypeptides. In particular, this disclosure features circular polyribonucleotide encoding one or more immunogenic polypeptides.

### SEQUENCE LISTING

The instant application contains a Sequence Listing which has been submitted electronically in ASCII format and is hereby incorporated by reference in its entirety. Said ASCII copy, created on Sep. 2, 2021, is named 51509-045WO2_Sequence_Listing_9_2_21_ST25 and is 52,787 bytes in size.

### BACKGROUND

Vaccination has made an enormous contribution to both human and animal health. Since the invention of the first vaccine in 1796, vaccines have come to be considered the most successful method for preventing many infectious diseases by provoking an immune response in a subject. According to the World Health Organization, immunization currently prevents 2-3 million deaths every year across all age groups. Today, vaccines have been developed to prevent and control the spread of more than 20 infectious diseases, including diphtheria, tetanus, pertussis, influenza, and measles, and have led to the complete eradication of smallpox. There remains a need to develop new and improved immunogenic compositions and uses thereof.

### SUMMARY

This disclosure provides compositions, pharmaceutical preparations, and uses of polyribonucleotides (e.g., circular or linear polyribonucleotides) encoding one or more immunogens. In particular, the disclosure provides circular polyribonucleotides encoding multiple immunogens, circular polyribonucleotides encoding at least one immunogen and further encoding at least one adjuvant, and compositions including multiple circular polyribonucleotides. This disclosure further relates to methods of using the circular polyribonucleotides encoding one or more polypeptide immunogens. Compositions and pharmaceutical preparations of circular polyribonucleotides described herein may induce an immune response in a subject upon administration. Compositions and pharmaceutical preparations of circular polyribonucleotides described herein may be used to treat or prevent a disease, disorder, or condition in a subject.

In one aspect, provided herein is a circular polyribonucleotide including a plurality of sequences, each sequence encoding a polypeptide immunogen, wherein at least two (e.g., at least three, at least four, at least five, at least six, at least seven, at least eight, and at least nine) of the polypeptide immunogens have at least 90% (e.g., at least 91%, at least 92%, at least 93%, at least 94%, at least 95%, at least 96%, at least 97%, at least 98%, and at least 99%) amino acid sequence identity. In some embodiments, the at least two polypeptide immunogens have at least 95% (e.g., 96%, at least 97%, at least 98%, and at least 99%) amino acid sequence identity. In some embodiments, the at least two polypeptide immunogens have less than 100% amino acid sequence identity. In some embodiments, each of the polypeptide immunogens have at least 90% (e.g., at least 91%, at least 92%, at least 93%, at least 94%, at least 95%, at least 96%, at least 97%, at least 98%, and at least 99%) amino acid sequence identity. In some embodiments, each of the polypeptide immunogens have at least 95% (e.g., 96%, at least 97%, at least 98%, and at least 99%) amino acid sequence identity. In some embodiments, each of the polypeptide immunogens have less than 100% amino acid sequence identity.

In some embodiments, each of the polypeptide immunogens includes one or more epitopes that identifies a target. In some embodiments, the target is a pathogen. In some embodiments, the pathogen is a virus, a bacterium, a fungus, or a parasite. In some embodiments, the pathogen is a virus and each polypeptide immunogen includes one or more epitopes (e.g., two, three, four, five, six, seven, eight, nine, ten or more) corresponding to a viral immunogen. In some embodiments, the pathogen is a bacterium and each polypeptide immunogen includes one or more (e.g., two, three, four, five, six, seven, eight, nine, ten or more) epitopes corresponding to a bacterial immunogen. In some embodiments, the target is a cancer cell. In some embodiments, polypeptide immunogen includes one or more (e.g., two, three, four, five, six, seven, eight, nine, and ten or more) epitopes corresponding to a tumor antigen. In some embodiments, the target is a toxin or an allergen. In some embodiments, the target is associated with a disease, disorder, or condition. In some embodiments, the disease, disorder, or condition is a viral infection. In some embodiments, the disease, disorder, or condition is a bacterial infection. In some embodiments, the disease, disorder, or condition is a cancer.

In another aspect, the disclosure provides a circular polyribonucleotide including a plurality of sequences, each sequence encoding a polypeptide immunogen, wherein at least two (e.g., at least three, at least four, at least five, at least six, at least seven, at least eight, or at least nine) of the polypeptide immunogens identify different proteins, wherein each of the different proteins identifies the same target.

In some embodiments, each of the polypeptide immunogens identifies a different protein. In some embodiments, the target is a pathogen. In some embodiments, the pathogen is a virus, a bacterium, a fungus, or a parasite. In some embodiments, the pathogen is a virus and each of the different proteins is a viral protein associated with the virus. In some embodiments, the pathogen is a bacterium and each of the different proteins is a bacterial protein associated with the bacteria. In some embodiments the target is a cancer cell. In some embodiments, each of the different proteins is a different tumor antigen associate with the cancer cell. In some embodiments, the target is an allergen or a toxin.

In another aspect, the disclosure provides a circular polyribonucleotide including a plurality of sequences, each sequence encoding a polypeptide immunogen, wherein at least two (e.g., at least three, at least four, at least five, at least six, at least seven, at least eight, or at least nine) of the polypeptide immunogens identify different targets. In some embodiments, each of the polypeptide immunogens identifies a different target. In some embodiments, each target is a different pathogen. In some embodiments, each target is, independently, a cancer cell, a virus, a bacterium, a fungus, or a parasite.

In some embodiments, each target is a different virus. In some embodiments, each target is a different bacterium. In some embodiments, the targets include a virus and a bacterium.

In some embodiments of any one of the circular polyribonucleotides described herein, the circular polyribonucleotide includes between 500 and 20,000 ribonucleotides (e.g., between 500 and 10,000, 500 and 9,000, 500 and 8,000, 500 and 5,000, 500 and 4,000, 500 and 3,000, 1000 and 10,000, 1,000 and 8,000, 1,000 and 5,000, 3,000 and 8,000, 4,000 and 9,000, or 10,000 and 20,000)). In some embodiments, the circular polyribonucleotide includes between 500 and 5,000. In some embodiments, the circular polyribonucleotide includes between 1,000 and 5,000 ribonucleotides. In some embodiments, the circular polyribonucleotide includes between 5,000 and 10,000 ribonucleotides In some embodiments, the circular polyribonucleotide includes at least 500 ribonucleotides (e.g. at least 600, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, or at least 9500 ribonucleotides).

In some embodiments, the circular polyribonucleotide includes at least three, at least four, at least five, at least six, at least seven, at least eight, or at least nine sequences, each sequence encoding a polypeptide immunogen. In some embodiments, the circular polyribonucleotide includes two or three, between two and five (e.g., two, three or four), or between five and ten sequences (e.g., five, six, seven, eight, nine, or sequences), each sequence encoding a polypeptide immunogen.

In some embodiments, at least one sequence encoding a polypeptide immunogen further encodes a signal sequence. In some embodiments, each sequence encoding a polypeptide immunogen further encodes a signal sequence. In some embodiments, each of the sequences encoding each of the polypeptide immunogens is operably linked to an internal ribosomal entry site (IRES). In some embodiments, the circular polyribonucleotide includes a single IRES. In some embodiments, each of the polypeptide immunogens is encoded by a single open-reading frame operably linked to the single IRES, wherein the expression of the open reading frame produces a polypeptide including the amino acid sequence of each the polypeptide immunogens.

In some embodiments, the polypeptide immunogens are each separated by a polypeptide linker.

In some embodiments, the polypeptide immunogens are each separated by a cleavage domain. In some embodiments, each stagger element is a 2A self-cleaving peptide. In some embodiments, the circular polyribonucleotide includes a plurality of IRESs. In some embodiments, each IRES is operably linked to an open reading frame including a sequence encoding a polypeptide immunogen.

In some embodiments, the circular polyribonucleotide further includes at least one sequence (e.g., at least two, at least three, at least four, at least five, at least six, at least seven, at least, eight, at least nine, and at least ten sequences) encoding an adjuvant. In some embodiments, the at least one (e.g., at least two, at least three, at least four, at least five, at least six, at least seven, at least, eight, at least nine, and at least ten sequences) sequence encoding the adjuvant is operably linked to an IRES

In another aspect the disclosure provides a circular polyribonucleotide including a sequence encoding a polypeptide immunogen and a sequence encoding an adjuvant or a sequence that is an innate immune system stimulator. In some embodiments, the sequence encoding the polypeptide immunogen is operably linked to an IRES. In some embodiments, the sequence encoding the adjuvant is operably linked to an IRES. In some embodiments, the polypeptide immunogen and the adjuvant polypeptide are encoded by a single open-reading frame operably linked to the single IRES, wherein the expression of the open reading frame produces a polypeptide including the amino acid sequence of each of the polypeptide immunogen and the adjuvant polypeptide. In some embodiments, the polypeptide immunogen and the adjuvant polypeptide are each separated by a polypeptide linker. In some embodiments, the polypeptide immunogen and the adjuvant polypeptide are each separated by a cleavage domain. In some embodiments, each stagger element is a 2A self-cleaving peptide. In some embodiments, the circular polyribonucleotide includes a plurality of IRESs. In some embodiments, a first IRES is operably linked to the sequence encoding the polypeptide immunogen and a second IRES is operably linked to the sequence encoding the adjuvant polypeptide.

In some embodiments, wherein the circular polyribonucleotide encodes at least one polypeptide immunogen and at least one adjuvant (e.g., a polypeptide adjuvant), the expression of the sequence encoding the polypeptide immunogen is at least 2-fold, 5-fold, 10-fold, 20-fold, 50-fold, or 100-fold greater than the expression of the sequence encoding the adjuvant. In some embodiments, the expression of the sequence encoding the polypeptide immunogen is substantially the same as the expression of the sequence encoding the adjuvant.

In another aspect the disclosure provides a composition including a plurality of circular polyribonucleotides, wherein the composition includes at least a first circular polyribonucleotide including a sequence encoding a polypeptide immunogen and at least a second circular polyribonucleotide including a sequence encoding an adjuvant or a sequence that is an innate immune system stimulator. In some embodiments, the polypeptide immunogen and the adjuvant are each operably linked to an IRES.

In some embodiments, the adjuvant is a polypeptide. In some embodiments, the adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the cytokine is a pro-inflammatory cytokine. In some embodiments, the pro-inflammatory cytokine is selected from GM-CSF, IL-1 alpha, IL-1 beta, TGF-beta, TNF-alpha, and TNF-beta. In some embodiments, cytokine is a Th-1 inducing cytokine. In some embodiments, the Th-1 inducing cytokine is selected from IFN-gamma, IL-2, IL-12, IL-15, and IL-18. In some embodiments, the cytokine is a Th-2 inducing cytokine. In some embodiments, the Th-2 inducing cytokine is selected from IFN-gamma, IL-4, IL-5, IL-6, IL-10, and IL-13. In some embodiments, the chemokine is selected from MCP-1, MIP-1 alpha, MIP-1 beta, Rantes, and TCA-3. In some embodiments, the costimulatory molecule is selected from CD80, CD86, CD40-L, CD70, and CD27. In some embodiments, the innate immune stimulator is selected from STING (e.g., a constitutively active STING, such as STING^{V155M}, STING^{R284M}, STING^{R284M/V147L/N154S/V155M}, or STING^{V147L/N154S/V155M}), TLR3, TLR4, TLR9, TLR7, TLR8, TLR7, RIG-I/DDX58, and MDA-5/IFIH1. In some embodiments, the innate immune stimulator is a constitutively active mutant. In some embodiments, the signaling molecule is selected from STING (e.g., a constitutively active STING, such as STINGV^{155M}, STING^{R284M}STING^{R284M/V147L/N154S/V155M}, or STING^{V147L/N154S/V155M}), TRIF, TRAM, MyD88, IPS1, ASC, MAVS, MAPKs, IKK-alpha, IKK complex, TBK1, B-catenin, and caspase 1. In some embodiments, the transcriptional activator is selected from AP1, NF-kappa B, IRF3, IRF7, IRF1, and IRFS. In some embodiments, the cytokine receptor is selected from IL-2betaR, IFN-gammaR, and IL-6R. In some embodiments, the bacterial component is selected from flagellin and MBL.

In some embodiments of any one of the circular polyribonucleotides described herein, at least one sequence encoding the adjuvant further encodes a signal sequence. In some embodiments, each sequence encoding an immunogen further encodes a signal sequence. In some embodiments, at least one sequence encoding an immunogen does not encode a signal sequence. In some embodiments, none of the sequences encoding an immunogen further encodes a signal sequence.

In some embodiments, the circular polyribonucleotide further includes a sequence that is an innate immune system stimulator. In some embodiments, the sequence that is an innate immune system stimulator is a GU-rich motif, an AU-rich motif, a structured region including dsRNA, or an aptamer.

In some embodiments of any one of the circular polyribonucleotides described herein, wherein the circular polyribonucleotide includes a first polyribonucleotide with a 5′ end and a 3′ end, wherein 5′ end and 3′ end are each hybridized to a second polynucleotide, there by linking the 5′ end and the 3′ end of the first polyribonucleotide to form a circular polyribonucleotide. In some embodiments, the circular polyribonucleotide is produced by splint ligation. In some embodiments, the circular polyribonucleotide is produced by providing a linear polyribonucleotide having a 3′ end and a 5′ end, wherein the 3′ end and the 5′ end each include a portion of an intron, and wherein the intron portion of the 3′ end and the intron portion of the 5′ end catalyze a splicing reaction thereby covalently conjugating the 5′ end and the 3′ end to produce a circular polyribonucleotide. In some embodiments, the intron is a Group I self-splicing intron.

In another aspect the disclosure provides, a composition including a plurality of circular polyribonucleotides, each including a sequence encoding a polypeptide immunogen. In some embodiments, each of the plurality of circular polyribonucleotides is a circular polyribonucleotide described herein. In some embodiments, the composition includes at least a first circular polyribonucleotide including a sequence encoding a first polypeptide immunogen and at least a second circular polyribonucleotide including a sequence encoding a second polypeptide immunogen, wherein the first and second polypeptide immunogens have at least 90% (e.g., at least 91%, at least 92%, at least 93%, at last 94%, at least 95%, at least 96%, at least 97%, at least 98%, and at least 99%) amino acid sequence identity. In some embodiments, the first and second polypeptide immunogens have at least 95% (at least 96%, at least 97%, at least 98%, and at least 99%) amino acid sequence identity. In some embodiments, the first and second polypeptide immunogens have less than 100% amino acid sequence identity. In some embodiments, each of the polypeptide immunogens includes one or more epitopes that identifies a target. In some embodiments, the composition includes at least a first circular polyribonucleotide including a sequence encoding a first polypeptide immunogen and at least a second circular polyribonucleotide including a sequence encoding a second polypeptide immunogen, wherein the first and second polypeptide immunogens identify different proteins, wherein each different protein identifies the same target. In some embodiments, the composition includes at least a first circular polyribonucleotide including a sequence encoding a first polypeptide immunogen and at least a second circular polyribonucleotide including a sequence encoding a second polypeptide immunogen, wherein the first polypeptide immunogens identifies a first target and the second polypeptide immunogen identifies a second target. In some embodiments, each target is, independently, a cancer cell, a virus, a bacterium, a fungus, a parasite, a toxin, or an allergen. In some embodiments, the target is a pathogen. In some embodiments, the pathogen is a virus, a bacterium, a fungus, or a parasite. In some embodiments, the target is a cancer cell, an allergen, or a toxin. In some embodiments, each polypeptide immunogen is operably linked to an IRES.

In another aspect, the disclosure provides a pharmaceutical preparation of a circular polyribonucleotide, wherein the circular polyribonucleotide includes a sequence encoding a polypeptide immunogen, and wherein the circular polyribonucleotide constitutes at least 25% (w/w) (e.g., at least 30% (w/w), 40% (w/w), 50% (w/w), 60% (w/w), 70% (w/w), 75% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w) or 99% (w/w)) of total ribonucleotide molecules in the pharmaceutical preparation. In some embodiments, the circular polyribonucleotide constitutes at least 70% (w/w), 75% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w) or 99% (w/w) of total ribonucleotide molecules in the pharmaceutical preparation. In some embodiments, the pharmaceutical preparation includes no more than 75% (w/w), 60% (w/w), 50% (w/w), 40% (w/w), 30% (w/w), 20% (w/w), 15% (w/w), 10% (w/w), 5% (w/w), or 1% (w/w) linear polyribonucleotide molecules of the total ribonucleotide molecules in the pharmaceutical preparation. In some embodiments, the pharmaceutical preparation includes no more than 0.5% (w/w) (e.g., 0.4% (w/w), 0.3% (w/w), 0.2% (w/w), 0.1 (w/w), and 0.05% (w/w) linear polyribonucleotide molecules of the total ribonucleotide molecules in the pharmaceutical preparation. In some embodiments, the pharmaceutical preparation includes at least 1% (w/w) linear polyribonucleotide. In some embodiments, the pharmaceutical preparation includes between 30%-40% (w/w) circular polyribonucleotide and between 1%-10% (w/w), 10%-20% (w/w), 20%-30% (w/w), 30%-40% (w/w), 40%-50% (w/w), 50%-60% (w/w), or 60%-70% (w/w) linear polyribonucleotide. In some embodiments, the pharmaceutical preparation includes between 40%-60% (w/w) circular polyribonucleotide and between 1%-10% (w/w), 10%-20% (w/w), 20%-30% (w/w), 30%-40% (w/w), 40%-50% (w/w), 50%-60% (w/w) linear polyribonucleotide. In some embodiments, the pharmaceutical preparation includes between 60%-80% (w/w) circular polyribonucleotide and between 1%-10% (w/w), 10%-20% (w/w), 20%-30% (w/w), 30%-40% (w/w) linear polyribonucleotide.

In another aspect, the disclosure provides a pharmaceutical preparation of a circular polyribonucleotide, wherein the circular polyribonucleotide includes a sequence encoding a polypeptide immunogen, and wherein the pharmaceutical preparation includes no more than 75% (w/w), 60% (w/w), 50% (w/w), 40% (w/w), 30% (w/w), 20% (w/w), 15% (w/w), 10% (w/w), 5% (w/w), or 1% (w/w) linear polyribonucleotide molecules of the total ribonucleotide molecules in the pharmaceutical preparation. In some embodiments, the pharmaceutical preparation includes no more than 20% (w/w), 15% (w/w), 10% (w/w), 5% (w/w), 1% (w/w), or 0.5% (w/w) linear polyribonucleotide molecules of the total ribonucleotide molecules in the pharmaceutical preparation.

In some embodiments, the preparation includes no more than 15% (w/w) (e.g., 15% (w/w), 10% (w/w), 9% (w/w), 8% (w/w), 7% (w/w), 6% (w/w), 5% (w/w), 4% (w/w), 3% (w/w), 2% (w/w), 1% (w/w), and 0.5% (w/w)) nicked polyribonucleotide molecules of the total ribonucleotide molecules in the pharmaceutical preparation. In some embodiments, the preparation includes no more than 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation.

In another aspect, the disclosure provides a pharmaceutical preparation of a circular polyribonucleotide, wherein the circular polyribonucleotide includes a sequence encoding a polypeptide immunogen, and wherein the pharmaceutical preparation includes no more than 15% (w/w) (e.g., 15% (w/w), 10% (w/w), 9% (w/w), 8% (w/w), 7% (w/w), 6% (w/w), 5% (w/w), 4% (w/w), 3% (w/w), 2% (w/w), 1% (w/w), and 0.5% (w/w)) nicked polyribonucleotide molecules of the total ribonucleotide molecules in the pharmaceutical preparation. In some embodiments, the preparation includes no more than 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation.

In some embodiments of an one of the pharmaceutical preparations described herein the pharmaceutical preparation includes less than 10 EU/kg (e.g., 9 EU/kg, 8 EU/kg, 7 EU/kg, 6 EU/kg, 5 EU/kg, 4 EU/kg, 3 EU/KG, 2 EU/kg, and 1 EU/kg) or lacks endotoxin as measured by a Limulus amebocyte lysate test. In some embodiments, the pharmaceutical preparation includes a bioburden of less than 100 CFU/100 ml or less than 10 CFU/100 ml before sterilization. In some embodiments, the pharmaceutical preparation is a sterile pharmaceutical preparation, e.g., supports growth of fewer than 100 viable microorganisms as tested under aseptic conditions. In some embodiments, the pharmaceutical preparation meets the standard of USP <71>. In some embodiments, the pharmaceutical preparation meets the standard of USP <85>. In some embodiments, the pharmaceutical preparation is an intermediate pharmaceutical preparation of a final drug product.

In some embodiments, the pharmaceutical preparation is a final drug product for administration to a subject. In some embodiments, the pharmaceutical preparation includes a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL circular polyribonucleotide molecules. In some embodiments, the pharmaceutical preparation includes no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL of deoxyribonucleotide molecules. In some embodiments, the pharmaceutical preparation includes a protein contamination (e.g. an enzyme) of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules. In some embodiments, the pharmaceutical preparation includes an A260/A280 absorbance ratio of from about 1.6 to 2.3 (e.g., 1.7, 1.8, 1.9, 2.0, 2.1, and 2.2) as measured by a spectrophotometer.

In some embodiments, the pharmaceutical preparation is substantially free of a process-related impurity selected from a cell protein, a cell deoxyribonucleic acid, an enzyme, a reagent component, a gel component, or a chromatographic material. In some embodiments, the pharmaceutical preparation has a reduced level of one or more markers of an immune or inflammatory response after purification compared to prior to purification. In some embodiments, the circular polyribonucleotide is a circular polyribonucleotide described herein.

In some embodiments, the circular polyribonucleotide is produced according to a method including the steps of: providing a plurality of linear polyribonucleotide molecules; circularizing the linear polyribonucleotide molecules to provide a preparation of circular polyribonucleotide molecules; and processing the preparation to remove linear polyribonucleotide molecules from the preparation. In some embodiments, the method further includes evaluating the amount of linear polyribonucleotide molecules in the preparation remaining after the processing step. In some embodiments, the method further includes further processing the preparation, wherein further processing includes: processing the preparation to remove nicked polyribonucleotide molecules from the preparation; and/or processing the preparation to remove deoxyribonucleotide molecules from the preparation; and/or processing the preparation to remove protein contaminants from the preparation; and/or processing the preparation to remove endotoxin from the preparation.

In another aspect the disclosure provides a pharmaceutical composition including any one of the circular polyribonucleotides, compositions, or pharmaceutical preparations described herein and a pharmaceutically acceptable excipient. In some embodiments, the pharmaceutical composition includes any one of the circular polyribonucleotides, compositions, or pharmaceutical preparations described herein and protamine or a protamine salt (e.g., protamine sulfate). In some embodiments, the pharmaceutical composition further includes an adjuvant. In some embodiments, the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant. In some embodiments, the adjuvant is an inorganic adjuvant. In some embodiments, the inorganic adjuvant is an aluminum salt or a calcium salt. In some embodiments, the adjuvant is a small molecule. In some embodiments, the small molecule is imiquimod, resiquimod, or gardiquimod. In some embodiments, the adjuvant is an oil in water emulsion. In some embodiments, the oil in water emulsion is Squalene, MF59, AS03, a Montanide formulation, optionally Montanide ISA 51 or Montanide ISA 720, or Incomplete Freunds Adjuvant an oil in water emulsion. In some embodiments, the adjuvant is a lipid or polymer. In some embodiments, the lipid or polymer is a polymeric nanoparticle, optionally PLGA, PLG, PLA, PGA, or PHB, a liposome, optionally a Virosomes or CAF01, a lipid nanoparticle or a component thereof, a lipopolysaccharide (LPS), optionally monophosphoryl lipid A (MPLA) or glucopyranosyl Lipid A (GLA), a lipopeptide, optionally Pam2 (Pam2CSK4) or Pam3 (Pam3CSK4), or a glycolipid, optionally, trehalose dimycolate. In some embodiments, the adjuvant is a peptide or peptidoglycan. In some embodiments, the peptide or peptidoglycan corresponds to all or a portion of a synthetic or purified gram negative or gram positive bacteria, optionally N-acetyl-muramyl-L-alanyl-D-isoglutamine (MDP), a flagellin-fusion protein, Mannose-binding Lectin (MBL), a cytokines, or a chemokine. In some embodiments, the adjuvant is a carbohydrate or polysaccharide. In some embodiments, the carbohydrate or polysaccharide is dextran (branched microbial polysaccharide), dextran-sulfate, Lentinan, zymosan, Beta-glucan, Deltin, Mannan, or Chitin. In some embodiments, the adjuvant is a saponin. In some embodiments, the saponin is a glycoside or a polycyclic aglycones attached to one or more sugar side chains, optionally ISCOMS, ISCOMS matrix, or QS-21. In some embodiments, the adjuvant is an RNA-based adjuvant. In some embodiments, the RNA-based adjuvant is Poly IC, Poly IC:LC, a hairpin RNAs, optionally with a 5′PPP containing sequence, a viral sequence, a polyU containing sequences, dsRNA, a natural or synthetic immunostimulatory RNA sequence, a nucleic acid analogs, optionally cyclic GMP-AMP or a cyclic dinucleotide such as cyclic di-GMP, or an immunostimulatory base analog, optionally a C8-substituted or an N7,C8-disubstituted guanine ribonucleotide. In some embodiments, the adjuvant is a DNA-based adjuvant. In some embodiments, the DNA-based adjuvant is a CpG, dsDNA, or a natural or synthetic immunostimulatory DNA sequence. In some embodiments, the adjuvant is a viral particle. In some embodiments, the viral particle is a virosome, optionally, a phospholipid cell membrane bilayer. In some embodiments, the adjuvant is a bacterial adjuvant. In some embodiments, the bacterial adjuvant is flagellin, LPS, or a bacterial toxin, optionally an enterotoxin, a heat-labile toxin, or a Cholera toxin. In some embodiments, the adjuvant is a hybrid molecule. In some embodiments, the adjuvant is CpG conjugated to Imiquimod. In some embodiments, the adjuvant is a fungal or oocyte microbe-associated molecular pattern (MAMP). In some embodiments, the fungal or oocyte MAMP is chitin or beta-glucan. In some embodiments, the adjuvant is an inorganic nanoparticle. In some embodiments, the inorganic nanoparticle is a gold nanorod, a silica-based nanoparticle, optionally a mesoporous silica nanoparticle (MSN). In some embodiments, the adjuvant is a multi-component adjuvant. In some embodiments, the multi-component adjuvant is AS01, AS03, AS04, Complete Freunds Adjuvant, or CAF01.

In another aspect, the disclosure provides a pharmaceutical composition including a mixture including any one of the circular polyribonucleotides, the compositions, or the pharmaceutical preparations described herein, and an alcohol, wherein the alcohol constitutes about 0.3% v/v to about 75% v/v (e.g., 0.5% v/v, 1% v/v, 5% v/v, 10% v/v, 15% v/v, 20% v/v, 25% v/v, 30% v/v, 35% v/v, 40% v/v, 45% v/v, 50% v/v, 55% v/v, 60% v/v, 65% v/v, and 70% v/v) of the mixture.

In another aspect, the disclosure provides a pharmaceutical composition including a mixture including a linear polyribonucleotide that includes at least one sequence encoding a polypeptide immunogen and an alcohol, wherein the alcohol constitutes about 0.3% v/v to about 75% v/v (e.g., 0.5% v/v, 1% v/v, 5% v/v, 10% v/v, 15% v/v, 20% v/v, 25% v/v, 30% v/v, 35% v/v, 40% v/v, 45% v/v, 50% v/v, 55% v/v, 60% v/v, 65% v/v, and 70% v/v) of the mixture.

In some embodiments, the linear polyribonucleotide includes a plurality of sequences, each sequence encoding a polypeptide immunogen, wherein at least two (e.g., at least three, at least four, at least five, at least six, at least seven, at least eight, and at least nine) of the polypeptide immunogens have at least 90% (e.g., at least 91%, at least 92%, at least 93%, at least 94%, at least 95%, at least 96%, at least 97%, at least 98%, and at least 99%) amino acid sequence identity. In some embodiments, the at least two polypeptide immunogens have at least 95% (e.g., 96%, at least 97%, at least 98%, and at least 99%) amino acid sequence identity. In some embodiments, the at least two polypeptide immunogens have less than 100% amino acid sequence identity. In some embodiments, each of the polypeptide immunogens includes one or more epitopes that identifies a target. In some embodiments, the target is a pathogen. In some embodiments, the pathogen is a virus, a bacterium, a fungus, or a parasite. In some embodiments, the pathogen is a virus and each polypeptide immunogen includes one or more epitopes (e.g., two, three, four, five, six, seven, eight, nine, ten, or more) corresponding to a viral immunogen. In some embodiments, the pathogen is a bacterium and each polypeptide immunogen includes one or more (e.g., two, three, four, five, six, seven, eight, nine, ten, or more) epitopes corresponding to a bacterial immunogen. In some embodiments, the target is a cancer cell. In some embodiments, polypeptide immunogen includes one or more (e.g., two, three, four, five, six, seven, eight, nine, ten, or more) epitopes corresponding to a tumor antigen. In some embodiments, the target is a toxin or an allergen. In some embodiments, the target is associated with a disease, disorder, or condition. In some embodiments, the disease, disorder, or condition is a viral infection. In some embodiments, the disease, disorder, or condition is a bacterial infection. In some embodiments, the disease, disorder, or condition is a cancer.

In some embodiments, the linear polyribonucleotide includes a plurality of sequences, each sequence encoding a polypeptide immunogen, wherein at least two (e.g., at least three, at least four, at least five, at least six, at least seven, at least eight, and at least nine) of the polypeptide immunogens identify different proteins, wherein each of the different proteins identifies the same target. In some embodiments, each of the polypeptide immunogens identifies a different protein. In some embodiments, the target is a pathogen. In some embodiments, the pathogen is a virus, a bacterium, a fungus, or a parasite. In some embodiments, the pathogen is a virus and each of the different proteins is a viral protein associated with the virus. In some embodiments, the pathogen is a bacterium and each of the different proteins is a bacterial protein associated with the bacteria. In some embodiments the target is a cancer cell. In some embodiments, each of the different proteins is a different tumor antigen associate with the cancer cell. In some embodiments, the target is an allergen or a toxin.

In some embodiments, the linear polyribonucleotide includes a plurality of sequences, each sequence encoding a polypeptide immunogen, wherein at least two (e.g., at least three, at least four, at least five, at least six, at least seven, at least eight, ant at least nine) of the polypeptide immunogens identify different targets. In some embodiments, each of the polypeptide immunogens identifies a different target. In some embodiments, each target is a different pathogen. In some embodiments, each target is, independently, a cancer cell, a virus, a bacterium, a fungus, or a parasite. In some embodiments, each target is a different virus. In some embodiments, each target is a different bacterium. In some embodiments, the targets include a virus and a bacterium.

In some embodiments, the linear polyribonucleotide includes at least three, at least four, at least five, at least six, at least seven, at least eight, or at least nine sequences, each sequence encoding a polypeptide immunogen. In some embodiments, the linear polyribonucleotide includes between two and three, between two and five (e.g., three and four), or between five and ten sequences (e.g., six, seven, eight, and nine sequences), each sequence encoding a polypeptide immunogen.

In some embodiments, the linear polyribonucleotide includes further includes at least one sequence (e.g., at least two, at least three, at least four, at least five, at least six, at least seven, at least, eight, at least nine, or at least ten sequences) encoding an adjuvant. In some embodiments, the at least one (e.g., at least two, at least three, at least four, at least five, at least six, at least seven, at least, eight, at least nine, or at least ten sequences) sequence encoding the adjuvant is operably linked to an IRES.

In some embodiments, the linear polyribonucleotide includes a sequence encoding a polypeptide immunogen and a sequence encoding an adjuvant or a sequence that is an innate immune system stimulator. In some embodiments, the sequence encoding the polypeptide immunogen is operably linked to an IRES. In some embodiments, the sequence encoding the adjuvant is operably linked to an IRES.

In some embodiments, the adjuvant is a polypeptide. In some embodiments, the adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the cytokine is a pro-inflammatory cytokine. In some embodiments, the pro-inflammatory cytokine is selected from GM-CSF, IL-1 alpha, IL-1 beta, TGF-beta, TNF-alpha, and TNF-beta. In some embodiments, cytokine is a Th-1 inducing cytokine. In some embodiments, the Th-1 inducing cytokine is selected from IFN-gamma, IL-2, IL-12, IL-15, and IL-18. In some embodiments, the cytokine is a Th-2 inducing cytokine. In some embodiments, the Th-2 inducing cytokine is selected from IFN-gamma, IL-4, IL-5, IL-6, IL-10, and IL-13. In some embodiments, the chemokine is selected from MCP-1, MIP-1 alpha, MIP-1 beta, Rantes, and TCA-3. In some embodiments, the costimulatory molecule is selected from CD80, CD86, CD40-L, CD70, and CD27. In some embodiments, the innate immune stimulator is selected from STING (e.g., constitutively active STING (caSTING), such as STINGV^{155M}, STING^{R284M}, STING^{R284MN147L/N154SN155M}, or STING^{V147L/N154S/V155M}), TLR3, TLR4, TLR9, TLR7, TLR8, TLR7, RIG-I/DDX58, and MDA-5/IFIH1. In some embodiments, the innate immune stimulator is a constitutively active mutant. In some embodiments, the signaling molecule is selected from STING (e.g., caSTING, such as STING^{V155M}, STING^{R284M}, STING^{R284MN147L/N154SN155M}, or STING^{V147L/N154SN155M}), TRIF, TRAM, MyD88, IPS1, ASC, MAVS, MAPKs, IKK-alpha, IKK complex, TBK1, B-catenin, and caspase 1. In some embodiments, the transcriptional activator is selected from AP1, NF-kappa B, IRF3, IRF7, IRF1, and IRFS. In some embodiments, the cytokine receptor is selected from IL-2betaR, IFN-gammaR, and IL-6R. In some embodiments, the bacterial component is selected from flagellin and MBL.

In some embodiments, the linear polyribonucleotide includes a sequence that is an innate immune system stimulator. In some embodiments, the sequence that is an innate immune system stimulator is a GU-rich motif, an AU-rich motif, a structured region including dsRNA, or an aptamer.

In some embodiments, the alcohol constitutes about 0.3% v/v to about 60% v/v (e.g., 0.5% v/v, 1% v/v, 5% v/v, 10% v/v, 15% v/v, 20% v/v, 25% v/v, 30% v/v, 35% v/v, 40% v/v, 45% v/v, 50% v/v, and 55% v/v), about 0.3% v/v to about 50% v/v (e.g., 0.5% v/v, 1% v/v, 5% v/v, 10% v/v, 15% v/v, 20% v/v, 25% v/v, 30% v/v, 35% v/v, 40% v/v, and 45% v/v), about 0.3% v/v to about 40% v/v (e.g., 0.5% v/v, 1% v/v, 5% v/v, 10% v/v, 15% v/v, 20% v/v, 25% v/v, 30% v/v, and 35% v/v), about 0.3% v/v to about 20% v/v, about 0.3% v/v to about 15% v/v (e.g., 0.5% v/v, 1% v/v, 5% v/v, 10% v/v, and 15% v/v), about 0.3% v/v to about 10% v/v (e.g., 0.5% v/v, 1% v/v, 5% v/v, and 10% v/v), about 0.3% v/v to about 5% v/v (e.g., 0.5% v/v, 1% v/v, 2% v/v, 3% v/v, and 4% v/v), about 0.3% v/v to about 1% v/v (e.g., 0.4% v/v, 0.5% v/v, 0.6% v/v, 0.7% v/v, 0.8% v/v, and 0.9% v/v) or about 0.3% v/v to about 0.5% v/v of the mixture. In some embodiments, the alcohol constitutes about 0.5% v/v to about 75% v/v, about 1% v/v to about 75% v/v, about 5% v/v to about 75% v/v, about 10% v/v to about 75% v/v, about 15% v/v to about 75% v/v, about 20% v/v to about 75% v/v, about 30% v/v to about 75% v/v, about 40% v/v to about 75% v/v, about 50% v/v to about 75% v/v, about 60% v/v to about 75% v/v, or about 70% v/v to about 75% v/v of the mixture.

In some embodiments, the alcohol is selected from methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol is ethanol.

In some embodiments, the pharmaceutical composition is formulated for topical administration.

In some embodiments, the pharmaceutical composition is a liquid, gel, lotion, paste, cream, foam, or stick. In some embodiments, the pharmaceutical composition has a pH of about 6-8 (e.g., of about 6.5-7.5, or of about 7). In some embodiments, the pharmaceutical composition has a viscosity that is about the same as water.

In some embodiments, the pharmaceutical composition is substantially free of hydrophobic or lipophilic groups. In some embodiments, the pharmaceutical composition is substantially free of hydrocarbons. In some embodiments, the pharmaceutical composition is substantially free of cationic liposomes. In some embodiments, the pharmaceutical composition is substantially free of fatty acids, lipids, liposomes, cholesterol, or any combination thereof. In some embodiments, the composition further including an adjuvant. In some embodiments, the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant. In some embodiments, the composition further includes a cell-penetrating agent.

In another aspect, the disclosure provides, a method of delivering a polyribonucleotide to a subject including topically applying a circular polyribonucleotide described herein, an immunogenic composition described herein, a pharmaceutical preparation descried herein to a surface area of the subject.

In another aspect, the disclosure provides, a method of delivering a polyribonucleotide to a subject including: (a) applying a sterilizing agent to a surface area of the subject; and (b) applying a composition including a circular polyribonucleotide described herein, a immunogenic composition described herein, a pharmaceutical preparation descried herein, or the pharmaceutical composition described herein and a diluent to the surface area of the subject. In some embodiments, the composition of (b) does not comprise a carrier. In some embodiments, the sterilizing agent is an alcohol, UV light, laser light, or heat.

In another aspect, the disclosure provides a method of delivering a polyribonucleotide to a subject including: (a) applying an alcohol to a surface area of the subject; and (b) applying a composition including a circular polyribonucleotide described herein, a immunogenic composition described herein, a pharmaceutical preparation descried herein, or the pharmaceutical composition described herein and a diluent to the surface area of the subject. In some embodiments, the alcohol is selected from the group consisting of: methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol is ethanol.

In some embodiments (e.g., in some embodiments of topical delivery described herein), the surface area of the subject is selected from a surface area of the skin, oral cavity, nasal cavity, gastrointestinal tract, respiratory tract, or any combination thereof.

In another aspect the disclosure provides a method of treating or preventing a disease, disorder, or condition in a subject, the method including administering to the subject any one of the circular polyribonucleotides, the compositions, pharmaceutical preparations, or the pharmaceutical compositions described herein. In some embodiments, the disease, disorder, or condition is a viral infection, a bacterial infection, or a fungal infection. In some embodiments, the disease, disorder, or condition is a cancer. In some embodiments, the disease, disorder, or condition is associated with exposure to an allergen. In some embodiments, disease, disorder, or condition is associated with exposure to a toxin.

In another aspect, the disclosure provides a method of inducing an immune response in a subject, the method including administering to the subject any one of the circular polyribonucleotides, the compositions, pharmaceutical preparations, or pharmaceutical compositions described herein. In some embodiments, the subject is a mammal. In some embodiments, the subject is a human. In some embodiments, the method is a non-human mammal. In some embodiments, the non-human mammal is a cow, a sheep, a goat, a pig, a dog, a horse, or a cat. In some embodiments, the subject is a bird. In some embodiments, the bird is a hen, a rooster, a turkey, or a parrot. In some embodiments, the method further includes administering an adjuvant to the subject. In some embodiments, the adjuvant is a separate molecular entity from the circular polyribonucleotide, linear polyribonucleotide, or preparation or composition thereof. In some embodiments, the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant. In some embodiments, the adjuvant is a polypeptide adjuvant. In some embodiments, the polypeptide adjuvant is a cytokine, a chemokine, a costimulatory molecule, an innate immune stimulator, a signaling molecule, a transcriptional activator, a cytokine receptor, a bacterial component, or a component of the innate immune system. In some embodiments, the adjuvant is an innate immune system stimulator. In some embodiments, the innate immune system stimulator is selected from an RNA including a GU-rich motif, an AU-rich motif, a structured region including dsRNA, or an aptamer.

In some embodiments, any one of the circular polyribonucleotides, compositions, pharmaceutical preparations, or pharmaceutical compositions described herein is administered to the subject as a single dose. In some embodiments, any one of the circular polyribonucleotides, compositions, pharmaceutical preparations, or pharmaceutical compositions described herein is administered to the subject two or more times, three or more times, four or more times, or five or more times. In some embodiments, administration of any one of the circular polyribonucleotides, compositions, pharmaceutical preparations, or pharmaceutical compositions described herein occurs about weekly, about every two weeks, about every three weeks, about every month, about every two months, about every three months, about every four months, about every five months, about every six months, about every year, about every two years, about every three years, about every four years, about every five years, or about every ten years.

In some embodiments, the method further comprises administering to the subject a polypeptide immunogen (e.g., a protein subunit comprising a polypeptide immunogen). In some embodiments the polypeptide immunogen corresponds to (e.g., shares 90%, 95%, 96%, 97%, 98%, or 100% amino acid sequence identity with a polypeptide immunogen encoded by a sequence of the circular polyribonucleotide. In some embodiments, the polypeptide immunogen is administered to the subject after administering any one of the circular polyribonucleotides, immunogenic compositions, pharmaceutical preparations, or pharmaceutical compositions described herein. In some embodiments, the polypeptide immunogen maintains or enhances an immune response in the subject against the polypeptide immunogen.

In another aspect, the disclosure provides a method of maintaining or enhancing an immune response in a subject comprising (i) administering to the subject a circular polyribonucleotide encoding a polypeptide immunogen and (ii) administering to the subject the polypeptide immunogen, wherein step (ii) occurs between 1 week and 6 months (e.g., between 1 month and 5 months, 2 months and 3 months, 2 weeks and 3 months, or 3 months and 6 months) after step (i), and wherein administration of the polypeptide immunogen of step (ii) maintains or enhances the immune response in the subject against the polypeptide immunogen. In some embodiments, the polypeptide immunogen comprises one or more epitopes that identifies a target. In some embodiments, the target is a pathogen. In some embodiments, the target is a cancer cell, an allergen, or a toxin.

### Numbered Embodiments

[1] A circular polyribonucleotide comprising a plurality of sequences, each sequence encoding a polypeptide immunogen, wherein at least two of the polypeptide immunogens have at least 90% amino acid sequence identity.

[2] The circular polyribonucleotide of paragraph [1], wherein the at least two polypeptide immunogens have at least 95% amino acid sequence identity.

[3] The circular polyribonucleotide of paragraph [1] or [2], wherein the at least two polypeptide immunogens have less than 100% amino acid sequence identity.

[4] The circular polyribonucleotide of paragraph [1], wherein each of the polypeptide immunogens have at least 90% amino acid sequence identity.

[5] The circular polyribonucleotide of paragraph [4], wherein each of the polypeptide immunogens have at least 95% amino acid sequence identity.

[6] The circular polyribonucleotide of paragraph [4] or [5], wherein each of the polypeptide immunogens have less than 100% amino acid sequence identity.

[7] The circular polyribonucleotide of any one of paragraphs [1] to [6], wherein each of the polypeptide immunogens comprises one or more epitopes that identifies a target.

[8] The circular polyribonucleotide of paragraph [7], wherein the target is a pathogen.

[9] The circular polyribonucleotide of paragraph [8], wherein the pathogen is a virus, a bacterium, a fungus, or a parasite.

[10] The circular polyribonucleotide of paragraph [9], wherein the pathogen is a virus and each polypeptide immunogen comprises one or more epitopes corresponding to a viral immunogen.

[11] The circular polyribonucleotide of paragraph [9], wherein the pathogen is a bacterium and each polypeptide immunogen comprises one or more epitopes corresponding to a bacterial immunogen.

[12] The circular polyribonucleotide of paragraph [7], wherein the target is a cancer cell.

[13] The circular polyribonucleotide of paragraph [12], wherein each polypeptide immunogen comprises one or more epitopes corresponding to a tumor antigen.

[14] The circular polyribonucleotide of paragraph [7], wherein the target is a toxin or an allergen.

[15] The circular polyribonucleotide of any one of paragraphs [7] to [14], wherein the target is associated with a disease, disorder, or condition.

[16] The circular polyribonucleotide of paragraph [15], wherein the disease, disorder, or condition is a viral infection.

[17] The circular polyribonucleotide of paragraph [15], wherein the disease, disorder, or condition is a bacterial infection.

[18] The circular polyribonucleotide of paragraph [15], wherein the disease, disorder, or condition is a cancer.

[19] A circular polyribonucleotide comprising a plurality of sequences, each sequence encoding a polypeptide immunogen, wherein at least two of the polypeptide immunogens identify different proteins, wherein each of the different proteins identifies the same target.

[20] The circular polyribonucleotide of paragraph [19], wherein each of the polypeptide immunogens identifies a different protein.

[21] The circular polyribonucleotide of paragraph [19] or [20], wherein the target is a pathogen.

[22] The circular polyribonucleotide of paragraph [21], wherein the pathogen a virus, a bacterium, a fungus, or a parasite.

[23] The circular polyribonucleotide of paragraph [22], wherein the pathogen is a virus and each of the different proteins is a viral protein associated with the virus.

[24] The circular polyribonucleotide of paragraph [22], wherein the pathogen is a bacterium and each of the different proteins is a bacterial protein associated with the bacteria.

[25] The circular polyribonucleotide of paragraph [19] or [20], wherein the target is a cancer cell

[26] The circular polyribonucleotide of paragraph [25], wherein and each of the different proteins is a different tumor antigen associate with the cancer cell.

[27] The circular polyribonucleotide of paragraph [19] or [20], wherein the target is an allergen or a toxin.

[28] A circular polyribonucleotide comprising a plurality of sequences, each sequence encoding a polypeptide immunogen, wherein at least two of the polypeptide immunogens identify different targets.

[29] The circular polyribonucleotide of paragraph [28], wherein each of the polypeptide immunogens identifies a different target.

[30] The circular polyribonucleotide of paragraph [28] or [29], wherein each target is a different pathogen.

[31] The circular polyribonucleotide of paragraph [30], wherein each target is, independently, a cancer cell, a virus, a bacterium, a fungus, or a parasite.

[32] The circular polyribonucleotide of paragraph [31], wherein each target is a different virus.

[33] The circular polyribonucleotide of paragraph [31], wherein each target is a different bacterium.

[34] The circular polyribonucleotide of paragraph [31], wherein the targets include a virus and a bacterium.

[35] The circular polyribonucleotide of any one of paragraphs [1] to [34], wherein the circular polyribonucleotide comprises between 500 and 20,000 ribonucleotides.

[36] The circular polyribonucleotide of paragraph [35], wherein the circular polyribonucleotide comprises between 500 and 10,000 ribonucleotides.

[37] The circular polyribonucleotide of paragraph [36], wherein the circular polyribonucleotide comprises between 500 and 5,000 ribonucleotides.

[38] The circular polyribonucleotide of any one of paragraphs [1] to [34], wherein the circular polyribonucleotide comprises at least 500 ribonucleotides.

[39] The circular polyribonucleotide of paragraph [38], wherein the circular polyribonucleotide comprises at least 1,000 ribonucleotides.

[40] The circular polyribonucleotide of paragraph [39], wherein the circular polyribonucleotide comprises at least 5,000 ribonucleotides.

[41] The circular polyribonucleotide of any one of paragraphs [1] to [40], wherein the circular polyribonucleotide comprises at least three, at least four, at least five, at least six, at least seven, at least eight, or at least nine sequences, each sequence encoding a polypeptide immunogen.

[42] The circular polyribonucleotide of any one of paragraphs [1] to [40], wherein the circular polyribonucleotide comprises between two and three, between two and five, or between five and ten sequences, each sequence encoding a polypeptide immunogen.

[43] The circular polyribonucleotide of any one of paragraphs [1] to [40], wherein at least one sequence encoding a polypeptide immunogen further encodes a signal sequence.

[44] The circular polyribonucleotide of any one of paragraphs [1] to [40], wherein each sequence encoding a polypeptide immunogen further encodes a signal sequence.

[45] The circular polyribonucleotide of any one of paragraphs [1] to [44], wherein each of the sequences encoding each of the polypeptide immunogens is operably linked to an internal ribosomal entry site (IRES).

[46] The circular polyribonucleotide of paragraph [45], wherein the circular polyribonucleotide comprises a single IRES.

[47] The circular polyribonucleotide of paragraph [46], wherein each of the polypeptide immunogens is encoded by a single open-reading frame operably linked to the single IRES, wherein the expression of the open reading frame produces a polypeptide comprising the amino acid sequence of each the polypeptide immunogens.

[48] The circular polyribonucleotide of paragraph [47], wherein the polypeptide immunogens are each separated by a polypeptide linker.

[49] The circular polyribonucleotide of paragraph [47], wherein the polypeptide immunogens are each separated by a cleavage domain.

[50] The circular polyribonucleotide of paragraph [49], wherein each stagger element a 2A self-cleaving peptide.

[51] The circular polyribonucleotide of paragraph [45], wherein the circular polyribonucleotide comprises a plurality of IRESs.

[52] The circular polyribonucleotide of paragraph [51], wherein each IRES is operably linked to an open reading frame comprising a sequence encoding a polypeptide immunogen.

[53] The circular polyribonucleotide of any one of paragraphs [1] to [52], wherein the circular polyribonucleotide further comprises at least one sequence encoding an adjuvant.

[54] The circular polyribonucleotide of paragraph [53], wherein the at least one sequence encoding the adjuvant is operably linked to an IRES.

[55] The circular polyribonucleotide of paragraph [53] or [54], wherein the adjuvant is a polypeptide.

[56] The circular polyribonucleotide of paragraph [55], wherein the adjuvant is a cytokine, a chemokine, a costimulatory molecule, an innate immune stimulator, a signaling molecule, a transcriptional activator, a cytokine receptor, a bacterial component, or a component of the innate immune system.

[57] The circular polyribonucleotide of paragraph [56], wherein the cytokine is a pro-inflammatory cytokine.

[58] The circular polyribonucleotide of paragraph [57], wherein the pro-inflammatory cytokine is selected from GM-CSF, IL-1 alpha, IL-1 beta, TGF-beta, TNF-alpha, and TNF-beta.

[59] The circular polyribonucleotide of paragraph [57], wherein the cytokine is a Th-1 inducing cytokine.

[60] The circular polyribonucleotide of paragraph [59], wherein the Th-1 inducing cytokine is selected from IFN-gamma, IL-2, IL-12, IL-15, and IL-18.

[61] The circular polyribonucleotide of paragraph [56], wherein the cytokine is a Th-2 inducing cytokine.

[62] The circular polyribonucleotide of paragraph [61], wherein the Th-2 inducing cytokine is selected from IFN-gamma, IL-4, IL-5, IL-6, IL-10, and IL-13.

[63] The circular polyribonucleotide of paragraph [56], wherein the chemokine is selected from MCP-1, MIP-1alpha, MIP-1 beta, Rantes, and TCA-3.

[64] The circular polyribonucleotide of paragraph [56], wherein the costimulatory molecule is selected from CD80, CD86, CD40-L, CD70, and CD27.

[65] The circular polyribonucleotide of paragraph [56], wherein the innate immune stimulator is selected from STING (e.g., caSTING), TLR3, TLR4, TLR9, TLR7, TLR8, TLR7, RIG-I/DDX58, and MDA-5/IFIH1.

[66] The circular polyribonucleotide of paragraph [65], wherein the innate immune stimulator is a constitutively active mutant.

[67] The circular polyribonucleotide of paragraph [567], wherein the signaling molecule is selected from STING (e.g., caSTING), TRIF, TRAM, MyD88, IPS1, ASC, MAVS, MAPKs, IKK-alpha, IKK complex, TBK1, B-catenin, and caspase 1.

[68] The circular polyribonucleotide of paragraph [56], wherein the transcriptional activator is selected from AP1, NF-kappa B, IRF3, IRF7, IRF1, and IRFS.

[69] The circular polyribonucleotide of paragraph [56], wherein the cytokine receptor is selected from IL-2betaR, IFN-gammaR, and IL-6R.

[70] The circular polyribonucleotide of paragraph [56], wherein the bacterial component is selected from flagellin and MBL.

[71] The circular polyribonucleotide of any one of paragraphs [53] to [70], wherein at least sequence encoding the adjuvant further encodes a signal sequence.

[72] The circular polyribonucleotide of any one of paragraphs [1] to [70], wherein the circular polyribonucleotide further comprises a sequence that is an innate immune system stimulator.

[73] The circular polyribonucleotide of paragraph [72], wherein the sequence that is an innate immune system stimulator is a GU-rich motif, an AU-rich motif, a structured region comprising dsRNA, or an aptamer.

[74] A circular polyribonucleotide comprising (a) a sequence encoding a polypeptide immunogen and (b) a sequence encoding an adjuvant or a sequence that is an innate immune system stimulator.

[75] The circular polyribonucleotide of paragraph [74], wherein the sequence encoding the polypeptide immunogen is operably linked to an IRES.

[76] The circular polyribonucleotide of paragraph [74] or [75], wherein the sequence encoding the adjuvant is operably linked to an IRES.

[77] The circular polyribonucleotide of any one of paragraphs [74] to [76], wherein the adjuvant is a polypeptide.

[78] The circular polyribonucleotide of paragraph [77], wherein the polypeptide immunogen and the adjuvant polypeptide are encoded by a single open-reading frame operably linked to the single IRES, wherein the expression of the open reading frame produces a polypeptide comprising the amino acid sequence of each of the polypeptide immunogen and the adjuvant polypeptide.

[79] The circular polyribonucleotide of paragraph [78], wherein the polypeptide immunogen and the adjuvant polypeptide are each separated by a polypeptide linker.

[80] The circular polyribonucleotide of paragraph [78], wherein the polypeptide immunogen and the adjuvant polypeptide are each separated by a cleavage domain.

[81] The circular polyribonucleotide of paragraph [80], wherein each stagger element is a 2A self-cleaving peptide.

[82] The circular polyribonucleotide of paragraph [77], wherein the circular polyribonucleotide comprises a plurality of IRESs.

[83] The circular polyribonucleotide of paragraph [82], wherein a first IRES is operably linked to the sequence encoding the polypeptide immunogen and a second IRES is operably linked to the sequence encoding the adjuvant polypeptide.

[84] The circular polyribonucleotide of ant one of paragraphs [74] to [83], wherein the expression of the sequence encoding the polypeptide immunogen is 2-fold greater than the expression of the sequence encoding the adjuvant.

[85] The circular polyribonucleotide of ant one of paragraphs [74] to [84], wherein the expression of the sequence encoding the polypeptide immunogen is 5-fold, 10-fold, or 100-fold greater than the expression of the sequence encoding the adjuvant.

[86] The circular polyribonucleotide of any one of paragraphs [74] to [85], wherein the adjuvant is a cytokine, a chemokine, a costimulatory molecule, an innate immune stimulator, a signaling molecule, a transcriptional activator, a cytokine receptor, a bacterial component, or a component of the innate immune system.

[87] The circular polyribonucleotide of paragraph [86], wherein the cytokine is a pro-inflammatory cytokine.

[88] The circular polyribonucleotide of paragraph [87], wherein the pro-inflammatory cytokine is selected from GM-CSF, IL-1 alpha, IL-1 beta, TGF-beta, TNF-alpha, and TNF-beta.

[89] The circular polyribonucleotide of paragraph [86], wherein the cytokine is a Th-1 inducing cytokine.

[90] The circular polyribonucleotide of paragraph [89], wherein the Th-1 inducing cytokine is selected from IFN-gamma, IL-2, IL-12, IL-15, and IL-18.

[91] The circular polyribonucleotide of paragraph [86], wherein the cytokine is a Th-2 inducing cytokine.

[92] The circular polyribonucleotide of paragraph [91], wherein the Th-2 inducing cytokine is selected from IFN-gamma, IL-4, IL-5, IL-6, IL-10, and IL-13.

[93] The circular polyribonucleotide of paragraph [86], wherein the chemokine is selected from MCP-1, MIP-1alpha, MIP-1 beta, Rantes, and TCA-3.

[94] The circular polyribonucleotide of paragraph [93], wherein the costimulatory molecule is selected from CD80, CD86, CD40-L, CD70, and CD27.

[95] The circular polyribonucleotide of paragraph [86], wherein the innate immune stimulator is selected from STING (e.g., caSTING), TLR3, TLR4, TLR9, TLR7, TLR8, TLR7, RIG-I/DDX58, and MDA-5/IFIH1.

[96] The circular polyribonucleotide of paragraph [95], wherein the innate immune stimulator is a constitutively active mutant.

[97] The circular polyribonucleotide of paragraph [86], wherein the signaling molecule is selected from STING (e.g., caSTING), TRIF, TRAM, MyD88, IPS1, ASC, MAVS, MAPKs, IKK-alpha, IKK complex, TBK1, B-catenin, and caspase 1.

[98] The circular polyribonucleotide of paragraph [86], wherein the transcriptional activator is selected from AP1, NF-kappa B, IRF3, IRF7, IRF1, and IRFS.

[99] The circular polyribonucleotide of paragraph [86], wherein the cytokine receptor is selected from IL-2betaR, IFN-gammaR, and IL-6R.

[100] The circular polyribonucleotide of paragraph [86], wherein the bacterial component is selected from flagellin and MBL.

[101] The circular polyribonucleotide of any one of paragraphs [74] to [100], wherein the sequence encoding the adjuvant further encodes a signal sequence.

[102] The circular polyribonucleotide of any one of paragraphs [74] to [101], wherein the sequence encoding the polypeptide immunogen further encodes a signal sequence.

[103] The circular polyribonucleotide of any one of paragraph [74] to [101], wherein the sequence encoding the polypeptide immunogen does not encode a signal sequence.

[104] The circular polyribonucleotide of paragraph [74] or [75], wherein the sequence that is an innate immune system stimulator is selected from a GU-rich motif, an AU-rich motif, a structured region comprising dsRNA, or an aptamer.

[105] The circular polyribonucleotide of any one of paragraphs [1] to [104], wherein the circular polyribonucleotide comprises a first polyribonucleotide with a 5′ end and a 3′ end, wherein 5′ end and 3′ end are each hybridized to a second polynucleotide, there by linking the 5′ end and the 3′ end of the first polyribonucleotide to form a circular polyribonucleotide.

[106] The circular polyribonucleotide of paragraph [105], wherein the circular polyribonucleotide is produced by splint ligation.

[107] The circular polyribonucleotide of any one of paragraphs [1] to [104], wherein the circular polyribonucleotide is produced by providing a linear polyribonucleotide having a 3′ end and a 5′ end, wherein the 3′ end and the 5′ end each comprise a portion of an intron, and wherein the intron portion of the 3′ end and the intron portion of the 5′ end catalyze a splicing reaction thereby covalently conjugating the 5′ end and the 3′ end to produce a circular polyribonucleotide.

[108] The circular polyribonucleotide of paragraph [107], wherein the intron is a Group I self-splicing intron.

[109] An immunogenic composition comprising a plurality of circular polyribonucleotides, each comprising a sequence encoding a polypeptide immunogen.

[11] The immunogenic composition of paragraph [109], wherein each of the plurality of circular polyribonucleotides is a circular polyribonucleotide described by any one of paragraphs [1] to [108].

[110] The immunogenic composition of paragraph [109], wherein the composition comprises (a) at least a first circular polyribonucleotide comprising a sequence encoding a first polypeptide immunogen and (b) at least a second circular polyribonucleotide comprising a sequence encoding a second polypeptide immunogen, wherein the first and second polypeptide immunogens have at least 90% amino acid sequence identity.

[111] The immunogenic composition of paragraph [111], wherein the first and second polypeptide immunogens have at least 95% amino acid sequence identity.

[112] The immunogenic composition of paragraph [111] or [112], wherein the first and second polypeptide immunogens have less than 100% amino acid sequence identity.

[113] The immunogenic composition of any one of paragraphs [111] to [113], wherein the each of the polypeptide immunogens comprises one or more epitopes that identifies a target.

[114] The immunogenic composition of paragraph [114], wherein the target is a pathogen.

[115] The immunogenic composition of paragraph [115], wherein the target is a cancer cell, an allergen, or a toxin.

[116] The immunogenic composition of paragraph [109], wherein the composition comprises (a) at least a first circular polyribonucleotide comprising a sequence encoding a first polypeptide immunogen and (b) at least a second circular polyribonucleotide comprising a sequence encoding a second polypeptide immunogen, wherein the first and second polypeptide immunogens identify different proteins, wherein each different protein identifies the same target.

[117] The immunogenic composition of paragraph [117], wherein the target is a pathogen.

[119] The immunogenic composition of paragraph [118], wherein the pathogen is a virus, a bacterium, a fungus, or a parasite.

[120] The immunogenic composition of paragraph [119], wherein the pathogen is a cancer cell, an allergen, or a toxin.

[121] The immunogenic composition of paragraph [109], wherein the composition comprises (a) at least a first circular polyribonucleotide comprising a sequence encoding a first polypeptide immunogen and (b) at least a second circular polyribonucleotide comprising a sequence encoding a second polypeptide immunogen, wherein the first polypeptide immunogens identifies a first target and the second polypeptide immunogen identifies a second target.

[122] The immunogenic composition of paragraph [121], wherein each target is a pathogen.

[123] The immunogenic composition of paragraph [121] or [122], wherein each target is, independently, a cancer cell, a virus, a bacterium, a fungus, a parasite, a toxin, or an allergen.

[124] The immunogenic composition of any one of paragraphs [109] to [123], wherein each polypeptide immunogen is operably linked to an IRES.

[125] An immunogenic composition comprising a plurality of circular polyribonucleotides, wherein the composition comprises (a) at least a first circular polyribonucleotide comprising a sequence encoding a polypeptide immunogen and (b) at least a second circular polyribonucleotide comprising a sequence encoding an adjuvant or a sequence that is an innate immune system stimulator.

[126] The immunogenic composition of paragraph [125], wherein the adjuvant is a polypeptide.

[127] The immunogenic composition of paragraph [126], wherein the adjuvant is a cytokine, a chemokine, a costimulatory molecule, an innate immune stimulator, a signaling molecule, a transcriptional activator, a cytokine receptor, a bacterial component, or a component of the innate immune system.

[128] The immunogenic composition of paragraph [127], wherein the cytokine is a pro-inflammatory cytokine.

[129] The immunogenic composition of paragraph [128], wherein the pro-inflammatory cytokine is selected from GM-CSF, IL-1 alpha, IL-1 beta, TGF-beta, TNF-alpha, and TNF-beta.

[130] The immunogenic composition of paragraph [127], wherein the cytokine is a Th-1 inducing cytokine.

[131] The immunogenic composition of paragraph [130], wherein the Th-1 inducing cytokine is selected from IFN-gamma, IL-2, IL-12, IL-15, and IL-18.

[132] The immunogenic composition of paragraph [127], wherein the cytokine is a Th-2 inducing cytokine.

[133] The immunogenic composition of paragraph [132], wherein the Th-2 inducing cytokine is selected from IFN-gamma, IL-4, IL-5, IL-6, IL-10, and IL-13.

[134] The immunogenic composition of paragraph [127], wherein the chemokine is selected from MCP-1, MIP-1alpha, MIP-1 beta, Rantes, and TCA-3.

[135] The immunogenic composition of paragraph [134], wherein the costimulatory molecule is selected from CD80, CD86, CD40-L, CD70, and CD27.

[135] The immunogenic composition of paragraph [127], wherein the innate immune stimulator is selected from STING (e.g., caSTING), TLR3, TLR4, TLR9, TLR7, TLR8, TLR7, RIG-I/DDX58, and MDA-5/IFIH1.

[137] The immunogenic composition of paragraph [136], wherein the innate immune stimulator is a constitutively active mutant.

[138] The immunogenic composition of paragraph [127], wherein the signaling molecule is selected from STING (e.g., caSTING), TRIF, TRAM, MyD88, IPS1, ASC, MAVS, MAPKs, IKK-alpha, IKK complex, TBK1, B-catenin, and caspase 1.

[139] The immunogenic composition of paragraph [127], wherein the transcriptional activator is selected from AP1, NF-kappa B, IRF3, IRF7, IRF1, and IRFS.

[140] The immunogenic composition of paragraph [127], wherein the cytokine receptor is selected from IL-2betaR, IFN-gammaR, and IL-6R.

[141] The immunogenic composition of paragraph [127], wherein the bacterial component is selected from flagellin and MBL.

[142] The immunogenic composition of any one of paragraphs [125] to [141], wherein the polypeptide immunogen and the adjuvant are each operably linked to an IRES.

[143] The immunogenic composition of paragraph [125], wherein the sequence that is an innate immune system stimulator is a GU-rich motif, an AU-rich motif, a structured region comprising dsRNA, or an aptamer.

[144] The immunogenic composition of any one of paragraphs [1] to [143], wherein the circular polyribonucleotide comprises a first polyribonucleotide with a 5′ end and a 3′ end, wherein 5′ end and 3′ end are each hybridized to a second ribonucleotide, there by linking the 5′ end and the 3′ end of the first polyribonucleotide to form a circular polyribonucleotide.

[145] The circular polyribonucleotide of paragraphs [1] to [144], wherein the circular polyribonucleotide is produced by splint ligation.

[146] The immunogenic composition of any one of paragraphs [1] to [143], wherein the circular polyribonucleotide is produced by providing a linear polyribonucleotide having a 3′ end and a 5′ end, wherein the 3′ end and the 5′ end each comprise a portion of an intron, and wherein the intron portion of the 3′ end and the intron portion of the 5′ end catalyze a splicing reaction thereby covalently conjugating the 5′ end and the 3′ end to produce a circular polyribonucleotide.

[147] The immunogenic composition of paragraph [146], wherein the intron is a Group I self-splicing intron.

[148] A pharmaceutical preparation of a circular polyribonucleotide, wherein the circular polyribonucleotide comprises a sequence encoding a polypeptide immunogen, and wherein the circular polyribonucleotide constitutes at least 25% (w/w) of total ribonucleotide molecules in the pharmaceutical preparation.

[149] The pharmaceutical preparation of paragraph [148], wherein the circular polyribonucleotide constitutes at least 30% (w/w), 40% (w/w), 50% (w/w), 60% (w/w), 70% (w/w), 75% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w) or 99% (w/w) of total ribonucleotide molecules in the pharmaceutical preparation.

[150] The pharmaceutical preparation of any one of paragraphs [148] or [149], wherein the pharmaceutical preparation comprises no more than 75% (w/w), 60% (w/w), 50% (w/w), 40% (w/w), 30% (w/w), 20% (w/w), 15% (w/w), 10% (w/w), 5% (w/w), or 1% (w/w) linear polyribonucleotide molecules of the total ribonucleotide molecules in the pharmaceutical preparation.

[151] The pharmaceutical preparation of paragraph [150], wherein the pharmaceutical preparation comprises no more than 0.5% (w/w) linear polyribonucleotide molecules of the total ribonucleotide molecules in the pharmaceutical preparation.

[152] The pharmaceutical preparation of any one of paragraphs [148] to [151], wherein the preparation comprises no more than 15% (w/w) nicked polyribonucleotide molecules of the total ribonucleotide molecules in the pharmaceutical preparation.

[153] The pharmaceutical preparation of any one of paragraphs [148] to [152], wherein the preparation comprises no more than 10% (w/w), 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation.

[154] The pharmaceutical preparation of any one of paragraph [148] or [149], wherein the pharmaceutical preparation comprises at least 1% (w/w) linear polyribonucleotide.

[155] The pharmaceutical composition of paragraph [154], wherein the pharmaceutical preparation comprises between 30%-40% (w/w) circular polyribonucleotide and between 1%-10% (w/w), 10%-20% (w/w), 20%-30% (w/w), 30%-40% (w/w), 40%-50% (w/w), 50%-60% (w/w), or 60%-70% (w/w) linear polyribonucleotide.

[156] [156]. The pharmaceutical composition of paragraph [154], wherein the pharmaceutical preparation comprises between 40%-60% (w/w) circular polyribonucleotide and between 1%-10% (w/w), 10%-20% (w/w), 20%-30% (w/w), 30%-40% (w/w), 40%-50% (w/w), 50%-60% (w/w) linear polyribonucleotide.

[157] The pharmaceutical composition of paragraph [154], wherein the pharmaceutical preparation comprises between 60%-80% (w/w) circular polyribonucleotide and between 1%-10% (w/w), 10%-20% (w/w), 20%-30% (w/w), 30%-40% (w/w) linear polyribonucleotide.

[158] A pharmaceutical preparation of a circular polyribonucleotide, wherein the circular polyribonucleotide comprises a sequence encoding a polypeptide immunogen, and wherein the pharmaceutical preparation comprises no more than 75% (w/w) linear polyribonucleotide molecules of the total ribonucleotide molecules in the pharmaceutical preparation.

[159] The pharmaceutical preparation of paragraph [158], wherein the pharmaceutical preparation comprises no more than 60% (w/w), 50% (w/w), 40% (w/w), 30% (w/w), 20% (w/w), 15% (w/w), 10% (w/w), 5% (w/w), 1% (w/w), or 0.5% (w/w) linear polyribonucleotide molecules of the total ribonucleotide molecules in the pharmaceutical preparation.

[160] The pharmaceutical preparation of paragraph [158] or [159], wherein the preparation comprises no more than 15% (w/w) nicked polyribonucleotide molecules of the total ribonucleotide molecules in the pharmaceutical preparation.

[161] The pharmaceutical preparation of any one of paragraphs [158] to [160], wherein the preparation comprises no more than 10% (w/w), 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation.

[162] A pharmaceutical preparation of a circular polyribonucleotide, wherein the circular polyribonucleotide comprises a sequence encoding a polypeptide immunogen, and wherein the pharmaceutical preparation comprises no more than 15% (w/w) nicked polyribonucleotide molecules of the total ribonucleotide molecules in the pharmaceutical preparation.

[163] The pharmaceutical preparation of paragraph [162], wherein the preparation comprises no more than 10% (w/w), 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation.

[164] The pharmaceutical preparation of any one of paragraphs [148] to [163], wherein the pharmaceutical preparation comprises less than 10 EU/kg or lacks endotoxin as measured by a Limulus amebocyte lysate test.

[165] The pharmaceutical preparation of any one of paragraphs [148] to [163], wherein the pharmaceutical preparation comprises a bioburden of less than 100 CFU/100 ml or less than 10 CFU/100 ml before sterilization.

[166] The pharmaceutical preparation of any one of paragraphs [148] to [165], wherein the pharmaceutical preparation is a sterile pharmaceutical preparation, e.g., supports growth of fewer than 100 viable microorganisms as tested under aseptic conditions.

[167] The pharmaceutical preparation of any one of paragraphs [148] to [166], wherein the pharmaceutical preparation meets the standard of USP <71>.

[168] The pharmaceutical preparation of any one of paragraphs [148] to [67], wherein the pharmaceutical preparation meets the standard of USP <85>.

[169] The pharmaceutical preparation of any one of paragraphs [148] to [168], wherein the pharmaceutical preparation is an intermediate pharmaceutical preparation of a final drug product.

[170] The pharmaceutical preparation of any one of paragraphs [148] to [169], wherein the pharmaceutical preparation is a final drug product for administration to a subject.

[171] The pharmaceutical preparation of any one of paragraphs [148] to [170], wherein the pharmaceutical preparation comprises a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL circular polyribonucleotide molecules.

[172] The pharmaceutical preparation of any one of paragraphs [148] to [171], wherein the pharmaceutical preparation comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL of deoxyribonucleotide molecules.

[173] The pharmaceutical preparation of any one of paragraphs [148] to [172], wherein the pharmaceutical preparation comprises a protein contamination (e.g. an enzyme) of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules.

[174] The pharmaceutical preparation of any one of paragraphs [148] to [173], wherein the pharmaceutical preparation comprises an A260/A280 absorbance ratio of from about 1.6 to 2.3 as measured by a spectrophotometer.

[175] The pharmaceutical preparation of any one of paragraphs [148] to [174], wherein the pharmaceutical preparation is substantially free of a process-related impurity selected from a cell protein, a cell deoxyribonucleic acid, an enzyme, a reagent component, a gel component, or a chromatographic material.

[176] The pharmaceutical preparation of any one of paragraphs [148] to [175], wherein the pharmaceutical preparation has a reduced level of one or more markers of an immune or inflammatory response after purification compared to prior to purification.

[177] The pharmaceutical preparation of any one of paragraphs [148] to [176], wherein the circular polyribonucleotide is a circular polyribonucleotide of any one of paragraphs [1] to [108].

[178] The pharmaceutical preparation of any one of paragraphs [148] to [177], wherein the circular polyribonucleotide is produced according to a method comprising the steps of:
(a) providing a plurality of linear polyribonucleotide molecules;
b) circularizing the linear polyribonucleotide molecules to provide a preparation of circular polyribonucleotide molecules; and
c) processing the preparation to remove linear polyribonucleotide molecules from the preparation.

[178] The pharmaceutical preparation of paragraph [178], wherein the method further comprises
d) evaluating the amount of linear polyribonucleotide molecules in the preparation remaining after the processing step.

[179] The pharmaceutical preparation of paragraph [178] or [179], wherein the method further comprises e) further processing the preparation, wherein further processing comprises:
processing the preparation to remove nicked polyribonucleotide molecules from the preparation; and/or
processing the preparation to remove deoxyribonucleotide molecules from the preparation; and/or
processing the preparation to remove protein contaminants from the preparation; and/or
processing the preparation to remove endotoxin from the preparation.

[181] A pharmaceutical composition comprising the circular polyribonucleotide of any one of paragraphs [1] to [108], the immunogenic composition of any one of paragraphs [109] to [147], or the pharmaceutical preparation of any one of paragraphs [148] to [180], and a pharmaceutically acceptable excipient.

[182] The pharmaceutical composition of paragraph [181], further comprising an adjuvant.

[183] The pharmaceutical composition of paragraph [182], wherein the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant.

[184] The pharmaceutical composition of paragraph [183], wherein the adjuvant is an inorganic adjuvant.

[185] The pharmaceutical composition of paragraph [184], wherein the inorganic adjuvant is an aluminum salt or a calcium salt.

[186] The pharmaceutical composition of paragraph [183], wherein the adjuvant is a small molecule.

[187] The pharmaceutical composition of paragraph [183], wherein the small molecule is imiquimod, resiquimod, or gardiquimod.

[188] The pharmaceutical composition of paragraph [183], wherein the adjuvant is an oil in water emulsion.

[189] The pharmaceutical composition of paragraph [188], wherein the oil in water emulsion is Squalene, MF59, AS03, a Montanide formulation, optionally Montanide ISA 51 or Montanide ISA 720, or Incomplete Freunds Adjuvant an oil in water emulsion.

[190] The pharmaceutical composition of paragraph [183], wherein the adjuvant is a lipid or polymer.

[191] The pharmaceutical composition of paragraph [190], wherein the lipid or polymer is a polymeric nanoparticle, optionally PLGA, PLG, PLA, PGA, or PHB, a liposome, optionally a Virosomes or CAF01, a lipid nanoparticle or a component thereof, a lipopolysaccharide (LPS), optionally monophosphoryl lipid A (MPLA) or glucopyranosyl Lipid A (GLA), a lipopeptide, optionally Pam2 (Pam2CSK4) or Pam3 (Pam3CSK4), or a glycolipid, optionally, trehalose dimycolate.

[192] The pharmaceutical composition of paragraph [187], wherein the adjuvant is a peptide or peptidoglycan.

[193] The pharmaceutical composition of paragraph [192], wherein the peptide of peptidoglycan corresponds to all or a portion of a synthetic or purified gram negative or gram positive bacteria, optionally N-acetyl-muramyl-L-alanyl-D-isoglutamine (MDP), a flagellin-fusion protein, Mannose-binding Lectin (MBL), a cytokines, or a chemokine.

[194] The pharmaceutical composition of paragraph [183], wherein the adjuvant is a carbohydrate or polysaccharide.

[195] The pharmaceutical composition of paragraph [194], wherein the carbohydrate or polysaccharide is dextran (branched microbial polysaccharide), dextran-sulfate, Lentinan, zymosan, Beta-glucan, Deltin, Mannan, or Chitin.

[196] The pharmaceutical composition of paragraph [183], wherein the adjuvant is a saponin.

[197] The pharmaceutical composition of paragraph [196], wherein the saponin is a glycoside or a polycyclic aglycones attached to one or more sugar side chains, optionally ISCOMS, ISCOMS matrix, or QS-21.

[198] The pharmaceutical composition of paragraph [183], wherein the adjuvant is an RNA-based adjuvant.

[199] The pharmaceutical composition of paragraph [198], wherein the RNA-based adjuvant is Poly IC, Poly IC:LC, a hairpin RNAs, optionally with a 5′PPP containing sequence, a viral sequence, a polyU containing sequences, dsRNA, a natural or synthetic immunostimulatory RNA sequence, a nucleic acids analogs, optionally cyclic GMP-AMP or a cyclic dinucleotide such as cyclic di-GMP, or an immunostimulatory base analog, optionally a C8-substitued or an N7,C8-disubstituted guanine ribonucleotide.

[200] The pharmaceutical composition of paragraph [183], wherein the adjuvant is a DNA-based adjuvant.

[201] The pharmaceutical composition of paragraph [200], wherein the DNA-based adjuvant is a CpG, dsDNA, or a natural or synthetic immunostimulatory DNA sequence.

[202] The pharmaceutical composition of paragraph [183], wherein the adjuvant is a viral particle.

[203] The pharmaceutical composition of paragraph [202], wherein the viral particle is a virosome, optionally, a phospholipid cell membrane bilayer.

[204] The pharmaceutical composition of paragraph [183], wherein the adjuvant is a bacterial adjuvant.

[205] The pharmaceutical composition of paragraph [204], wherein the bacterial adjuvant is flagellin, LPS, or a bacterial toxin, optionally an enterotoxins, a heat-labile toxin, or a Cholera toxin

[206] The pharmaceutical composition of paragraph [183], wherein the adjuvant is a hybrid molecule.

[207] The pharmaceutical composition of paragraph [206], wherein the adjuvant is CpG conjugated to Imiquimod.

[208] The pharmaceutical composition of paragraph [183], wherein the adjuvant is a fungal or oocyte microbe-associated molecular pattern (MAMP).

[209] The pharmaceutical composition of paragraph [208], wherein the fungal or oocyte MAMP is chitin or beta-glucan.

[210] The pharmaceutical composition of paragraph [183], wherein the adjuvant is an inorganic nanoparticle.

[211] The pharmaceutical composition of paragraph [210], wherein the inorganic nanoparticle is a gold nanorod, a silica-based nanoparticle, optionally a mesoporous silica nanoparticles (MSN).

[212] The pharmaceutical composition of paragraph [183], wherein the adjuvant is a multi-component adjuvant.

[213] The pharmaceutical composition of paragraph [212], wherein the multi-component adjuvant is AS01, AS03, AS04, Complete Freunds Adjuvant, or CAF01.

[214] A pharmaceutical composition comprising a mixture comprising (a) a polyribonucleotide comprising a sequence encoding a polypeptide immunogen and (b) an alcohol, wherein the alcohol constitutes about 0.3% v/v to about 75% v/v of the mixture.

[215] The pharmaceutical composition of paragraph [214], wherein the polyribonucleotide is a circular polyribonucleotide.

[216] A pharmaceutical composition comprising a mixture comprising (a) the circular polyribonucleotide of any one of paragraphs [1] to [108], the immunogenic composition of any one of paragraphs [109] to [147], or the pharmaceutical preparation of any one of paragraphs [148] to [180], and (b) an alcohol, wherein the alcohol constitutes about 0.3% v/v to about 75% v/v of the mixture.

[217] The pharmaceutical composition of paragraph [214], wherein the polyribonucleotide is a linear polyribonucleotide.

[218] The pharmaceutical composition of paragraph [217], wherein the linear polyribonucleotide comprises a plurality of sequences, each sequence encoding a polypeptide immunogen, wherein at least two of the polypeptide immunogens have at least 90% amino acid sequence identity.

[219] The pharmaceutical composition of paragraph [218], wherein the at least two polypeptide immunogens have less than 100% amino acid sequence identity.

[220] The pharmaceutical composition of paragraph [217], wherein the linear polyribonucleotide comprises a plurality of sequences, each sequence encoding a polypeptide immunogen, wherein at least two of the polypeptide immunogens identify different proteins, wherein each of the different proteins identifies the same target.

[221] The pharmaceutical composition of paragraph [217], wherein the linear polyribonucleotide comprises a plurality of sequences, each sequence encoding a polypeptide immunogen, wherein at least two of the polypeptide immunogens identify different targets.

[222] The pharmaceutical composition of any one of paragraphs [217] to [221], wherein the linear polyribonucleotide comprises at least three, at least four, at least five, at least six, at least seven, at least eight, or at least nine sequences, each sequence encoding a polypeptide immunogen.

[223] The pharmaceutical composition of paragraph [215], wherein the circular polyribonucleotide further comprises at least one sequence encoding an adjuvant.

[224] The pharmaceutical composition of paragraph [223], wherein the adjuvant is a polypeptide.

[225] The pharmaceutical composition of paragraph [224], wherein the adjuvant is a cytokine, a chemokine, a costimulatory molecule, an innate immune stimulator, a signaling molecule, a transcriptional activator, a cytokine receptor, a bacterial component, or a component of the innate immune system.

[226] The pharmaceutical composition of any one of paragraphs [215] or [223] to [225], wherein the circular polyribonucleotide further comprises a sequence that is an innate immune system stimulator.

[227] The pharmaceutical composition of paragraph [226], wherein the sequence that is an innate immune system stimulator is a GU-rich motif, an AU-rich motif, a structured region comprising dsRNA, or an aptamer.

[228] The pharmaceutical composition of any one of paragraphs [214] to [227], wherein the alcohol constitutes about 0.3% v/v to about 60% v/v, about 0.3% v/v to about 50% v/v, about 0.3% v/v to about 40% v/v about 0.3% v/v to about 20% v/v, about 0.3% v/v to about 15% v/v, about 0.3% v/v to about 10% v/v, about 0.3% v/v to about 5% v/v, about 0.3% v/v to about 1% v/v, or about 0.3% v/v to about 0.5% v/v of the mixture.

[229] The pharmaceutical composition of any one of paragraphs [214] to [227], wherein the alcohol constitutes about 0.5% v/v to about 75% v/v, about 1% v/v to about 75% v/v, about 5% v/v to about 75% v/v, about 10% v/v to about 75% v/v, about 15% v/v to about 75% v/v, about 20% v/v to about 75% v/v, about 30% v/v to about 75% v/v, about 40% v/v to about 75% v/v, about 50% v/v to about 75% v/v, about 60% v/v to about 75% v/v, or about 70% v/v to about 75% v/v of the mixture.

[230] The method of any one of paragraphs [214] to [229], wherein the alcohol is selected from methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[231] The method of paragraph [230], wherein the alcohol is ethanol.

[232] The pharmaceutical composition of any one of paragraphs [214] to [231], wherein the pharmaceutical composition is formulated for topical administration.

[233] The pharmaceutical composition of any one of paragraphs [214] to [232], wherein the pharmaceutical composition is a liquid, gel, lotion, paste, cream, foam, or stick.

[234] The pharmaceutical composition of any one of paragraphs [214] to [233], wherein the pharmaceutical composition has a pH of about 6-8.

[235] The pharmaceutical composition of any one of paragraphs [214] to [234], wherein the pharmaceutical composition has a viscosity that is about the same as water.

[236] The pharmaceutical composition of any one of paragraphs [214] to [235], wherein the pharmaceutical composition is substantially free of hydrophobic or lipophilic groups.

[237] The pharmaceutical composition of any one of paragraphs [214] to [236], wherein the pharmaceutical composition is substantially free of hydrocarbons.

[238] The pharmaceutical composition of any one of paragraphs [214] to [237], wherein the pharmaceutical composition is substantially free of cationic liposomes.

[239] The pharmaceutical composition of any one of paragraphs [214] to [238], wherein the pharmaceutical composition is substantially free of fatty acids, lipids, liposomes, cholesterol, or any combination thereof.

[240] The pharmaceutical composition of any one of paragraphs [214] to [239], the composition further comprising an adjuvant.

[241] The pharmaceutical composition of paragraph [240], wherein the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant.

[242] A method of delivering a polyribonucleotide to a subject comprising topically applying the pharmaceutical composition of any one of paragraphs [214] to [241] to a surface area of the subject.

[243] A method of delivering a polyribonucleotide to a subject comprising:
a) applying a sterilizing agent to a surface area of the subject; and
b) applying a composition comprising the circular polyribonucleotide of any one of paragraphs [1] to [108], the immunogenic composition of any one of paragraphs [109] to −[147], the pharmaceutical preparation of any one of paragraphs [148] to [180], or the pharmaceutical composition of any one of paragraphs [181] to [242] and a diluent to the surface area of the subject.

[244] The method of paragraph [243], wherein the composition of (b) does not comprise a carrier.

[245] The method of paragraph [243] or [244], wherein the sterilizing agent is an alcohol, UV light, laser light, or heat.

[246] A method of delivering a polyribonucleotide to a subject comprising
a) applying an alcohol to a surface area of the subject; and
b) applying a composition comprising the circular polyribonucleotide of any one of paragraphs [1] to [108], the immunogenic composition of any one of paragraphs [109] to [147], the pharmaceutical preparation of any one of paragraphs [148] to [180], or the pharmaceutical composition of any one of paragraphs [181] to [241] and a diluent to the surface area of the subject.

[247] The method of paragraph [246], wherein the alcohol is selected from the group consisting of: methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[248] The method of paragraph [247], wherein the alcohol is ethanol.

[249] The method of any one of paragraphs [243] to [248], wherein the surface area of the subject is selected from a surface area of the skin, oral cavity, nasal cavity, gastrointestinal tract, respiratory tract, or any combination thereof.

[250] A lipid nanoparticle (LNP) comprising the circular polyribonucleotide of any one of paragraphs [1] to [108] or the immunogenic composition of any one of paragraphs [109] to [147].

[251] The LNP of paragraph [250], comprising an ionizable lipid.

[252] The LNP of paragraph [250], comprising a cationic lipid.

[253] The LNP of paragraph [252], wherein the cationic lipid has a structure according to:

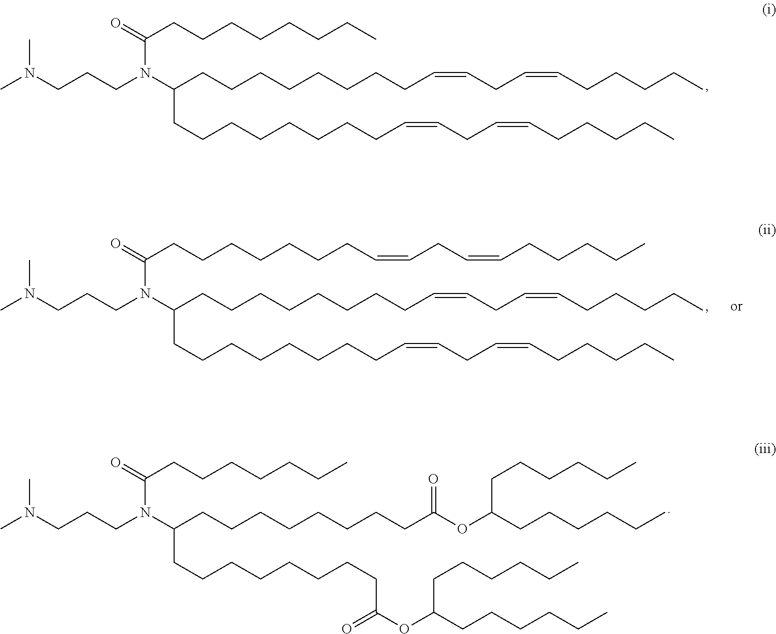

[254] The LNP of any one of paragraphs [250] to [253], further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

[255] A method of treating or preventing a disease, disorder, or condition in a subject, the method comprising administering to the subject the circular polyribonucleotide of any one of paragraphs [1] to [108], the immunogenic composition of any one of paragraphs [109] to [147], the pharmaceutical preparation of any one of paragraphs [148] to [180], the pharmaceutical composition of any one of paragraphs [181] to [241], or the LNP of any one of paragraphs [250] to [254].

[256] The method of paragraph [255], wherein the disease, disorder, or condition is a viral infection, a bacterial infection, or a fungal infection.

[257] The method of paragraph [255], wherein the disease, disorder, or condition is a cancer.

[258] The method of paragraph [255], wherein the disease, disorder, or condition is associated with exposure to an allergen.

[259] The method of paragraph [255], wherein the disease, disorder, or condition is associated with exposure to a toxin.

[260] A method of inducing an immune response in a subject, the method comprising administering to the subject the circular polyribonucleotide of any one of paragraphs [1] to [108], the immunogenic composition of any one of paragraphs [109] to [147], the pharmaceutical preparation of any one of paragraphs [148] to [180], the pharmaceutical composition of any one of paragraphs [181] to [241] or the LNP of any one of paragraphs [250] to [254].

[261] The method of any one of paragraphs [255] to [260], wherein the subject is a mammal.

[262] The method of paragraph [261], wherein the subject is a human.

[263] The method of paragraph [261], wherein the method is a non-human mammal.

[264] The method of paragraph [261], wherein in the non-human mammal is a cow, a sheep, a goat, a pig, a dog, a horse, or a cat.

[265] The method of any one of paragraphs [255] to [264], wherein the subject is a bird.

[266] The method of paragraph [265], wherein in the bird is a hen, a rooster, a turkey, or a parrot.

[267] The method of any one of paragraphs [255] to [266], wherein the method further comprises administering an adjuvant to the subject.

[268] The method of paragraph [267], wherein the adjuvant is an inorganic adjuvant, a small molecule adjuvant, and oil in water emulsion, a lipid or polymer, a peptide or peptidoglycan, a carbohydrate or polysaccharide, a saponin, an RNA-based adjuvant, a DNA-based adjuvant, a viral particle, a bacterial adjuvant, a hybrid molecule, a fungal or oocyte microbe-associated molecular pattern (MAMP), an inorganic nanoparticle, or a multi-component adjuvant.

[269] The method of paragraph [267], wherein the adjuvant is a polypeptide adjuvant.

[270] The method of paragraph [269], wherein the polypeptide adjuvant is a cytokine, a chemokine, a costimulatory molecule, an innate immune stimulator, a signaling molecule, a transcriptional activator, a cytokine receptor, a bacterial component, or a component of the innate immune system.

[271] The method of paragraph [267], wherein the adjuvant is an innate immune system stimulator.

[272] The method of paragraph [271], wherein the innate immune system stimulator is selected from an RNA including a GU-rich motif, an AU-rich motif, a structured region comprising dsRNA, or an aptamer.

[273] The method of any one of paragraphs [255] to [272], wherein the circular polyribonucleotide of any one of paragraphs [1] to [110], the immunogenic composition of any one of paragraphs [111] to [151], the pharmaceutical preparation of any one of paragraphs [152] to [184], the pharmaceutical composition of any one of paragraphs [185] to [245], or the LNP of any one of paragraphs [250] to [254] is administered to the subject as a single dose.

[274] The method of any one of paragraphs [255] to [272], wherein the circular polyribonucleotide of any one of paragraphs [1] to [108], the immunogenic composition of any one of paragraphs [109] to [147], the pharmaceutical preparation of any one of paragraphs [148] to [180], the pharmaceutical composition of any one of paragraphs [181] to [241], or the LNP of any one of paragraphs [250] to [254] is administered to the subject two or more times, three or more times, four or more times, or five or more times.

[275] The method of paragraph [274], wherein administration of the circular polyribonucleotide of any one of paragraphs [1] to [108], the immunogenic composition of any one of paragraphs [109] to [147], the pharmaceutical preparation of any one of paragraphs [148] to [180], the pharmaceutical composition of any one of paragraphs [181] to [241], or the LNP of any one of paragraphs [250] to [254] occurs about weekly, about every two weeks, about every three weeks, about every month, about every two months, about every three months, about every four months, about every five months, about every six months, about every year, about every two years, about every three years, about every four years, about every five years, or about every ten years.

[276] The method any one of paragraphs [255] to [275], wherein the method further comprises administering to the subject a polypeptide immunogen (e.g., a protein subunit comprising a polypeptide immunogen).

[277] The method of paragraph [276] wherein the polypeptide immunogen corresponds to (e.g., shares 90%, 95%, 96%, 97%, 98%, or 100% amino acid sequence identity with a polypeptide immunogen encoded by a sequence of the circular polyribonucleotide.

[278] The method of paragraph [276], wherein the polypeptide immunogen is administered to the subject after administering the circular polyribonucleotide of any one of paragraphs [1] to [108], the immunogenic composition of any one of paragraphs [109] to [147], the pharmaceutical preparation of any one of paragraphs [148] to [180], the pharmaceutical composition of any one of paragraphs [181] to [241], or the LNP of any one of paragraphs [250] to [254].

[279] The method of any one of paragraphs [276] to [278], wherein the polypeptide immunogen maintains or enhances an immune response in the subject against the polypeptide immunogen.

[280] A method of maintaining or enhancing an immune response in a subject, the method comprising (i) administering to the subject a circular polyribonucleotide encoding a polypeptide immunogen and (ii) administering to the subject the polypeptide immunogen, wherein step (ii) occurs between 1 week and 6 months after step (i), and wherein administration of the polypeptide immunogen of step (ii) maintains or enhances the immune response in the subject against the polypeptide immunogen.

The method of paragraph [280], wherein the polypeptide immunogen comprises one or more epitopes that identifies a target.

The method of paragraph [281], wherein the target is a pathogen.

The method of paragraph [281], wherein the target is a cancer cell, an allergen, or a toxin.

### Definitions

The present disclosure will be described with respect to particular embodiments and with reference to certain figures, but the disclosure is not limited thereto but only by the claims. Terms as set forth hereinafter are generally to be understood in their common sense unless indicated otherwise.

As used herein, the term “adaptive immune response” means either a humoral or cell-mediated immune response. For purposes of the present disclosure, a “humoral immune response” refers to an immune response mediated by antibody molecules, while a “cellular immune response” is one mediated by T-lymphocytes and/or other white blood cells.

As used herein, the term “adjuvant” refers to a composition (e.g., a compound, polypeptide, nucleic acid, or lipid) that increases an immune response, for example, increases a specific immune response against an immunogen. Increasing an immune response includes intensification or broadening the specificity of either or both antibody and cellular immune responses.

The term “alcohol” means any organic compound in which the hydroxyl functional group (—OH) is bound to a carbon. In some embodiments, an alcohol includes a hydroxyl bound to a saturated carbon, which is in turn bound to other hydrogen or carbon atoms. An alcohol as discussed herein can include, but is not limited to, monohydric alcohols, polyhydric alcohols, unsaturated aliphatic alcohols, and alicyclic alcohols. In some cases, an alcohol can refer to ethanol. In some cases, an alcohol can include, but is not limited to, methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

As used herein, the term “associated with” a disease, disorder, or condition refers to a relationship, either causative or correlative, between an entity and the occurrence or severity of a disease, disorder, or condition in a subject. For example, if a target is associated with a disease, disorder, or condition, the target may be the causative agent of the disease, disorder, or condition. For example, a virus may be the causative agent in a viral infection, bacteria may be the causative agent in a bacterial infection, a fungus may be the causative agent in a fungal infection, or a parasite may be the causative agent in a parasitic infection, a cancer cell may be the causative agent of a cancer, a toxin may be the causative agent of toxicity, or an allergen may the causative agent of an allergic reaction. The target associated with a disease, disorder, or condition may also or alternately be correlated with an increased likelihood of occurrence or an increase severity of a disease disorder, or condition.

As used herein, the term “carrier” means a compound, composition, reagent, or molecule that facilitates the transport or delivery of a composition (e.g., a linear or a circular polyribonucleotide) into a subject, a tissue, or a cell. Non-limiting examples of carriers include carbohydrate carriers (e.g., an anhydride-modified phytoglycogen or glycogen-type material), nanoparticles (e.g., a nanoparticle that encapsulates or is covalently linked binds to the circular or linear polyribonucleotide), liposomes, fusosomes, ex vivo differentiated reticulocytes, exosomes, protein carriers (e.g., a protein covalently linked to the polyribonucleotide), or cationic carriers (e.g., a cationic lipopolymer or transfection reagent).

As used herein, the term “cell-penetrating agent” means an agent that, when contacted to a cell, facilitates entry into the cell. In some cases, a cell-penetrating agent facilitates direct penetration of the cell membrane, for instance, via direct electrostatic interaction with negatively charged phospholipids of the cell membrane, or transient pore formation by inducing configurational changes in membrane proteins or the phospholipid bilayer. In some cases, a cell-penetrating agent facilitates endocytosis-mediated translocation into the cell. For example, under certain situation, the cell-penetrating agent can stimulate the cell to undergo the endocytosis process, by which the cell membrane can fold inward into the cell. In certain embodiments, a cell-penetrating agent helps form a transitory structure that transports across the cell membrane. Without wishing to be bound to a particular theory, a cell-penetrating agent as provided herein can increase the permeability of the cell membrane or increase internalization of a molecule into the cell, as a result of which, delivery into the cell can be more efficient when the cell is contacted with the cell-penetrating agent simultaneously as compared to otherwise identical delivery without the cell-penetrating agent.

As used herein, the terms “circRNA” or “circular polyribonucleotide” or “circular RNA” or “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently-closed) or non-covalent bonds.

As used herein, the term “circularization efficiency” is a measurement of resultant circular polyribonucleotide versus its non-circular starting material.

As used herein, the terms “circRNA preparation” or “circular polyribonucleotide preparation” or “circular RNA preparation” are used interchangeably and mean a composition including circRNA molecules and a diluent, carrier, first adjuvant, or a combination thereof.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc.

The wording “compound, composition, product, etc. for use in . . . ” or “use of a compound, composition, product, etc. in the manufacture of a medicament, pharmaceutical composition, veterinary composition, diagnostic composition, etc. for . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”.

The term “diluent” means a vehicle including an inactive solvent in which a composition described herein (e.g., a composition including a circular or linear polyribonucleotide) may be diluted or dissolved.

A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the terms “disease,” “disorder,” and “condition” each refer to a state of sub-optimal health, for example, a state that is or would typically be diagnosed or treated by a medical professional.

As used herein, the term “epitope” refers to a portion or the whole of an immunogen that is recognized, targeted, or bound by an antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope includes nucleic acids that form an epitope in the folded conformation of an immunogenic sequence based on its secondary structure or tertiary structure.

As used herein, the term “encryptogen” is a nucleic acid sequence or structure of the circular polyribonucleotide that aids in reducing, evading, and/or avoiding detection by an immune cell and/or reduces induction of an immune response against the circular or linear polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide can include a plurality of nucleotide triads, each of which can code for an amino acid and is termed as a “codon”.

As used herein, the terms “identify” or “identifies” refer to indicating, establishing, or recognizing the identity of an entity. For example, an immunogen or an epitope thereof may identify a target, meaning that the target includes the immunogen or epitope thereof, that the immunogen or epitope thereof is derived from the target, and/or the immunogen or epitope thereof shares a high degree of similarity with a portion or the whole of the target. Recognition or binding of an antibody or a T cell receptor to an immunogen or an epitope thereof can identify a target. Where an immunogen or an epitope thereof identifies a target, the immunogen or epitope thereof distinguishes the target from one or more other targets. Likewise, a polypeptide immunogen can identify a protein. Otherwise put, the polypeptide immunogen is a component of, a portion of, is derived from, or shares a high degree of similarity to the protein or a portion of the protein, in particular to an epitope of a protein.

As used herein, the term “innate immune system stimulator,” refers to a substance that induces an innate immunological response, in part, by inducing expression of one or more genes involved in innate immunity, including, but not limited to, a type I interferon (e.g., IFNα, INFβ, and/or IFNγ), a pro-inflammatory cytokine (e.g., IL-1, IL-12, IL-18, TNF-α, and/or GM-CSF), retinoic-acid inducible gene-I (RIG-1, also known as DDX58), melanoma-differentiation-associated gene 5 (MDA5, also known as IFIH1), 2′-5′ oligoadenylate synthase 1 (OAS 1), OAS-like protein (OASL), and/or protein kinase R (PKR). An innate immune system stimulator may act as an adjuvant, e.g., when administered in combination with or formulated with, a ribonucleotide that encodes an immunogen. An innate immune system stimulator may be a separate molecule entity (e.g., not encoded by or incorporated as a sequence in a polyribonucleotide), for example, STING (e.g., caSTING), TLR3, TLR4, TLR9, TLR7, TLR8, TLR7, RIG-I/DDX58, and MDA-5/IFIH1 or a constitutively active mutant thereof. An innate immune system stimulator may be encoded by (e.g., expressed from) a polyribonucleotide. A polyribonucleotide may alternately or further include a ribonucleotide sequence that acts as an innate immune system stimulator (e.g., GU-rich motif, an AU-rich motif, a structured region comprising dsRNA, or an aptamer).

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular or linear polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the term “immunogen,” refers to an any molecule or molecular structure that includes one or more epitopes recognized, targeted, or bound by an antibody or a T cell receptor. In particular, an immunogen induces an immune response in a subject (e.g., is immunogenic as defined herein). An immunogen is capable of inducing an immune response in a subject, wherein the immune response refers to a series of molecular, cellular, and organismal events that are induced when an immunogen is encountered by the immune system. The immune response may be humoral and/or cellular immune response. These may include the production of antibodies and the expansion of B- and T-cells. To determine whether an immune response has occurred and to follow its course, the immunized subject can be monitored for the appearance of immune reactants directed at the specific immunogen. Immune responses to most immunogen induce the production of both specific antibodies and specific effector T cells. In some embodiments, the immunogen is foreign to a host. In some embodiments, the immunogen is not foreign to a host. An immunogen may include all or a portion of a polypeptide, a polysaccharide, a polynucleotide, or a lipid. An immunogen may also be a mixed polypeptide, polysaccharide, polynucleotide, and/or lipid. For example, an immunogen maybe a polypeptide that has been translationally modified. A “polypeptide immunogen” refers to an immunogen that includes a polypeptide. A polypeptide immunogen may also include one or more post-translational modifications, and/or may form a complex with one or more additional molecules, and/or may adopt a tertiary or quaternary structure, each of which may determine or affect the immunogenicity of the polypeptide.

As used herein, the term “immunogenic” is a potential to induce a response to a substance in a particular immune response assay above a pre-determined threshold. The assay can be, e.g., expression of certain inflammatory markers, production of antibodies, or an assay for immunogenicity as described herein. In some embodiments, an immune response may be induced when an immune system of an organism or a certain type of immune cells are exposed to an immunogen.

An immunogenic response may be assessed may evaluating the antibodies in the plasma or serum of a subject using a total antibody assay, a confirmatory test, titration and isotyping of the antibodies, and neutralizing antibody assessment. A total antibody assay measures the all the antibodies generated as part of the immune response in the serum or plasma of a subject that has been administered the immunogen. The most commonly used test to detect antibodies is an ELISA (enzyme-linked immunosorbent assay), which detects antibodies in the tested serum that bind to the antibody of interest, including IgM, IgD, IgG, IgA, and IgE. An immunogenic response can be further assessed by a confirmatory assay. Following a total antibody assessment, a confirmatory assay may be used to confirm the results of the total antibody assay. A competition assay may be used to confirm that antibody is specifically binding to target and that the positive finding in the screening assay is not a result of nonspecific interactions of the test serum or detection reagent with other materials in the assay.

An immunogenic response can be assessed by isotyping and titration. An isotyping assay may be used to assess only the relevant antibody isotypes. For example, the expected isotypes may be IgM and IgG which may be specifically detected and quantified by isotyping and titration, and then compared to the total antibodies present.

An immunogenic response can be assessed by a neutralizing antibody assay (nAb). A neutralizing antibody assay (nAb) may be used to determine if the antibodies produced in response to the immunogen neutralized the immunogen thereby inhibiting the immunogen from having an effect on the target and leading to abnormal pharmacokinetic behaviors. An nAb assay is often a cell-based assay where the target cells are incubated with the antibody. A variety of cell based nAb assays may be used including but not limited to Cell Proliferation, Viability, Antibody-Dependent Cell-Mediated Cytotoxicity (ADCC), Complement-Dependent Cytotoxicity (CDC), Cytopathic Effect Inhibition (CPE), Apoptosis, Ligand Stimulated Cell Signaling, Enzyme Activity, Reporter Gene Assays, Protein Secretion, Metabolic Activity, Stress and Mitochondrial Function. Detection readouts include Absorbance, Fluorescence, Luminescence, Chemiluminescence, or Flow Cytometry. A ligand-binding assay may also be used to measure the binding affinity of an immunogen and an antibody in vitro to evaluate neutralization efficacy.

Furthermore, induction of a cellular immune response may be assessed by measuring T cell activation in a subject using cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40L, CD134, CD69, CD62L or CD44. T cell activation can also be assessed using the same methods in an in vivo animal model. This assay can also be performed by adding an immunogen to T cells in vitro (e.g., T cells obtained from a subject, animal model, repository, or commercial source) and measuring the aforementioned markers to evaluate T cell activation. Similar approaches can be used to assess the effect of an on activation of other immune cells, such as eosinophils (markers: CD35, CD11 b, CD66, CD69 and CD81), dendritic cells (makers: IL-8, MHC class II, CD40, CD80, CD83, and CD86), basophils (CD63, CD13, CD4, and CD203c), and neutrophils (CD11 b, CD35, CD66b and CD63). These markers can be assessed using flow cytometry, immunohistochemistry, in situ hybridization, and other assays that allow for measurement of cellular markers. Comparing results from before and after administration of an immunogen can be used to determine its effect.

As used herein, the term “inducing an immune response” refers to initiating, amplifying, or sustaining an immune response by a subject. Inducing an immune response may refer to an adaptive immune response or an innate immune response. The induction of an immune response may be measured as discussed above.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence identity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence identity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence identity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further includes a 5′ cap. In some embodiments, the linear counterpart further includes a poly adenosine tail. In some embodiments, the linear counterpart further includes a 3′ UTR. In some embodiments, the linear counterpart further includes a 5′ UTR.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization through, for example, splint ligation, or chemical, enzymatic, ribozyme- or splicing-catalyzed circularization methods.

As used herein, the term “mixture” means a material made of two or more different substances that are mixed. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, a liquid solution is a liquid-in-liquid solution (e.g., a liquid solute dissolved in a liquid solvent), a solid-in-liquid solution (e.g., a solid solute dissolved in a liquid solvent), or a gas-in-liquid solution (e.g., a solid solute dissolved in a liquid solvent). In some cases, there is more than one solvent and/or more than one solute. In some cases, a mixture is a colloid, liquid suspension, or emulsion. In some cases, a mixture is a solid mixture, e.g., the mixture is present in solid phase.

As used herein, the term “modified ribonucleotide” means a nucleotide with at least one modification to the sugar, the nucleobase, or the internucleoside linkage.

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular or linear polyribonucleotide is a formulation that includes a circular or linear polyribonucleotide without covalent modification and is free from a carrier.

As used herein, the terms “nicked RNA” or “nicked linear polyribonucleotide” or “nicked linear polyribonucleotide molecule” are used interchangeably and mean a polyribonucleotide molecule having a 5′ and 3′ end that results from nicking or degradation of a circular RNA.

As used herein, the term “non-circular RNA” means total nicked RNA and linear RNA.

The terms “obtainable by”, “producible by” or the like are used to indicate that a claim or embodiment refers to compound, composition, product, etc. per se, i.e. that the compound, composition, product, etc. can be obtained or produced by a method which is described for manufacture of the compound, composition, product, etc., but that the compound, composition, product, etc. may be obtained or produced by other methods than the described one as well. The terms “obtained by”, “produced by” or the like indicate that the compound, composition, product, is obtained or produced by a recited specific method. It is to be understood that the terms “obtainable by”, “producible by” and the like also disclose the terms “obtained by”, “produced by” and the like as a preferred embodiment of “obtainable by”, “producible by” and the like.

As used herein, the term “pathogen” refers to an infectious agent, which causes disease or disease symptoms in a subject, for example, by directly infecting the subject, by producing agents that cause disease or disease symptoms in the subject, and/or by eliciting an immune response in the subject.

As used herein, pathogens include, but are not limited to bacteria, protozoa, parasites, fungi, nematodes, insects, viroids, and viruses, or any combination thereof, wherein each pathogen is capable, either by itself or in concert with another pathogen, of eliciting disease or symptoms a subject.

As used herein, the term “payload” means any molecule delivered by the polyribonucleotide as disclosed herein. In some cases, a payload is a nucleic acid, a protein, a chemical, a ribonucleoprotein, or any combination thereof. In some cases, a payload is a nucleic acid sequence directly contained within the polyribonucleotide as disclosed herein. In some cases, a payload is attached to or associated with the polyribonucleotide as disclosed herein, for instance via complementary hybridization, or via protein-nucleic acid interactions. In certain cases, the payload is a protein encoded by a nucleic acid sequence contained within, attached to, or associated with the polyribonucleotide. In some cases, the “attachment” means covalent bond or non-covalent interaction between two molecules. In some cases, the “association” when used in the context of the interaction between a payload and a polyribonucleotide means that the payload is indirectly linked to the polyribonucleotide via one or more other molecules in between. In some cases, the attachment or association can be transient. In some cases, a payload is attached to or associated with the polyribonucleotide under one condition but not under another condition, for instance, depending on the ambient pH condition or the presence or absence of a stimulus or a binding partner.

The term “pharmaceutical composition” is intended to also disclose that the circular or linear polyribonucleotide included within a pharmaceutical composition can be used for the treatment of the human or animal body by therapy. It is thus meant to be equivalent to the “a circular or linear polyribonucleotide for use in therapy”.

The term “polynucleotide” as used herein means a molecule comprising one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 Jul.; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer, or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “prevent,” means to reduce the likelihood of developing a disease, disorder, or condition, or alternatively, to reduce the severity of a subsequently developed disease or disorder. A therapeutic agent can be administered to a subject who is at increased risk of developing a disease or disorder relative to a member of the general population in order to prevent the development of, or lessen the severity of, the disease or condition. A therapeutic agent can be administered as a prophylactic, e.g., before development of any symptom or manifestation of a disease or disorder.

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular or linear polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motif useful for replication or that initiate transcription of the circular polyribonucleotide.

As used herein, the term “surface area” of a subject body means any area of a subject that is or has a potential to be exposed to an exterior environment subject body. A surface area of a subject body, e.g., a mammal body, e.g., a human body, can include skin, surface areas of oral cavity, nasal cavity, ear cavity, gastrointestinal tract, respiratory tract, vaginal, cervical, inter uterine, urinary tract, and eye. In some cases, a surface area of a subject body can often refer to the outer area under which epithelial cells are lined up. Skin, for example, can be one type of surface area as discussed herein and can be composed of epidermis and dermis, the former of which forms the outermost layers of kin and can include organized assembly of epithelial cells among many other types of cells.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPG P, where x=any amino acid. In some embodiments, the stagger element may include a chemical moiety, such as glycerol, a non-nucleic acid linking moiety, a chemical modification, a modified nucleic acid, or any combination thereof.

As used herein, the term “substantially free” is the level of a component in a composition, preparation, or product, or any intermediate thereof that is lower than the level required to induce a biological, chemical, physical, and/or pharmacological effect. In some embodiments, a composition, preparation, or product is substantially free of a component if the level of the component is detectable only in trace amounts or the level is less than the level detectable by a relevant detection technique (e.g., chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre or post separation derivatization methodologies using detection techniques based on mass spectrometry, UV-visible, fluorescence, light scattering, refractive index, or that use silver or dye stains or radioactive decay for detection. Alternatively, whether a composition, preparation, or product is substantially free of a component may be determined without the use of separation technologies by mass spectrometry, by microscopy, by circular dichroism (CD) spectroscopy, by UV or UV-vis spectrophotometry, by fluorometry (e.g., Qubit), by RNase H analysis, by surface plasmon resonance (SPR), or by methods that utilize silver or dye stains or radioactive decay for detection).

As used herein, the term “substantially resistant” is one that has at least 50%, 55%, 60%, 65%, 70%, 75%, 80%, 85%, 90%, 95%, 96%, 97%, 98% or 99% resistance to an effector as compared to a reference.

As used herein, the term “sterilizing agent” means any agent that is bacteriostatic, bactericidal, and/or actively kills microorganisms, inactivates microorganisms, or prevents microorganisms from growing. A sterilizing agent that kills microorganisms can be antimicrobial and/or antiseptic. In some embodiments, the sterilizing agent is a liquid, such as an alcohol, iodine, or hydrogen peroxide. In some embodiments, the sterilizing agent, is UV light or a laser light. In some embodiments, the sterilizing agent is heat delivered electrically or through other means (e.g., vapor, contact).

As used herein, the term “stoichiometric translation” is a substantially equivalent production of expression products translated from the circular or linear polyribonucleotide. For example, for a circular or linear polyribonucleotide having two expression sequences, stoichiometric translation of the circular or linear polyribonucleotide means that the expression products of the two expression sequences have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%, or any percentage therebetween.

As used herein, the term “systemic delivery” or “systemic administration” means a route of administration of pharmaceutical compositions or other substances into the circulatory system (e.g., blood or lymphoid system). The systemic administration can include oral administration, parenteral administration, intranasal administration, sublingual administration, rectal administration, transdermal administration, or any combinations thereof. As used herein, the term “non-systemic delivery” or “non-systemic administration” can refer to any other routes of administration than systemic delivery of pharmaceutical compositions or other substances, e.g., the delivered substances do not enter the circulation systems (e.g., blood and lymphoid system) of the subject body.

As used herein, the term “sequence identity” is determined by alignment of two peptide or two nucleotide sequences using a global or local alignment algorithm. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively or additionally, percent identity may be determined by searching against databases, using algorithms such as FASTA, BLAST, etc. Sequence identity refers to the sequence identity over the entire length of the sequence.

A “signal sequence” refers to a polypeptide sequence, e.g., between 10 and 30 amino acids in length, that is present at the N-terminus of a polypeptide sequence of a nascent protein which targets the polypeptide sequence to the secretory pathway.

As used herein, the term “target” refers to any entity that includes one or more epitopes. For example, a target may be a chemical moiety, a portion of a molecule, a molecule (e.g., an allergen or a toxin), a macromolecule (e.g., a polypeptide, a nucleic acid, or carbohydrate), a post-translational modification state of a macromolecule (e.g., a macromolecule that is phosphorylated, glycosylated, acylated, alkylated, and the like), a higher-order macromolecular structure (e.g., a complex of two or more polypeptides), a cell (e.g., a cancer cell), a portion of a cell (e.g., a tumor antigen), a receptor on the surface of a cell, a pathogen (e.g., a virus or a portion or a virus; a bacterium or a portion of a bacterium; a fungus or a portion of a fungus; or a parasite or a portion of a parasite), or a tissue-type.

As used herein, the term “treat,” or “treating,” refers to a therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment.

As used herein, the term “termination element” is a moiety, such as a nucleic acid sequence, that terminates translation of the expression sequence in the circular or linear polyribonucleotide.

As used herein, the term “topical delivery” means delivery of a substance to skin or an epithelial layer accessible though non-invasive means, e.g., the intestinal and other gastrointestinal (GI) epithelia or the vaginal epithelium. Topical delivery of a pharmaceutical composition can have a local pharmacodynamic effect on the subject, e.g., the topically delivered pharmaceutical composition has a pharmacodynamic effect at or proximate to the particular part of the body (e.g. skin) where the pharmaceutical composition is delivered. In some other embodiments, topical delivery of a pharmaceutical composition as discussed herein is used only to refer to the delivery mode (locally to, e.g., a specific surface area), whereas the pharmaceutical composition can have either a local or systemic pharmacodynamic effect. For instance, the pharmaceutical composition can either stay local at or proximate to the administration site, or can enter a circulation system (e.g., blood or lymphoid system) of the subject body, through which the pharmaceutical composition can be transported to remote parts of the body that are typically not reachable by the pharmaceutical composition via routes other than the circulation systems.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “translation initiation sequence” is a nucleic acid sequence that initiates translation of an expression sequence in the circular or linear polyribonucleotide.

### BRIEF DESCRIPTION OF THE DRAWINGS

FIGS. 1A-1B are schematics of exemplary circular RNAs that include two expression sequences, where each expression sequence encodes either an immunogen or an adjuvant. FIG. 1A depicts a circular polyribonucleotide that includes two open reading frames (ORFs), each ORF encoding an expression sequence, where each ORF is operably linked to an IRES. FIG. 1B depicts a circular polyribonucleotide that includes two expression sequences separated by a 2A sequence, all operably linked to an IRES.

FIG. 2 shows a schematic of a circular RNA that includes an ORF that encodes an immunogen and a polynucleotide adjuvant sequence (e.g., a nucleotide sequence that stimulates the innate immune system).

FIG. 3 shows a schematic of a plurality of polyribonucleotides, where each polynucleotide includes an ORF that encodes either an immunogen or an adjuvant.

FIG. 4 shows an RBD immunogen encoded by a circular RNA was detected in BJ Fibroblasts and HeLa cells and was not detected in BJ Fibroblasts and HeLa cells with the vehicle control.

FIG. 5 shows that sustainable anti-RBD antibody response was attained following administration of a circular RNA encoding a SARS-CoV-2 RBD immunogen, formulated with a cationic polymer (e.g., protamine), in a mouse model.

FIG. 6 shows that an anti-Spike response was attained following administration of a circular RNA encoding a SARS-CoV-2 RBD antigen, formulated with a cationic polymer (e.g., protamine), in a mouse model.

FIG. 7 shows anti-RBD IgG2a and IgG1 isotype levels that were obtained after administration of a circular RNA encoding a SARS-CoV-2 RBD immunogen, formulated with a cationic polymer (e.g. protamine), in a mouse model.

FIG. 8 shows protein expression from circular RNA in vivo for prolonged periods of time after intramuscular injection of circular RNA preparations (Trans-IT formulated, protamine formulated, unformulated), protamine vehicle only, and uninjected control mice.

FIG. 9 shows protein expression from circular RNA in vivo for prolonged periods of time after simultaneous intramuscular delivery of Addavax™ adjuvant with (i) unformulated circular RNA preparations (left graph), (ii) circular RNA formulated with TransIT (middle graph), and (iii) circular RNA formulated with protamine (right graph). In each case, Addavax™ adjuvant was delivered as an individual injection at 0 and 24 h.

FIG. 10 shows protein expression from circular RNA in vivo for prolonged periods of time after intradermal delivery of (i) circular RNA formulated with protamine, (ii) circular RNA formulated with protamine, with an injection of Addavax™ adjuvant at 24 hours, (iii) protamine vehicle only, and (iv) an uninjected control mice.

FIG. 11 shows the binding of probes to circular and linear RNA and subsequent degradation of the RNA by RNase H. Circular RNA is detected as a single cleaved linear band compared to linear and concatemeric RNA, which is detected as multiple bands. Degradation was detected by running samples on a denaturing polyacrylamide gel and comparing degradation bands with or without addition of RNase H.

FIG. 12 shows linear RNA content quantified on a denaturing polyacrylamide gel by comparing linear RNA band intensity to a linear RNA standard.

FIG. 13 shows the quantification of RNA extracted from different bands of a denaturing polyacrylamide gel.

FIG. 14 shows Gaussia Luciferase activity in cells at 6, 24, 48, 72, 96, and 120 hours post-transfection in experiments using circular RNA of 84% purity, circular RNA of 71% purity, and vehicle only.

FIG. 15 shows cells transfected with the gel purified circular RNA preparation only showed minimal expression of innate immune genes such as RIG-I, MDA-5, OAS, and IFN-B compared to cells transfected with both the combined circular RNA and linear counterpart RNA, which exhibited upregulation of these innate immune genes in a dose dependent manner.

FIG. 16 is an image showing a protein blot of expression products from circular RNA or linear RNA with a stagger element.

FIG. 17 shows generation of exemplary circular RNA by self-splicing.

FIG. 18 illustrates an example in which both exemplary linear and circular RNA were delivered topically to the ear skin of mice and their RNA levels in the ear tissue were examined over the 6 hours to 3 days after delivery period.

FIGS. 19A and 19B are plots summarizing qPCR results from ear punches of mice 6 hours, 1 day, 3 days, or 12 days after topical delivery of linear or circular RNAs with the aid of Boost (ethanol).

FIG. 19A shows results from qPCR assays using primers that for detection of both the linear and circular RNAs. FIG. 19B shows results from qPCR assays using primers for detection of the circular RNA, but not the linear RNA.

FIG. 20 shows fluorescent images (B/W) from topical administration of circRNA-Cy5 results in RNA delivery to tissue.

FIG. 21 shows quantification of fluorescent images from topical administration of circRNA-Cy5 results in RNA delivery to tissue.

FIG. 22 shows topical administration of circular RNA results in RNA delivery to tissue at day 1 and day 4 after administration when tissue is wiped with an ethanol wipe prior to application.

FIG. 23 shows topical administration of circular RNA results in RNA delivery to tissue at day 1 and day 4 after administration when the tissue is wiped with an isopropyl alcohol wipe prior to application.

FIG. 24 shows topical administration of circRNA results in RNA delivery to tissue when circRNA is administered with 10% ethanol.

FIG. 25 is an image showing a protein blot of expression products from circular RNA or linear RNA.

FIG. 26 shows experimental data demonstrating increased persistence of Gaussia luciferase expression in mice following redosing with a circular polyribonucleotide (“Endless”) as compared to a linear polyribonucleotide counterpart (“Linear”).

FIG. 27 shows experimental data demonstrating increased persistence of Gaussia luciferase expression in mice following staggered dosing with a circular polyribonucleotide (“Endless 3 doses”) as compared to staggered dosing a linear polyribonucleotide counterpart (“Linear 3 doses”), or a single dose with the circular polyribonucleotide (“Endless”), or a single dose with a linear polyribonucleotide counterpart (“Linear”).

FIG. 28 shows experimental data demonstrating increased persistence of Gaussia luciferase expression in mice following a single dose of a circular polyribonucleotide (“Endless RNA”) as compared to a single dose of a linear polyribonucleotide counterpart (“Linear RNA”), staggered dosing with a linear polyribonucleotide counterpart (“3 doses Linear RNA”) as compared to a single dose (“Linear RNA”), or staggered dosing with a circular polyribonucleotide (“3 doses Endless RNA”) as compared a single dose (“Endless RNA”).

FIG. 29 shows circular polyribonucleotide administered intramuscularly, without a carrier, expressed protein in vivo for prolonged periods of time, with levels of protein activity in the plasma at multiple days post injection.

FIG. 30 shows circular polyribonucleotide administered intravenously, expressed protein in vivo for prolonged periods of time, with levels of protein activity in the plasma at multiple days post injection and could be re-dosed at least 5 times.

FIG. 31A shows multi-immunogen expression from a circular polyribonucleotide. RBD immunogen expression was detected from circular RNAs encoding a SARSs-CoV-2 RBD immunogen and a GLuc polypeptide.

FIG. 31B shows multi-immunogen expression from a circular polyribonucleotide. GLuc activity was detected from circular RNAs encoding a SARSs-CoV-2 RBD immunogen and a GLuc polypeptide.

FIG. 32A shows immunogenicity of multiple immunogens from circular RNAs in mouse model. Mice were vaccinated with a first circular RNA encoding a SARS-CoV-2 RBD immunogen and a second circular RNA encoding a GLuc polypeptide. Anti-RBD antibodies were obtained at 17 days after injection.

FIG. 32B shows immunogenicity of multiple immunogens from circular RNAs in mouse model.

Mice were vaccinated with a first circular RNA encoding a SARS-CoV-2 RBD immunogen and a second circular RNA encoding a GLuc polypeptide. GLuc activity was detected at 2 days after injection.

FIG. 33A shows immunogenicity of multiple immunogens from circular RNAs in mouse model.

Mice were vaccinated with a first circular RNA encoding a SARS-CoV-2 RBD immunogen and a second circular RNA encoding Influenza hemagglutinin (HA) immunogen. Anti-RBD antibodies were obtained at 17 days after injection.

FIG. 33B shows immunogenicity of multiple immunogens from circular RNAs in mouse model.

Mice were vaccinated with a first circular RNA encoding a SARS-CoV-2 RBD immunogen and a second circular RNA encoding Influenza hemagglutinin (HA) immunogen. Anti-HA antibodies were obtained at 17 days after injection.

FIG. 34A shows immunogenicity of multiple immunogens from circular RNAs in mouse model.

Mice were vaccinated with a first circular RNA encoding a SARS-CoV-2 Spike immunogen and a second circular RNA encoding Influenza hemagglutinin (HA) immunogen. Anti-RBD (domain of Spike) antibodies were obtained at 17 days after injection.

FIG. 34B shows immunogenicity of multiple immunogens from circular RNAs in mouse model.

Mice were vaccinated with a first circular RNA encoding a SARS-CoV-2 Spike immunogen and a second circular RNA encoding Influenza hemagglutinin (HA) immunogen. Anti-HA antibodies were obtained at 17 days after injection.

FIG. 35 shows an anti-HA antibody response in mice administered circular RNA encoding multiple immunogens. Mice were administered a circular RNA encoding: a SARS-CoV-2 RBD immunogen, a SARS-CoV-2 Spike immunogen, an Influenza HA immunogen, a SARS-CoV-2 RBD immunogen and an Influenza HA immunogen, a SARS-CoV-2 RBD immunogen and a GLuc polypeptide, or a SARS-CoV-2 RBD immunogen and a SARS-CoV-2 Spike immunogen. A hemagglutination inhibition assay (HAI) was used to measure anti-Influenza HA antibodies. FIG. 24 shows HAI titer in samples that were administered circular RNA preparations encoding the Influenza HA immunogen when it was administered alone or when administered in combination with SARS-CoV-2 immunogens, e.g., RBD or Spike.

FIG. 36 shows Gaussia luciferase (Gluc) activity in mice at 2 days post-prime with: (i) a circular RNA encoding a Gluc polypeptide, or (ii) a combination of a first circular RNA encoding a Gluc polypeptide and a second circular RNA encoding a SARS-CoV-2 RBD immunogen, or (iii) a circular RNA encoding both a Gluc polypeptide and a SARS-CoV-2 RBD immunogen.

FIG. 37A shows anti-RBD antibody response in mice at 14 days post-prime with: (i) a circular RNA encoding a SARS-CoV-2 RBD immunogen, or (ii) a combination of a first circular RNA encoding a GLuc polypeptide and a second circular RNA encoding a SARS-CoV-2 RBD immunogen, or (iii) a circular RNA encoding both a Gluc polypeptide and a SARS-CoV-2 RBD immunogen.

FIG. 37B shows anti-RBD neutralizing antibodies, as evaluated by a Plaque Reduction Neutralization Test (PRNT), in mice administered: (i) a circular RNA encoding a SARS-CoV-2 RBD immunogen, or (ii) a combination of a first circular RNA encoding a GLuc polypeptide and a second circular RNA encoding a SARS-CoV-2 RBD immunogen, or (iii) a circular RNA encoding both a Gluc polypeptide and a SARS-CoV-2 RBD immunogen.

FIG. 38 shows a circular RNA encoding a Gluc polypeptide and a SARS-CoV-2 RBD immunogen primes RBD-specific T cell responses as determined by ELISpot assay.

FIG. 39 shows IL-12, measured using an IL-12 specific ELISA, was expressed from circular RNA in mammalian cells. A circular RNA encoding a SARS-CoV-2 RBD immunogen was included as a negative control.

FIG. 40A shows IL-12 expression was detected in serum at 2 days after injection with a circular RNA preparation including a first circular RNA encoding IL-12 and a second circular RNA encoding a SARS-CoV-2 RBD immunogen, in a mouse model. Injection with PBS or with a preparation including only the circular RNA encoding a SARS-CoV-2 RBD immunogen were included as controls.

FIG. 40B shows an increase in serum IFN-γ (directly downstream of IL12 signaling) was detected in serum at 2 days after injection with a circular RNA preparation including a first circular RNA encoding IL-12 and a second circular RNA encoding a SARS-CoV-2 RBD immunogen, in a mouse model. Injection with PBS or with a preparation including only the circular RNA encoding a SARS-CoV-2 RBD immunogen were included as controls.

FIG. 41A shows that administration of a circular RNA preparation including a first circular RNA encoding IL-12 and a second circular RNA encoding a SARS-CoV-2 RBD immunogen increased the number of SARS-CoV-2 RBD specific CD4 T cells. Administration of PBS or a preparation including only the circular RNA encoding a SARS-CoV-2 RBD immunogen were included as controls. Asterisks denotes statistical significance as determined by a two-way RM ANOVA protected Tukey's post hoc test.

FIG. 41B shows that administration of a circular RNA preparation including a first circular RNA encoding IL-12 and a second circular RNA encoding a SARS-CoV-2 RBD immunogen produced no change in the number of RBD specific CD8 T cells. Administration of PBS or a preparation including only the circular RNA encoding a SARS-CoV-2 RBD immunogen were included as controls.

FIG. 41C shows that administration of a circular RNA preparation including a first circular RNA encoding IL-12 and a second circular RNA encoding SARS-CoV-2 RBD immunogen increased the amount of IFN-γ production by CD4 T cells. Administration of PBS or a preparation including only the circular RNA encoding a SARS-CoV-2 RBD immunogen were included as controls. Asterisks denotes statistical significance as determined by unpaired t-test.

FIG. 41D shows that administration of a circular RNA preparation including a first circular RNA encoding IL-12 and a second circular RNA encoding a SARS-CoV-2 RBD immunogen increased the amount of IFN-γ production by CD8 T cells. Administration of PBS or a preparation including only the circular RNA encoding a SARS-CoV-2 RBD immunogen were included as controls. Asterisks denotes statistical significance as determined by unpaired t-test.

FIG. 42 shows that a circular RNA expressing Ovalbumin (OVA) combined with circular RNA expressing IL12 induces a stronger CD8 T cell response when compared to the control (circular RNA expressing OVA only). This demonstrates that circular RNA expressing IL12 acts as an adjuvant.

### DETAILED DESCRIPTION

This disclosure provides compositions and pharmaceutical preparations of circular or linear polyribonucleotides encoding one or more polypeptide immunogens and uses thereof. In particular, the disclosure provides circular or linear polyribonucleotides encoding multiple immunogens, circular or linear polyribonucleotides encoding at least one immunogen and further encoding at least one adjuvant, and immunogenic compositions including multiple circular or linear polyribonucleotides. This disclosure further features pharmaceutical compositions and preparations including one or more circular or linear polyribonucleotides encoding one or more immunogens. Compositions and pharmaceutical preparations of circular or linear polyribonucleotides described herein may induce an immune response in a subject upon administration. Compositions and pharmaceutical preparations of circular or linear polyribonucleotides described herein may be used to treat or prevent a disease, disorder, or condition in a subject.

### Polyribonucleotides

The polyribonucleotide includes the elements as described below as well as the in addition to one or more immunogens as described herein. In particular embodiments, the polyribonucleotide is a circular polyribonucleotide.

In some embodiments, the polyribonucleotide (e.g., the circular polyribonucleotide) is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the polyribonucleotide (e.g. the circular polyribonucleotide) may be of a sufficient size to accommodate a binding site for a ribosome. In some embodiments, the maximum size of a circular polyribonucleotide can be as large as is within the technical constraints of producing a circular polyribonucleotide, and/or using the circular polyribonucleotide. Without wishing to be bound by any particular theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, such as immunogens or an epitopes thereof of the disclosure, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful. In some embodiments, the maximum size of the circular polyribonucleotide is a length sufficient to encode one or more immunogens (e.g., two or more, three or more, four or more, and five or more). In some embodiments, the maximum size of the circular polyribonucleotide is a length sufficient to encode between two and five (e.g., three, four, and five) immunogens. In some embodiments, the maximum size of the circular polyribonucleotide is a length sufficient to encode an immunogen and an adjuvant. In some embodiments, the maximum size of the circular polyribonucleotide is a length sufficient to encode one or more immunogens (e.g., two or more, three or more, and four or more) and one or more adjuvants (e.g., two or more, three or more, and four or more).

### Circular Polyribonucleotide Elements

In some embodiments, the circular polyribonucleotide includes one or more of the elements as described herein in addition to including a sequence encoding an immunogen. In some embodiments, the circular polyribonucleotide lacks a poly-A sequence, lacks a free 3′ end, lacks an RNA polymerase recognition motif, or any combination thereof. In some embodiments, the circular polyribonucleotide includes any feature or any combination of features as disclosed in International Patent Publication No. WO2019/118919, which is hereby incorporated by reference in its entirety.

### Immunogens

The circular or linear polyribonucleotides described herein includes at least one sequence encoding an immunogen and optionally further includes at least one sequence encoding an adjuvant. An immunogen includes one or more epitopes that is recognized, targeted, or bound by a given antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope includes nucleic acids that form an epitope in the folded conformation of an immunogenic sequence based on its secondary structure or tertiary structure.

In some embodiments, an immunogen includes all or a part of a protein, a peptide, a glycoprotein, a lipoprotein, a phosphoprotein, a ribonucleoprotein, a carbohydrate (e.g., a polysaccharide), a lipid (e.g., a phospholipid or triglyceride), or a nucleic acid (e.g., DNA, RNA).

In other embodiments, an immunogen includes a protein immunogen or epitope (e.g., a peptide immunogen or peptide epitope from a protein, glycoprotein, lipoprotein, phosphoprotein, or ribonucleoprotein). An immunogen can include an amino acid, a sugar, a lipid, a phosphoryl, or a sulfonyl group, or a combination thereof.

In a particular embodiment, the immunogen is a polypeptide immunogen.

A polypeptide immunogen may include a post-translational modification, for example, glycosylation, ubiquitination, phosphorylation, nitrosylation, methylation, acetylation, amidation, hydroxylation, sulfation, or lipidation.

In some embodiments, an immunogen includes an epitope including at least 4, at least 5, at least 6, at least 7, at least 8, at least 9, at least 10, at least 11, at least 12, at least 13, at least 14, at least 15, at least 16, at least 17, at least 18, at least. 19, at least 20, at least 21, at least 22, at least 23, at least 24, at least 25, at least 26, at least 27, at least 28, at least 29, or at least 30 amino acids, or more. In some embodiments, an epitope includes or contains at most 4, at most 5, at most 6, at most 7, at most 8, at most 9, at most 10, at most 11, at most 12, at most 13, at most 14, at most 15, at most 16, at most 17, at most 18, at most. 19, at most 20, at most 21, at most 22, at most 23, at most 24, at most 25, at most 26, at most 27, at most 28, at most 29, or at most 30 amino acids, or less. In some embodiments, an epitope includes or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, an epitope contains 5 amino acids. In some embodiments, an epitope contains 6 amino acids. In some embodiments, an epitope contains 7 amino acids. In some embodiments, an epitope contains 8 amino acids. In some embodiments, an epitope can be about 8 to about 11 amino acids. In some embodiments, an epitope can be about 9 to about 22 amino acids.

The immunogens may include immunogens recognized by B cells, immunogens recognized by T cells, or a combination thereof. In some embodiments, the immunogens include immunogens recognized by B cells. In some embodiments, the immunogens are immunogens recognized by B cells. In some embodiments, the immunogens include immunogens recognized by T cells. In some embodiments, the immunogens are immunogens recognized by T cells.

The epitopes may include epitopes recognized by B cells, epitopes recognized by T cells, or a combination thereof. In some embodiments, the epitopes include epitopes recognized by B cells. In some embodiments, the epitopes are epitopes recognized by B cells. In some embodiments, the epitopes include epitopes recognized by T cells. In some embodiments, the epitopes are epitopes recognized by T cells.

Techniques for identifying immunogens and epitopes in silico have been disclosed, for example, in Sanchez-Trincado J L, et al. (Fundamentals and methods for T- and B-cell epitope prediction, J. Immunol. Res., 2017:2680160. doi: 10.1155/2017/2680160 (2017)); Grifoni, A, et al. (A Sequence Homology and Bioinformatic Approach Can Predict Candidate Targets for Immune Responses to SARS-CoV-2, Cell Host Microbe, 27(4):671-680 (2020)); Russi R C et al. (In silico prediction of epitopes recognized by T cells and B cells in PmpD: First step towards to the design of a Chlamydia trachomatis vaccine, Biomedical J., 41(2):109-117 (2018)); Baruah V, et al. (Immunoinformatics-aided identification of T cell and B cell epitopes in the surface glycoprotein of 2019-nCoV, J. Med. Virol., 92(5), doi: 10.1002/jmv.25698 (2020)); each of which is incorporated herein by reference in its entirety.

In some embodiments, an immunogen includes a polynucleotide. In some embodiments, an immunogen is a polynucleotide. In some embodiments, an immunogen includes an RNA. In some embodiments, an immunogen is an RNA. In some embodiments, an immunogen includes a DNA. In some embodiments, an immunogen is a DNA. In some embodiments, the polynucleotide is encoded in the circular or linear polyribonucleotide.

A circular or linear polyribonucleotide of the disclosure includes or encodes any number of immunogens. In a particular embodiment, a circular or linear polyribonucleotide includes or encodes at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more of immunogens.

In some embodiments, a circular or linear polyribonucleotide includes or encodes, for example, at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less immunogens.

In some embodiments, a circular or linear polyribonucleotide includes or encodes about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 of immunogens.

In some embodiments, the circular or linear polyribonucleotide encodes a plurality of immunogens. In some embodiments, a circular or linear polyribonucleotide includes or encodes between 1 and 100 immunogens. In some embodiments, a circular or linear polyribonucleotide includes or encodes between 1 and 50 immunogens. In some embodiments, a circular or linear polyribonucleotide includes or encodes between 1 and 10 immunogens; for example, a circular or linear polyribonucleotide encodes 1, 2, 3, 4, 5, 6, 7, 8, 9, or 10 immunogens. In some embodiments, a circular or linear polyribonucleotide includes or encodes 2 immunogens. In some embodiments, a circular or linear polyribonucleotide includes or encodes 3 immunogens. In some embodiments, a circular or linear polyribonucleotide includes or encodes 4 immunogens. In some embodiments, a circular or linear polyribonucleotide includes or encodes 5 immunogens.

In some embodiments, the plurality of immunogens each identify the same target. Otherwise put, a single target may include each of the plurality of immunogens, each of the plurality of immunogens may be derived from the same target, and/or each of the plurality of immunogens may share a high degree of similarity with a portion or the whole of the target. For example, a target may be a cell and each of the immunogens may correspond to a protein of that cell. For example, the target may a particular cancer cell and each of the immunogens may correspond to a tumor antigen associate with that cancer. Accordingly, in some embodiments, each of the plurality of immunogens are derived from different proteins from the same target.

In some embodiments, the plurality of immunogens are derived from different targets. In some embodiments, the plurality of immunogens may be derived various capsid proteins of a given virus. For example, the one immunogen may be derived from Orthopoxvirus, another immunogen may be derived Hepadnavirus, and a third immunogen may be derived Flavivirus. For example, a polyribonucleotide may encode multiple immunogens, where each immunogen is derived from yellow fever virus, Chikungunya virus, Zika, Hepatitis A, or Hepatitis B. A polyribonucleotide may encode an immunogen from each of yellow fever virus, Chikungunya virus, Zika, Hepatitis A, and Hepatitis B. A polyribonucleotide may encode multiple immunogens, where each immunogen is derived from Japanese encephalitis, Chikungunya virus, Zika, Hepatitis A, or Hepatitis B. A polyribonucleotide may encode an immunogen from each of Japanese encephalitis, Chikungunya virus, Zika, Hepatitis A, and Hepatitis B. A polyribonucleotide may encode multiple immunogens, where each immunogen is derived from SARS-CoV2, a poxvirus, respiratory syncytial virus, or human papilloma virus. A polyribonucleotide may encode an immunogen from each of SARS-CoV2, a poxvirus, respiratory syncytial virus, and human papilloma virus. A polyribonucleotide may encode multiple immunogens, where each immunogen is derived from a herpes virus (CMV, EBV, or VZV). A polyribonucleotide may encode an immunogen from each of the following herpes viruses: CMV, EBV, or VZV. A polyribonucleotide may encode multiple immunogens, where each immunogen is derived Singles or West Nile Virus. A polyribonucleotide may encode an immunogen from each of Shingles and West Nile Virus.

In some embodiments, the plurality of immunogens have at least 90%, 91%, 92%, 93%, 94%, 95%, 96%, 97%, 98%, or 99% sequence identity. In some embodiments, the plurality of immunogens also have less than 100% sequence identity. This may be indicative of immunogens related to one another by genetic drift, as such, a single circular or linear polyribonucleotide composition or immunogenic composition may be able to induce an immune response against a target that exists in various mutational states in a population or may induce an immune response against multiple targets having the same immunogen where the immunogen is related by genetic drift. For example, the immunogens may be related to one another by genetic drift of a target virus. In some embodiments, the plurality of immunogens may be derived from receptor-binding domains (RBD) from unique but related viruses.

In some embodiments, a circular or linear polyribonucleotide encodes variants of an immunogen. Variants can be naturally-occurring variants (for example, variants identified in sequence data from different viral genera, species, isolates, or quasispecies), or can be derivative sequences as disclosed herein that have been generated in silico (for example, immunogens or epitopes with one or more amino acid insertions, deletions, substitutions, or a combination thereof compared to a wild-type immunogen or epitope).

An immunogen is from, for example, a virus, such as a viral surface protein, a viral membrane protein, a viral envelope protein, a viral capsid protein, a viral nucleocapsid protein, a viral spike protein, a viral entry protein, a viral membrane fusion protein, a viral structural protein, a viral non-structural protein, a viral regulatory protein, a viral accessory protein, a secreted viral protein, a viral polymerase protein, a viral DNA polymerase, a viral RNA polymerase, a viral protease, a viral glycoprotein, a viral fusogen, a viral helical capsid protein, a viral icosahedral capsid protein, a viral matrix protein, a viral replicase, a viral transcription factor, or a viral enzyme.

In some embodiments, the immunogen is from one of these viruses:

Orthomyxovirus: Useful immunogens can be from an influenza A, B or C virus, such as the hemagglutinin, neuraminidase, or matrix M2 proteins. Where the immunogen is an influenza A virus hemagglutinin it may be from any subtype e.g. H1, H2, H3, H4, H5, H6, H7, H8, H9, H10, H11, H12, H13, H14, H15, or H16.

Paramyxoviridae viruses: Viral immunogens include, but are not limited to, those derived from Pneumoviruses (e.g. respiratory syncytial virus (RSV)), Rubulaviruses (e.g. mumps virus), Paramyxoviruses (e.g. parainfluenza virus), Metapneumoviruses and Morbilliviruses (e.g. measles virus), Henipaviruses (e.g. Nipah virus).

Poxviridae: Viral immunogens include, but are not limited to, those derived from Orthopoxvirus such as Variola vera, including but not limited to, Variola major and Variola minor.

Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.

Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: Viral immunogens include, but are not limited to, those derived from a Heparnavirus, such as hepatitis A virus (HAV).

Filovirus: Viral immunogens include, but are not limited to, those derived from a filovirus, such as an Ebola virus (including a Zaire, Ivory Coast, Reston, or Sudan ebolavirus) or a Marburg virus.

Togavirus: Viral immunogens include, but are not limited to, those derived from a Togavirus, such as a Rubivirus, an Alphavirus, or an Arterivirus. This includes rubella virus.

Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus, Zika virus.

Pestivirus: Viral immunogens include, but are not limited to, those derived from a Pestivirus, such as Bovine viral diarrhea (BVDV), Classical swine fever (CSFV) or Border disease (BDV).

Hepadnavirus: Viral immunogens include, but are not limited to, those derived from a Hepadnavirus, such as Hepatitis B virus. The hepatitis B virus immunogen may be a hepatitis B virus surface immunogen (HBsAg).

Other hepatitis viruses: Viral immunogens include, but are not limited to, those derived from a hepatitis C virus, delta hepatitis virus, hepatitis E virus, or hepatitis G virus.

Rhabdovirus: Viral immunogens include, but are not limited to, those derived from a Rhabdovirus, such as a Lyssavirus {e.g. a Rabies virus) and Vesiculovirus (VSV).

Caliciviridae: Viral immunogens include, but are not limited to, those derived from Calciviridae, such as Norwalk virus (Norovirus), and Norwalk-like Viruses, such as Hawaii Virus and Snow Mountain Virus.

Retrovirus: Viral immunogens include, but are not limited to, those derived from an Oncovirus, a Lentivirus (e.g. HIV-1 or HIV-2) or a Spumavirus.

Reovirus: Viral immunogens include, but are not limited to, those derived from an Orthoreovirus, a Rotavirus, an Orbivirus, or a Coltivirus.

Parvovirus: Viral immunogens include, but are not limited to, those derived from Parvovirus B19.

Bocavirus: Viral immunogens include, but are not limited to, those derived from bocavirus.

Herpesvirus: Viral immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.

Orthohantaviruses: Viral immunogens include, but are not limited to, those derived from hantaviruses.

Arenavirus: Viral immunogens include, but are not limited to, those derived from Guanarito virus, Junin virus, Lassa virus, Lujo virus, Machupo virus, Sabia virus, or Whitewater Arroyo virus.

Adenovirus: Viral immunogens include those derived from adenovirus serotype 36 (Ad-36).

Community acquired respiratory viruses: Viral immunogens include those derived from community acquired respiratory viruses.

Coronavirus: Viral immunogens include, but are not limited to, those derived from a SARS coronavirus (e.g., SARS-CoV-1 and SARS-CoV-2), MERS coronavirus, avian infectious bronchitis (IBV), Mouse hepatitis virus (MHV), and Porcine transmissible gastroenteritis virus (TGEV). The coronavirus immunogen may be a spike polypeptide or a receptor binding domain (RBD) of a spike protein. The coronavirus immunogen may also be an envelope polypeptide, a membrane polypeptide or a nucleocapsid polypeptide.

In some embodiments, the immunogen is from a virus which infects fish. In some embodiments, the immunogen elicits an immune response against a virus which infects fish. For example, the virus which infects fish is selected from infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

In some embodiments, an immunogen is from a host subject cell. For example, antibodies that block viral entry can be generated by using an immunogen or epitope from a component of a host cell that a virus uses as an entry factor.

An immunogen is from, for example, a bacteria, such as a bacterial surface protein, a bacterial membrane protein, a bacterial envelope protein, a bacterial inner membrane protein, a bacterial outer membrane protein, a bacterial periplasmic protein, a bacterial entry protein, a bacterial membrane fusion protein, a bacterial structural protein, a bacterial non-structural protein, a secreted bacterial protein, a bacterial polymerase protein, a bacterial DNA polymerase, a bacterial RNA polymerase, a bacterial protease, a bacterial glycoprotein, bacterial transcription factor, a bacterial enzyme, or a bacterial toxin.

In some embodiments, the immunogen elicits an immune response from one of these bacteria: Streptococcus agalactiae (also known as group B streptococcus or GBS)); Streptococcus pyogenes (also known as group A Streptococcus (GAS)); Staphylococcus aureus; Methicillin-resistant Staphylococcus aureus (MRSA); Staphylococcus epidermis; Treponema pallidum; Francisella tularensis; Rickettsia species; Yersinia pestis; Neisseria meningitidis: Immunogens include, but are not limited to, membrane proteins such as adhesins, autotransporters, toxins, iron acquisition proteins, and factor H binding protein; Streptococcus pneumoniae; Moraxella catarrhalis; Bordetella pertussis: Immunogens include, but are not limited to, pertussis toxin or toxoid (PT), filamentous haemagglutinin (FHA), pertactin, and agglutinogens 2 and 3; Clostridium tetani: the typical immunogen is tetanus toxoid; Cornynebacterium diphtheriae: the typical immunogen is diphtheria toxoid; Haemophilus influenzae; Pseudomonas aeruginosa; Chlamydia trachomatis; Chlamydia pneumoniae; Helicobacter pylori; Escherichia coli (Immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli(ExPEC) and/or enterohemorrhagic E. coli(EHEC)). ExPEC strains include uropathogenic E. coli (UPEC) and meningitis/sepsis-associated E. coli (MNEC). Also included are Bacillus anthracis; Clostridium perfringens or Clostridium botulinums; Legionella pneumophila; Coxiella burnetiid; Brucella species, such as B. abortus, B. canis, B. melitensis, B. neotomae, B. ovis, B. suis, and B. pinnipediae. Francisella species, such as F. novicida, F. philomiragia, and F. tularensis; Neisseria gonorrhoeae; Haemophilus ducreyi; Enterococcus faecalis or Enterococcus faecium; Staphylococcus saprophyticus; Yersinia enterocolitica; Mycobacterium tuberculosis; Listeria monocytogenes; Vibrio cholerae; Salmonella typhi; Borrelia burgdorferi; Porphyromonas gingivalis; and Klebsiella species.

An immunogen is from, for example, fungus, such as a fungal surface protein, a fungal membrane protein, a fungal envelope protein, a fungal inner membrane protein, a fungal outer membrane protein, a fungal periplasmic protein, a fungal entry protein, a fungal membrane fusion protein, a fungal structural protein, a fungal non-structural protein, a secreted fungal protein, a fungal polymerase protein, a fungal DNA polymerase, a fungal RNA polymerase, a fungal protease, a fungal glycoprotein, fungal transcription factor, a fungal enzyme, or a fungal toxin.

In some embodiments, the fungal immunogen is derived from Dermatophytes, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

An immunogen is from, for example, a eukaryotic parasite surface protein, eukaryotic parasite membrane protein, a eukaryotic parasite envelope protein, a eukaryotic parasite entry protein, a eukaryotic parasite membrane fusion protein, a eukaryotic parasite structural protein, a eukaryotic parasite non-structural protein, a secreted eukaryotic parasite protein, a eukaryotic parasite polymerase protein, a eukaryotic parasite DNA polymerase, a eukaryotic parasite RNA polymerase, a eukaryotic parasite protease, a eukaryotic parasite glycoprotein, eukaryotic parasite transcription factor, a eukaryotic parasite enzyme, or a eukaryotic parasite toxin.

In some embodiments, the immunogen elicits an immune response against a parasite from the Plasmodium genus, such as P. falciparum, P. vivax, P. malariae, or P. ovale. In some embodiments, the immunogen elicits an immune response against a parasite from the Caligidae family, particularly those from the Lepeophtheirus and Caligus genera, e.g., sea lice such as Lepeophtheirus salmonis or Caligus rogercresseyi. In some embodiments, the immunogen elicits an immune response against the parasite Toxoplasma gondii.

In some embodiments, the immunogens are cancer immunogens (e.g., neoepitopes). For example, an immunogen is a neoantigen and/or neoepitope that is associated with acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or any combination thereof.

In some embodiments, the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUMI (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcino embryonic antigen (associated with, e.g., breast cancer, lung cancer, and cancers of the gastrointestinal tract such as colorectal cancer); (d) shared antigens, for example, melanoma-melanocyte differentiation antigens such as MART-I/Melan A, gplOO, MC1R, melanocyte-stimulating hormone receptor, tyrosinase, tyrosinase related protein-1/TRPI and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example); (g) neoantigens. In certain embodiments, tumor immunogens include, but are not limited to, pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pl80erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29YBCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY-CO-1, RCAS1, SDCCAGi6, TA-90 (Mac-2 binding protein cyclophilin C-associated protein), TAAL6, TAG72, TLP, TPS, and the like.

In some embodiments, the immunogen elicits an immune response against: pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g., Lepidoglyphys, Glycyphagus and Tyrophagus, those from cockroaches, midges and fleas e.g., Blatella, Periplaneta, Chironomus, and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments, the immunogen is derived from, for example, toxin in a venom, such as a venom from a snake (e.g., most species of rattlesnakes (e.g., eastern diamondback rattlesnake), species of brown snakes (e.g., king brown snake and eastern brown snake), russel's viper, cobras (e.g., Indian cobra, king cobra), certain species of kraits (e.g., common krait), mambas (e.g., black mamba), saw-scaled viper, boomslang, dubois sea snake, species of taipans (e.g., coastal taipan and inland taipan snake), species of lanceheads (e.g., fer-de-lance and terciopelo), bushmasters, copperhead, cottonmouth, coral snakes, death adders, Belcher's sea snake, tiger snakes, Australian black snakes), spider (e.g., brown recluse, black widow spider, Brazilian wandering spider, funnel-web spider, button spider, Australian redback spider, katipo, false black widow, Chilean recluse spider, mouse spider, species of Macrothele, species of Sicarius, species of Hexpthalma, certain species of tarantulas), scorpion and other arachnids (e.g., fat-tailed scorpion, deathstalker scorpion, Indian red scorpion, species of Centruroides, species of Tityus such as the Brazilian yellow scorpion), insects (e.g., species of bees, species of wasps, certain ants such as fire ants, some species of lepidopteran caterpillars, certain species of centipede, remipede Xibalbanus tulumensis), fish (e.g., certain species of catfish (e.g., striped eel catfish and other eeltail catfishes), certain species of stingrays (e.g., blue-spotted stingray), lionfishes, stonefishes, scorpionfishes, toadfishes, rabbitfishes, goblinfishes, cockatoo waspfish, striped blenny, stargazers, chimaeras, weevers, dogfish sharks), cnidarians (e.g., certain species jellyfish (e.g., Irukanjdi jellyfish and box jellyfish), hydrozoans (e.g., Portuguese Man o'War), sea anemones, certain species of coral), a lizard (e.g., a gila monster, Mexican bearded lizard, certain species of Varanus (e.g., Komodo dragon), perentie, and lace monitor), a mammal (e.g., Southern short-tailed shrew, duck-billed platypus, European mole, Eurasian water shrew, Mediterranean water shrew, Northern short-tailed shrew, Elliot's short-tailed shrew, certain species of solenodon (e.g., Cuban solenodon, Hispaniolan solenodon), slow loiris), mollusks (e.g., certain species of cone snail), cephalopods (e.g., certain species of octopus (e.g., blue-ringed octopus), squid, and cuttlefish), amphibians (e.g., frogs such as poison dart frogs, Bruno's casque-headed frog, Greening's frog, salamanders (e.g., Fire salamander, Iberian ribbed newt).

In some embodiments, the toxin is from a plant or fungi (e.g., a mushroom).

In some embodiments, the toxin immunogen is derived from a toxin such as a cyanotoxins, dinotoxins, myotoxins, cytotoxins (e.g., ricin, apitoxin, mycotoxins (e.g., aflatoxin), ochratoxin, citrinin, ergot alkaloid, patulin, fusarium, fumonisins, trichothecenes, cardiotoxin), tetrodotoxin, batrachotoxin, botulinum toxin A, tetanus toxin A, diptheria toxin, dioxin, muscarine, bufortoxin, sarin, hemotoxins, phototoxins, necrotoxins, nephrotoxins, and neurotoxins (e.g., calciseptine, cobrotoxin, calcicludine, fasciculin-I, calliotoxin).

Immunogens from any number of microorganisms or cancers can be utilized in the circular or linear polyribonucleotides. In some cases, the immunogens are associated with or expressed by one microorganism disclosed above. In some embodiments, the immunogens are associated with or expressed by two or more microorganisms disclosed above. In some cases, the immunogens are associated with or expressed by one cancer disclosed above. In some embodiments, immunogens are associated with or expressed by two or more cancers disclosed above. In some embodiments, the immunogens are derived from toxins as disclosed above. In some embodiments, the immunogens are from two or more toxins disclosed above.

The two or more microorganisms are related or unrelated. In some cases, two or more microorganisms are phylogenetically related. For example, the circular or linear polyribonucleotides of the disclosure include or encode immunogens from two or more viruses, two or more members of a viral family, two or more members of a viral class, two or more members of a viral order, two or more members of a viral genus, two or more members of a viral species, two or more bacterial pathogens. In some embodiments, the two or more microorganisms are not phylogenetically related.

In some cases, two or more microorganisms are phenotypically related. For example, the circular or linear polyribonucleotides of the disclosure include or encode immunogens from two or more respiratory pathogens, two or more select agents, two or more microorganisms associated with severe disease, two or more microorganisms associated with adverse outcomes in immunocompromised subjects, two or more microorganisms associated with adverse outcomes related to pregnancy, two or more microorganisms associated with hemorrhagic fever.

An immunogen of the disclosure may include a wild-type sequence. When describing an immunogen, the term “wild-type” refers to a sequence (e.g., a nucleic acid sequence or an amino acid sequence) that is naturally occurring and encoded by a genome (e.g., a viral genome). A species (e.g., microorganism species) can have one wild-type sequence, or two or more wild-type sequences (for example, with one canonical wild-type sequence present in a reference microorganism genome, and additional variant wild-type sequences present that have arisen from mutations).

When describing an immunogen, the terms “derivative” and “derived from” refers to a sequence (e.g., nucleic acid sequence or amino acid sequence) that differs from a wild-type sequence by one or more nucleic acids or amino acids, for example, containing one or more nucleic acid or amino acid insertions, deletions, and/or substitutions relative to a wild-type sequence.

An immunogen derivative sequence is a sequence that has at least 60%, 70%, 75%, 80%, 85%, 90%, 91%, 92%, 93%, 94%, 95%, 96%, 97%, 98%, 99%, or more sequence identity to a wild-type sequence, for example, a wild-type nucleic acid, protein, immunogen, or epitope sequence.

In some embodiments, an immunogen contains one or more amino acid insertions, deletions, substitutions, or a combination thereof that affect the structure of an encoded protein. In some embodiments, an immunogen contains one or more amino acid insertions, deletions, substitutions, or a combination thereof that affect the function of an encoded protein. In some embodiments, an immunogen contains one or more amino acid insertions, deletions, substitutions, or a combination thereof that affect the expression or processing of an encoded protein by a cell.

In some embodiments, an immunogen contains one or more nucleic acid insertions, deletions, substitutions, or a combination thereof that affect the structure of an encoded immunogenic nucleic acid.

Amino acid insertions, deletions, substitutions, or a combination thereof can introduce a site for a post-translational modification (for example, introduce a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof remove a site for a post-translational modification (for example, remove a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof modify a site for a post-translational modification (for example, modify a site to alter the efficiency or characteristics of glycosylation, ubiquitination, phosphorylation, nitrosylation, methylation, acetylation, amidation, hydroxylation, sulfation, or lipidation site, or cleavage).

An amino acid substitution can be a conservative or a non-conservative substitution. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val(V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group 1: ala, pro, gly, gln, asn, ser, thr; Group II: cys, ser, tyr, thr; Group III: val, ile, leu, met, ala, phe; Group IV: lys, arg, his; Group V: phe, tyr, trp, his; and Group VI: asp, glu.

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 amino acid deletions relative to a sequence disclosed herein (e.g., a wild-type sequence).

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a sequence disclosed herein (e.g., a wild-type sequence).

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a sequence disclosed herein (e.g., a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid substitutions relative to a sequence disclosed herein (e.g., a wild-type sequence).

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid substitutions relative to a sequence disclosed herein (e.g., a wild-type sequence).

The one or more amino acid substitutions can be at the N-terminus, the C-terminus, within the amino acid sequence, or a combination thereof. The amino acid substitutions can be contiguous, non-contiguous, or a combination thereof.

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 120, at most 140, at most 160, at most 180, or at most 200 amino acid deletions relative to a sequence disclosed herein (e.g., a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, 20-25, 20-30, 30-50, 50-100, or 100-200 amino acid deletions relative to a wild-type sequence.

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid deletions relative to a wild-type sequence.

The one or more amino acid deletions can be at the N-terminus, the C-terminus, within the amino acid sequence, or a combination thereof. The amino acid deletions can be contiguous, non-contiguous, or a combination thereof.

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild-type sequence.

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid insertions relative to a wild-type sequence.

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid insertions relative to a wild-type sequence.

The one or more amino acid insertions can be at the N-terminus, the C-terminus, within the amino acid sequence, or a combination thereof. The amino acid insertions can be contiguous, non-contiguous, or a combination thereof.

In some embodiments, the immunogen is expressed by the circular or linear polyribonucleotide.

In some embodiments, the immunogen is a product of rolling circle amplification of the circular or linear polyribonucleotide.

The immunogen may be produced in substantial amounts. As such, the immunogen may be any proteinaceous molecule that can be produced. An immunogen can be a polypeptide that can be secreted from a cell, or localized to the cytoplasm, nucleus, or membrane compartment of a cell. In some embodiments, a polypeptide encoded by a circular or linear polyribonucleotide of the disclosure includes a fusion protein including two or more immunogens disclosed herein. In some embodiments, a polypeptide encoded by a circular or linear polyribonucleotide of the disclosure includes an epitope. In some embodiments, a polypeptide encoded by a circular or linear polyribonucleotide of the disclosure includes a fusion protein including two or more epitopes disclosed herein, for example, an artificial peptide sequence including a plurality of predicted epitopes from one or more microorganisms of the disclosure.

In some embodiments, an immunogen that can be expressed from the circular or linear polyribonucleotide is a membrane protein, for example, including a polypeptide sequence that is generally found as a membrane protein, or a polypeptide sequence that is modified to be a membrane protein. In some embodiments, exemplary immunogens that can be expressed from the circular or linear polyribonucleotide disclosed herein include an intracellular immunogen or cytosolic immunogen.

In some embodiments, the immunogen has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, less than about 250 amino acids, less than about 200 amino acids, less than about 150 amino acids, less than about 140 amino acids, less than about 130 amino acids, less than about 120 amino acids, less than about 110 amino acids, less than about 100 amino acids, less than about 90 amino acids, less than about 80 amino acids, less than about 70 amino acids, less than about 60 amino acids, less than about 50 amino acids, less than about 40 amino acids, less than about 30 amino acids, less than about 25 amino acids, less than about 20 amino acids, less than about 15 amino acids, less than about 10 amino acids, less than about 5 amino acids, any amino acid length therebetween or less may be useful.

In some embodiments, the circular or linear polyribonucleotide includes one or more immunogen sequences and is configured for persistent expression in a cell of a subject in vivo. In some embodiments, the circular or linear polyribonucleotide is configured such that expression of the one or more expression sequences in the cell at a later time point is equal to or higher than an earlier time point.

In such embodiments, the expression of the one or more immunogen sequences can be either maintained at a relatively stable level or can increase over time. The expression of the immunogen sequences can be relatively stable for an extended period of time. The expression of the immunogen sequences can be relatively stable transiently or for only a limited amount of time, for example, at most 1, 2, 3, 4, 5, 6, 7, 8, 9, or 10 days.

In some embodiments, the circular or linear polyribonucleotide expresses one or more immunogens in a subject, e.g., transiently or long term. In certain embodiments, expression of the immunogens persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, expression of the immunogens persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or any time therebetween.

The immunogen expression includes translating at least a region of the circular or linear polyribonucleotide provided herein. For example, a circular or linear polyribonucleotide can be translated in a subject to generate polypeptides that include one or more immunogens of the disclosure, thereby stimulating production of an adaptive immune response (e.g., antibody response and/or T cell response) in the subject. In some embodiments, a circular or linear polyribonucleotide of the disclosure is translated to produce one or more immunogens in a human or animal subject, thereby stimulating production of an adaptive immune response (e.g., antibody response and/or T cell response) in a human or animal subject.

In some embodiments, the methods for immunogen expression includes translation of at least 10%, at least 20%, at least 30%, at least 40%, at least 50%, at least 60%, at least 70%, at least 80%, at least 90%, or at least 95% of the total length of the circular or linear polyribonucleotide into polypeptides.

In some embodiments, the methods for immunogen expression includes translation of the circular or linear polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression includes translation of the circular or linear polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods include translation of the circular or linear polyribonucleotide into continuous polypeptides as provided herein, discrete polypeptides as provided herein, or both.

In some embodiments, the methods for immunogen expression include modification, folding, or other post-translation modification of the translation product. In some embodiments, the methods for immunogen expression include post-translation modification in vivo, e.g., via cellular machinery.

### Signal Sequence

In some embodiments, exemplary immunogens that can be expressed from a circular or linear polyribonucleotide disclosed herein include a secreted protein, for example, a protein (e.g., immunogen) that naturally includes a signal sequence, or one that does not usually encode a signal sequence, but is modified to contain one. In some embodiments, the immunogen(s) encoded for by the circular or linear polyribonucleotide includes a secretion signal. For example, the secretion signal may be the naturally encoded secretion signal for a secreted protein. In another example, the secretion signal may be a modified secretion signal for a secreted protein. In other embodiments, the immunogen(s) encoded for by the circular or linear polyribonucleotide do not include a secretion signal.

In some embodiments, a circular or linear polyribonucleotide encodes multiple copies of the same immunogen (e.g., one, two, three, four, five, six, seven, eight, nine, ten, or more) copies of the same immunogen. In some embodiments, at least one copy of the immunogen includes a signal sequence and at least one copy of the immunogen does not include a signal sequence. In some embodiments, a circular or linear polyribonucleotide encodes plurality of immunogens (e.g., a plurality of different immunogens or a plurality of immunogens having less than 100% sequence identity), where at least one of the plurality of immunogens includes a signal sequence and at least one copy of the plurality of immunogens does not include a signal sequence.

In some embodiments, the signal sequence is a wild-type signal sequence that is present on the N-terminus of the corresponding wild-type immunogen, e.g., when expressed endogenously. In some embodiments, the signal sequence is heterologous to the immunogen, e.g., is not present when the wild-type immunogen is expressed endogenously. A polyribonucleotide sequence encoding an immunogen may be modified to remove the nucleotide sequence encoding a wild-type signal sequence and/or add a sequence encoding a heterologous signal sequence.

The circular or linear polyribonucleotide may further include one or more adjuvants, each with or without a signal sequence. In some embodiments, the circular or linear polyribonucleotide encodes at least one adjuvant and at least one immunogen. In some embodiments, the at least one encoded adjuvant includes a signal sequence and the at least one encoded immunogen does not include a signal sequence. In some embodiments, the at least one encoded adjuvant includes a signal sequence and the at least one encoded immunogen includes a signal sequence. In some embodiments, the at least one encoded adjuvant does not include a signal sequence and the at least one encoded immunogen includes a signal sequence. In some embodiments, neither the encoded adjuvant nor the encoded immunogen includes a signal sequence.

In some embodiments, the signal sequence is a wild-type signal sequence that is present on the N-terminus of the corresponding wild-type adjuvant, e.g., when expressed endogenously. In some embodiments, the signal sequence is heterologous to the adjuvant, e.g., is not present when the wild-type adjuvant is expressed endogenously. A polyribonucleotide sequence encoding an adjuvant may be modified to remove the nucleotide sequence encoding a wild-type signal sequence and/or add a sequence encoding a heterologous signal sequence.

A polypeptide encoded by a polyribonucleotide (e.g., immunogen or an adjuvant encoded by a polyribonucleotide) may include a signal sequence that directs the immunogen or adjuvant to the secretory pathway. In some embodiments, the signal sequence may direct the immunogen or adjuvant to reside in certain organelles (e.g., the endoplasmic reticulum, Golgi apparatus, or endosomes). In some embodiments, the signal sequence directs the immunogen or adjuvant to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane.

In some embodiments, an adjuvant encoded by a polyribonucleotide includes a secretion signal sequence. In some embodiments, an immunogen encoded by a polyribonucleotide includes either a secretion signal sequence, a transmembrane insertion signal sequence, or does not include a signal sequence.

### Regulatory Element

In some embodiments, a circular or linear polyribonucleotide includes a regulatory element, e.g., a sequence that modifies expression of an expression sequence within the circular or linear polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more immunogen(s) and/or adjuvant(s) encoded by a circular or linear polyribonucleotide. Likewise, a regulatory element may be used to decrease the expression of one or more immunogen(s) and/or adjuvant(s) encoded by a circular or linear polyribonucleotide. In some embodiments, a regulatory element may be used to increase expression of an immunogen and/or adjuvant and another regulatory element may be used to decrease expression of another immunogen and/or adjuvant on the same circular or linear polyribonucleotide. In addition, one regulatory element can increase an amount of products (e.g., an immunogen or adjuvants) expressed for multiple expression sequences attached in tandem. Hence, one regulatory element can enhance the expression of one or more expression sequences (e.g., immunogens or adjuvants). Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular or linear polyribonucleotide, for instance, certain riboswitch aptazymes. A regulatory element can also include a selective degradation sequence. As used herein, the term “selective degradation sequence” refers to a nucleic acid sequence that initiates degradation of the circular or linear polyribonucleotide, or an expression product of the circular or linear polyribonucleotide. In some embodiments, the regulatory element is a translation modulator. A translation modulator can modulate translation of the expression sequence in the circular or linear polyribonucleotide. A translation modulator can be a translation enhancer or suppressor. In some embodiments, a translation initiation sequence can function as a regulatory element. Further examples of regulatory elements are described in paragraphs [0154]-[0161] of International Patent Publication No. WO2019/118919, which is hereby incorporated by reference in its entirety.

Nucleotides flanking a codon that initiates translation, such as, but not limited to, a start codon or an alternative start codon, are known to affect the translation efficiency, the length, and/or the structure of the circular or linear polyribonucleotide. (See e.g., Matsuda and Mauro PLoS ONE, 2010 5: 11; the contents of which are herein incorporated by reference in its entirety). Masking any of the nucleotides flanking a codon that initiates translation may be used to alter the position of translation initiation, translation efficiency, length and/or structure of the circular or linear polyribonucleotide.

In one embodiment, a masking agent may be used near the start codon or alternative start codon in order to mask or hide the codon to reduce the probability of translation initiation at the masked start codon or alternative start codon. In another embodiment, a masking agent may be used to mask a start codon of the circular or linear polyribonucleotide in order to increase the likelihood that translation will initiate at an alternative start codon.

### Translation Initiation Sequence

In some embodiments, a circular or linear polyribonucleotide encodes an immunogen and includes a translation initiation sequence, e.g., a start codon. In some embodiments, the translation initiation sequence includes a Kozak or Shine-Dalgarno sequence. In some embodiments, the translation initiation sequence includes a Kozak sequence. In some embodiments, the circular or linear polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular or linear polyribonucleotide includes at least one translation initiation sequence adjacent to an expression sequence. In some embodiments, the translation initiation sequence provides conformational flexibility to the circular or linear polyribonucleotide. In some embodiments, the translation initiation sequence is within a substantially single stranded region of the circular or linear polyribonucleotide. Further examples of translation initiation sequences are described in paragraphs [0163]-[0165] of International Patent Publication No. WO2019/118919, which is hereby incorporated by reference in its entirety.

The circular or linear polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular or linear polyribonucleotide may initiate at a codon which is not the first start codon, e.g., AUG. Translation of the circular or linear polyribonucleotide may initiate at an alternative translation initiation sequence, such as those described in [0164] of International Patent Publication No. WO2019/118919A1, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

### IRES

In some embodiments, a circular or linear polyribonucleotide described herein includes an internal ribosome entry site (IRES) element. In some embodiments, a circular or linear polyribonucleotide described herein includes more than one (e.g., 2, 3, 4, and 5) internal ribosome entry site (IRES) element. In some embodiments, the circular or linear polyribonucleotide includes one or more IRES sequences on one or both sides of each expression sequence, leading to separation of the resulting peptide(s) and or polypeptide(s). In some embodiments, the IRES flanks both sides of at least one (e.g., 2, 3, 4, 5 or more) expression sequence. A suitable IRES element to include in a circular or linear polyribonucleotide can be an RNA sequence capable of engaging a eukaryotic ribosome. In some embodiments, the IRES is an encephalomyocarditis virus (EMCV) IRES. In some embodiments, the IRES is a Coxsackievirus (CVB3) IRES. Further examples of an IRES are described in paragraphs [0166]-[0168] of International Patent Publication No. WO2019/118919, which is hereby incorporated by reference in its entirety.

### Cleavage Domains

A circular or linear polyribonucleotide of the disclosure can include a cleavage domain (e.g., a stagger element or a cleavage sequence).

The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPGP, where x=any amino acid (SEQ ID NO: 7). In some embodiments, the stagger element may include a chemical moiety, such as glycerol, a non-nucleic acid linking moiety, a chemical modification, a modified nucleic acid, or any combination thereof.

In some embodiments, a circular or linear polyribonucleotide includes at least one stagger element adjacent to an expression sequence. In some embodiments, the circular or linear polyribonucleotide includes a stagger element adjacent to each expression sequence. In some embodiments, the stagger element is present on one or both sides of each expression sequence, leading to separation of the expression products, e.g., immunogen(s) and/or adjuvant(s). In some embodiments, the stagger element is a portion of the one or more expression sequences. In some embodiments, the circular or linear polyribonucleotide includes one or more expression sequences (e.g., immunogen(s) and/or adjuvant(s)), and each of the one or more expression sequences is separated from a succeeding expression sequence (e.g., immunogen(s) and/or adjuvant(s) by a stagger element on the circular or linear polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

In some embodiments, the plurality of immunogens encoded by a circular or linear ribonucleotide may be separated by an IRES between each immunogen. The IRES may be the same IRES between all immunogens. The IRES may be different between different immunogens. In other embodiments, the plurality of immunogens and/or adjuvants may be separated by a 2A self-cleaving peptide. Furthermore, the plurality of immunogens and/or adjuvants encoded by the circular or linear ribonucleotide may be separated by both IRES and 2A sequences. For example, an IRES may be between one immunogens and/or adjuvant and a second immunogen and/or adjuvant while a 2A peptide may be between the second immunogen and/or adjuvant and the third immunogen and/or adjuvant. The selection of a particular IRES or 2A self-cleaving peptide may be used to control the expression level of immunogen and/or adjuvant under control of the IRES or 2A sequence. For example, depending on the IRES and or 2A peptide selected, expression on the polypeptide may be higher or lower.

To avoid production of a continuous expression product, e.g., immunogen and/or adjuvant, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular or linear polyribonucleotide. The stagger element may include, but is not limited to a 2A-like, or CHYSEL (SEQ ID NO: 8) (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid (SEQ ID NO: 9). In some embodiments, this sequence includes a non-conserved sequence of amino-acids with a strong alpha-helical propensity followed by the consensus sequence -D(V/I)ExNPGP, where x=any amino acid (SEQ ID NO: 7). Some non-limiting examples of stagger elements includes GDVESNPGP (SEQ ID NO: 10), GDIEENPGP (SEQ ID NO: 11), VEPNPGP (SEQ ID NO: 12), IETNPGP (SEQ ID NO: 13), GDIESNPGP (SEQ ID NO: 14), GDVELNPGP (SEQ ID NO: 15), GDIETNPGP (SEQ ID NO: 16), GDVENPGP (SEQ ID NO: 17), GDVEENPGP (SEQ ID NO: 18), GDVEQNPGP (SEQ ID NO: 19), IESNPGP (SEQ ID NO: 20), GDIELNPGP (SEQ ID NO: 21), HDIETNPGP (SEQ ID NO: 22), HDVETNPGP (SEQ ID NO: 23), HDVEMNPGP (SEQ ID NO: 24), GDMESNPGP (SEQ ID NO: 25), GDVETNPGP (SEQ ID NO: 26), GDIEQNPGP (SEQ ID NO: 27), and DSEFNPGP (SEQ ID NO: 28).

In some embodiments, a stagger element described herein cleaves an expression product, such as between G and P of the consensus sequence described herein. As one non-limiting example, the circular or linear polyribonucleotide includes at least one stagger element to cleave the expression product. In some embodiments, the circular or linear polyribonucleotide includes a stagger element adjacent to at least one expression sequence. In some embodiments, the circular or linear polyribonucleotide includes a stagger element after each expression sequence. In some embodiments, the circular or linear polyribonucleotide includes a stagger element is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g. to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular or linear polyribonucleotide in other forms. For example, in some exemplary circular or linear polyribonucleotides, a stagger element includes a termination element of a first expression sequence in the circular or linear polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular or linear polyribonucleotide. In some cases, the first expression sequence and the expression sequence succeeding the first expression sequence are two separate expression sequences in the circular or linear polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular or linear polyribonucleotide including the first stagger element upstream of the first translation initiation sequence of the succeeding sequence in the circular or linear polyribonucleotide is continuously translated, while a corresponding circular or linear polyribonucleotide including a stagger element of a second expression sequence that is upstream of a second translation initiation sequence of an expression sequence succeeding the second expression sequence is not continuously translated. In some cases, there is only one expression sequence in the circular or linear polyribonucleotide, and the first expression sequence and its succeeding expression sequence are the same expression sequence. In some exemplary circular or linear polyribonucleotides, a stagger element includes a first termination element of a first expression sequence in the circular or linear polyribonucleotide, and a nucleotide spacer sequence that separates the termination element from a downstream translation initiation sequence. In some such examples, the first stagger element is upstream of (5′ to) a first translation initiation sequence of the first expression sequence in the circular or linear polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular or linear polyribonucleotide including the first stagger element upstream of the first translation initiation sequence of the first expression sequence in the circular or linear polyribonucleotide is continuously translated, while a corresponding circular or linear polyribonucleotide including a stagger element upstream of a second translation initiation sequence of a second expression sequence in the corresponding circular or linear polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular or linear polyribonucleotide than a distance between the first stagger element and the first translation initiation in the circular or linear polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular or linear polyribonucleotide includes more than one expression sequence.

In some embodiments, a circular or linear polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular or linear polyribonucleotide includes between 2 and 10 cleavage sequences. In some embodiments, the circular or linear polyribonucleotide includes between 2 and 5 cleavage sequences. In some embodiments, the multiple cleavage sequences are between multiple expression sequences; for example, a circular or linear polyribonucleotide may include three expression sequences two cleavage sequences such that there is a cleavage sequence in between each expression sequence. In some embodiments, the circular or linear polyribonucleotide includes a cleavage sequence, such as in an immolating circRNA or cleavable circRNA or self-cleaving circRNA. In some embodiments, the circular or linear polyribonucleotide includes two or more cleavage sequences, leading to separation of the circular or linear polyribonucleotide into multiple products, e.g., miRNAs, linear RNAs, smaller circular or linear polyribonucleotide, etc.

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a polyribonucleotide may encode two or more immunogens, e.g., where the two or more immunogens are encoded by a single open-reading frame (ORF). For example, two or more immunogens may be encoded by a single open-reading frame, the expression of which is controlled by an IRES. In some embodiments, the ORF further encodes a polypeptide linker, e.g., such that the expression product of the ORF encodes two or more immunogens each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the polyribonucleotide to that subject). In such embodiments, a single expression product including the amino acid sequence of two or more immunogens is cleaved upon expression, such that the two or more immunogens are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease, urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular or linear polyribonucleotide described herein is an immolating circular or linear polyribonucleotide, a cleavable circular or linear polyribonucleotide, or a self-cleaving circular or linear polyribonucleotide. A circular or linear polyribonucleotide can deliver cellular components including, for example, RNA, lncRNA, lincRNA, miRNA, tRNA, rRNA, snoRNA, ncRNA, siRNA, or shRNA. In some embodiments, a circular or linear polyribonucleotide includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes.

### Regulatory Elements and Ratio of Expression Products

In some embodiments, a circular or linear polyribonucleotide includes one or more regulatory nucleic acid sequences or includes one or more expression sequences that encode regulatory nucleic acid, e.g., a nucleic acid that modifies expression of an endogenous gene and/or an exogenous gene. In some embodiments, the expression sequence of a circular or linear polyribonucleotide as provided herein can include a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

Exemplary regulatory nucleic acids are described in paragraphs [0177]-[0194] of International Patent Publication No. WO2019/118919, which is hereby incorporated by reference in its entirety.

In some embodiments, the translation efficiency of a circular polyribonucleotide as provided herein is greater than a reference, e.g., a linear counterpart, a linear expression sequence, or a linear polyribonucleotide for circularization. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, a circular or linear polyribonucleotide produces stoichiometric ratios of expression products. Rolling circle translation continuously produces expression products at substantially equivalent ratios. In some embodiments, the circular or linear polyribonucleotide has a stoichiometric translation efficiency, such that expression products are produced at substantially equivalent ratios. In some embodiments, the circular or linear polyribonucleotide has a stoichiometric translation efficiency of multiple expression products, e.g., products from 2, 3, 4, 5, 6, 7, 8, 9, 10, 11, 12, or more expression sequences. In some embodiments, the circular or linear polyribonucleotide produces substantially different ratios of expression products. For example, the translation efficiency of multiple expression products may have a ratio of 1:10,000; 1:7000, 1:5000, 1:1000, 1:700, 1:500, 1:100, 1:50, 1:10, 1:5, 1:4, 1:3 or 1:2. In some embodiments, the ratio of multiple expression products may be modified using a regulatory element.

### Translation

In some embodiments, once translation of a circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of a circular polyribonucleotide leads to generation of polypeptide product that is translated from more than one round of translation of the circular polyribonucleotide (“continuous” expression product). In some embodiments, the circular polyribonucleotide includes a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the circular polyribonucleotide is configured such that at least 99% of the total polypeptides are discrete polypeptides. In some embodiments, the amount ratio of the discrete products over the total polypeptides is tested in an in vitro translation system. In some embodiments, the in vitro translation system used for the test of amount ratio includes rabbit reticulocyte lysate. In some embodiments, the amount ratio is tested in an in vivo translation system, such as a eukaryotic cell or a prokaryotic cell, a cultured cell, or a cell in an organism.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Termination Sequence

A circular polyribonucleotide can include one or more expression sequences (e.g., encoding an immunogen and/or encoding an adjuvant), and each expression sequence may or may not have a termination element. Further examples of termination elements are described in paragraphs [0169]-[0170] of International Patent Publication No. WO2019/118919, which is hereby incorporated by reference in its entirety.

In some embodiments, the circular polyribonucleotide includes a poly-A sequence. In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety.

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, expression efficiency, etc.

### Regulatory Nucleic Acids

In some embodiments, a circular polyribonucleotide includes one or more expression sequences that encode regulatory nucleic acid, e.g., that modifies expression of an endogenous gene and/or an exogenous gene. In some embodiments, the expression sequence of a circular polyribonucleotide as provided herein can include a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

In one embodiment, the regulatory nucleic acid targets a gene such as a host gene. The regulatory nucleic acids may include any of the regulatory nucleic acids described in [0177] and [0181]-[0189] of International Patent Publication No. WO2019/118919A1, which is incorporated herein by reference in its entirety.

In some embodiments, an expression sequence includes one or more of the features described herein, e.g., a sequence encoding one or more peptides or proteins, one or more regulatory element, one or more regulatory nucleic acids, e.g., one or more non-coding RNAs, other expression sequences, and any combination thereof.

In some embodiments, a circular polyribonucleotide includes one or more RNA binding sites. microRNAs (or miRNA) are short noncoding RNAs that bind to the 3′UTR of nucleic acid molecules and down-regulate gene expression either by reducing nucleic acid molecule stability or by inhibiting translation. The circular polyribonucleotide may include one or more microRNA target sequences, microRNA sequences, or microRNA seeds. Such sequences may correspond to any known microRNA, such as those taught in US Publication US2005/0261218 and US Publication US2005/0059005, the contents of which are incorporated herein by reference in their entirety. A microRNA sequence includes a “seed” region, i.e., a sequence in the region of positions 2-8 of the mature microRNA, which sequence has perfect Watson-Crick complementarity to the miRNA target sequence. A microRNA seed may include positions 2-8 or 2-7 of the mature microRNA. In some embodiments, a microRNA seed may include 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may include 6 nucleotides (e.g., nucleotides 2-7 of the mature microRNA), wherein the seed-complementary site in the corresponding miRNA target is flanked by an adenine (A) opposed to microRNA position 1. See for example, Grimson et al; Mol Cell. 2007 6; 27:91-105; herein incorporated by reference in its entirety.

### Protein-Binding

In some embodiments, a circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, a circular polyribonucleotide includes at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present disclosure, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences including a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG (SEQ ID NO: 29), where R is a purine (adenine or guanine) three bases upstream of the start codon (AUG), which is followed by another ‘G’. 5′UTR also have been known to form secondary structures which are involved in elongation factor binding.

In some embodiments, a circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRINI, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF 5, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Encryptogen

As described herein, the circular polyribonucleotide includes an encryptogen to reduce, evade or avoid the innate immune response of a cell. In one aspect, provided herein are circular polyribonucleotide which when delivered to cells, results in a reduced immune response from the host as compared to the response triggered by a reference compound, e.g. a linear polynucleotide corresponding to the described circular polyribonucleotide or a circular polyribonucleotide lacking an encryptogen. In some embodiments, the circular polyribonucleotide has less immunogenicity (e.g., a lower level of one or more marker of an immune or inflammatory response) than a counterpart lacking an encryptogen.

In some embodiments, an encryptogen enhances stability. There is growing body of evidence about the regulatory roles played by the UTRs in terms of stability of a nucleic acid molecule and translation. The regulatory features of a UTR may be included in the encryptogen to enhance the stability of the circular polyribonucleotide.

In some embodiments, 5′- or 3′-UTRs can constitute encryptogens in a circular polyribonucleotide. For example, removal or modification of UTR AU rich elements (AREs) may be useful to modulate the stability or immunogenicity (e.g., the modulate the level of one or more marker of an immune or inflammatory response) of the circular polyribonucleotide.

In some embodiments, removal of modification of AU rich elements (AREs) in expression sequence, e.g., translatable regions, can be useful to modulate the stability or immunogenicity (e.g., modulate the level of one or more marker of an immune or inflammatory response) of the circular polyribonucleotide.

In some embodiments, an encryptogen includes miRNA binding site or binding site to any other non-coding RNAs. For example, incorporation of miR-142 sites into the circular polyribonucleotide described herein may not only modulate expression in hematopoietic cells, but also reduce or abolish immune responses to a protein encoded in the circular polyribonucleotide.

In some embodiments, an encryptogen includes one or more protein binding sites that enable a protein, e.g., an immunoprotein, to bind to the RNA sequence. By engineering protein binding sites into the circular polyribonucleotide, the circular polyribonucleotide may evade or have reduced detection by the host's immune system, have modulated degradation, or modulated translation, by masking the circular polyribonucleotide from components of the host's immune system. In some embodiments, the circular polyribonucleotide includes at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen includes one or more modified nucleotides. Exemplary modifications can include any modification to the sugar, the nucleobase, the internucleoside linkage (e.g. to a linking phosphate/to a phosphodiester linkage/to the phosphodiester backbone), and any combination thereof that can prevent or reduce immune response against the circular polyribonucleotide. Some of the exemplary modifications are provided herein.

In some embodiments, a circular polyribonucleotide includes one or more modifications as described elsewhere herein to reduce an immune response from the host as compared to the response triggered by a reference compound, e.g. a circular polyribonucleotide lacking the modifications. In particular, the addition of one or more inosine has been shown to discriminate RNA as endogenous versus viral. See for example, Yu, Z. et al. (2015) RNA editing by ADAR1 marks dsRNA as “self”. Cell Res. 25, 1283-1284, which is incorporated by reference in its entirety.

In some embodiments, a circular polyribonucleotide includes one or more expression sequences for shRNA or an RNA sequence that can be processed into siRNA, and the shRNA or siRNA targets RIG-I and reduces expression of RIG-I. RIG-1 can sense foreign circular RNA and leads to degradation of foreign circular RNA. Therefore, a circular polynucleotide harboring sequences for RIG-1-targeting shRNA, siRNA or any other regulatory nucleic acids can reduce immunity, e.g., host cell immunity, against the circular polyribonucleotide.

In some embodiments, a circular polyribonucleotide lacks a sequence, element, or structure, that aids the circular polyribonucleotide in reducing, evading, or avoiding an innate immune response of a cell.

In some such embodiments, the circular polyribonucleotide may lack a polyA sequence, a 5′ end, a 3′ end, phosphate group, hydroxyl group, or any combination thereof.

### Nucleotide Spacer Sequences

In some embodiments, a circular polyribonucleotide includes a spacer sequence. In some embodiments, the circular polyribonucleotide includes at least one spacer sequence. In some embodiments, the circular polyribonucleotide includes 1, 2, 3, 4, 5, 6, 7, or more spacer sequences.

In some embodiments, a circular polyribonucleotide includes a spacer sequence. In some embodiments, elements of a polyribonucleotide may be separated from one another by a spacer sequence or linker. Exemplary spacer sequences are described in paragraphs [0293]-[0302] of International Patent Publication No. WO2019/118919, which is hereby incorporated by reference in its entirety.

### Non-Nucleic Acid Linkers

A circular polyribonucleotide described herein may include a non-nucleic acid linker. In some embodiments, the circular polyribonucleotide has a non-nucleic acid linker between one or more of the sequences or elements described herein. In one embodiment, one or more sequences or elements described herein are linked with the linker. The non-nucleic acid linker may be a chemical bond, e.g., one or more covalent bonds or non-covalent bonds. In some embodiments, the non-nucleic acid linker is a peptide or protein linker. Such a linker may be between 2-30 amino acids, or longer. The circular polyribonucleotide described herein may also include a non-nucleic acid linker. Exemplary non-nucleic acid linkers are described in paragraphs [0303]-[0307] of International Patent Publication No. WO2019/118919, which is hereby incorporated by reference in its entirety.

In some embodiments, a circular polyribonucleotide further includes another nucleic acid sequence. In some embodiments, the circular polyribonucleotide may include other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In some embodiments, the circular polyribonucleotide includes an siRNA to target a different locus of the same gene expression product as the circular polyribonucleotide. In some embodiments, the circular polyribonucleotide includes an siRNA to target a different gene expression product than a gene expression product that is present in the circular polyribonucleotide.

### Stability and Half-Life

In some embodiments, a circular polyribonucleotide includes particular sequence characteristics. For example, the circular polyribonucleotide may include a particular nucleotide composition. In some such embodiments, the circular polyribonucleotide may include one or more purine (adenine and/or guanosine) rich regions. In some such embodiments, the circular polyribonucleotide may include one or more purine poor regions. In some embodiments, the circular polyribonucleotide may include one or more AU rich regions or elements (AREs). In some embodiments, the circular polyribonucleotide may include one or more adenine rich regions.

In some embodiments, a circular polyribonucleotide may include one or more repetitive elements described elsewhere herein. In some embodiments, the circular polyribonucleotide includes one or more modifications described elsewhere herein.

A circular polyribonucleotide may include one or more substitutions, insertions and/or additions, deletions, and covalent modifications with respect to reference sequences. For example, circular polyribonucleotides with one or more insertions, additions, deletions, and/or covalent modifications relative to a parent polyribonucleotide are included within the scope of this disclosure. Exemplary modifications are described in paragraphs [0310]-[0325] of International Patent Publication No. WO2019/118919, which is hereby incorporated by reference in its entirety.

In some embodiments, a circular polyribonucleotide includes a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, one or more sequences of a circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, a circular polyribonucleotide is substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, the circular polyribonucleotide includes at least one of a U-rich or A-rich sequence or a combination thereof.

In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure.

In some embodiments, the circular polyribonucleotide has an intramolecular triple quasi-helix structure that aids in stabilization.

In some embodiments, a circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, a circular polyribonucleotide includes a tertiary structure with one or more motifs, e.g., a pseudoknot, a g-quadruplex, a helix, and coaxial stacking.

Further examples of structure of circular polyribonucleotides as disclosed herein are described in paragraphs [0326]-[0333] of International Patent Publication No. WO2019/118919, which is hereby incorporated by reference in its entirety.

As a result of its circularization, a circular polyribonucleotide may include certain characteristics that distinguish it from linear RNA. For example, the circular polyribonucleotide is less susceptible to degradation by exonuclease as compared to linear RNA. As such, a circular polyribonucleotide can be more stable than a linear RNA, especially when incubated in the presence of an exonuclease. The increased stability of the circular polyribonucleotide compared with linear RNA can make the circular polyribonucleotide more useful as a cell transforming reagent to produce polypeptides (e.g., immunogens and/or adjuvants). The increased stability of the circular polyribonucleotide compared with linear RNA can make the circular polyribonucleotide easier to store for longer than linear RNA. The stability of the circular polyribonucleotide treated with exonuclease can be tested using methods standard in art which determine whether RNA degradation has occurred (e.g., by gel electrophoresis).

Moreover, unlike linear RNA, a circular polyribonucleotide can be less susceptible to dephosphorylation when the circular polyribonucleotide is incubated with phosphatase, such as calf intestine phosphatase.

In some embodiments, a circular polyribonucleotide preparation provided herein has an increased half-life over a reference, e.g., a linear polyribonucleotide having the same nucleotide sequence but is not circularized (e.g., linear counterpart). In some embodiments, the circular polyribonucleotide is resistant to degradation, e.g., exonuclease. In some embodiments, the circular polyribonucleotide is resistant to self-degradation. In some embodiments, the circular polyribonucleotide lacks an enzymatic cleavage site, e.g., a dicer cleavage site. In some embodiments, the circular polyribonucleotide has a half-life at least about 5%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 150%, at least about 160%, at least about 180%, at least about 200%, at least about 300%, at least about 400%, at least about 500%, at least about 600%, at least about 700% at least about 800%, at least about 900%, at least about 1000% or at least about 10000%, longer than a reference, e.g., a linear counterpart.

In some embodiments, the circular polyribonucleotide persists in a cell during cell division. In some embodiments, the circular polyribonucleotide persists in daughter cells after mitosis. In some embodiments, the circular polyribonucleotide is replicated within a cell and is passed to daughter cells. In some embodiments, the circular polyribonucleotide includes a replication element that mediates self-replication of the circular polyribonucleotide. In some embodiments, the replication element mediates transcription of the circular polyribonucleotide into a linear polyribonucleotide that is complementary to the circular polyribonucleotide (linear complementary). In some embodiments, the linear complementary polyribonucleotide can be circularized in vivo in cells into a complementary circular polyribonucleotide. In some embodiments, the complementary polyribonucleotide can further self-replicate into another circular polyribonucleotide, which has the same or similar nucleotide sequence as the starting circular polyribonucleotide. One exemplary self-replication element includes HDV replication domain (as described by Beeharry et al, Virol, 2014, 450-451:165-173). In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

Further examples of stability and half-life of circular polyribonucleotides as disclosed herein are described in paragraphs [0308]-[0309] of International Patent Publication No. WO2019/118919, which is hereby incorporated by reference in its entirety.

### Modifications

A circular polyribonucleotide may include one or more substitutions, insertions and/or additions, deletions, and covalent modifications with respect to reference sequences, in particular, the parent polyribonucleotide, are included within the scope of this disclosure.

In some embodiments, a circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197). In some embodiments, the first isolated nucleic acid includes messenger RNA (mRNA). In some embodiments, the mRNA includes at least one nucleoside selected from the group such as those described in [0311] of International Patent Publication No. WO2019/118919A1, which is incorporated herein by reference in its entirety.

A circular polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a circular polyribonucleotide includes at least one N(6)methyladenosine (m6A) modification to increase translation efficiency. In some embodiments, the N(6)methyladenosine (m6A) modification can reduce immunogenicity (e.g., reduce the level of one or more marker of an immune or inflammatory response) of the circular polyribonucleotide.

In some embodiments, a modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of a circular polyribonucleotide may enhance immune evasion. The circular polyribonucleotide may be synthesized and/or modified by methods well established in the art, such as those described in “Current protocols in nucleic acid chemistry,” Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotide, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the circular polyribonucleotide may, as well as backbone modifications, include modification or replacement of the phosphodiester linkages. Specific examples of circular polyribonucleotide include, but are not limited to, circular polyribonucleotide including modified backbones or no natural internucleoside linkages such as internucleoside modifications, including modification or replacement of the phosphodiester linkages. Circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylenephosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(I-thiophosphate)-cytidine (a-thio-cytidine), 5′-O-(I-thiophosphate)-guanosine, 5′-O-(I-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, a circular polyribonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-I-(tetrahydrofuran-2-yl)pyrimidine-2,4(IH,3H)-dione), troxacitabine, tezacitabine, 2′-deoxy-2′-methylidenecytidine (DMDC), and 6-mercaptopurine. Additional examples include fludarabine phosphate, N4-behenoyl-I-beta-D-arabinofuranosylcytosine, N4-octadecyl-1-beta-D-arabinofuranosylcytosine, N4-palmitoyl-I-(2-C-cyano-2-deoxy-beta-D-arabino-pentofuranosyl) cytosine, and P-4055 (cytarabine 5′-elaidic acid ester).

A circular polyribonucleotide may or may not be uniformly modified along the entire length of the molecule. For example, one or more or all types of nucleotide (e.g., naturally-occurring nucleotides, purine or pyrimidine, or any one or more or all of A, G, U, C, 1, pU) may or may not be uniformly modified in the circular polyribonucleotide, or in a given predetermined sequence region thereof. In some embodiments, the circular polyribonucleotide includes a pseudouridine. In some embodiments, the circular polyribonucleotide includes an inosine, which may aid in the immune system characterizing the circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in a circular polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a circular polyribonucleotide. One of ordinary skill in the art will appreciate that the nucleotide analogs or other modification(s) may be located at any position(s) of the circular polyribonucleotide, such that the function of the circular polyribonucleotide is not substantially decreased. A modification may also be a non-coding region modification. The circular polyribonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Structure

In some embodiments, a circular polyribonucleotide includes a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, the circular polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides.

In some embodiments, one or more sequences of a circular polyribonucleotide include substantially single stranded vs double stranded regions. In some embodiments, the ratio of single stranded to double stranded may influence the functionality of the circular polyribonucleotide.

In some embodiments, one or more sequences of a circular polyribonucleotide are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, a circular polyribonucleotide has at least one binding site, e.g., at least one protein binding site, at least one miRNA binding site, at least one lncRNA binding site, at least one tRNA binding site, at least one rRNA binding site, at least one snRNA binding site, at least one siRNA binding site, at least one piRNA binding site, at least one snoRNA binding site, at least one snRNA binding site, at least one exRNA binding site, at least one scaRNA binding site, at least one Y RNA binding site, at least one hnRNA binding site, and/or at least one tRNA motif.

In some embodiments, a circular polyribonucleotide is configured to include a higher order structure, such as those described in International Patent Publication No. WO2019/118919A1, which is incorporated herein by reference in its entirety.

### Production Methods

In some embodiments, a circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can include a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins including multiple immunogens). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

A circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, U.S. Pat. Nos. 5,712,128, 5,426,180, US Publication No. US20100137407, International Publication No. WO1992001813 and International Publication No. WO2010/084371; the contents of each of which are herein incorporated by reference in their entireties).

### Circularization

In some embodiments, a linear polyribonucleotide for circularization may be cyclized, or concatemerized. In some embodiments, the linear polyribonucleotide for circularization may be cyclized in vitro prior to formulation and/or delivery. In some embodiments, the linear polyribonucleotide for circularization may be cyclized within a cell.

### Extracellular Circularization

In some embodiments, a linear polyribonucleotide for circularization is cyclized, or concatemerized using a chemical method to form a circular polyribonucleotide. In some chemical methods, the 5′-end and the 3′-end of the nucleic acid (e.g., a linear polyribonucleotide for circularization) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase is used to enzymatically link a 5′-phosphorylated nucleic acid molecule (e.g., a linear polyribonucleotide for circularization) to the 3′-hydroxyl group of a nucleic acid (e.g., a linear nucleic acid) forming a new phosphorodiester linkage. In an example reaction, a linear polyribonucleotide for circularization is incubated at 37° C. for 1 hour with 1-10 units of T4 RNA ligase (New England Biolabs, Ipswich, MA) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. In some embodiments, either the 5′- or 3′-end of the linear polyribonucleotide for circularization can encode a ligase ribozyme sequence such that during in vitro transcription, the resultant linear polyribonucleotide for circularization includes an active ribozyme sequence capable of ligating the 5′-end of the linear polyribonucleotide for circularization to the 3′-end of the linear polyribonucleotide for circularization. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear polyribonucleotide for circularization is cyclized or concatermerized by using at least one non-nucleic acid moiety. In one aspect, the at least one non-nucleic acid moiety may react with regions or features near the 5′ terminus and/or near the 3′ terminus of the linear polyribonucleotide for circularization in order to cyclize or concatermerize the linear polyribonucleotide for circularization. In another aspect, the at least one non-nucleic acid moiety may be located in or linked to or near the 5′ terminus and/or the 3′ terminus of the linear polyribonucleotide for circularization. The non-nucleic acid moieties contemplated may be homologous or heterologous. As a non-limiting example, the non-nucleic acid moiety may be a linkage such as a hydrophobic linkage, ionic linkage, a biodegradable linkage, and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In some embodiments, a linear polyribonucleotide for circularization is cyclized or concatermerized due to a non-nucleic acid moiety that causes an attraction between atoms, molecular surfaces at, near or linked to the 5′ and 3′ ends of the linear polyribonucleotide for circularization. As a non-limiting example, one or more linear polyribonucleotides for circularization may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, a linear polyribonucleotide for circularization may include a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′terminus may associate with each other causing a linear polyribonucleotide for circularization to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present disclosure or a non-exhaustive listing of methods to incorporate and/or covalently link peptides are described in US patent application No. US20030082768, the contents of which is here in incorporated by reference in its entirety.

In some embodiments, a linear polyribonucleotide for circularization may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear polyribonucleotide for circularization into a 5′ monophosphate may occur by a two-step reaction including: (a) contacting the 5′ nucleotide of the linear polyribonucleotide for circularization with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%. In some embodiments, the circularization method provided has a circularization efficiency of between about 10% and about 100%; for example, the circularization efficiency may be about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, the circularization efficiency is between about 20% and about 80%. In some embodiments, the circularization efficiency is between about 30% and about 60%. In some embodiments the circularization efficiency is about 40%.

### Splicing Element

In some embodiment, a circular polyribonucleotide includes at least one splicing element. Exemplary splicing elements are described in paragraphs [0270]-[0275] of International Patent Publication No. WO2019/118919, which is hereby incorporated by reference in its entirety.

In some embodiments, a circular polyribonucleotide includes at least one splicing element. In a circular polyribonucleotide as provided herein, a splicing element can be a complete splicing element that can mediate splicing of the circular polyribonucleotide. Alternatively, the splicing element can also be a residual splicing element from a completed splicing event. For instance, in some cases, a splicing element of a linear polyribonucleotide can mediate a splicing event that results in circularization of the linear polyribonucleotide, thereby the resultant circular polyribonucleotide includes a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, a circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, A/u elements in flanking introns, and motifs found in (suptable4 enriched motifs) cis-sequence elements proximal to backsplice events such as sequences in the 200 bp preceding (upstream of) or following (downstream from) a backsplice site with flanking exons.

In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, a circular polyribonucleotide may include canonical splice sites that flank head-to-tail junctions of the circular polyribonucleotide.

In some embodiments, a circular polyribonucleotide may include a bulge-helix-bulge motif, including a 4-base pair stem flanked by two 3-nucleotide bulges. Cleavage occurs at a site in the bulge region, generating characteristic fragments with terminal 5′-hydroxyl group and 2′, 3′-cyclic phosphate.

Circularization proceeds by nucleophilic attack of the 5′-OH group onto the 2′, 3′-cyclic phosphate of the same molecule forming a 3′, 5′-phosphodiester bridge.

In some embodiments, a circular polyribonucleotide may include a multimeric repeating RNA sequence that harbors a HPR element. The HPR includes a 2′,3′-cyclic phosphate and a 5′-OH terminus.

The HPR element self-processes the 5′- and 3′-ends of the linear polyribonucleotide for circularization, thereby ligating the ends together.

In some embodiments, a circular polyribonucleotide may include a self-splicing element. For example, the circular polyribonucleotide may include an intron from the cyanobacteria Anabaena.

In some embodiments, a circular polyribonucleotide may include a sequence that mediates self-ligation. In one embodiment, the circular polyribonucleotide may include a HDV sequence (e.g., HDV replication domain conserved sequence, GGCUCAUCUCGACAAGAGGCGGCAGUCCUCAGUACUCUUACUCUUUUCUGUAAAGAGGAGACUG CUGGACUCGCCGCCCAAGUUCGAGCAUGAGCC (SEQ ID NO: 5) or GGCUAGAGGCGGCAGUCCUCAGUACUCUUACUCUUUUCUGUAAAGAGGAGACUGCUGGACUCGC CGCCCGAGCC (SEQ ID NO: 6) to self-ligate. In one embodiment, the circular polyribonucleotide may include loop E sequence (e.g., in PSTVd) to self-ligate. In another embodiment, the circular polyribonucleotide may include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear polyribonucleotides for circularization may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, the circular polyribonucleotide includes between 1 and 10 (e.g., 2, 3, 4, 5, 6, 7, 8, 9, and 10) repetitive nucleic acid sequences that hybridize to a complementary repetitive nucleic acid sequence in another segment of the circular polyribonucleotide, with the hybridized segment forming an internal double strand. In some embodiments, the circular polyribonucleotide includes 2 repetitive nucleic acid sequences that hybridize to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear polyribonucleotides for circularization. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide-based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motif useful for replication.

Exemplary replication elements are described in paragraphs [0280]-[0286] of International Patent Publication No. WO2019/118919, which is hereby incorporated by reference in its entirety.

### Purification of Circular Polyribonucleotides

In some embodiments, the circular polyribonucleotide is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Detection of Linear and Circular RNA

The presence of linear RNA in pharmaceutical circular RNA preparations can have unexpected and sometimes undesirable effects. Thus, circular RNAs may be enriched, separated, and/or purified relative to linear RNA; methods (e.g., methods of manufacturing circular RNA preparations) whereby linear RNAs can be monitored, evaluated and/or controlled; and methods of using such pharmaceutical compositions and preparations, e.g., to deliver an effector, such as a therapeutic effector or scaffold (e.g., an aptamer sequence), to a cell, tissue or subject. In some embodiments, a circular RNA preparation has no more than a threshold level of linear RNA, e.g., a circular RNA preparation is enriched over linear RNA or purified to reduce linear RNA.

Generally, detection and quantitation of an element in a pharmaceutical preparation includes the use of a reference standard that is either the component of interest (e.g., circular RNA, linear RNA, fragment, impurity, etc.) or is a similar material (e.g., using a linear RNA structure of the same sequence as a circular RNA structure as a standard for circular RNA), or includes the use of an internal standard or signal from a test sample. In some embodiments, the standard is used to establish the response from a detector for a known or relative amount of material (response factor). In some embodiments, the response factor is determined from a standard at one or multiple concentrations (e.g., using linear regression analysis). In some embodiments, the response factor is then used to determine the amount of the material of interest from the signal due to that component. In some embodiments, the response factor is a value of one or is assumed to have a value of one.

In some embodiments, detection and quantification of linear versus circular RNA in the pharmaceutical composition is determined using a comparison to a linear version of the circular polyribonucleotides. In some embodiments, the mass of total ribonucleotide in the pharmaceutical composition is determined using a standard curve generated using a linear version of the circular polyribonucleotide and assuming a response factor of one. In some embodiments, a w/w percentage of circular polyribonucleotide in the pharmaceutical preparation is determined by a comparison of a standard curve generated by band intensities of multiple known amounts of a linear version of the circular polyribonucleotide to a band intensity of the circular polyribonucleotide in the pharmaceutical preparation.

In some embodiments, the bands are produced during gel-base electrophoresis, and the band intensities are measured by a gel imager (e.g., an E-gel Imager). In some embodiments, a circular polyribonucleotide preparation includes less than a threshold amount (e.g., where the threshold amount is a reference criterion, e.g., a pharmaceutical release specification for the circular polyribonucleotide preparation) of linear polyribonucleotide molecules when evaluated as described herein.

In some embodiments, detection and quantification of nicked versus total RNA in the pharmaceutical composition is determined by sequencing after gel extraction of the preparation including the circular RNA. In some embodiments, detection and quantification of nicked versus linear RNA in the pharmaceutical composition is determined by sequencing after gel extraction of the preparation including the circular RNA. In some embodiments, a circular polyribonucleotide preparation includes less than a threshold amount (e.g., where the threshold amount is a reference criterion, e.g., a pharmaceutical release specification for the circular polyribonucleotide preparation) of nicked RNA, linear RNA, or combined linear and nicked RNA when evaluated as described herein. For example, the reference criterion for the amount of linear polyribonucleotide molecules present in the preparation is no more than 30%, 20%, 15%, 10%, 1%, 0.5%, or 0.1% linear polyribonucleotide molecules, or any percentage therebetween, relative to total ribonucleotide molecules in the preparation. In some embodiments, the reference criterion for the amount of nicked polyribonucleotide molecules present in the preparation is no more than 30%, 20%, 15%, 10%, 1%, 0.5%, or 0.1%, or any percentage therebetween, nicked polyribonucleotide molecules relative to total ribonucleotide molecules in the preparation. In some embodiments, the reference criterion for the amount of linear and nicked polyribonucleotide molecules present in the preparation is no more than 40%, 30%, 20%, 15%, 10%, 1%, 0.5%, or 0.1%, or any percentage therebetween, combined linear polyribonucleotide and nicked polyribonucleotide molecules relative to total ribonucleotide molecules in the preparation.

In some embodiments, the standard is run under the same conditions as the sample. For example, the standard is run with the same type of gel, same buffer, and same exposure as the sample. In further embodiments, the standard is run in parallel with the sample. In some embodiments, a quantification of an element is repeated (e.g., twice or in triplicate) in a plurality of samples from the subject preparation to obtain a mean result. In some embodiments, quantitation of a linear RNA is measured using parallel capillary electrophoresis (e.g., using a Fragment Analyzer or analytical HPLC with UV detection).

### Methods of Purification

Circular polyribonucleotides may be separated, enriched, or purified from unwanted substances (such as unwanted (e.g., linear) RNA, enzymes, DNA). In some embodiments, the unwanted substances are present in, or originating from, a process of making and/or manufacturing the circular polyribonucleotides. Circular polyribonucleotides described herein may be enriched and/or purified prior to formulation in a pharmaceutical preparation, pharmaceutical composition, pharmaceutical drug substance, or pharmaceutical drug product. Circular polyribonucleotides described herein may be enriched and/or purified during or after formulation in a pharmaceutical preparation, pharmaceutical composition, pharmaceutical drug substance, or pharmaceutical drug product.

In some embodiments, a circular polyribonucleotide may be purified during or after production to remove undesirable elements, e.g., linear RNA, or nicked RNA, as well as recognized impurities, e.g., free ribonucleic acids (e.g., monoribonucleic acids, diribonucleic acids, or triribonucleic acids), DNA (e.g., cell DNA, such as host cell DNA), cell or process-related protein impurities (e.g., cell or process-related impurities), etc. In some embodiments, an impurity is a process-related impurity. In some embodiments, the process-related impurity is a protein (e.g., a cell protein), a nucleic acid (e.g., a cell nucleic acid), a buffer or buffer reagent, an enzyme, a media/reagent component (e.g., media, media additive, transition metal, or vitamin), a preparatory or analytical gel component (e.g., acrylamide debris), DNA, or a chromatographic material. A buffer reagent can be MgCl2, DTT, ATP, SDS, Na, glycogen, Tris-HCL, or EtOH. A buffer reagent can include, but is not limited to, acetate, Tris, bicarbonate, phosphate, citric acid, lactate, or TEA. An enzyme can be a ligase. A ligase can be T4 RNA ligase 2. In some embodiments, an impurity is a buffer reagent, a media/reagent component, a salt, a ligase, a nuclease, an RNase inhibitor, RNase R, linear polyribonucleotide molecules, deoxyribonucleotide molecules, acrylamide debris, or mononucleotide molecules.

In some embodiments, circular polyribonucleotides may be enriched or purified by any known method commonly used in the art. Examples of non-limiting purification methods include column chromatography, gel excision, size exclusion, etc.

In some embodiments, a circular polyribonucleotide is purified by gel. For example, a circular RNA may be resolved on a denaturing PAGE and bands corresponding to the circular RNAs may be excised and the circular RNA may be eluted from the band using known methods. The eluted circular RNA may then be analyzed.

In some embodiments, a circular polyribonucleotide is purified by chromatography, e.g., hydrophobic interaction chromatography (HIC), mixed-mode chromatography, liquid chromatography, e.g., reverse-phase ion-pair chromatography (IP-RP), ion-exchange chromatography (IE), affinity chromatography (AC), and size-exclusion chromatography (SEC), and any combinations thereof.

In some embodiments, a circular polyribonucleotide is purified by utilizing a structural feature of the circular polyribonucleotide to separate it from a linear RNA or an impurity. In some embodiments, the circular polyribonucleotide is purified by utilizing a structural feature (e.g., a lack of free ends). For example, circular RNA is enriched from a preparation including a mixed pool of circular RNA and linear RNA counterpart containing the same nucleotide sequences using polyadenylation of the linear RNA counterpart or fragments thereof. The 3′ end of the linear RNA counterpart or fragments thereof can be polyadenylated using poly(A) polymerase, resulting in the addition of a 3′ polyadenine tail. In some embodiments, the 3′ polyadenine tail enables a pulldown of the linear RNA and fragments thereof using a column, such as an affinity column, to enrich for the circular RNA. Poly(A) polymerase can also incorporate modified adenines such as the biotinylated N6-ATP analog. This addition biotinylated N6-ATP analog into the 3′ polyadenine tail of enables a pulldown of the linear RNA and fragments thereof in a system such as a biotin-streptavidin binding system. In contrast, circularized RNA does not have a 3′ end, and therefore is not polyadenylated by the poly(A) polymerase, does not have a polyadenylated tail for conjugation, and is not captured in the pulldown. Therefore, the circular RNA is enriched in the preparation after the pulldown.

In some embodiments, a circular polyribonucleotide is purified by utilizing a structural feature of the linear RNA (e.g., presence of free ends). For example, circular RNA is enriched from a preparation including a mixed pool of circular RNA and linear RNA counterpart containing the same nucleotide sequences using polyadenylation of the linear RNA counterpart. Exonucleases can be added to the mixed pool to hydrolyze the linear RNA. In some embodiments, an exonuclease can be 3′ exonuclease or a 5′ exonuclease. In some embodiments, a 3′ exonuclease and a 5′ exonuclease can be used.

In an embodiment, a circular polyribonucleotide preparation (e.g., a circular polyribonucleotide pharmaceutical preparation or composition or an intermediate in the production of the circular polyribonucleotide preparation) is at least 25% (w/w), 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), or 100% (w/w) pure on a mass basis. Purity may be measured by any one of a number of analytical techniques known to one skilled in the art, such as, but not limited to, the use of separation technologies such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of a separation technology by mass spectrometry, by microscopy, by circular dichroism (CD) spectroscopy, by UV or UV-vis spectrophotometry, by fluorometry (e.g., Qubit), by RNase H analysis, by surface plasmon resonance (SPR), or by methods that utilize silver or dye stains or radioactive decay for detection.

In some embodiments, purity can be measured by biological test methodologies (e.g., cell-based or receptor-based tests). In some embodiments, at least 25% (w/w), 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w) or 100% (w/w) of the total of mass ribonucleotide in the a preparation described herein is contained in circular polyribonucleotide molecules.

The percent may be measured by any one of a number of analytical techniques known to one skilled in the art such as, but not limited to, the use of a separation technology such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of separation technologies by mass spectrometry, by microscopy, by circular dichroism (CD) spectroscopy, by UV or UV-vis spectrophotometry, by fluorometry (e.g., Qubit), by RNase H analysis, by surface plasmon resonance (SPR), or by methods that utilize silver or dye stains or radioactive decay for detection.

In an embodiment, a circular polyribonucleotide preparation (e.g., a circular polyribonucleotide pharmaceutical preparation or composition or an intermediate in the production of the circular polyribonucleotide preparation) has a circular polyribonucleotide concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, 100,000 μg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) is substantially free of mononucleotide or has a mononucleotide content of no more than 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL. In an embodiment, a circular polyribonucleotide preparation (e.g., a circular polyribonucleotide pharmaceutical preparation or composition or an intermediate in the production of the circular polyribonucleotide preparation) has a mononucleotide content from the limit of detection up to 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) has mononucleotide content no more than 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), or any percentage therebetween of total nucleotides on a mass basis, wherein total nucleotide content is the total mass of deoxyribonucleotide molecules and ribonucleotide molecules.

In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 6 Ong/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/mL, 10 mg/mL, 50 mg/mL, 100 mg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, from the limit of detection of up to 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 6 Ong/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/ml, 10 mg/ml, 50 mg/ml, 100 mg/ml, 200 mg/ml, 300 mg/ml, 400 mg/ml, 500 mg/ml, 600 mg/ml, 650 mg/ml, 700 mg/ml, or 750 mg/ml.

In an embodiment, a circular polyribonucleotide preparation (e.g., a circular polyribonucleotide pharmaceutical preparation or composition or an intermediate in the production of the circular polyribonucleotide preparation) has a nicked RNA content of no more than 10% (w/w), 9.9% (w/w), 9.8% (w/w), 9.7% (w/w), 9.6% (w/w), 9.5% (w/w), 9.4% (w/w), 9.3% (w/w), 9.2% (w/w), 9.1% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a nicked RNA content that as low as zero or is substantially free of nicked RNA.

In an embodiment, a circular polyribonucleotide preparation (e.g., a circular polyribonucleotide pharmaceutical preparation or composition or an intermediate in the production of the circular polyribonucleotide preparation) has a combined linear RNA and nicked RNA content of no more than 70% (w/w), 60% (w/w), 50% (w/w), 40% (w/w), 30% (w/w), 25% (w/w), 20% (w/w), 15% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a combined nicked RNA and linear RNA content that is as low as zero or is substantially free of nicked and linear RNA.

In some embodiments, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than the detection limit of analytical methodologies, such as methods utilizing mass spectrometry, UV spectroscopic or fluorescence detectors, light scattering techniques, surface plasmon resonance (SPR) with or without the use of methods of separation including HPLC, by HPLC, chip or gel based electrophoresis with or without using either pre or post separation derivatization methodologies, methods of detection that use silver or dye stains or radioactive decay, or microscopy, visual methods or a spectrophotometer.

In an embodiment, a circular polyribonucleotide preparation (e.g., a circular polyribonucleotide pharmaceutical preparation or composition or an intermediate in the production of the circular polyribonucleotide preparation) has no more than 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of linear RNA, e.g., as measured by the methods in Example 2.

In some embodiments, the linear polyribonucleotide molecules of a circular polyribonucleotide preparation include the linear counterpart or a fragment thereof of the circular polyribonucleotide molecule. In some embodiments, the linear polyribonucleotide molecules of the circular polyribonucleotide preparation include the linear counterpart (e.g., a pre-circularized version). In some embodiments, the linear polyribonucleotide molecules of the circular polyribonucleotide preparation include a non-counterpart or fragment thereof to the circular polyribonucleotide. In some embodiments, the linear polyribonucleotide molecules of the circular polyribonucleotide preparation include a non-counterpart to the circular polyribonucleotide. In some embodiments, the linear polyribonucleotide molecules include a combination of the counterpart of the circular polyribonucleotide and a non-counterpart or fragment thereof of the circular polyribonucleotide. In some embodiments, the linear polyribonucleotide molecules include a combination of the counterpart of the circular polyribonucleotide and a non-counterpart of the circular polyribonucleotide. In some embodiments, a linear polyribonucleotide molecule fragment is a fragment that is at least 1, 2, 3, 4, 5, 6, 7, 8, 9, 10, 20, 30, 40, 50, 60, 70, 80, 90, 100, 200, 300, 400, 500, 1000, 2000, 3000, 4000, 5000, 6000, 7000, 8000, 9000, 10000, 11000, 12000, or more nucleotides in length, or any nucleotide number therebetween.

In some embodiments, a circular polyribonucleotide preparation (e.g., a circular polyribonucleotide pharmaceutical preparation or composition or an intermediate in the production of the circular polyribonucleotide preparation) has an A260/A280 absorbance ratio from about 1.6 to about 2.3, e.g., as measured by spectrophotometer. In some embodiments, the A260/A280 absorbance ratio is about 1.4, 1.5, 1.6, 1.7, 1.8, 1.9, 2.0, 2.1, 2.2, 2.3, 2.4, 2.5, or any number therebetween. In some embodiments, a circular polyribonucleotide (e.g., a circular polyribonucleotide pharmaceutical preparation or composition or an intermediate in the production of the circular polyribonucleotide) has an A260/A280 absorbance ratio greater than about 1.8, e.g., as measured by spectrophotometer. In some embodiments, the A260/A280 absorbance ratio is about 1.6, 1.7, 1.8, 1.9, 2.0, 2.1, 2.2, 2.3, 2.4, 2.5, 2.6, 2.7, 2.8, 2.9, or greater.

In some embodiments, a circular polyribonucleotide preparation (e.g., a circular polyribonucleotide pharmaceutical preparation or composition or an intermediate in the production of the circular polyribonucleotide preparation) is substantially free of an impurity. In various embodiments, the level of at least one impurity in a composition including the circular polyribonucleotide is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity. In some embodiments, the level of at least one process-related impurity is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity. In some embodiments, the level of at least one product-related substance is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the a composition prior to purification or treatment to remove the impurity. In some embodiments, a circular polyribonucleotide preparation (e.g., a circular polyribonucleotide pharmaceutical preparation or composition or an intermediate in the production of the circular polyribonucleotide preparation) is further substantially free of a process-related impurity. In some embodiments, the process-related impurity includes a protein (e.g., a cell protein, such as a host cell protein), a deoxyribonucleic acid (e.g., a cell deoxyribonucleic acid, such as a host cell deoxyribonucleic acid), monodeoxyribonucleotide or dideoxyribonucleotide molecules, an enzyme (e.g., a nuclease, such as an endonuclease or exonuclease, or ligase), a reagent component, a gel component, or a chromatographic material. In some embodiments, the impurity is selected from: a buffer reagent, a ligase, a nuclease, RNase inhibitor, RNase R, deoxyribonucleotide molecules, acrylamide gel debris, and monodeoxyribonucleotide molecules. In some embodiments, the pharmaceutical preparation includes protein (e.g., cell protein, such as a host cell protein) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination per milligram (mg) of the circular polyribonucleotide molecules.

In an embodiment, a circular polyribonucleotide preparation (e.g., a circular polyribonucleotide pharmaceutical preparation or composition or an intermediate in the production of the circular polyribonucleotide preparation) is substantially free of DNA content e.g., template DNA or cell DNA (e.g., host cell DNA), has a DNA content, as low as zero, or has a DNA content of no more than 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) is substantially free of DNA content, has a DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis, wherein total nucleotide molecules is the total mass of deoxyribonucleotide content and ribonucleotide molecules. In an embodiment, a circular polyribonucleotide preparation (e.g., a circular polyribonucleotide pharmaceutical preparation or composition or an intermediate in the production of the circular polyribonucleotide preparation) is substantially free of DNA content, has DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis as measured after a total DNA digestion by enzymes that digest nucleosides by quantitative liquid chromatography-mass spectrometry (LC-MS), in which the content of DNA is back calculated from a standard curve of each base (i.e., A, C, G, T) as measured by LC-MS.

In an embodiment, a circular polyribonucleotide preparation (e.g., a circular polyribonucleotide pharmaceutical preparation or composition or an intermediate in the production of the circular polyribonucleotide preparation) has a protein (e.g., cell protein (CP), e.g., enzyme, a production-related protein, e.g., carrier protein) contamination of no more than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination from the limit of detection of up to 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, a circular polyribonucleotide preparation (e.g., a circular polyribonucleotide pharmaceutical preparation or composition or an intermediate in the production of the circular polyribonucleotide preparation) has a protein (e.g., production-related protein such as a cell protein (CP), e.g., enzyme) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination from the level of detection up to 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide.

In an embodiment, a circular polyribonucleotide preparation (e.g., a circular polyribonucleotide pharmaceutical preparation or composition or an intermediate in the production of the circular polyribonucleotide preparation) has low levels or is substantially absent of endotoxins, e.g., as measured by the Limulus amebocyte lysate (LAL) test. In some embodiments, the pharmaceutical preparation or compositions or an intermediate in the production of the circular polyribonucleotides includes less than 20 EU/kg (weight), 10 EU/kg, 5 EU/kg, 1 EU/kg, or lacks endotoxin as measured by the Limulus amebocyte lysate test. In an embodiment, a circular polyribonucleotide composition has low levels or absence of a nuclease or a ligase.

In some embodiments, a circular polyribonucleotide preparation (e.g., a circular polyribonucleotide pharmaceutical preparation or composition or an intermediate in the production of the circular polyribonucleotide preparation) includes no greater than about 50% (w/w), 45% (w/w), 40% (w/w), 35% (w/w), 30% (w/w), 25% (w/w), 20% (w/w), 19% (w/w), 18% (w/w), 17% (w/w), 16% (w/w), 15% (w/w), 14% (w/w), 13% (w/w), 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w) of at least one enzyme, e.g., polymerase, e.g., RNA polymerase.

In an embodiment, a circular polyribonucleotide preparation (e.g., a circular polyribonucleotide pharmaceutical preparation or composition or an intermediate in the production of the circular polyribonucleotide preparation) is sterile or substantially free of microorganisms, e.g., the composition or preparation supports the growth of fewer than 100 viable microorganisms as tested under aseptic conditions, the composition or preparation meets the standard of USP <71>, and/or the composition or preparation meets the standard of USP <85>. In some embodiments, the pharmaceutical preparation includes a bioburden of less than 100 CFU/100 ml, 50 CFU/100 ml, 40 CFU/100 ml, 30 CFU/100 ml, 200 CFU/100 ml, 10 CFU/100 ml, or 10 CFU/100 ml before sterilization.

In some embodiments, a circular polyribonucleotide preparation can be further purified using known techniques in the art for removing impurities, such as column chromatography or pH/vial inactivation.

In some embodiments, a circular polyribonucleotide preparation produces a reduced level of one more markers of an immune or inflammatory response after administration to a subject when the circular polyribonucleotide preparation has undergone a purification step (or a plurality of purification steps) compared to prior to the purification step(s). In some embodiments, the one or more markers of an immune or inflammatory response is a cytokine or immune response related gene. In some embodiments, the one or more markers of an immune or inflammatory response is expression of a gene, such as RIG-I, MDA5, PKR, IFN-beta, OAS, and OASL.

In an embodiment, a circular polyribonucleotide preparation (e.g., a circular polyribonucleotide pharmaceutical preparation or composition or an intermediate in the production of the circular polyribonucleotide preparation) expresses an expression product, e.g., protein, e.g., in-vitro translation activity.

### Delivery

A circular or linear polyribonucleotide described herein may be included in pharmaceutical compositions with a carrier or without a carrier.

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

In some embodiments, circular or linear polyribonucleotides may be delivered in a “naked” delivery formulation. A naked delivery formulation delivers a circular polyribonucleotide to a cell without the aid of a carrier and without covalent modification of the circular or linear polyribonucleotide or partial or complete encapsulation of the circular or linear polyribonucleotide.

A naked delivery formulation is a formulation that is free from a carrier and wherein the circular or linear polyribonucleotide is without a covalent modification that binds a moiety that aids in delivery to a cell and the circular or linear polyribonucleotide is not partially or completely encapsulated. In some embodiments, an circular or linear polyribonucleotide without covalent modification that binds to a moiety that aids in delivery to a cell may be a polyribonucleotide that is not covalently bound to a moiety, such as a protein, small molecule, a particle, a polymer, or a biopolymer that aids in delivery to a cell. In some embodiments, circular or linear polyribonucleotides may be delivered in a delivery formulation with protamine or a protamine salt (e.g., protamine sulfate).

A polyribonucleotide without covalent modification that binds to a moiety that aids in delivery to a cell may not contain a modified phosphate group. For example, a polyribonucleotide without covalent modification that binds to a moiety that aids in delivery to a cell may not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may include a non-carrier excipient. In some embodiments, a non-carrier excipient may include an inactive ingredient that does not exhibit an active cell-penetrating effect. In some embodiments, a non-carrier excipient may include a buffer, for example PBS. In some embodiments, a non-carrier excipient may be a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may include a diluent, such as a parenterally acceptable diluent. A diluent (e.g., a parenterally acceptable diluent) may be a liquid diluent or a solid diluent. In some embodiments, a diluent (e.g., a parenterally acceptable diluent) may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the pharmaceutical preparation as disclosed herein, the pharmaceutical composition as disclosed herein, the pharmaceutical drug substance of as disclosed, or the pharmaceutical drug product as disclosed herein is in parenteral nucleic acid delivery system. The parental nucleic acid delivery system may include the pharmaceutical preparation as disclosed herein, the pharmaceutical composition as disclosed herein, the pharmaceutical drug substance of as disclosed, or the pharmaceutical drug product as disclosed herein, and a parenterally acceptable diluent. In some embodiments, the pharmaceutical preparation as disclosed herein, the pharmaceutical composition as disclosed herein, the pharmaceutical drug substance of as disclosed, or the pharmaceutical drug product as disclosed herein in the parenteral nucleic acid delivery system is free of any carrier.

The disclosure is further directed to a host or host cell including the circular or linear polyribonucleotide described herein. In some embodiments, the host or host cell is a vertebrate, mammal (e.g., human), or other organism or cell.

In some embodiments, the circular polyribonucleotide has a decreased, or fails to produce a, undesired response by the host's immune system as compared to the response triggered by a reference compound, e.g., a linear polynucleotide corresponding to the described circular polyribonucleotide or a circular polyribonucleotide lacking an encryptogen. In embodiments, the circular polyribonucleotide is non-immunogenic in the host. Some immune responses include, but are not limited to, humoral immune responses (e.g. production of immunogen-specific antibodies) and cell-mediated immune responses (e.g., lymphocyte proliferation).

In some embodiments, a host or a host cell is contacted with (e.g., delivered to or administered to) the circular polyribonucleotide or linear. In some embodiments, the host is a mammal, such as a human. The amount of the circular polyribonucleotide or linear, expression product, or both in the host can be measured at any time after administration. In certain embodiments, a time course of host growth in a culture is determined. If the growth is increased or reduced in the presence of the circular polyribonucleotide or linear, the circular polyribonucleotide or expression product or both is identified as being effective in increasing or reducing the growth of the host.

A method of delivering a circular or linear polyribonucleotide molecule as described herein to a cell, tissue, or subject, includes administering the pharmaceutical composition, pharmaceutical drug substance or pharmaceutical drug product as described herein to the cell, tissue, or subject.

In some embodiments, the cell is a eukaryotic cell. In some embodiments, the cell is a mammalian cell. In some embodiments, the cell is an ungulate cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.).

In some embodiments, the method of delivering is an in vivo method. For example, a method of delivery of a circular polyribonucleotide as described herein includes parenterally administering to a subject in need thereof, the pharmaceutical composition, pharmaceutical drug substance or pharmaceutical drug product as described herein to the subject in need thereof. As another example, a method of delivering a circular polyribonucleotide to a cell or tissue of a subject, includes administering parenterally to the cell or tissue the pharmaceutical composition, pharmaceutical drug substance or pharmaceutical drug product as described herein. In some embodiments, the circular polyribonucleotide is in an amount effective to elicit a biological response in the subject. In some embodiments, the circular polyribonucleotide is an amount effective to have a biological effect on the cell or tissue in the subject. In some embodiments, the pharmaceutical composition, pharmaceutical drug substance or pharmaceutical drug product as described herein includes a carrier. In some embodiments the pharmaceutical composition, pharmaceutical drug substance or pharmaceutical drug product as described herein includes a diluent and is free of any carrier.

In some embodiments the pharmaceutical composition, the pharmaceutical drug substance, or the pharmaceutical drug product is administered parenterally. In some embodiments the pharmaceutical composition, the pharmaceutical drug substance, or the pharmaceutical drug product is administered intravenously, intraarterially, intraperitoneally, intradermally, intracranially, intrathecally, intralymphaticly, subcutaneously, or intramuscularly. In some embodiments, parenteral administration is intravenously, intramuscularly, ophthalmically, subcutaneously, intradermally or topically.

In some embodiments, the pharmaceutical composition, pharmaceutical drug substance or pharmaceutical drug product as described herein is administered intramuscularly. In some embodiments, the pharmaceutical composition, pharmaceutical drug substance or pharmaceutical drug product as described herein is administered subcutaneously. In some embodiments, the pharmaceutical composition, pharmaceutical drug substance or pharmaceutical drug product as described herein is administered topically. In some embodiments, the pharmaceutical composition, the pharmaceutical drug substance, or the pharmaceutical drug product is administered intratracheally.

In some embodiments the pharmaceutical composition, pharmaceutical drug substance or pharmaceutical drug product is administered by injection. The administration can be systemic administration or local administration. In some embodiments, any of the methods of delivery as described herein are performed with a carrier. In some embodiments, any methods of delivery as described herein are performed without the aid of a carrier or cell penetrating agent.

In some embodiments, the circular polyribonucleotide or a product translated from the circular polyribonucleotide is detected in the cell, tissue, or subject at least 1 day, at least 2 days, at least 3 days, at least 4 days, or at least 5 days after the administering step. In some embodiments, the presence of the circular polyribonucleotide or a product translated from the circular polyribonucleotide is evaluated in the cell, tissue, or subject before the administering step. In some embodiments, the presence of the circular polyribonucleotide or a product translated from the circular polyribonucleotide is evaluated in the cell, tissue, or subject after the administering step.

### Formulations

In some embodiments, a pharmaceutical formulation disclosed herein can include: (i) a compound (e.g., circular polyribonucleotide) disclosed herein; (ii) a buffer; (iii) a non-ionic detergent; (iv) a tonicity agent; and/or (v) a stabilizer. In some embodiments, a pharmaceutical formulation disclosed herein can include: (i) a compound (e.g., linear polyribonucleotide) disclosed herein; (ii) a buffer; (iii) a non-ionic detergent; (iv) a tonicity agent; and/or (v) a stabilizer. In some embodiments, the pharmaceutical formulation disclosed herein is a stable liquid pharmaceutical formulation. In some embodiments, the pharmaceutical formulation disclosed herein includes protamine or a protamine salt (e.g., protamine sulfate).

The disclosure provides immunogenic compositions including a circular polyribonucleotide described herein. The disclosure provides immunogenic compositions including a linear polyribonucleotide described herein. Immunogenic compositions of the disclosure may include a diluent or a carrier, adjuvant, or any combination thereof. Immunogenic compositions of the disclosure may also include one or more immunoregulatory agents, e.g., one or more adjuvants. The adjuvants may include a TH1 adjuvant and/or a TH2 adjuvant, further discussed below. In some embodiments, the immunogenic composition includes a diluent free of any carrier and is used for naked delivery of the circular polyribonucleotide to a subject. In some embodiments, the immunogenic composition includes a diluent free of any carrier and is used for naked delivery of the linear polyribonucleotide to a subject.

Immunogenic compositions of the disclosure are used to raise an immune response in a subject. The immune response is preferably protective and preferably involves an antibody response (usually including IgG) and/or a cell-mediated immune response. For example, a subject is immunized with an immunogenic composition including a circular polyribonucleotide of the disclosure to induce an immune response. In another example, a subject is immunized with an immunogenic composition including a linear polyribonucleotide including an immunogen to stimulate production of antibodies that bind to the immunogen. By raising an immune response in the subject by these uses and methods, the subject can be protected against various diseases and/or infections e.g. against bacterial and/or viral diseases as discussed above. In certain embodiments, the immunogenic compositions are vaccine compositions. Vaccines according to the disclosure may either be prophylactic (i.e. to prevent infection) or therapeutic (i.e. to treat infection) but will typically be prophylactic. In some embodiments, the subject is a mammal. In some embodiments, the subject is an animal, preferably a mammal, e.g., a human. In one embodiment, the subject is a human. In other embodiments the subject is a non-human mammal, e.g., selected from a cow (e.g., dairy and beef cattle), a sheep, a goat, a pig, a horse, a dog, or a cat. In other embodiments the subject is a bird, e.g., a hen or rooster, turkey, parrot. In some embodiments, the animal is not a mouse or a rabbit or a cow. In a particular embodiment, where the immunogenic composition is for prophylactic use, the human is a child (e.g. a toddler or infant) or a teenager. In another embodiment, where the immunogenic composition is for therapeutic use, the human is a teenager or an adult. An immunogenic composition intended for children may also be administered to adults e.g. to assess safety, dosage, immunogenicity, etc.

Immunogenic composition prepared according to the disclosure may be used to treat both children and adults. A human subject may be less than 1 year old, less than 5 years old, 1-5 years old, 5-15 years old, 15-55 years old, or at least 55 years old. In a particular embodiment, a human subject for receiving the immunogenic compositions are the elderly (e.g., >50 years old, >60 years old, and >65 years), the young (e.g., 55 years old), hospitalized patients, healthcare workers, armed service and military personnel, pregnant women, the chronically ill, or immunodeficient patients. The immunogenic compositions are not suitable solely for these groups, however, and may be used more generally in a population.

In some embodiments, the subject is further immunized with an adjuvant. In some embodiments the subject is further immunized with a vaccine.

### Immunization

In some embodiments, methods of the disclosure include immunizing a subject with an immunogenic composition including a circular polyribonucleotide as disclosed herein. In some embodiments, an immunogen is expressed from the circular polyribonucleotide. In some embodiments, immunization induces an immune response in a subject against the immunogen expressed from the circular polyribonucleotide. In some embodiments, immunization induces an immune response in a subject (e.g., induces the production of antibodies that bind to the immunogen expressed from the circular polyribonucleotide). In some embodiments, an immunogenic composition includes the circular polyribonucleotide and a diluent, carrier, first adjuvant or a combination thereof in a single composition. In some embodiments, the subject is further immunized with a second adjuvant. In some embodiments, the subject is further immunized with a vaccine.

In some embodiments, methods of the disclosure include immunizing a subject with an immunogenic composition including a linear polyribonucleotide as disclosed herein. In some embodiments, an immunogen is expressed from the linear polyribonucleotide. In some embodiments, immunization induces an immune response in a subject against the immunogen expressed from the linear polyribonucleotide. In some embodiments, immunization induces the production of antibodies that bind to the immunogen expressed from the linear polyribonucleotide. In some embodiments, immunization induces a cell-mediated immune response. In some embodiments, an immunogenic composition includes the linear polyribonucleotide and a diluent, carrier, first adjuvant or a combination thereof in a single composition. In some embodiments, the subject is further immunized with a second adjuvant. In some embodiments, the subject is further immunized with a vaccine.

The subject is immunized with one or more immunogenic composition(s) including any number of circular polyribonucleotides. The subject is immunized with, for example, one or more immunogenic composition(s) including at least 1 circular polyribonucleotide. A non-human animal having a non-humanized immune system is immunized with, for example, one or more immunogenic composition(s) including at least 2, at least 3, at least 4, at least 5, at least 6, at least 7, at least 8, at least 9, at least 10, at least 11, at least 12, at least 13, at least 14, at least 15, at least 20 different circular polyribonucleotides, or more different circular polyribonucleotides. In some embodiments, a subject is immunized with one or more immunogenic composition(s) including at most 1 circular polyribonucleotide.

In some embodiments, a non-human animal having a humanized immune system is immunized with one or more immunogenic composition(s) including at most 2, at most 3, at most 4, at most 5, at most 6, at most 7, at most 8, at most 9, at most 10, at most 11, at most 12, at most 13, at most 14, at most 15, at most 20 different circular polyribonucleotides, or less than 21 different circular polyribonucleotides. In some embodiments, a subject is immunized with one or more immunogenic composition(s) including about 1 circular polyribonucleotide. In some embodiments, a non-human animal having a humanized immune system is immunized with one or more immunogenic composition(s) including about 2, about 3, about 4, about 5, about 6, about 7, about 8, about 9, about 10, about 11, about 12, about 13, about 14, about 15, or about 20 different circular polyribonucleotides. In some embodiments, a subject is immunized with one or more immunogenic composition(s) including about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different circular polyribonucleotides. Different circular polyribonucleotides have different sequences from each other. For example, they can include or encode different immunogens, overlapping immunogens, similar immunogens, or the same immunogens (for example, with the same or different regulatory elements, initiation sequences, promoters, termination elements, or other elements of the disclosure). In cases where a subject is immunized with one or more immunogenic composition(s) including two or more different circular polyribonucleotides, the two or more different circular polyribonucleotides can be in the same or different immunogenic compositions and immunized at the same time or at different times. The immunogenic compositions including two or more different circular polyribonucleotides can be administered to the same anatomical location or different anatomical locations.

The subject can be immunized with one or more immunogenic composition(s) including any number of linear polyribonucleotides. The subject is immunized with, for example, one or more immunogenic composition(s) including at least 1 linear polyribonucleotide. A non-human animal having a non-humanized immune system is immunized with, for example, one or more immunogenic composition(s) including at least 2, at least 3, at least 4, at least 5, at least 6, at least 7, at least 8, at least 9, at least 10, at least 11, at least 12, at least 13, at least 14, at least 15, at least 20 different linear polyribonucleotides, or more different linear polyribonucleotides. In some embodiments, a subject is immunized with one or more immunogenic composition(s) including at most 1 linear polyribonucleotide. In some embodiments, a non-human animal having a humanized immune system is immunized with one or more immunogenic composition(s) including at most 2, at most 3, at most 4, at most 5, at most 6, at most 7, at most 8, at most 9, at most 10, at most 11, at most 12, at most 13, at most 14, at most 15, at most 20 different linear polyribonucleotides, or less than 21 different linear polyribonucleotides. In some embodiments, a subject is immunized with one or more immunogenic composition(s) including about 1 linear polyribonucleotide. In some embodiments, a non-human animal having a humanized immune system is immunized with one or more immunogenic composition(s) including about 2, about 3, about 4, about 5, about 6, about 7, about 8, about 9, about 10, about 11, about 12, about 13, about 14, about 15, or about 20 different linear polyribonucleotides. In some embodiments, a subject is immunized with one or more immunogenic composition(s) including about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different linear polyribonucleotides. Different linear polyribonucleotides can have different sequences from each other. For example, they can include or encode different immunogens, overlapping immunogens, similar immunogens, or the same immunogens (for example, with the same or different regulatory elements, initiation sequences, promoters, termination elements, or other elements of the disclosure). In cases where a subject is immunized with one or more immunogenic composition(s) including two or more different linear polyribonucleotides, the two or more different linear polyribonucleotides can be in the same or different immunogenic compositions and immunized at the same time or at different times. The immunogenic compositions including two or more different linear polyribonucleotides can be administered to the same anatomical location or different anatomical locations.

The two or more different linear polyribonucleotides can include or encode immunogens from the same source, different source, or different combinations of sources disclosed herein. The two or more different linear polyribonucleotides can include or encode immunogens from the same virus or from different viruses, for example, different isolates.

In some embodiments, the subject is immunized with one or more immunogenic composition(s) including any number of circular polyribonucleotides and one or more immunogenic composition(s) including any number of linear polyribonucleotides as disclosed herein. In some embodiments, an immunogenic composition disclosed herein includes one or more circular polyribonucleotides and one or more linear polyribonucleotides as disclosed herein.

In some embodiments, an immunogenic composition includes a circular polyribonucleotide and a diluent, a carrier, a first adjuvant, or a combination thereof. In a particular embodiment, an immunogenic composition includes a circular polyribonucleotide described herein and a carrier or a diluent free of any carrier. In some embodiments, an immunogenic composition including a circular polyribonucleotide with a diluent free of any carrier is used for naked delivery of the circular polyribonucleotide to a subject. In another particular embodiment, an immunogenic composition includes a circular polyribonucleotide described herein and a first adjuvant.

In certain embodiments, a subject is further administered a second adjuvant. An adjuvant enhances the innate immune response, which in turn, enhances the adaptive immune response in a subject. An adjuvant can be any adjuvant as discussed below. In certain embodiments, an adjuvant is formulated with the circular polyribonucleotide as a part of an immunogenic composition. In certain embodiments, an adjuvant is not part of an immunogenic composition including the circular polyribonucleotide. In certain embodiments, an adjuvant is administered separately from an immunogenic composition including the circular polyribonucleotide. In this aspect, the adjuvant is co-administered (e.g., administered simultaneously) or administered at a different time than an immunogenic composition including the circular polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the circular polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the circular polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition including the circular polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition including the circular polyribonucleotide. The adjuvant is administered to the same anatomical location or different anatomical location as the immunogenic composition including the circular polyribonucleotide.

In some embodiments, an immunogenic composition includes a linear polyribonucleotide and a diluent, a carrier, a first adjuvant, or a combination thereof. In a particular embodiment, an immunogenic composition includes a linear polyribonucleotide described herein and a carrier or a diluent free of any carrier. In some embodiments, an immunogenic composition including a linear polyribonucleotide with a diluent free of any carrier is used for naked delivery of the linear polyribonucleotide to a subject. In another particular embodiment, an immunogenic composition includes a linear polyribonucleotide described herein and a first adjuvant.

In certain embodiments, a subject is further administered a second adjuvant. An adjuvant enhances the innate immune response, which in turn, enhances the adaptive immune response in a subject. An adjuvant can be any adjuvant as discussed below. In certain embodiments, an adjuvant is formulated with the linear polyribonucleotide as a part of an immunogenic composition. In certain embodiments, an adjuvant is not part of an immunogenic composition including the linear polyribonucleotide. In certain embodiments, an adjuvant is administered separately from an immunogenic composition including the linear polyribonucleotide. In this aspect, the adjuvant is co-administered (e.g., administered simultaneously) or administered at a different time than an immunogenic composition including the linear polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the linear polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the linear polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition including the linear polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition including the linear polyribonucleotide. The adjuvant is administered to the same anatomical location or different anatomical location as the immunogenic composition including the linear polyribonucleotide.

In some embodiments, a subject is further immunized with a second agent, e.g., a vaccine (as described below) that is not a circular polyribonucleotide. The vaccine is co-administered (e.g., administered simultaneously) or administered at a different time than an immunogenic composition including the circular polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the circular polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the circular polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition including the circular polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition including the circular polyribonucleotide.

In some embodiments, a subject is further immunized with a second agent, e.g., a vaccine (as described below) that is not a linear polyribonucleotide. The vaccine is co-administered (e.g., administered simultaneously) or administered at a different time than an immunogenic composition including the linear polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the linear polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the linear polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition including the linear polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition including the linear polyribonucleotide.

A subject can be immunized with an immunogenic composition, adjuvant, vaccine (e.g., protein subunit vaccine), or a combination thereof any suitable number of times to achieve a desired response.

For example, a prime-boost immunization strategy can be utilized to elicit systemic and/or mucosal immunity. A subject can be immunized with an immunogenic composition, adjuvant, vaccine (e.g., protein subunit vaccine), or a combination thereof, of the disclosure, for example, at least 1, at least 2, at least 3, at least 4, at least 5, at least 6, at least 7, at least 8, at least 9, at least 10, or at least 15 times, or more.

In some embodiments, a subject can be immunized with an immunogenic composition, adjuvant, vaccine (e.g., protein subunit vaccine), or a combination thereof, of the disclosure at most 2, at most 3, at most 4, at most 5, at most 6, at most 7, at most 8, at most 9, at most 10, at most 15, or at most 20 times, or less.

In some embodiments, a subject can be immunized with an immunogenic composition, adjuvant, vaccine (e.g., protein subunit vaccine), or a combination thereof, of the disclosure about 1, 2, 3, 4, 5, 6, 7, 8, 9, 10, 15, or 20 times.

In some embodiments, a subject can be immunized with an immunogenic composition, adjuvant, vaccine (e.g., protein subunit vaccine), or a combination thereof, of the disclosure once. In some embodiments, a subject can be immunized with an immunogenic composition, adjuvant, vaccine (e.g., protein subunit vaccine), or a combination thereof, of the disclosure twice. In some embodiments, a subject can be immunized with an immunogenic composition, adjuvant, vaccine (e.g., protein subunit vaccine), or a combination thereof, of the disclosure three times. In some embodiments, a subject can be immunized with an immunogenic composition, adjuvant, vaccine (e.g., protein subunit vaccine), or a combination thereof, of the disclosure four times. In some embodiments, a subject can be immunized with an immunogenic composition, adjuvant, vaccine (e.g., protein subunit vaccine), or a combination thereof, of the disclosure five times. In some embodiments, a subject can be immunized with an immunogenic composition, adjuvant, vaccine (e.g., protein subunit vaccine), or a combination thereof, of the disclosure seven times.

Suitable time intervals can be selected for spacing two or more immunizations. The time intervals can apply to multiple immunizations with the same immunogenic composition, adjuvant, or vaccine (e.g., protein subunit vaccine), or combination thereof, for example, the same immunogenic composition, adjuvant, or vaccine (e.g., protein subunit vaccine), or combination thereof, can be administered in the same amount or a different amount, via the same immunization route or a different immunization route. The time intervals can apply to multiple immunizations with a different immunogenic composition, adjuvant, or vaccine (e.g., protein subunit vaccine), or combination thereof, for example, a different immunogenic composition, adjuvant, or vaccine (e.g., protein subunit vaccine), or combination thereof, can be administered in the same amount or a different amount, via the same immunization route or a different immunization route. The time intervals can apply to immunizations with different agents, for example, a first immunogenic composition including a first circular polyribonucleotide and a second immunogenic composition including a second circular polyribonucleotide. The time intervals can apply to immunizations with different agents, for example, a first immunogenic composition including a first circular polyribonucleotide and a second immunogenic composition including a protein immunogen (e.g., a protein subunit). The time intervals can apply to a first immunogenic composition including a first linear polyribonucleotide and a second immunogenic composition including a second linear polyribonucleotide.

For regimens including three or more immunizations, the time intervals between immunizations can be the same or different. In some examples, about 1, 2, 3, 4, 5, 6, 7, 8, 9, 10, 11, 12, 14, 16, 17, 18, 20, 22, 24, 26, 28, 30, 32, 34, 36, 40, 48, or 72 hours elapse between two immunizations. In some embodiments, about 1, 2, 3, 4, 5, 6, 7, 8, 9, 10, 11, 12, 14, 16, 17, 18, 20, 21, 24, 28, or 30 days elapse between two immunizations. In some embodiments, about 1, 2, 3, 4, 5, 6, 7, or 8 weeks elapse between two immunizations. In some embodiments, about 1, 2, 3, 4, 5, 6, 7, or 8 months elapse between two immunizations.

In some embodiments, at least 1, at least 2, at least 3, at least 4, at least 5, at least 6, at least 7, at least 8, at least 9, at least 10, at least 15, at least 20, at least 24, at least 36, or at least 72 hours, or more elapse between two immunizations. In some embodiments, at most 1, at most 2, at most 3, at most 4, at most 5, at most 6, at most 7, at most 8, at most 9, at most 10, at most 15, at most 20, at most 24, at most 36, or at most 72 hours, or less elapse between two immunizations.

In some embodiments, at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, elapse between two immunizations. In some embodiments, at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 32, at most 34, or at most 36 days, or less elapse between two immunizations.

In some embodiments, at least 1, at least 2, at least 3, at least 4, at least 5, at least 6, at least 7, or at least 8 weeks, or more elapse between two immunizations. In some embodiments, at most 2, at most 3, at most 4, at most 5, at most 6, at most 7, at most 8 weeks, or less elapse between two immunizations.

In some embodiments, at least 1, at least 2, at least 3, at least 4, at least 5, at least 6, at least 7, or at least 8 months, or more elapse between two immunizations. In some embodiments, at most 2, at most 3, at most 4, at most 5, at most 6, at most 7, at most 8 months, at most 9 months, at most 10 months, at most 11 months, or at most 12 months or less elapse between two immunizations.

In some embodiments, the method includes pre-administering to the subject an agent to improve immunogenic responses to a circular polyribonucleotide including a sequence encoding an immunogen. In some embodiments, the agent is the immunogen as disclosed herein (e.g., a protein immunogen). For example, the method includes administering the protein immunogen from 1 to 7 days prior to administration of the circular polyribonucleotide including the sequence encoding the protein immunogen.

In some embodiments, the protein immunogen is administered 1, 2, 3, 4, 5, 6, or 7 days prior to administration of the circular polyribonucleotide including the sequence encoding the protein immunogen. For example, the method includes administering the protein immunogen from 1 to 7 days prior to administration of the linear polyribonucleotide including the sequence encoding the protein immunogen. In some embodiments, the protein immunogen is administered 1, 2, 3, 4, 5, 6, or 7 days prior to administration of the linear polyribonucleotide including the sequence encoding the protein immunogen. The protein immunogen may be administered as a protein preparation, encoded in a plasmid (pDNA), presented in a virus-like particle (VLP), formulated in a lipid nanoparticle, or the like.

In some embodiments, the method includes administering to the subject an agent to improve immunogenic responses to a circular polyribonucleotide including a sequence encoding an immunogen after the subject has been administered the circular polyribonucleotide including a sequence encoding an immunogen. In some embodiments, the agent is the immunogen as disclosed herein (e.g., a protein immunogen). In some embodiments, the circular polyribonucleotide includes a sequence encoding a protein immunogen. For example, the method includes administering the protein immunogen within 1 year (e.g., within 11 months, 10 months, 9 months, 8 months, 7 months, 6 months, 5 months, 4 months, 3 months, 2 months, and 1 month) of administering the circular polyribonucleotide including a sequence encoding the immunogen to the subject. In some embodiments, the method includes administering any one of the circular polyribonucleotides described herein or any one of the immunogenic compositions described herein and a protein subunit to the subject.

In some embodiments, the protein immunogen has the same amino acid sequence as the immunogen encoded by circular polyribonucleotide. For example, the polypeptide immunogen may correspond to (e.g., shares 90%, 95%, 96%, 97%, 98%, or 100%) amino acid sequence identity with a polypeptide immunogen encoded by a sequence of the circular polyribonucleotide. In some embodiments, the protein immunogen has a different amino acid sequence from the amino acid sequence of the immunogen encoded by the circular polyribonucleotide. For example, the polypeptide immunogen may share less than 90% (e.g., 80%, 70%, 30%, 20%, or 10%) amino acid sequence identity with the polypeptide immunogen encoded by a sequence of the circular polyribonucleotide.

A subject can be immunized with an immunogenic composition, an adjuvant, or a vaccine (e.g., protein subunit vaccine), or a combination thereof, at any suitable number anatomical sites. The same immunogenic composition, an adjuvant, a vaccine (e.g., protein subunit vaccine), or a combination thereof can be administered to multiple anatomical sites, different immunogenic compositions including the same or different circular polyribonucleotides, adjuvants, vaccines (e.g., protein subunit vaccine) or a combination thereof can be administered to different anatomical sites, different immunogenic compositions including the same or different circular polyribonucleotides, adjuvants, vaccines (e.g., protein subunit vaccines) or a combination thereof can be administered to the same anatomical site, or any combination thereof. For example, an immunogenic composition including a circular polyribonucleotide can be administered in to two different anatomical sites, and/or an immunogenic composition including a circular polyribonucleotide can be administered to one anatomical site, and an adjuvant can be administered to a different anatomical site. The same immunogenic composition, an adjuvant, a vaccine (e.g., protein subunit vaccine), or a combination thereof can be administered to multiple anatomical sites, different immunogenic compositions including the same or different linear polyribonucleotides, adjuvants, vaccines (e.g., protein subunit vaccine) or a combination thereof can be administered to different anatomical sites, different immunogenic compositions including the same or different linear polyribonucleotides, adjuvants, vaccines (e.g., protein subunit vaccines) or a combination thereof can be administered to the same anatomical site, or any combination thereof. For example, an immunogenic composition including a linear polyribonucleotide can be administered in to two different anatomical sites, and/or an immunogenic composition including a linear polyribonucleotide can be administered to one anatomical site, and an adjuvant can be administered to a different anatomical site.

Immunization at any two or more anatomical routes can be via the same route of immunization (e.g., intramuscular) or by two or more routes of immunization. In some embodiments, an immunogenic composition including a circular polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject. In some embodiments, an immunogenic composition including a circular polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition including a circular polyribonucleotide or an adjuvant of the disclosure is immunized to 1, 2, 3, 4, 5, 6, 7, 8, 9, 10 anatomical sites of a subject. In some embodiments, an immunogenic composition including a linear polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject. In some embodiments, an immunogenic composition including a linear polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition including a linear polyribonucleotide or an adjuvant of the disclosure is immunized to 1, 2, 3, 4, 5, 6, 7, 8, 9, 10 anatomical sites of a subject.

Immunization can be by any suitable route. Non-limiting examples of immunization routes include intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal, e.g., injection and infusion. In some cases, immunization can be via inhalation. Two or more immunizations can be done by the same route or by different routes.

Any suitable amount of a circular polyribonucleotide can be administered to a subject of the disclosure. For example, a subject can be immunized with at least about 1 ng, at least about 10 ng, at least about 100 ng, at least about 1 μg, at least about 10 μg, at least about, at least about 100 μg, at least about 1 mg, at least about 10 mg, at least about 100 mg, or at least about 1 g of a circular polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a circular polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a circular polyribonucleotide.

In some embodiments, the method further includes evaluating the subject for antibody response to the immunogen. In some embodiments, the evaluating is before and/or after administration of the circular polyribonucleotide including a sequence encoding an immunogen. In some embodiments, the evaluating is before and/or after administration of the linear polyribonucleotide including a sequence encoding an immunogen.

In some embodiments, the circular polyribonucleotide, immunogenic composition, pharmaceutical preparation, or pharmaceutical composition described herein is administered to a subject between birth and 15 months according to the dosing schedule provided in Table 1 or is administered to a subject between 18 months and 18 years according to the dosing schedule of Table 2. Dosing may be performed according to dosing scheduled known in the art, for example, as described by the Centers of Disease Control and Prevention (CDC) or the National Institutes of Health (NIH). Tables 1 and 2 provide an abbreviated summary of the dosing schedules for vaccination for certain disorders indicated on the CDC website as of Aug. 29, 2020.

TABLE 1
Dosing birth to 15 months

| Indication | | | | | | | | |
|---|---|---|---|---|---|---|---|---|
| (Vaccine) | Birth | 1 mo | 2 mos | 4 mos | 6 mos | 9 mos | 12 mos | 15 mos |
| Hepatitis B | 1^{st} dose | 2^{nd} dose | | | 3^{rd} dose | | | |
| (HepB) | | | | | | | | |
| Measles, | | | | | | | 1^{st} dose | |
| mumps, rubella | | | | | | | | |
| (MMR); varicella | | | | | | | | |
| (VAR) | | | | | | | | |
| Hepatitis A | | | | | | | 2 dose series | |
| (HepA) | | | | | | | | |
| Rotavirus | | | 1^{st} dose | 2^{nd} dose | 3^{rd} dose* | | | |
| (RV1 or RV5) | | | | | | | | |
| Diphtheria, | | | 1^{st} dose | 2^{nd} dose | 3^{rd} dose | | | 4^{th} dose |
| tetanus, | | | | | | | | |
| acellular | | | | | | | | |
| pertussis (DTap) | | | | | | | | |

| | | | | |
|---|---|---|---|---|
| Haemophilus | 1^{st} dose | 2^{nd} dose | 3^{rd} dose* | 3^{rd} or 4^{th} dose |
| Influenzae B | | | | |
| (Hib) | | | | |
| Pneumococcal | 1^{st} dose | 2^{nd} dose | 3^{rd} dose | 4^{th} dose |
| conjugate | | | | |
| (PCV13) | | | | |

| | | | |
|---|---|---|---|
| Inactivated polio | 1^{st} dose | 2^{nd} dose | 3^{rd} dose |
| (IPV) | | | |
| Influenza (IIV) | | | Annual vaccination 1 or 2 doses |

*optional

TABLE 2
Dosing 18 months to 18 years

| | 18 | 19-23 | 2-3 | 4-6 | 4-6 | 7-10 | 11-12 | 13-15 | 16 | 17-18 |
|---|---|---|---|---|---|---|---|---|---|---|
| Vaccine | mos | yrs | yrs | yrs | yrs | yrs | yrs | yrs | yrs | yrs |
| Hep B | 3^{rd} dose | | | | | | | | | |
| (HepB) | | | | | | | | | | |
| Diphtheria, | 4^{th} dose | | 5^{th} dose | | | | | | | |
| tetanus, | | | | | | | | | | |
| acellular | | | | | | | | | | |
| pertussis (DTap) | | | | | | | | | | |
| Inactivated polio | 3^{rd} dose | | 4^{th} dose | | | | | | | |
| (IPV) | | | | | | | | | | |
| Influenza (IIV) | Annual vaccination 1 or 2 doses | | | | | Annual vaccination 1 dose only | | | | |
| Influenza | | | Annual vaccine 1 or 2 doses | | | Annual vaccination 1 dose only | | | | |
| (LAIV) | | | | | | | | | | |
| Hepatitis A | 2 dose series | | | | | | | | | |
| (HepA) | | | | | | | | | | |
| Meningococcal | | | | | | | 1^{st} dose | | 2^{nd} dose | |
| (MenACWY-D; | | | | | | | | | | |
| MenACWY-CRM) | | | | | | | | | | |
| Measles, | | | | 2^{nd} dose | | | | | | |
| mumps, rubella | | | | | | | | | | |
| (MMR); varicella | | | | | | | | | | |
| (VAR) | | | | | | | | | | |
| Diphtheria, | | | | | | | TDap | | | |
| tetanus, | | | | | | | | | | |
| acellular | | | | | | | | | | |
| pertussis (DTap) | | | | | | | | | | |

### Topical Delivery

In some aspects, the present disclosure provides compositions and pharmaceutical compositions for topically delivering a polyribonucleotide that produces an immune response in the subject. The compositions and pharmaceutical compositions can comprise an alcohol (e.g., ethanol) for delivering the polyribonucleotide to a cell. The compositions and pharmaceutical compositions can comprise a cell-penetrating agent. The cell-penetrating agent is configured to enhance delivery of the polyribonucleotide into a cell. The polyribonucleotide can be present in either linear or circular form.

In some embodiments, the compositions or pharmaceutical compositions described herein are directly administered to a surface area (e.g., a topical surface area). In some embodiments, the compositions or pharmaceutical compositions described herein are applied to a surface area of a subject after application of a sterilizing agent. In some embodiments, the compositions or pharmaceutical compositions described herein are applied to a surface area of a subject after application of an alcohol.

The compositions disclosed herein can include a mixture including a polyribonucleotide and an alcohol, such as ethanol. The methods disclosed herein can include delivering a polyribonucleotide in a composition comprising a mixture of the polyribonucleotide and an alcohol, such as ethanol. In some aspects, the present disclosure provides a kit comprising a polyribonucleotide and an alcohol (e.g., ethanol) for delivering the polyribonucleotide into a cell. In some embodiments, the kit comprises a sterilizing agent. In a particular embodiment, the kit comprises a polyribonucleotide and an alcohol wipe (e.g., ethanol wipe, isopropyl wipe).

The compositions disclosed herein can include a mixture including a polyribonucleotide and a cell-penetrating agent. The methods disclosed herein can include delivering a polyribonucleotide in a composition comprising a mixture of the polyribonucleotide and a cell-penetrating agent. In some aspects, the present disclosure provides a kit comprising a polyribonucleotide and a cell-penetrating agent for delivery of the polyribonucleotide into a cell. In some embodiments, the kit comprises a sterilizing agent.

The compositions disclosed herein can be a composition free of any carrier comprising a polyribonucleotide and a diluent. This composition can be used in a method of delivery to an epithelial cell.

In some embodiments, the compositions, therapeutic compositions, or pharmaceutical compositions described herein are directly administered to a surface area (e.g., a topical surface area). In some embodiments, the compositions, therapeutic compositions, or pharmaceutical compositions described herein are applied to a surface area of a subject after application of a sterilizing agent.

The compositions, methods, and kits provided herein can offer a simple and effective solution in which to deliver polyribonucleotides into cells. A polyribonucleotide can be delivered into a cell more efficiently in the presence of the cell-penetrating agent than in the absence of the cell-penetrating agent. In some cases, the cell-penetrating agent described herein can increase the efficiency of delivery of the polynucleotide by at least about 10%, 15%, 20%, 30%, 40%, 50%, 60%, 70%, 80%, 90%, 100%, 120%, 150%, 200%, 250%, 300%, 400%, 500%, 600%, 700%, 800%, 900%, 1000%, 2000%, 5000%, 8000%, 10,000%, 20,000%, or 50,000% as compared to the efficiency of delivery of the polynucleotide in the absence of the cell-penetrating agent.

As used herein, the term “topical delivery” means the delivery of a substance to skin or an epithelial layer accessible though non-invasive means. Topical delivery includes topical delivery to intestinal and other gastrointestinal (GI) epithelia or the vaginal epithelium. Topical delivery of a pharmaceutical composition can have a local pharmacodynamic effect on the subject, e.g., the topically delivered pharmaceutical composition has a pharmacodynamic effect at or proximate to the particular part of the body (e.g. skin) where the pharmaceutical composition is delivered. In some other embodiments, topical delivery of a pharmaceutical composition as discussed herein is used only to refer to the delivery mode (locally to, e.g., a specific surface area), whereas the pharmaceutical composition can have either a local or systemic pharmacodynamic effect. For instance, the pharmaceutical composition can either stay local at or proximate to the administration site, or can enter a circulation system (e.g., blood or lymphoid system) of the subject body, through which the pharmaceutical composition can be transported to remote parts of the body that are typically not reachable by the pharmaceutical composition via routes other than the circulation systems.

In some embodiments, a composition or pharmaceutical composition described herein is formulated for direct application on a skin area (e.g., percutaneous or dermal administration). In some embodiments, a composition or pharmaceutical composition described herein is formulated for administration via the nasal passages of a subject. In some embodiments, a composition or pharmaceutical composition described herein is formulated for oral administration. In some embodiments, a composition or pharmaceutical composition described herein is formulated for ophthalmic administration. In some embodiments, a composition or pharmaceutical composition described herein is formulated for administration as a suppository, for vaginal administration, or for mucosal administration.

A composition or pharmaceutical composition described herein can be applied to a surface after application of a sterilizing agent to that surface (e.g., pre-treatment of the surface with a sterilizing agent for delivery of the polyribonucleotide). The surface can be a surface area of a subject. The surface area of the subject comprises cells, such as epithelial cells.

A sterilizing agent can be any agent that is bactericidal, bacteriostatic, and/or actively kills microorganisms, inactivates microorganism, or prevents microorganisms from growing. In some embodiments, the sterilizing agent is an alcohol, iodine, or hydrogen peroxide. The sterilizing agent can be UV light or laser light. In some embodiments, the sterilizing agent is heat delivered electrically or through other means, such as by vapor or contact.

The sterilizing agent can be applied to the surface area of the subject by various non-invasive methods. For example, a sterilizing agent can be applied by a wipe or swab comprising the sterilizing agent. In some embodiments, the sterilizing agent is applied as a spray. Various devices can be used to apply the sterilizing agent. For example, a device that produces UV light or laser light can be used. In other embodiments, a device that produces heat can be used.

A composition or pharmaceutical composition described herein can be applied to a surface after application of an alcohol, such as ethanol (e.g., pre-treatment of the surface with an alcohol for delivery of the polyribonucleotide). The surface can be a surface area of a subject. The surface area of the subject comprises cells, such as epithelial cells. Alcohol used to pre-treat a surface prior to the administration (e.g., application) of a polyribonucleotide to that surface may act as a sterilizing agent and/or enhance the cell penetration of the polyribonucleotide. In some, alcohol is applied to the surface in the form of a wipe or a swap. In some embodiments, the alcohol in permitted to try on the surface prior to administration (e.g., application) of a polyribonucleotide.

The composition or pharmaceutical composition described herein can be a liquid preparation such as a suspension, syrup, or elixir. In some cases, aqueous solutions are packaged for use as is, or lyophilized, and the lyophilized preparation being combined with a sterile solution prior to administration.

The composition can be delivered as a solution or as a suspension. In general, formulations such as a gel (e.g., DMSO gel), jellies, creams, lotions (e.g., Johnson & Johnson lotion), suppositories and ointments can provide an area with more extended exposure to one or more active agents, while formulations in solution, e.g., sprays, can provide more immediate, short-term exposure.

The composition or pharmaceutical composition applied to the surface area after pre-treatment can be free of any carrier and comprise the polyribonucleotide and a diluent. The polyribonucleotide can be a linear polyribonucleotide or a circular polyribonucleotide.

In some cases, the compositions provided herein are suitable for in vivo use for eliciting an immune response against an immunogen of interest when the composition is administered to a subject. The polyribonucleotide can be present in either linear or circular form.

In a particular embodiment, the compositions and pharmaceutical compositions as described herein, e.g., formulated for topical delivery, can be used to induce an immune response against a viral immunogen and/or for the treatment or prevention of a viral infection, for example, a viral infection selected from influenza (e.g., seasonal influenza, pandemic influenza, influenza A, influenza H1N1 subtype), small pox, chikungunya virus (e.g. chikungunya virus), a coronavirus (e.g., SARS-CoV-2), dengue, zika, yellow fever virus, a coxsackievirus (e.g. hand, foot, and mouth disease), HIV, AIDS, ebolavirus (e.g., Ebola Hemorrhagic Fever), Marburg virus (e.g., Marburg Hemorrhagic Fever), viral Bronchiolitis, Respiratory Syncytial Virus (RSV), a hepatitis virus (e.g. Hepatitis B), a herpes virus (e.g., genital herpes), a rotavirus infection, a measles infection, a mumps infection, and a rubella infection.

In another embodiment, the compositions and pharmaceutical compositions as described herein, e.g., formulated for topical delivery, can be used to induce an immune response against a bacterial immunogen and/or for the treatment or prevention of a bacterial infection, for example, a bacterial infection selected from Q fever, smallpox, pertussis (whooping cough), Haemophilus influenzae, streptococcus (e.g., streptococcal pneumonia), an E. coli infection, bacterial diarrhea, Francisella tularensis bacteria (e.g., tularemia), diphtheria, and tetanus.

In another embodiment, the compositions and pharmaceutical compositions as described herein, e.g., formulated for topical delivery, can be used to induce an immune response against a parasite immunogen and/or for the treatment or prevention of a parasitic infection, for example, malaria.

In another embodiment, the compositions and pharmaceutical compositions as described herein, e.g., formulated for topical delivery, can be used to induce an immune response against a toxin and/or for the treatment or prevention of toxicity associated with exposure to a toxin, for example, a toxin selected from botulin toxin and anthrax.

In another embodiment, the compositions and pharmaceutical compositions as described herein, e.g., formulated for topical delivery, can be used to induce an immune response against an allergen and/or for the treatment or prevention of symptoms associated with exposure to an allergen, for example, an allergen selected from a peanut allergen, a food allergen, a birch pollen allergen, a grass pollen allergen, a cat allergen, a cedar pollen allergen, a dust mite allergen and a ragweed allergen.

In another embodiment, the compositions and pharmaceutical compositions as described herein may be formulated for transdermal delivery. In some embodiments, transdermal delivery may be used to induce an immune response against and/or for the treatment or prevention of any of the following: Q fever, smallpox, a peanut allergen, botulin toxin, viral Bronchiolitis, Respiratory Syncytial Virus (RSV), anthrax, tetanus, and a dust mite allergen.

In another embodiment, the compositions and pharmaceutical compositions as described herein may be formulated for cutaneous delivery. In some embodiments, cutaneous delivery may be used to induce an immune response against and/or for the treatment or prevention of any of the following: influenza (e.g., seasonal influenza, pandemic influenza, influenza A, influenza H1N1 subtype), small pox, chikungunya virus (e.g. chikungunya virus), a coronavirus (e.g., SARS-CoV-2), dengue, zika, yellow fever virus, a coxsackievirus (e.g. hand, foot, and mouth disease), HIV, AIDS, ebolavirus (e.g., Ebola Hemorrhagic Fever), Marburg virus (e.g., Marburg Hemorrhagic Fever), Respiratory Syncytial Virus (RSV), a hepatitis virus (e.g. Hepatitis B), a herpes virus (e.g., genital herpes), a rotavirus infection, a measles infection, a mumps infection, a rubella infection, pertussis (whooping cough), streptococcus (e.g., streptococcal pneumonia), an E. coli infection, bacterial diarrhea, Francisella tularensis bacteria (e.g., tularemia), diphtheria, tetanus, malaria, anthrax, a food allergen, a birch pollen allergen, a grass pollen allergen, a cat allergen, a cedar pollen allergen, and a dust mite allergen a ragweed allergen.

### Alcohol

Alcohol as described herein can be used for the delivery of a polyribonucleotide into a cell. An alcohol can be in a mixture with a polyribonucleotide as described herein for delivery of the polyribonucleotide into a cell. The mixture can comprise the alcohol in at least about 0.3% v/v alcohol to about 75% v/v. The alcohol can be ethanol. In some embodiments, the mixture is applied to a surface area of a subject. In some embodiments, the mixture is a pharmaceutical composition. An alcohol can be any alcohol that comprises one or more hydroxyl function groups. In some cases, the alcohol is, but is not limited to, a monohydric alcohol, a polyhydric alcohol, an unsaturated aliphatic alcohol, or an alicyclic alcohol. The alcohol can include one or more of methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the alcohol is ethanol.

In other cases, the compositions, pharmaceutical compositions, and methods provided herein only include an alcohol and do not have or use any other agent to enhance the delivery of the polyribonucleotide into a cell. In some cases, the alcohol is ethanol and the composition, pharmaceutical composition, and methods do not have or use any other agent to enhance delivery of polyribonucleotide into a cell. In some cases, the alcohol is a cell-penetrating agent. In some cases, the alcohol is not a cell-penetrating agent.

The composition disclosed herein can include a mixture comprising an alcohol and a polyribonucleotide. In some cases, the polyribonucleotide is present in a pre-mixed mixture with the alcohol. In some cases, the polyribonucleotides is provided separately from the alcohol prior to contact to a cell. In these instances, the polyribonucleotide is contacted with the alcohol when being applied to a cell and becomes mixed together for delivery of the polyribonucleotide into the cell. Without being bound to a certain theory, the concentration of the alcohol in the mixture can contribute to the efficiency of delivery. Therefore, in some cases, the alcohol is provided at a predetermined concentration in the mixture. In some other cases, when the alcohol and the polyribonucleotide are separate initially but mixed together when being applied for delivery, the alcohol is provided at a sufficient amount relative to the polyribonucleotide that would ensure it reach a minimum predetermined concentration in the mixture.

In some cases, the alcohol constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, the alcohol constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, the alcohol constitutes about 10%, about 20%, about 30%, about 40%, about 50%, about 60%, about 70%, about 80%, about 90%, about 95%, about 98%, or about 100% v/v of the mixture.

In some cases, the alcohol constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% weight per weight (w/w) of the mixture. In some cases, the alcohol constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% w/w of the mixture. In some cases, the cell-penetrating agent constitutes about 10%, about 20%, about 30%, about 40%, about 50%, about 60%, about 70%, about 80%, about 90%, about 95%, or about 98% w/w of the mixture. In some cases, the alcohol constitutes about 10% v/v of the mixture.

In some embodiments, the alcohol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture, or any percentage v/v therebetween. In some embodiments, the alcohol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture, or any percentage v/v therebetween.

In some cases, the mixture described herein is a liquid solution. For instance, the alcohol is a liquid substance itself. In these cases, the polyribonucleotide can also be dissolved in the liquid solution. In some cases, ethanol constitutes at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, ethanol constitutes at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, ethanol constitutes about 10%, about 20%, about 30%, about 40%, about 50%, about 60%, about 70%, about 80%, about 90%, about 95%, about 98%, or about 100% v/v of the mixture. In some cases, ethanol constitutes about 10% v/v of the mixture.

In some embodiments, the ethanol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture, or any percentage v/v therebetween. In some embodiments, the ethanol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture, or any percentage v/v therebetween.

### Cell-Penetrating Agents

The cell-penetrating agent described herein can include any substance that enhances delivery of a polyribonucleotide into a cell. The cell-penetrating agent can include an organic compound or an inorganic molecule. In some cases, the cell-penetrating agent is an organic compound having one or more functional groups such as, but not limited to, alkane, alkene, and arene; halogen-substituted alkane, alkenes, and arenes; alcohols, phenols (derivatives of benzene), ethers, aldehydes, ketones, and carboxylic acids; amines and nitriles; and organosulfurs (e.g., dimethyl sulfoxide). In some embodiments, the cell-penetrating agent is soluble in polar solvents. In some embodiments, the cell-penetrating agent is insoluble in polar solvents. The polyribonucleotide can be present in either linear or circular form.

The cell-penetrating agent can include organic compounds such as alcohols having one or more hydroxyl function groups. In some cases, the cell-penetrating agent includes an alcohol such as, but not limited to, monohydric alcohols, polyhydric alcohols, unsaturated aliphatic alcohols, and alicyclic alcohols. The cell-penetrating agent can include one or more of methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the cell-penetrating agent comprises ethanol.

In other cases, the compositions and methods provided herein only include an alcohol as the cell-penetrating agent, and do not have or use any other agent to enhance the delivery of the polyribonucleotide into a cell. In some cases, the cell-penetrating agent comprises ethanol and any other alcohol that can enhance delivery of polyribonucleotide into a cell. In some cases, the cell-penetrating agent comprises ethanol and any other organic or inorganic molecules that can enhance delivery of polyribonucleotide into a cell. In some cases, the cell-penetrating agent comprises ethanol and liposome or nanoparticles such as those described in International Publication Nos. WO2013/006825, WO2016/036735, WO2018/112282A1, and WO2012/031043A1, each of which is incorporated herein by reference in its entirety. In some cases, the cell-penetrating agent comprises ethanol and cell-penetrating peptides or proteins such as those described in Bechara et al, Cell-penetrating peptides: 20 years later, where do we stand? FEBS Letters 587(12):1693-1702 (2013); Langel, Cell-Penetrating Peptides: Processes and Applications (CRC Press, Boca Raton FL, 2002); El-Andaloussi et al., Curr. Pharm. Des. 11(28):3597-611 (2003); Deshayes et al, Cell. Mol. Life Sci. 62(16):1839-49 (2005), US Patent Publication Nos. US20130129726, US20130137644 and US20130164219, each of which is herein incorporated by reference in its entirety). In some cases, the ratio of ethanol versus other cell-penetrating agent is about 1:0.001, 1:0.002, 1:005, 1:008, 1:0.01, 1:0.02, 1:0.05, 1:0.08, 1:0.1, 1:0.2, 1:0.3, 1:0.4, 1:0.5, 1:0.6, 1:0.7, 1:0.8, 1:0.9, 1:1, 1:1.2, 1:1.5, 1:1.8, 1:2, 1:2.5, 1:3, 1:3.5, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:150, 1:200, 1:250, 1:500, or 1:1000. In some cases, the ratio of ethanol versus other cell-penetrating agent is at least about 1:0.001, 1:0.002, 1:005, 1:008, 1:0.01, 1:0.02, 1:0.05, 1:0.08, 1:0.1, 1:0.2, 1:0.3, 1:0.4, 1:0.5, 1:0.6, 1:0.7, 1:0.8, 1:0.9, 1:1, 1:1.2, 1:1.5, 1:1.8, 1:2, 1:2.5, 1:3, 1:3.5, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:150, 1:200, 1:250, or 1:500.

The composition disclosed herein can include a mixture of a cell-penetrating agent and a polyribonucleotide. In some cases, the polyribonucleotide is present in a pre-mixed mixture with the cell-penetrating agent. In some cases, the polyribonucleotides is provided separately from the cell-penetrating agent prior to contact to a cell. In these instances, the polyribonucleotide is contacted with the cell-penetrating agent when being applied to a cell and becomes mixed together for delivery of the polyribonucleotide into the cell. Without being bound to a certain theory, the concentration of the cell-penetrating agent in the mixture can contribute to the efficiency of delivery. Therefore, in some cases, the cell-penetrating agent is provided at a predetermined concentration in the mixture. In some other cases, when the cell-penetrating agent and the polyribonucleotide are separate initially but mixed together when being applied for delivery, the cell-penetrating agent is provided at a sufficient amount relative to the polyribonucleotide that would ensure it reach a minimum predetermined concentration in the mixture.

In some cases, the cell-penetrating agent constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, the cell-penetrating agent constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, the cell-penetrating agent constitutes about 10%, about 20%, about 30%, about 40%, about 50%, about 60%, about 70%, about 80%, about 90%, about 95%, about 98%, or about 100% v/v of the mixture.

In some cases, the cell-penetrating agent constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% weight per weight (w/w) of the mixture. In some cases, the cell-penetrating agent constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% w/w of the mixture. In some cases, the cell-penetrating agent constitutes about 10%, about 20%, about 30%, about 40%, about 50%, about 60%, about 70%, about 80%, about 90%, about 95%, or about 98% w/w of the mixture. In some cases, the cell-penetrating agent constitutes about 10% v/v of the mixture.

In some cases, the mixture described herein is a liquid solution. For instance, the cell-penetrating agent is a liquid substance itself. Alternatively, the cell-penetrating agent is a solid, liquid, or gas substance and dissolved in a liquid carrier, e.g., water. In these cases, the polyribonucleotide can also be dissolved in the liquid solution.

In some cases, ethanol constitutes at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, ethanol constitutes at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, ethanol constitutes about 10%, about 20%, about 30%, about 40%, about 50%, about 60%, about 70%, about 80%, about 90%, about 95%, about 98%, or about 100% v/v of the mixture. In some cases, ethanol constitutes about 10% v/v of the mixture.

### Preservatives

A composition or pharmaceutical composition provided herein can comprise material for a single administration, or can comprise material for multiple administrations (e.g., a “multidose” kit). The polyribonucleotide can be present in either linear or circular form. The composition or pharmaceutical composition can include one or more preservatives such as thiomersal or 2-phenoxyethanol. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In ophthalmic products, e.g., such preservatives can be employed at a level of from 0.004% to 0.02%. In the compositions described herein the preservative, e.g., benzalkonium chloride, can be employed at a level of from 0.001% to less than 0.01%, e.g., from 0.001% to 0.008%, preferably about 0.005% by weight.

Polyribonucleotides can be susceptible to RNase that can be abundant in ambient environment. Compositions provided herein can include reagents that inhibit RNase activity, thereby preserving the polyribonucleotide from degradation. In some cases, the composition or pharmaceutical composition includes any RNase inhibitor known to one skilled in the art. Alternatively or additionally, the polyribonucleotide, and cell-penetrating agent and/or pharmaceutically acceptable diluents or carriers, vehicles, excipients, or other reagents in the composition provided herein can be prepared in RNase-free environment. The composition can be formulated in RNase-free environment.

In some cases, a composition provided herein can be sterile. The composition can be formulated as a sterile solution or suspension, in suitable vehicles, known in the art. The composition can be sterilized by conventional, known sterilization techniques, e.g., the composition can be sterile filtered.

### Salts

In some cases, a composition or pharmaceutical composition provided herein comprises one or more salts. For controlling the tonicity, a physiological salt such as sodium salt can be included a composition provided herein. Other salts can comprise potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can comprise those of the inorganic ions, such as, for example, sodium, potassium, calcium, magnesium ions, and the like. Such salts can comprise salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can comprise one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/surfactants

A composition or pharmaceutical composition provided herein can comprise one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, e.g., octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, an immunogenic composition of the disclosure includes a circular polyribonucleotide and a diluent. In some embodiments, an immunogenic composition of the disclosure includes a linear polyribonucleotide and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the circular polyribonucleotide may be delivered as a naked delivery formulation, such as including a diluent. A naked delivery formulation delivers a circular polyribonucleotide, to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without a covalent modification that binds a moiety that aids in delivery to a cell is a polyribonucleotide that is not covalently bound to a protein, small molecule, a particle, a polymer, or a biopolymer. A circular polyribonucleotide without covalent modification that binds a moiety that aids in delivery to a cell does not contain a modified phosphate group. For example, a circular polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In some embodiments, a naked delivery formulation is free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carriers

In some embodiments, an immunogenic composition of the disclosure includes a circular polyribonucleotide and a carrier. In some embodiments, an immunogenic composition of the disclosure includes a linear polyribonucleotide and a carrier.

In certain embodiments, an immunogenic composition includes a circular polyribonucleotide as described herein in a vesicle or other membrane-based carrier. In certain embodiments, an immunogenic composition includes a linear polyribonucleotide as described herein in a vesicle or other membrane-based carrier.

In other embodiments, an immunogenic composition includes the circular polyribonucleotide in or via a cell, vesicle or other membrane-based carrier. In other embodiments, an immunogenic composition includes the linear polyribonucleotide in or via a cell, vesicle or other membrane-based carrier. In one embodiment, an immunogenic composition includes the circular polyribonucleotide in liposomes or other similar vesicles. In one embodiment, an immunogenic composition includes the linear polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In certain embodiments, an immunogenic composition of the disclosure includes a circular polyribonucleotide and lipid nanoparticles, for example lipid nanoparticles described herein. In certain embodiments, an immunogenic composition of the disclosure includes a linear polyribonucleotide and lipid nanoparticles. Lipid nanoparticles are another example of a carrier that provides a biocompatible and biodegradable delivery system for a circular polyribonucleotide molecule as described herein. Lipid nanoparticles are another example of a carrier that provides a biocompatible and biodegradable delivery system for a linear polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide or a protein covalently linked to the linear polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular RNA composition or preparation described herein. Exosomes can be used as drug delivery vehicles for a linear polyribonucleotide composition or preparation described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular RNA composition or preparation described herein. Ex vivo differentiated red blood cells can also be used as a carrier for a linear polyribonucleotide composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in International Patent Publication No. WO2018/208728, can also be used as carriers to deliver a circular polyribonucleotide molecule described herein. Fusosome compositions, e.g., as described in WO2018/208728, can also be used as carriers to deliver a linear polyribonucleotide molecule described herein.

Virosomes and virus-like particles (VLPs) can also be used as carriers to deliver a circular polyribonucleotide molecule described herein to targeted cells. Virosomes and virus-like particles (VLPs) can also be used as carriers to deliver a linear polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the circular RNA composition or preparation described herein. Plant nanovesicles and plant messenger packs (PMPs) can also be used as carriers to deliver a linear polyribonucleotide composition or preparation described herein.

Microbubbles can also be used as carriers to deliver a circular polyribonucleotide molecule described herein. Microbubbles can also be used as carriers to deliver a linear polyribonucleotide molecule described herein. See, e.g., U.S. Pat. No. 7,115,583; Beeri, R. et al., Circulation. 2002 Oct. 1; 106(14):1756-1759; Bez, M. et al., Nat Protoc. 2019 April; 14(4): 1015-1026; Hernot, S. et al., Adv Drug Deliv Rev. 2008 Jun. 30; 60(10): 1153-1166; Rychak, J. J. et al., Adv Drug Deliv Rev. 2014 June; 72: 82-93. In some embodiments, microbubbles are albumin-coated perfluorocarbon microbubbles.

The carrier including the circular polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the circular polyribonucleotide into a cell. Deposition of the circular polyribonucleotide into certain cell types may favor different particle sizes. For example, the particle size may be optimized for deposition of the circular polyribonucleotide into antigen presenting cells. The particle size may be optimized for deposition of the circular polyribonucleotide into dendritic cells. Additionally, the particle size may be optimized for depositions of the circular polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

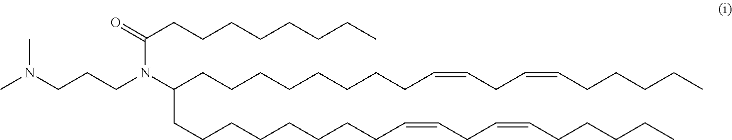 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a polyribonucleotide (e.g., a circular polyribonucleotide, a linear polyribonucleotide) composition described herein to cells.

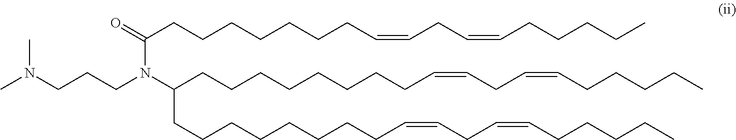 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a polyribonucleotide (e.g., a circular polyribonucleotide, a linear polyribonucleotide) composition described herein to cells.

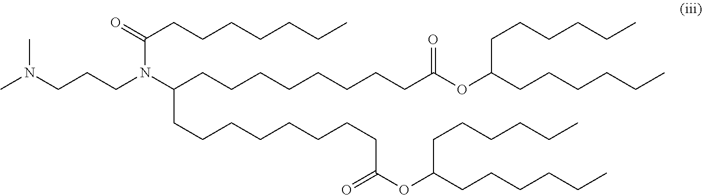 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a polyribonucleotide (e.g., a circular polyribonucleotide, a linear polyribonucleotide) composition described herein to cells.

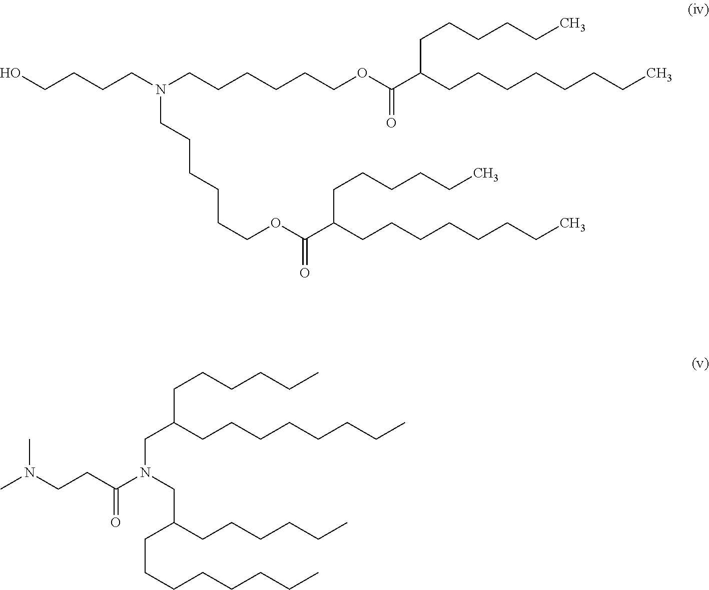

In some embodiments an LNP comprising Formula (v) is used to deliver a polyribonucleotide (e.g., a circular polyribonucleotide, a linear polyribonucleotide) composition described herein to cells.

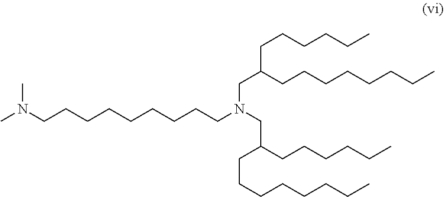 [SMILES: CCCCCCCCC(CCCCCC)CN(CCCCCCCCCN(C)C)CC(CCCCCC)CCCCCCCC]

In some embodiments an LNP comprising Formula (vi) is used to deliver a polyribonucleotide (e.g., a circular polyribonucleotide, a linear polyribonucleotide) composition described herein to cells.

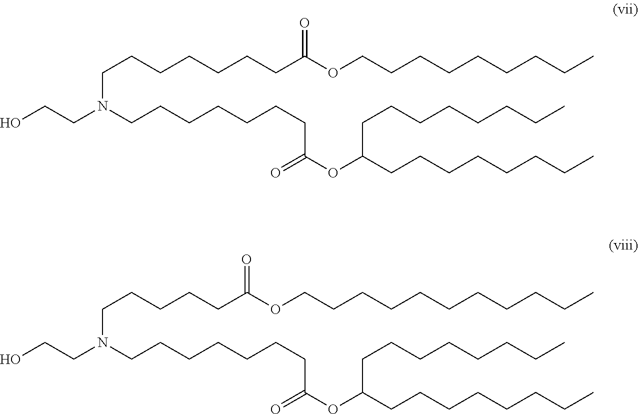 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a polyribonucleotide (e.g., a circular polyribonucleotide, a linear polyribonucleotide) composition described herein to cells.

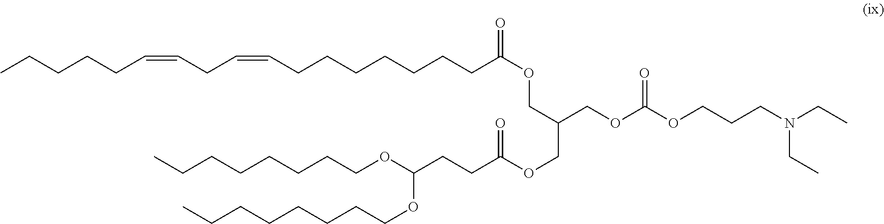 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a polyribonucleotide (e.g., a circular polyribonucleotide, a linear polyribonucleotide) composition described herein to cells.

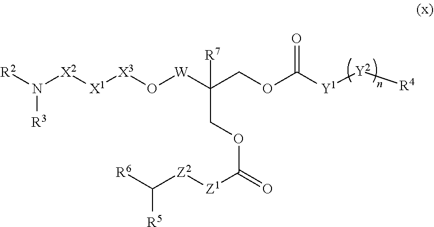

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

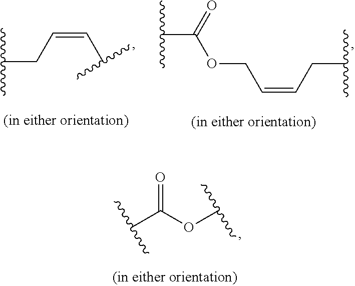 [SMILES: */C=C\C* ; *C/C=C\COC(*)=O ; *OC(*)=O]

n is 0 to 3, R⁴ is C1-15 alkyl, Z¹ is C1-6 alkylene or a direct bond,
Z² is

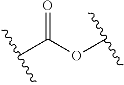 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and
R⁷ is H or Me, or a salt thereof, provided that if R³ and R² are C2 alkyls, X¹ is O, X² is linear C3 alkylene,
X³ is C(═O), Y¹ is linear Ce alkylene, (Y²)n-R⁴ is

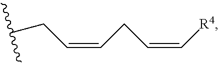

R⁴ is a linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a polyribonucleotide (e.g., a circular polyribonucleotide, a linear polyribonucleotide) composition described herein to cells.

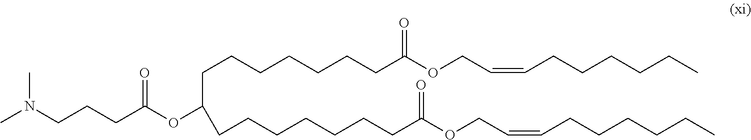 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a polyribonucleotide (e.g., a circular polyribonucleotide, a linear polyribonucleotide) composition described herein to cells.

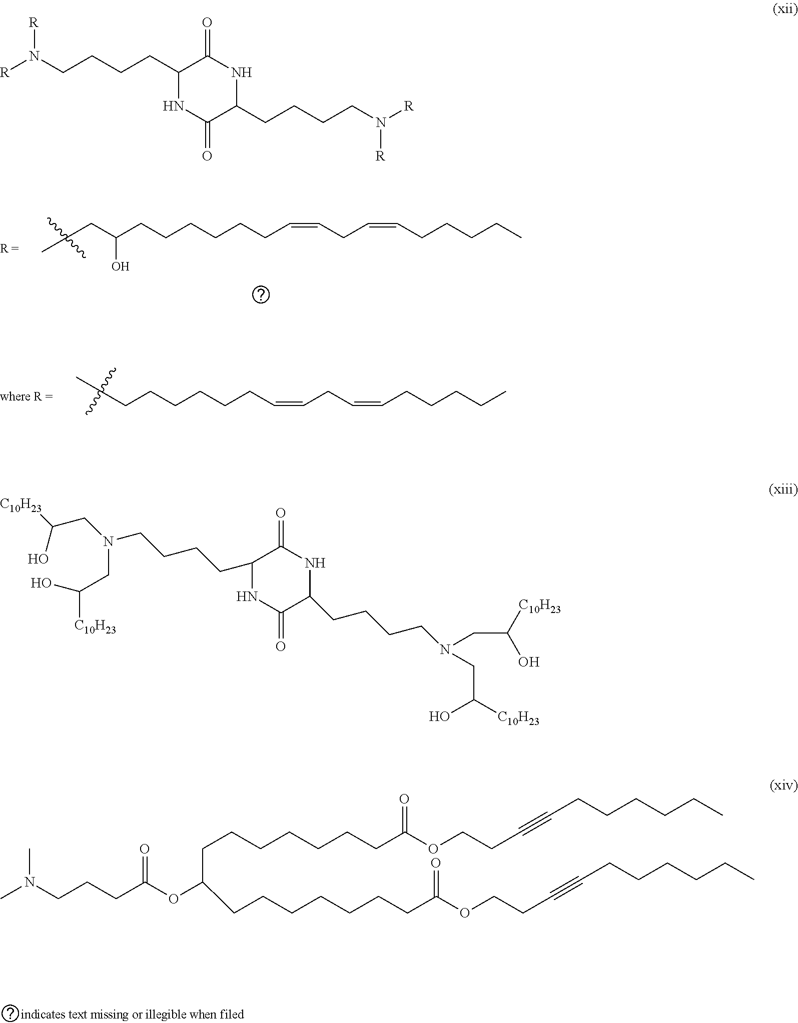

In some embodiments an LNP comprises a compound of Formula (xiii) and a compound of Formula (xiv).

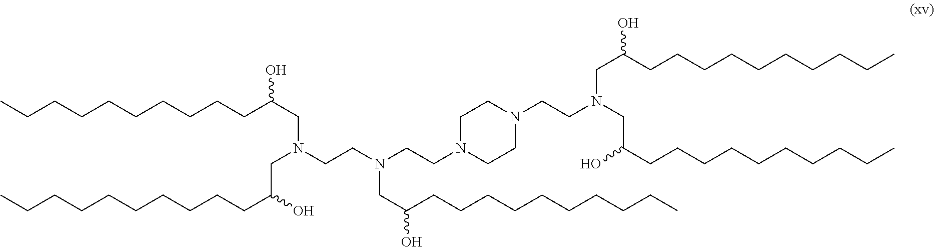 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a polyribonucleotide (e.g., a circular polyribonucleotide, a linear polyribonucleotide) composition described herein to cells.

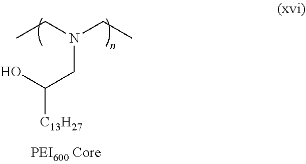

In some embodiments an LNP comprising a formulation of Formula (xvi) is used to deliver a polyribonucleotide (e.g., a circular polyribonucleotide, a linear polyribonucleotide) composition described herein to cells.

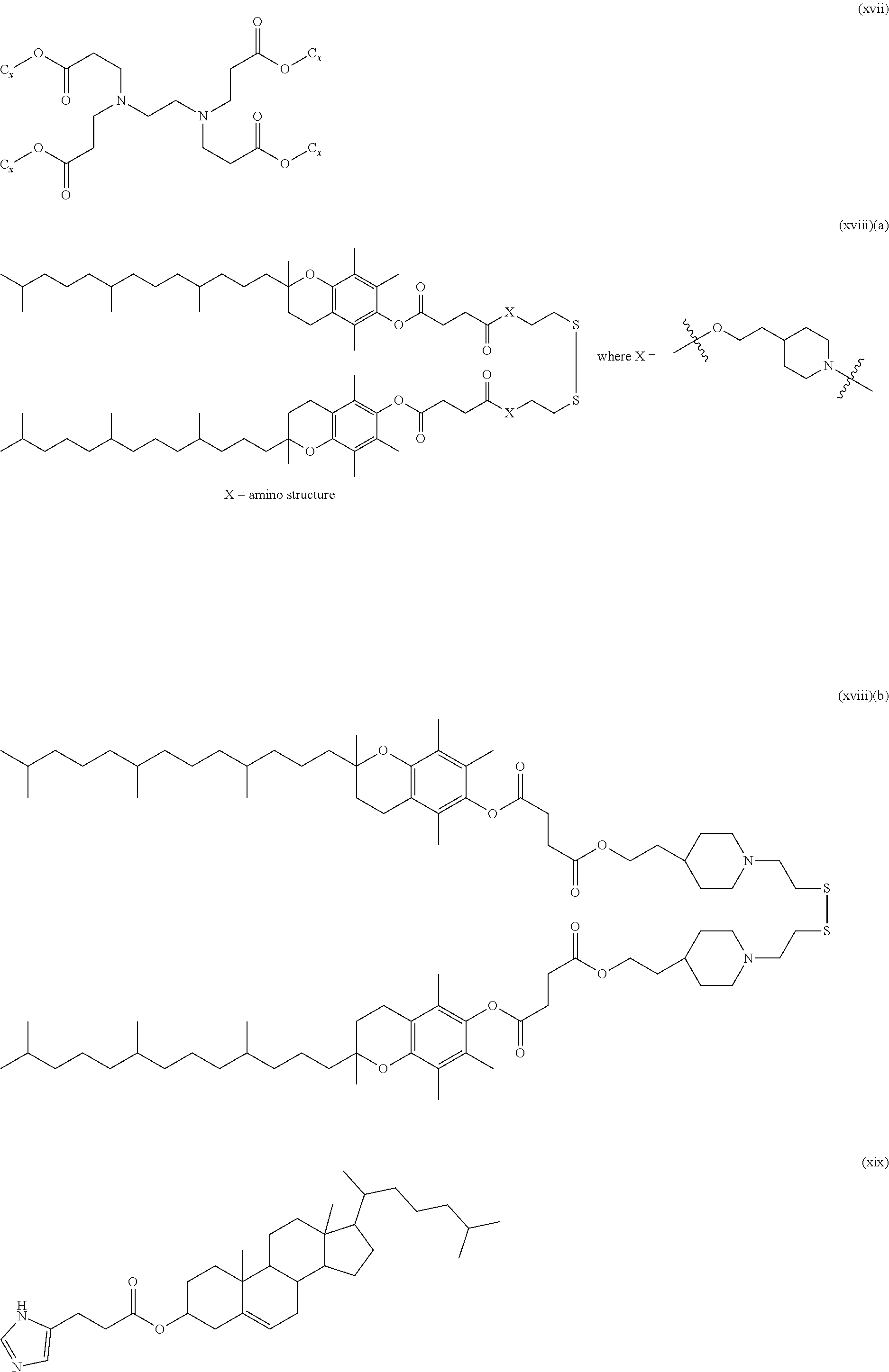

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

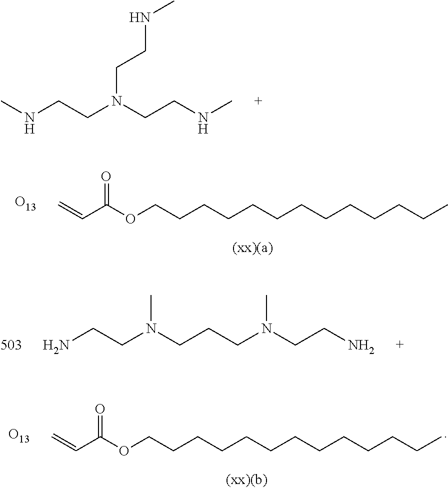

In some embodiments an LNP comprising Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

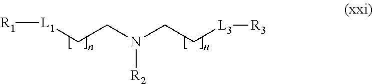

wherein
each n is independently an integer from 2-15; L₁ and L₃ are each independently —OC(O)-* or -C(O)O—*, wherein “*” indicates the attachment point to R₁ or R₃;
R₁ and R₃ are each independently a linear or branched C9-C20 alkyl or C9-C20 alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

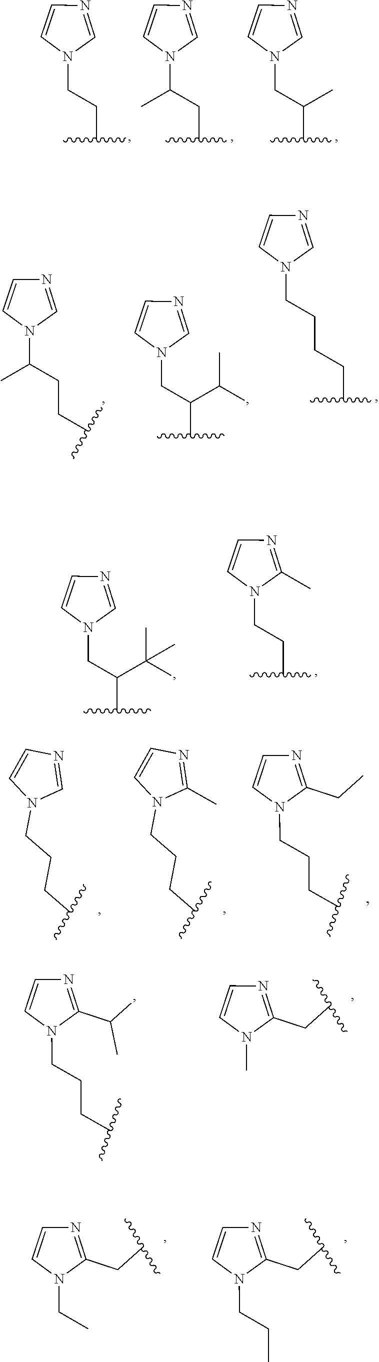

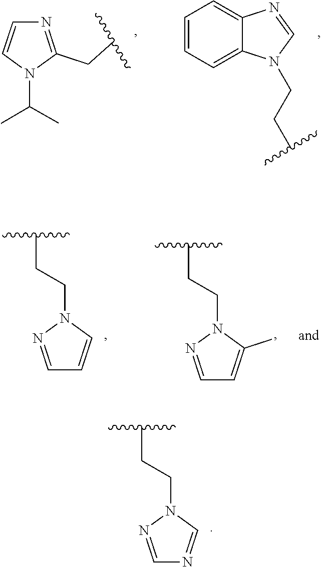

In some embodiments an LNP comprising Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

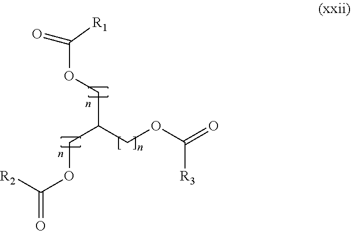

wherein
each n is independently an integer from 1-15,
R₁ and R₂ are each independently selected from a group consisting of:

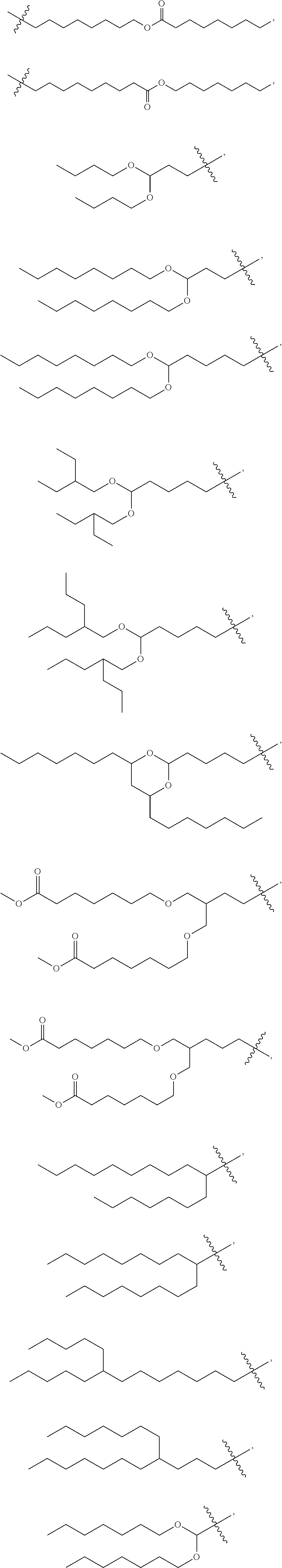

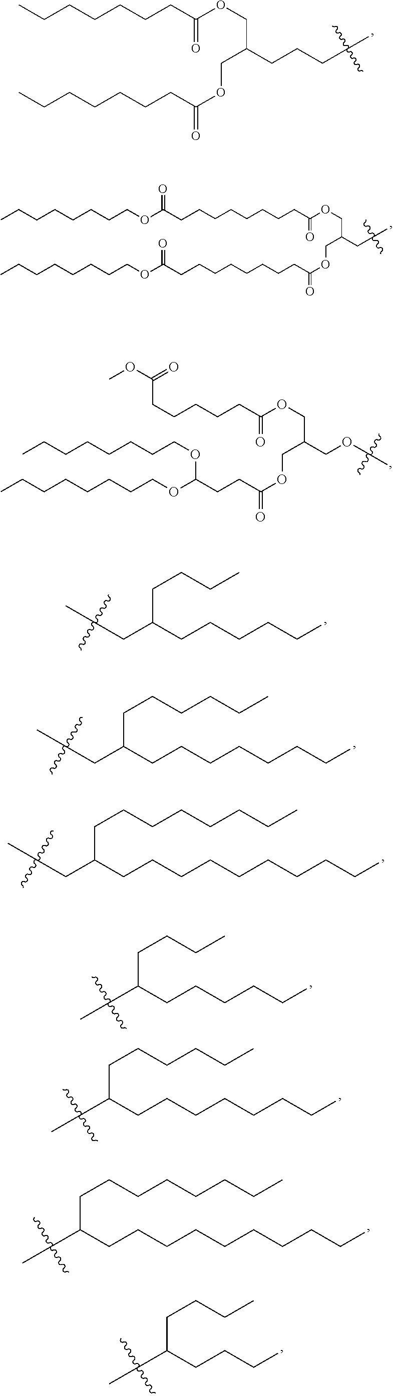

R₃ is selected from a group consisting of:

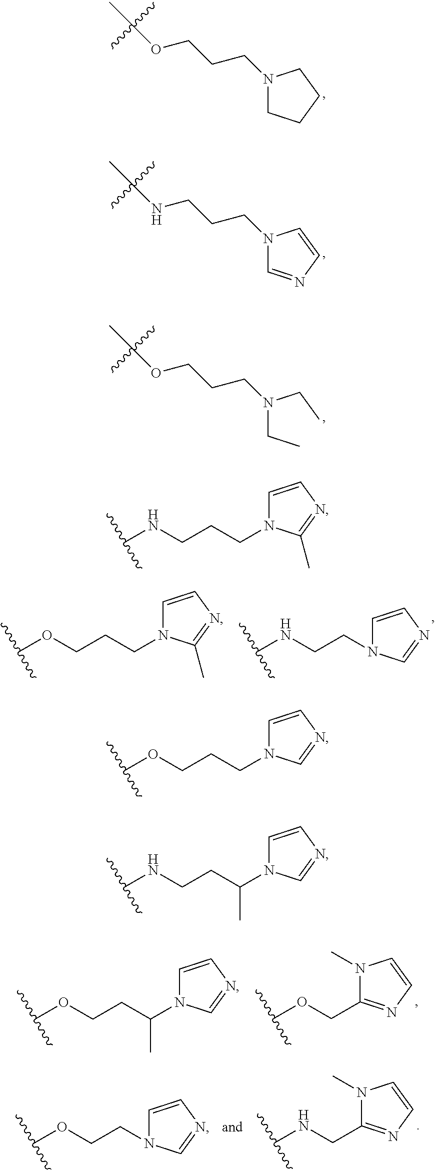

In some embodiments an LNP comprising Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

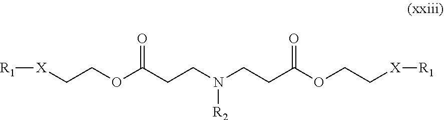

wherein
X is selected from —O—, —S, or —OC(O)—*, wherein * indicates the attachment point to R₁;
R₁ is selected rom a group consisting of:

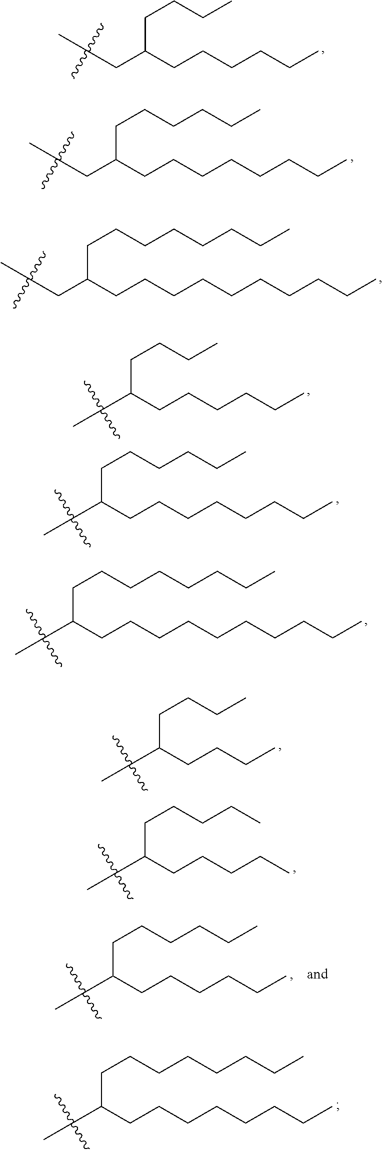

and R is selected from a group consisting of:

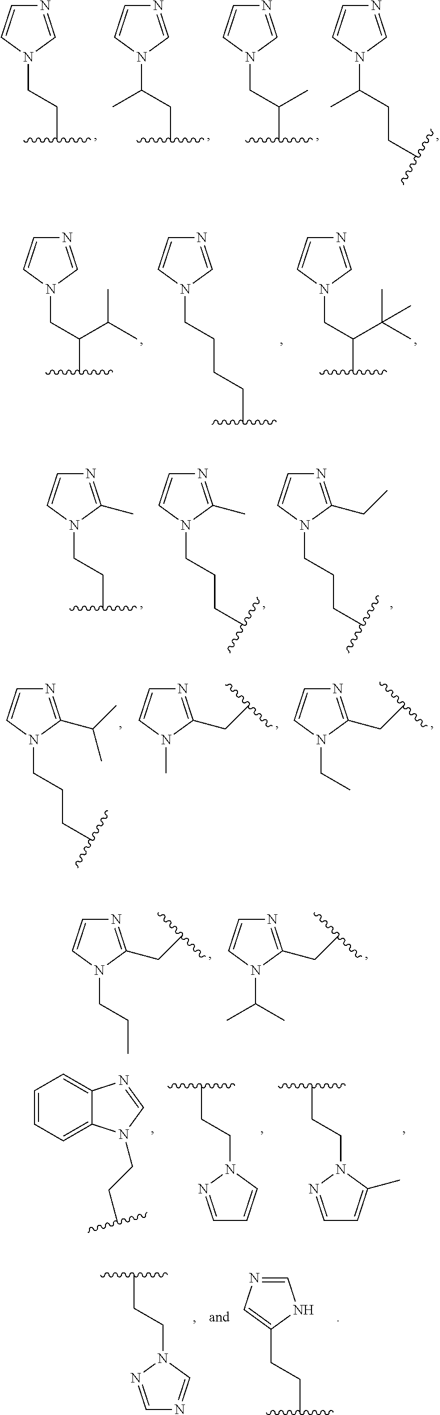

In some embodiments, a composition described herein (e.g., a nucleic acid (e.g., a circular polyribonucleotide, a linear polyribonucleotide) or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl)) 9-((4-diamethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO010/0535723 (incorporated by reference herein in its entirety). In some embodiments, the ionizable lipid is Imidazole cholesterol ester (ICE) lipid (3S,10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl)propanoate, e.g., Structure (1) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid.

In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 IZ)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle.

In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-1, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

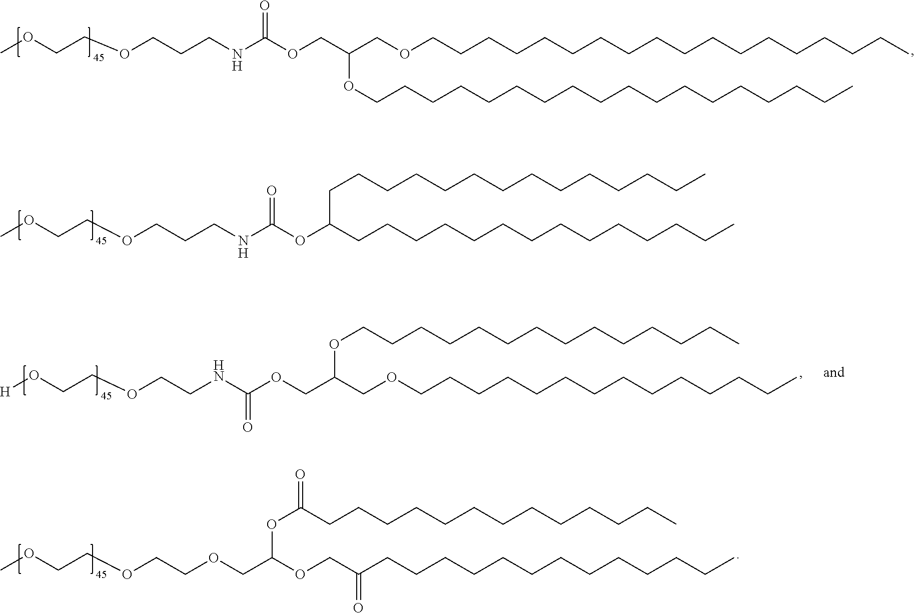

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition.

Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

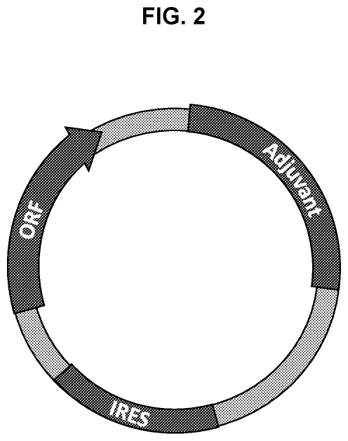

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TranslT-mRNA Transfection Reagent (Mirus Bio). In certain embodiments, LNPs are formulated using the GenVoy_ILM ionizable lipid mix (Precision NanoSystems).

In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA).

Exemplary dosing of AAV comprising a polyribonucleotide (e.g., a circular polyribonucleotide, a linear polyribonucleotide) may include an MOI of about 10¹¹, 10¹², 10¹³, and 10¹⁴ vg/kg.

### Adjuvants

An adjuvant enhances the immune responses (humoral and/or cellular) elicited in a subject who receives the adjuvant and/or an immunogenic composition including the adjuvant. In some embodiments, an adjuvant is administered to a subject as disclosed herein. In some embodiments, an adjuvant is used in the methods described herein to produce an immune response as described herein. In a particular embodiment, an adjuvant is used to promote an immune response in a subject against an immunogen expressed from a circular polyribonucleotide. In some embodiments, an adjuvant and polyribonucleotide are co-administered in separate compositions. In some embodiments, an adjuvant is mixed or formulated with a polyribonucleotide in a single composition and administered to a subject. In some embodiments, an adjuvant and circular or linear polyribonucleotide are co-administered in separate compositions. In some embodiments, an adjuvant is mixed or formulated with a linear or circular polyribonucleotide in a single composition to obtain an immunogenic composition that is administered to a subject.

An adjuvant may be a component of a circular or linear polyribonucleotide (e.g., a polyribonucleotide sequence), may be polypeptide adjuvant encoded by an expression sequence of a polyribonucleotide, may be a molecule (e.g., a small molecule, polypeptide, or nucleic acid molecule) that is not encoded by the polyribonucleotide. An adjuvant may be formulated with a polyribonucleotide in the same pharmaceutical composition. An adjuvant may be administered separately (e.g., as a separate pharmaceutical composition) in combination with a polyribonucleotide.

In some embodiments, the adjuvant is encoded by the circular or linear polyribonucleotide. In some embodiments, the circular or linear polyribonucleotide encodes more than one adjuvant. For example, the circular or linear polyribonucleotide encodes between 2 and 100 adjuvants. In some embodiments, the circular or linear polyribonucleotide encodes between 2 and 10 adjuvants. In some embodiments, the circular or linear polyribonucleotide encodes 2 adjuvants. One or more of the adjuvants encoded by a circular or linear polyribonucleotide may include an N-terminal signal sequence, e.g., that directs the expressed polypeptide adjuvant to the secretory pathway. In some embodiments, the polyribonucleotide encodes 3 adjuvants. In some embodiments, the polyribonucleotide encodes 4 adjuvants. In some embodiments, the polyribonucleotide encodes 5 adjuvants. In some embodiments, the adjuvant is encoded by the same polyribonucleotide that encodes one or more immunogens. The adjuvant(s) and immunogen(s) may be co-delivered on the same polyribonucleotide. In some embodiments, the adjuvant encoded by the polyribonucleotide is a sequence (e.g., a polyribonucleotide sequence) that is an innate immune system stimulator. The innate immune system stimulator sequence may include at least 5, at least 10, at least 20, at least 50, at least 100, or at least 500 ribonucleotides. The innate immune system stimulator sequence may include between 5 and 1000, between 10 and 500, between 20 and 500, between 10 and 100, between 20 and 100, between 20 and 50, between 100 and 500, between 500 and 1000, or between 10 and 1000 ribonucleotides. For example, a sequence that is an innate immune system stimulator may be selected from a GU-rich motif, an AU-rich motif, a structured region comprising dsRNA, or an aptamer.

Adjuvants may be a TH1 adjuvant and/or a TH2 adjuvant. Further adjuvants contemplated by this disclosure include, but are not limited to, one or more of the following:

Mineral-containing compositions. Mineral-containing compositions suitable for use as adjuvants in the disclosure include mineral salts, such as aluminum salts, and calcium salts. The disclosure includes mineral salts such as hydroxides (e.g. oxyhydroxides), phosphates (e.g. hydroxyphosphates, orthophosphates), sulphates, etc., or mixtures of different mineral compounds, with the compounds taking any suitable form (e.g. gel, crystalline, amorphous, etc.). Calcium salts include calcium phosphate (e.g., the “CAP”). Aluminum salts include hydroxides, phosphates, sulfates, and the like.

Oil emulsion compositions. Oil-emulsion compositions suitable for use as adjuvants in the disclosure include squalene-water emulsions, such as MF59 (5% Squalene, 0.5% Tween 80 and 0.5% Span, formulated into submicron particles using a microfluidizer), AS03 (α-tocopherol, squalene and polysorbate 80 in an oil-in-water emulsion), Montanide formulations (e.g. Montanide ISA 51, Montanide ISA 720), incomplete Freunds adjuvant (IFA), complete Freund's adjuvant (CFA), and incomplete Freund's adjuvant (IFA).

Small molecules. Small molecules suitable for use as adjuvants in the disclosure include imiquimod or 847, resiquimod or R₈₄₈, and gardiquimod.

Polymeric nanoparticles. Polymeric nanoparticles suitable for use as an adjuvant in the disclosure include poly(a-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

Saponin (i.e., a glycoside, polycyclic aglycones attached to one or more sugar side chains). Saponin formulations suitable for use as an adjuvant in the disclosure include purified formulations, such as QS21, as well as lipid formulations, such as ISCOMs and ISCOMs matrix. QS21 is marketed as STIMULON™. Saponin formulations may also include a sterol, such as cholesterol. Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent.

Lipopolysaccharides. Adjuvants suitable for use in the disclosure include non-toxic derivatives of enterobacterial lipopolysaccharide (LPS). Such derivatives include monophosphoryl lipid A (MPLA), glucopyranosyl lipid A (GLA) and 3-O-deacylated MPL (3dMPL). 3dMPL is a mixture of 3 De-O-acylated monophosphoryl lipid A with 4, 5 or 6 acylated chains. Other non-toxic LPS derivatives include monophosphoryl lipid A mimics, such as aminoalkyl glucosaminide phosphate derivatives e.g. RC-529.

Liposomes. Liposomes suitable for use as an adjuvant in the disclosure include virosomes and CAF01.

Lipid nanoparticles. Adjuvants suitable for use in the disclosure include lipid nanoparticles (LNPs) and their components.

Lipopeptides (i.e., compounds including one or more fatty acid residues and two or more amino acid residues). Lipopeptide suitable for use as an adjuvant in the disclosure include Pam2 (Pam2CSK4) and Pam3 (Pam3CSK4).

Glycolipids. Glycolipids suitable for use as an adjuvant in the disclosure include cord factor (trehalose dimycolate).

Peptides and peptidoglycans derived from (synthetic or purified) gram-negative or gram-positive bacteria, such as MDP (N-acetyl-muramyl-L-alanyl-D-isoglutamine) are suitable for use as an adjuvant in the disclosure Carbohydrates (carbohydrate containing) or polysaccharides suitable for use as an adjuvant include dextran (e.g., branched microbial polysaccharide), dextran-sulfate, lentinan, zymosan, beta-glucan, deltin, mannan, and chitin.

RNA based adjuvants. RNA based adjuvants suitable for use in the disclosure are poly IC, poly IC:LC, hairpin RNAs with or without a 5′triphosphate, viral sequences, polyU containing sequence, dsRNA natural or synthetic RNA sequences, and nucleic acid analogs (e.g., cyclic GMP-AMP or other cyclic dinucleotides e.g., cyclic di-GMP, immunostimulatory base analogs e.g., C8-substituted and N7,C8-disubstituted guanine ribonucleotides). In some embodiments, the adjuvant is the linear polyribonucleotide counterpart of the circular polyribonucleotide described herein.

DNA based adjuvants. DNA based adjuvants suitable for use in the disclosure include CpGs, dsDNA, and natural or synthetic immunostimulatory DNA sequences.

Proteins or peptides. Proteins and peptides suitable for use as an adjuvant in the disclosure include flagellin-fusion proteins, MBL (mannose-binding lectin), cytokines, and chemokines.

Viral particles. Viral particles suitable for use as an adjuvant include virosomes (phospholipid cell membrane bilayer).

An adjuvant for use in the disclosure may be bacterial derived, such as a flagellin, LPS, or a bacterial toxin (e.g., enterotoxins (protein), e.g., heat-labile toxin or cholera toxin). An adjuvant for use in the disclosure may be a hybrid molecule such as CpG conjugated to imiquimod. An adjuvant for use in the disclosure may be a fungal or oomycete microbe-associated molecular patterns (MAMPs), such as chitin or beta-glucan. In some embodiments, an adjuvant is an inorganic nanoparticle, such as gold nanorods or silica-based nanoparticles (e.g., mesoporous silica nanoparticles (MSN)). In some embodiments, an adjuvant is a multi-component adjuvant or adjuvant system, such as AS01, AS03, AS04 (MLP5+alum), CFA (complete Freund's adjuvant: IFA+peptiglycan+trehalose dimycolate), CAF01 (two component system of cationic liposome vehicle (dimethyl dioctadecyl-ammonium (DDA)) stabilized with a glycolipid immunomodulator (trehalose 6,6-dibehenate (TDB), which can be a synthetic variant of cord factor located in the mycobacterial cell wall).

Cytokines. An adjuvant may be a partial or full-length DNA encoding a cytokine such as, a pro-inflammatory cytokine (e.g., GM-CSF, IL-1 alpha, IL-1 beta, TGF-beta, TNF-alpha, TNF-beta), Th-1 inducing cytokines (e.g., IFN-gamma, IL-2, IL-12, IL-15, IL-18), or Th-2 inducing cytokines (e.g., IL-4, IL-5, IL-6, IL-10, IL-13).

Chemokines. An adjuvant may be a partial or full-length DNA or RNA (e.g., circRNA or mRNA) encoding a chemokine such as, MCP-1, MIP-1 alpha, MIP-1 beta, Rantes, or TCA-3.

An adjuvant may be a partial or full-length DNA encoding a costimulatory molecule, such as CD80, CD86, CD40-L, CD70, or CD27.

An adjuvant may be a partial or full length DNA or RNA (e.g., circRNA or mRNA) encoding for an innate immune system stimulator (partial, full-length, or mutated) such as TLR4, TLR3, TLR3, TLR9, TLR7, TLR8, TLR7, RIG-l/DDX58, or MDA-5/IFIH1; or a constitutively active (ca) innate immune stimulator, such as caTLR4, caTLR3, caTLR3, caTLR9, caTLR7, caTLR8, caTLR7, caRIG-l/DDX58, or caMDA-5/IFIH1.

An adjuvant may be a partial or full-length DNA or RNA (e.g., circRNA or mRNA) encoding for an adaptor or signaling molecule, such as STING (e.g., caSTING), TRIF, TRAM, MyD88, IPS1, ASC, MAVS, MAPKs, IKK-alpha, IKK complex, TBK1, beta-catenin, and caspase 1.

An adjuvant may be a partial or full-length DNA or RNA (e.g., circRNA or mRNA) encoding for a transcriptional activator, such as a transcription activator that can upregulate an immune response (e.g., AP1, NF-kappa B, IRF3, IRF7, IRF1, or IRF5). An adjuvant may be a partial or full-length DNA encoding for a cytokine receptor, such as IL-2beta, IFN-gamma, or IL-6.

An adjuvant may be a partial or full-length DNA or RNA (e.g., circRNA or mRNA) encoding for a bacterial component, such as flagellin or MBL.

An adjuvant may be a partial or full-length DNA or RNA (e.g., circRNA or mRNA) encoding for any component of the innate immune system.

In some embodiments, a subject is administered a circular or linear polyribonucleotide encoding one or more immunogens in combination with an adjuvant (e.g., an adjuvant that is a separate molecular entity from the circular polyribonucleotide or an adjuvant that is encoded on a separate polyribonucleotide). The term “in combination with” as used throughout the description includes any two compositions administered as part of a therapeutic regimen. This may include, for example, a polyribonucleotide and an adjuvant formulated as a single pharmaceutical composition. This also includes, for example, a polyribonucleotide and an adjuvant administered to a subject as separate compositions according to a defined therapeutic or dosing regimen. An adjuvant may be administered to a subject before, at substantially the same time, or after the administration of a polyribonucleotide. An adjuvant may be administered within 1 day, 2 days, 5 days, 10 days, 20 days, 1 month, 2 months, 3 months, 4 months, 5 months, or 6 months before or after administration of a polyribonucleotide. An adjuvant may be administered by the same route of administration (e.g., intramuscularly, subcutaneously, intravenously, intraperitoneally, topically, or orally) or a different route than a polyribonucleotide.

### Vaccines

In some embodiments of methods described herein, a second agent is also administered to the subject, e.g., a second vaccine is also administered to a subject. In some embodiments, a composition that is administered to a subject includes a circular polyribonucleotide described herein and a second vaccine. In some embodiments, a vaccine and circular polyribonucleotide are co-administered in separate compositions. The vaccine is simultaneously administered with the circular polyribonucleotide immunization, administered before the circular polyribonucleotide immunization, or after the circular polyribonucleotide immunization.

For example, in some embodiments, a subject is immunized with a non-circular polyribonucleotide vaccine (e.g., protein subunit vaccine) and an immunogenic composition including a circular polyribonucleotide. In some embodiments, a subject is immunized with a non-polyribonucleotide vaccine for a first microorganism (e.g., pneumococcus) and an immunogenic composition including a circular polyribonucleotide as disclosed herein. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

In some embodiments, a composition that is administered to a subject includes a linear polyribonucleotide and a vaccine. In some embodiments, a vaccine and linear polyribonucleotide are co-administered in separate compositions. The vaccine is simultaneously administered with the linear polyribonucleotide immunization, administered before the linear polyribonucleotide immunization, or after the linear polyribonucleotide immunization.

For example, in some embodiments, a subject is immunized with a polyribonucleotide (e.g., non-linear polyribonucleotide) vaccine (e.g., protein subunit vaccine) and an immunogenic composition including a linear polyribonucleotide as disclosed herein including a sequence encoding an immunogen.

In some embodiments, a subject is immunized with a non-polyribonucleotide vaccine for a first microorganism (e.g., pneumococcus) and an immunogenic composition including a linear polyribonucleotide as disclosed herein including a sequence encoding an immunogen. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

### Other Embodiments

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All publications, patents, and patent applications are herein incorporated by reference in their entirety to the same extent as if each individual publication, patent, or patent application was specifically and individually indicated to be incorporated by reference in its entirety.

### Examples

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: Design of Circular RNA Encoding Immunogens

This example describes the design of circular RNAs that encode immunogens. In this example, circular RNAs are designed to include an IRES, an ORF encoding an immunogen, and two spacer elements flanking the IRES-ORF. Circularization enables rolling circle translation, multiple ORFs with alternating stagger elements for discrete ORF expression and controlled protein stoichiometry, and an IRES that targets RNA for ribosomal entry. Exemplary immunogens that are encoded by a circular RNA are SARS-CoV-2 immunogens (RBD and Spike), influenza H1N1 immunogens, HPV immunogens, and tumor neoantigens.

### Example 2: Circular RNA Generation and Purification

In this example, circular RNAs are generated by one of two exemplary methods and purified again with the RNA purification system.

Exemplary Method 1: DNA-splint ligation

This exemplary method produces a circular RNA by splint-ligation. RppH-treated linear RNA is circularized using a splint DNA. Unmodified linear RNA is synthesized by in vitro transcription using T7 RNA polymerase from a DNA segment. Transcribed RNA is purified with an RNA purification system (New England Biolabs), treated with RNA 5′phosphohydrolase (RppH) (New England Biolabs, M0356) following the manufacturer's instructions. Alternately or in addition, the RNA was transcribed in an excess of GMP over GTP.

Splint-ligation is performed as follows: circular RNA is generated by treatment of the transcribed linear RNA and a DNA splint between 10 and 40 nucleotides in length using an RNA ligase. To purify the circular RNAs, ligation mixtures were resolved on 4% denaturing PAGE and RNA bands corresponding to each circular RNA were excised. Excised RNA gel fragments were crushed, and RNA eluted with gel elution buffer (0.5 M Sodium Acetate, 0.1% SDS, 1 mM EDTA) for one hour at 37° C. Alternately or in addition, the circular RNA was purified by column chromatography. Supernatant is harvested, and RNA is eluted again by adding gel elution buffer to the crushed gel and incubated for one hour. Gel debris is removed by centrifuge filters and is precipitated with ethanol. Agarose gel electrophoresis is used as a quality control measurement for validating purity and circularization.

Exemplary Method 2: Circularization by self-splicing intron

This exemplary method produces a circular RNA by self-splicing. The circular RNA is generated in vitro. Unmodified linear RNA is in vitro transcribed from a DNA template including all the motifs listed above. In vitro transcription reactions included 1 μg of template DNA T7 RNA polymerase promoter, 10× T7 reaction buffer, 7.5 mM ATP, 7.5 mM CTP, 7.5 mM GTP, 7.5 mM UTP, 10 mM DTT, 40U RNase Inhibitor, and T7 enzyme. Transcription is carried out at 37° C. for 4h. Transcribed RNA is DNase treated with 1 U of DNase I at 37° C. for 15 min. To favor circularization by self-splicing, additional GTP is added to a final concentration of 2 mM, incubated at 55° C. for 15 min. RNA is then column purified and visualized by UREA-PAGE.

### Example 3: Multi-Immunogen Expression from Circular RNA

This example describes expression of multiple immunogens from a circular RNA.

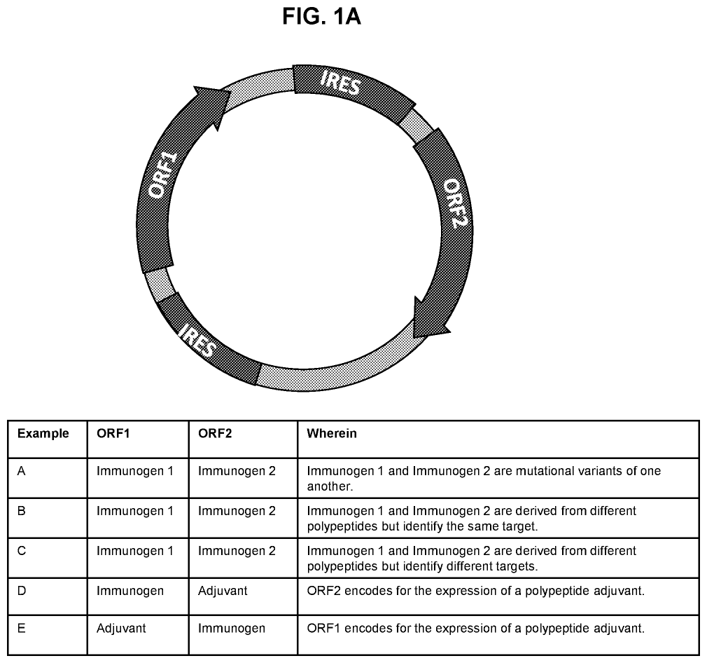

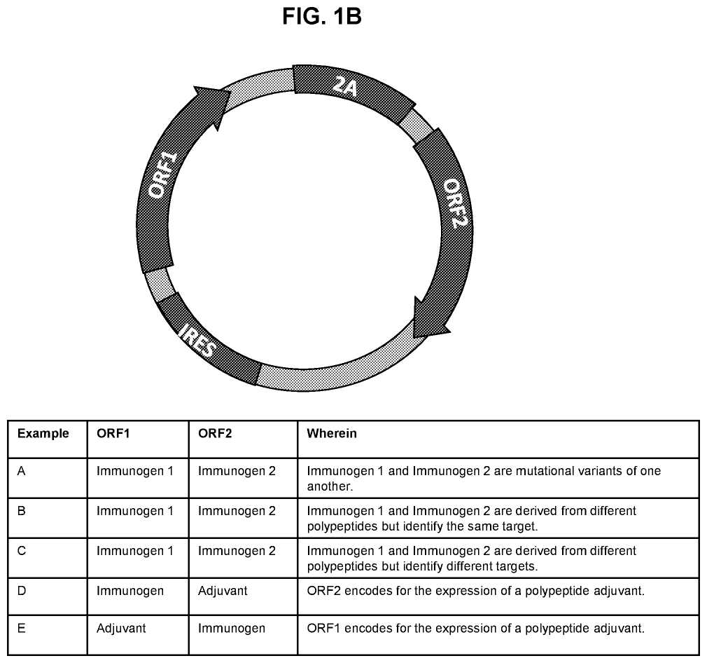

In this Example, one circular RNA is designed to include an IRES (SEQ ID NO: 1) followed by an ORF encoding immunogen 1, corresponding to a portion of hemagglutinin (HA) from a first strain of Influenza A H1N1, A/California/07/2009(H1N1) (SEQ ID NO: 2), a Stop codon, an IRES (SEQ ID NO: 1), another ORF encoding immunogen 2, corresponding to a portion of hemagglutinin (HA) from a second strain of Influenza A H1N1, A/Puerto Rico/8/1934 (SEQ ID NO: 3), a Stop codon, and a spacer (SEQ ID NO: 4), see FIG. 1A The circular RNAs are generated either in vitro or in cells according to the methods described herein.

Briefly, the circular RNA is incubated for 1.5-3 h in rabbit reticulocyte lysate (RRL; Promega, Fitchburg, WI, USA) at 30° C. The final composition of the reaction mixture includes 70% rabbit reticulocyte lysate, 20 μM Amino Acid Mixture (Promega; L446A), and 0.8 U/μL RNasin® Ribonuclease Inhibitor (Promega, N211 A). Hemoglobin is removed by trichloroacetic acid precipitation. After precipitation and centrifugation, the supernatant is discarded and the pellet is dissolved in 2×SDS sample buffer (Thermo) and incubated at 70° C. for 15 min. Samples are resolved on 4-12% gradient polyacrylamide/sodium dodecyl sulfate (SDS) gels (Thermo, NP0326BOX) followed by Western blotting. Proteins are electrotransferred to a polyvinylidene fluoride (PVDF) membrane (Thermo) using a semi-dry method, blotted, and probed with specific antibodies and visualized by chemiluminescence on a C-Digit scanner (LI-COR Biosciences). Image-Studio Lite (LI-COR Biosciences) is used for quantification of expression levels.

Additionally, immunogen 1 and immunogen 2 expression are measured by ELISA in culture supernatants from HeLa cells transfected with eRNA. Briefly, 0.1 pmol of eRNA is transfected into 10,000 HeLa cells using MessengerMax (Invitrogen; LMRNA015) in Opti-MEM (Invitrogen; 31985062). Cell supernatant is harvested at days 1 and 2. The ELISA is performed as follows: a capture antibody is coated onto ELISA plates (MaxiSorp 442404, 96-well) overnight at 4° C. in 100 μL PBS. After washing three times with TBS-T, the plates are blocked for 1 hour with blocking buffer (TBS with 2% FBS and 0.05% Tween 20). Supernatant dilutions are then added to each well in 100 μL blocking buffer and incubated at room temperature for 1 hour. After washing three times with TBS-T, plates are incubated with HRP detection antibody for 1 hour at room temperature. Tetramethylbenzene (Pierce 34021) is added to each well, allowed to react for 5-15 minutes and then quenched with 2N sulfuric acid. The optical density (OD) value will be determined at 450 nm.

### Example 4: Circular RNA Encoding an Immunogen and an Adjuvant

This example describes the ability of a circular RNA to express an immunogen and to further encode an adjuvant. The adjuvant may be expressed or may correspond to a non-expressed nucleic acid sequence, such as an innate immune stimulator sequence.

In a first instance, one circular RNA is designed to include an IRES followed by an ORF encoding an immunogen, a Stop codon, an IRES, another ORF encoding an adjuvant, a Stop codon, and a spacer, see FIG. 1A. The circular RNAs are generated either in vitro or in cells and expressed according to the methods described herein.

In a second instance, one circular RNA is designed to include an IRES followed by an ORF encoding an immunogen, a Stop codon, and further encodes a nucleic acid sequence that is not expressed and acts an adjuvant by stimulating the innate immune system (e.g., a GU-rich motif, an AU-rich motif, a structured region comprising dsRNA, or an aptamer), see FIG. 2. The circular RNAs are generated either in vitro or in cells and expressed according to the methods described herein.

### Example 5: Circular RNA Encoding a Plurality of Immunogens Derived from Mutational Variants

For this example, a circular RNA encodes two polypeptide immunogens that mutational variants of one another, such that they share at least 90% and less than 100% amino acid sequence identity (FIGS. 1A-1B). The polypeptide immunogens each include one or more epitopes corresponding to the same protein. The polypeptide immunogens may be endogenously occurring polypeptide immunogens that are related by genetic drift or modified versions thereof. The circular RNA is designed with a start-codon, expression sequences, stagger element(s), and an IRES (FIG. 1B). Circularization enables rolling circle translation of multiple expression sequences separated by a stagger element.

Mutational variants in the protein hemagglutinin associated with the influenza virus are known to those of skill in the art. Mutational variants are introduced into the haemagglutinin protein as a result of the low fidelity of the viral polymerase resulting in haemagglutinin proteins that no longer have identical sequences (Wong et al. (2011) J. Med. Virol., 83: 510-516). Therefore, a plurality of immunogens corresponding to the various mutational variants of haemagglutinin, where each of the plurality of immunogens shares at least 95% sequence identity is encoded by a circular RNA. The circular RNA includes a start-codon, a first ORF including a polypeptide immunogen derived from the haemagglutinin protein associated with head of the influenza virus, a stagger element, a second ORF including a polypeptide immunogen derived from a mutational variant of the hemagglutinin protein, and an optional IRES.

### Example 6: Circular RNA Encoding a Plurality of Immunogens Derived from the Same Target

For this example, a circular RNA encodes two polypeptide immunogens derived from two different proteins but where both proteins identify the same target. The circular RNA is designed with a start-codon, expression sequences, stagger element(s), and an IRES (FIG. 1B). Circularization enables rolling circle translation of multiple expression sequences separated by a stagger element.

Specifically, the circular RNA encodes a start codon, a first ORF including a polypeptide immunogen derived from HIV-1 envelope glycoprotein 120 (gp120), an optional stagger element, a second ORF including a polypeptide immunogen derived from HIV-1 envelope glycoprotein 41 (gp41), and an optional IRES, where the HIV-1 envelope protein is the target of the polypeptide immunogens. Three gp120s and three gp41s combine in a trimer of heterodimers where the trimer of gp120s are the head region and the trimer of gp41s are the tail region which together form the envelope spikes of HIV-1. Therefore, polypeptide immunogens derived from both gp120 are gp41 are included in the circular RNA to target the HIV-1 envelope protein.

### Example 7: Circular RNA Encoding a Plurality of Immunogens Derived from Different Targets

For this example, a circular RNA encodes two polypeptide immunogens derived from two different proteins that identify different targets from one another. The circular RNA is designed with a start-codon, expression sequences, stagger element(s), and an IRES (FIG. 1B). Circularization enables rolling circle translation of multiple expression sequences separated by a stagger element.

A plurality of immunogens derived from different targets are encoded by the circular RNA such that is designed to have a start codon, a ORF encoding a polypeptide immunogen derived from envelope glycoprotein 1 (gP1) from Varicella Zoster Virus, a stagger sequence, and a polypeptide immunogen derived from haemagglutinin. There are at least 6 envelope glycoproteins of Varicella Zoster Virus and glycoproteins gP1, gP2, gP3 can induce the body to produce neutralizing antibodies (Zweerink et al. 1981; 31(1):436-444). Likewise, two envelope glycoproteins, haemagglutinin and a fusion protein are known immunogens of Morbillivirus. Therefore, the circular RNA encoding a polypeptide immunogen derived from gP1 and a polypeptide immunogen derived from haemagglutinin both targets Varicella Zoster Virus and Morbillivirus.

### Example 8: Multi-Immunogen Administration from Circular RNA

This example describes expression of multiple immunogens in a subject by administrating multiple circular RNA molecules.

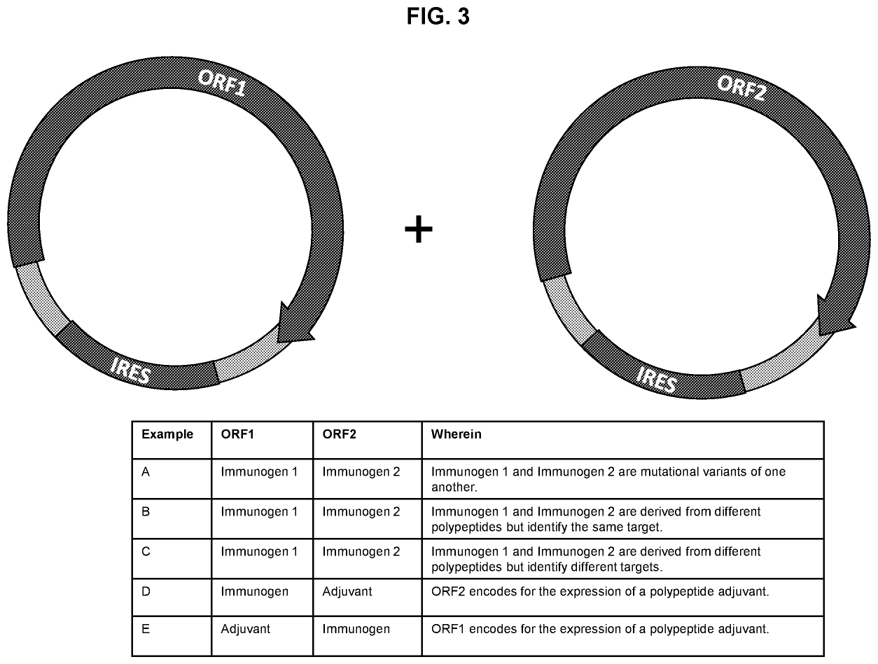

In this Example, circular RNA 1 is designed to include an IRES (SEQ ID NO: 1) followed by an ORF encoding immunogen 1, corresponding to a portion of hemagglutinin (HA) from a first strain of Influenza A H1N1, A/California/07/2009 (SEQ ID NO: 2), a Stop codon and a spacer (SEQ ID NO: 4), see FIG. 2. Circular RNA 2 is designed to include an IRES (SEQ ID NO: 1) followed by an ORF encoding immunogen 2, corresponding to a portion of hemagglutinin (HA) from a second strain of Influenza A H1N1, A/Puerto Rico/8/1934 (SEQ ID NO: 3), a Stop codon, and a spacer (SEQ ID NO: 4), see FIG. 3.

The circular RNAs are generated by in vitro transcription (Lucigen; AS3107) and RNA ligation using a RNA ligase as described by the methods provided herein.

Multiple circular RNAs encoding multiple different immunogens as described herein are formulated for administration to a mammalian subject.

The circular RNAs are formulated in any of the formulations included herein. These formulated RNAs are injected via a suitable route, either intradermal, subcutaneous, intramuscular, or intravenous route at Day 0.

Secreted immunogen expression is evaluated in blood or tissues collected from the mammalian subjects. Blood samples are collected anti-coagulant free tubes, at 1, 2, 7, 14, and 21 days post-dosing. Serum is isolated by centrifugation for 25 min at 1300 g at 4° C. and secreted protein expression is measured by ELISA. Briefly, a capture antibody is coated onto ELISA plates (MaxiSorp 442404, 96-well) overnight at 4C in 100 μL PBS. After washing three times with TBS-T, the plates are blocked for 1 hour with blocking buffer (TBS with 2% FBS and 0.05% Tween 20). Supernatant dilutions are then added to each well in 100 μL blocking buffer and incubated at room temperature for 1 hour. After washing three times with TBS-T, plates are incubated with HRP detection antibody for 1 hour at room temperature. Tetramethylbenzene (Pierce 34021) is added to each well, allowed to react for 5-15 minutes and then quenched with 2N sulfuric acid. The optical density (OD) value is determined at 450 nm.

### Example 9: Co-Administration of an Immunogen and an Adjuvant Encoded by a Plurality of Circular RNAs

This example demonstrates administering a plurality of circular RNAs to a subject to stimulate an immune response. One of circular RNAs administered to a subject encodes a polypeptide immunogen. Another circular RNA administered to a subject encodes an adjuvant (FIG. 3).

In this example, circular RNA encoding a polypeptide immunogen is designed, produced, purified, and prepared as a formulation. To stimulate the immune response, a separate circular RNA including a polyU, which is known to act as an adjuvant by one of skill in the art is designed, produced, purified, and prepared as a pharmaceutical formulation. The formulations of the circular RNA encoding the polypeptide immunogen and the polyU adjuvant are administered to the subject at the same time in different formulations from one another.

### Example 10: Co-Administration of an Immunogen Encoded by a Circular RNA and a Small Molecule Adjuvant

This example demonstrates administering a circular RNA in combination with a small molecule adjuvant to a subject to stimulate an immune response.

In this example, circular RNA encoding a polypeptide immunogen is designed, produced, purified, and prepared as a formulation. To stimulate the immune response, a small molecule adjuvant, such as MF5® adjuvant, is administered to the subject. Both the formulation of circular RNA encoding the polypeptide immunogen and the small molecule adjuvant are administered to the subject at the same time to the subject.

### Example 11: Co-Administration of an Immunogenic Composition Including a Plurality Circular RNAs Each Encoding a Polypeptide Immunogen Corresponding to a Different Target

For this Example, a plurality of circular RNAs each encoding a polypeptide immunogen are administered to a subject (FIG. 3).

One circular RNA encoding a polypeptide immunogen derived from the envelope protein haemagglutinin as is known in the art to identify a Morbillivirus target is administered to a subject. Another circular RNA encoding a polypeptide immunogen derived from the envelope protein glycoprotein E that is known in the art to identify a Varicella Zoster virus target is also administered to a subject. Both circular RNAs are designed, produced, purified, and prepared as a formulation. The formulation including both circular RNAs is administered to a subject.

### Example 12: In Vivo Induction of an Antibody Against an Immunogen in Mammals Using Circular RNA

The circular polynucleotide encoding an immunogen as described herein is formulated for administration to the mammalian subject. The formulation is either in saline or any of the formulations taught herein. The vaccine containing the circular polynucleotide optionally contains one or more dendritic targeting agent or moieties. The vaccine comprising the polynucleotide encoding the immunogen is injected via a suitable route, either intradermal, subcutaneous, intramuscular, or intravenous route at Day 0. A polynucleotide encoding an immunostimulatory agent or moiety can be co-administered with the polynucleotide encoding the immunogen to stimulate immune response. Additional challenges of the vaccine containing the circular polynucleotide encoding the immunogen are given on a weekly, bi-weekly, every three week, every four week, every five week, every six week, every seven week, or every eight week basis until detection of an antibody against the immunogen. Additional vaccine challenges are administered to boost the production of immunogen specific antibodies.

### Example 13: Detecting Expression of a Protein or Immunogen from Circular RNA in Mammalian Cells

To measure expression efficiency of non-secreted proteins or immunogens from the RNA constructs, circular RNA (0.1 picomole) encoding a protein or immunogen is produced and purified according to the methods described herein. Circular RNA is transfected into HEK293 (10,000 cells per well in a 96 well plate in serum-free media) using MessengerMax (Invitrogen, LMRNA).

For a non-secreted protein or immunogen, protein expression is measured using an immunogen-specific ELISA at 24, 48, and 72 hours. To measure expression, cells are lysed in each well at the appropriate timepoint, using a lysis buffer and a protease inhibitor. The cell lysate is retrieved and centrifuged at 12,000 rpm for 10 minutes. Supernatant is collected.

For a secreted protein or immunogen, immunogen expression is detected using an immunogen-specific Western blot at 24, 48, and 72 hours. Briefly, 80 μL of supernatant from mammalian cells is taken from each well. Protein levels in harvested media is measured by BCA protein assay method and same amount of protein is resolved on 4%-12% gradient Bis-Tris gel (Thermo Fisher Scientific) and transferred to nitrocellulose membrane using by iBlot2 transfer system (Thermo Fisher Scientific). Anti-immunogen antibody (Sino Biological) is used to detect the immunogen. The chemiluminescence signal from protein bands is monitored by iBright FL1500 imaging system (Invitrogen).

### Example 14: Expression of RBD Immunogen from Circular RNA in Mammalian Cells

This example demonstrates expression of RBD immunogens from circular RNA in mammalian cells.

In this example, circular RNAs encoding SARS-CoV-2 RBD immunogens were produced and purified according to the methods described herein.

The expression of RBD-encoding circular RNA was tested by immunoprecipitation coupled with Western blot (IP-Western). Briefly, circular RNA encoding an RBD immunogen (0.1 picomoles) was transfected into BJ Fibroblasts and HeLa cells (10,000 cells) using Lipofectamine MessengerMax (ThermoFisher, LMRNA015). MessengerMax alone was used as a control. Supernatant was collected at 24 hours and immunoprecipitation was performed with a rabbit antibody specific to the SARS-CoV-2 RBD-Spike Glycoprotein (Sino Biologicals, Cat: 40592-T62) coupled to Protein G-Dynabeads (Invitrogen, 10003D) and the same antibody was used to detect the immunoprecipitated products resolved by PAGE. A recombinant RBD (42 ng) Immunoprecipitation was used as control and to quantify cell protein expression. Membrane chemiluminescence was quantified using the Image Studio™ Lite western blot quantification software (Li-COR Biosciences).

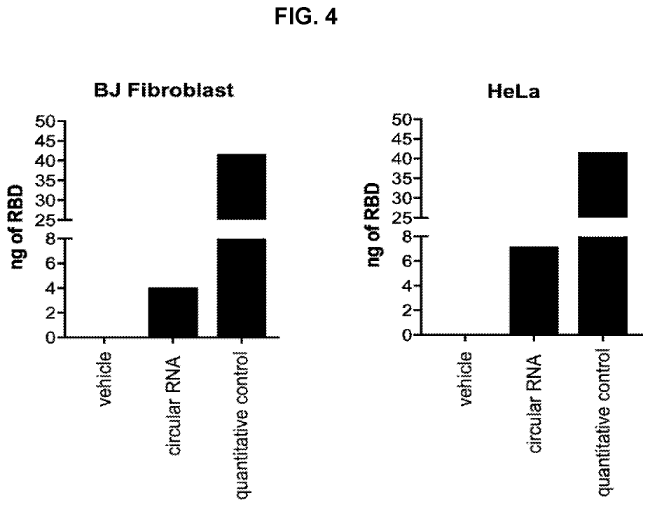

RBD immunogen encoded by circular RNA was detected in BJ Fibroblast and HeLa cell supernatants and not in the controls (FIG. 4).

This example shows that SAR-CoV-2 RBD immunogens (which are secreted proteins) were expressed from circular RNA in mammalian cells.

### Example 15: Immunogenicity of SARS-CoV-2 RBD Immunogens in Mouse Model

The immunogenicity of a circular RNA encoding a SARS-CoV-2 RBD immunogen, formulated with a cationic polymer (e.g., protamine), was evaluated in a mouse model. Production of antibodies to a SARS-CoV-2 RBD immunogen, formulated with the cationic polymer, was also evaluated in the mouse model.

In this example, circular RNA was designed with an IRES and ORF encoding a SARS-CoV-2 RBD immunogen and two spacer elements flanking the IRES-ORF. Circular RNAs were generated as follows. Unmodified linear RNA was synthesized by in vitro transcription with an excess of guanosine 5′ monophosphate using T7 RNA polymerase from a DNA segment. Transcribed RNA was purified with an RNA purification system (New England Biolabs, Inc.) following the manufacturer's instructions. Purified linear RNA was circularized using a splint DNA.

Circular RNA was generated by split-ligation as follows: Transcribed linear RNA and a DNA splint were mixed and annealed and treated with an RNA ligase. To purify the circular RNAs, ligation mixtures were resolved by reverse-phase chromatography. Circular RNA was selectively eluted from linear RNA by increasing the organic content of the mobile phase. Eluted RNA was fractionally collected and assayed for circular RNA purity. Selected fractions were combined and buffer exchanged to remove mobile phase salts and solvents. Acrylamide gel electrophoresis was used as a quality control measurement for validating purity and circularization.

The purified circular RNA was diluted in pure water to a concentration of 1100 ng/μL. Protamine sulfate was dissolved in Ringer's lactate solution (4000 ng/μL). While stirring, the protamine-Ringer lactate solution was added to half of the circular RNA solution until a weight ratio of RNA:protamine is 2:1.

The solution was stirred for another 10 minutes to ensure the formation of stable complexes. The remaining circular RNA was then added (i.e., remaining circular RNA to circular RNA:protamine solution) and the solution stirred briefly. The final concentration of the mixture (i.e., circular RNA mixture) was adjusted using Ringer's lactate solution to obtain a circular RNA preparation with a final RNA concentration of 2 ug or 10 ug of RNA in 50 μL.

Three mice per group were vaccinated intramuscularly or intradermally with a 2 ug or 10 ug dose of the circular RNA preparation, or a protamine vehicle control at day 0 and day 21. Addavax™ adjuvant (Invivogen) was administered once to each mouse, intramuscularly or intradermally, 24 hours after administration of the circular RNA preparation at day 0 and day 21. Addavax™ adjuvant was dosed at 50% in 1×PBS in 50 μL following to the manufacturer's instructions.

Blood collection from each mouse was by submolar drawing. Blood was collected into dry-anticoagulant free-tubes, at day 7, 14, 21, 23, 28, 35, 41, 49, 56, 63, 69, 77, 84, 108 and 115 days post-dosing of the circular RNA. Serum was separated from whole blood by centrifugation at 1200 g for 30 minutes at 4° C. The serum was heat-inactivated by heating at 56° C. for 1 hour. Individual heat-inactivated serum samples were assayed for the presence of RBD-specific IgG by enzyme-linked immunosorbent assay (ELISA). ELISA plates (MaxiSorp 44240496-well, Nunc) were coated overnight at 4° C. with SARS-CoV-2 RBD (Sino Biological, 40592-V08B; 100 ng) in 100 μL PBS. The plates were then blocked for 1 hour with blocking buffer (TBS with 2% FBS and 0.05% Tween 20). Serum dilutions were then added to each well in 100 μL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

The optical density of each serum sample was divided by that of the background (plates coated with RBD, incubated only with secondary antibody). The fold over background of each sample was plotted.

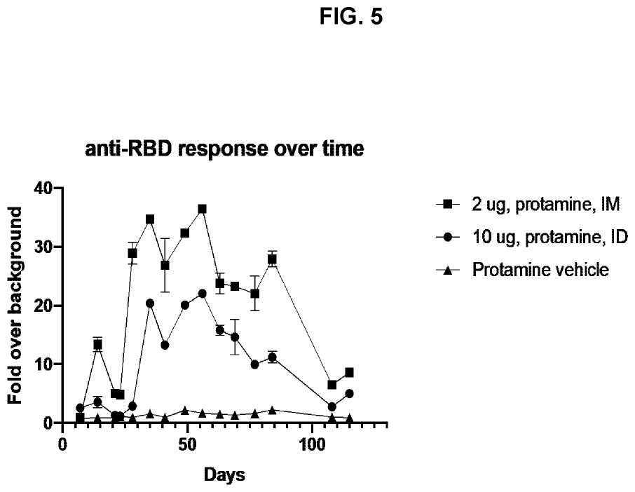

The results showed that anti-RBD responses were obtained at days 14, 21, 23, 28, 35, 41, 49, 56, 63, 69, 77, 84, 108 and 115 after injection with the circular RNA preparations (FIG. 5). Anti-RBD antibodies were not obtained after injection with the protamine vehicle. These results showed that circular RNA encoding the RBD immunogen induced an antigen-specific immune response in mice.

A similar ELISA was used to assay serum samples for the presence of Spike-specific IgG. ELISA plates (MaxiSorp 442404 96-well, Nunc) were coated overnight at 4° C. with SARS-CoV-2 Spike (Sino Biological, 40589-V08B1; 100 ng) in 100 μL PBS. The plates were then blocked for 1 hour with blocking buffer (TBS with 2% FBS and 0.05% Tween 20). Serum dilutions were then added to each well in 100 μL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

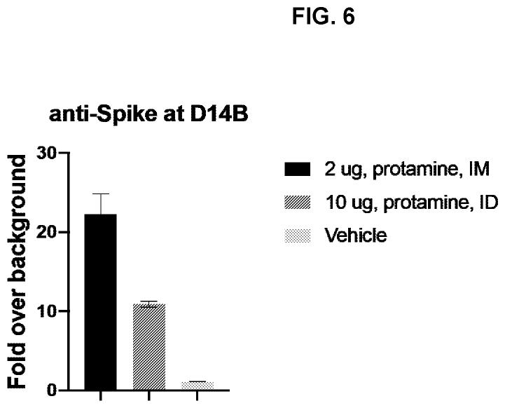

The results showed that anti-Spike antibodies were obtained at 35 days after injection with the circular RNA preparations (FIG. 6). Anti-Spike antibodies were not obtained after injection with vehicle.

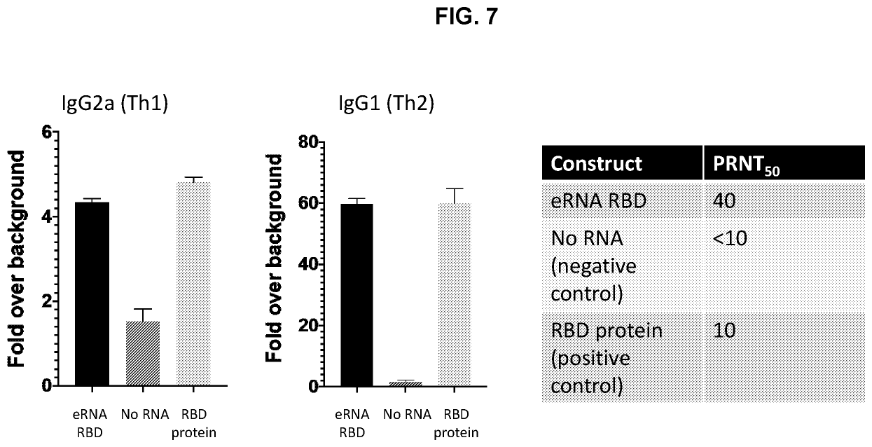

Serum antibodies at day 14 post-dosing were characterized using an assay to measure relative IgG1 vs IgG2a isotypes (FIG. 7), and the ability of serum antibodies to neutralize the virus was characterized using a PRNT neutralization assay. The results showed that 2 μg RBD circular RNA dosed intramuscularly with adjuvant had neutralizing ability.

### Example 16: Formulation of Circular RNA for Administration to Non-Human Animal

Circular RNAs encoding a SARS-CoV-2 immunogen or Gaussia Luciferase (GLuc), as a model immunogen, were designed and purified according to the methods described herein. After purification, the circular RNA or mRNA was formulated as follows:

A. circular RNA or mRNA was diluted in PBS to a final concentration of 2.5 or 25 picomoles in 50 μL, to obtain a circular or a linear RNA preparation (unformulated).

B. circular RNA or mRNA was formulated with a lipid carrier (e.g., TransIT (Mirus Bio)) and mRNA Boost Reagent (Mirus Bio) according to the manufacturer's instructions (15% TransIT, 5% Boost) to obtain a final RNA concentration of 2.5 or 25 picomoles in 50 μL, to obtain a circular or a linear RNA preparation.

C. circular RNA or mRNA was formulated with a cationic polymer (e.g., protamine). Briefly, circular RNA was diluted in pure water. Protamine sulfate was dissolved in Ringer lactate solution (4000 ng/μL). While stirring, the protamine-Ringer lactate solution was added to half of the circular RNA or mRNA solution until a weight ratio of RNA:protamine is 2:1. The solution was stirred for another 10 minutes to ensure the formation of stable complexes. The remaining circular RNA or mRNA was then added, and the solution stirred briefly. The final concentration of the solution was adjusted using Ringer lactate solution such that the final concentration of circular RNA or linear RNA preparation was 2.5 or 25 picomoles in per 50 μL.

D. circular RNA or mRNA was formulated with a lipid nanoparticle. Briefly, circular RNA or mRNA was diluted in 25 mM acetate buffer pH=4 (filtered through 0.2 um filter) to a concentration of 0.2 ug/uL. Lipid nanoparticles (LNPs) were formulated by first dissolving the ionizable lipid (e.g. ALC0315), cholesterol, DSPC, and DMG-PEG2000 in ethanol (filtered through 0.2 um sterile filter) in a molar ratio of 50/38.5/10/1.5 mol %. The final ionizable lipid/RNA weight ratio was 8/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 h to remove ethanol. The RNA concentration inside the LNPs and the encapsulation efficiency were measured using Ribogreen assay. If necessary, the LNPs were concentrated down to the desired RNA concentration using Amicon centrifugation filters, 100 kDa cut off. The size, concentration, and charge of the particles were measured using Zetasizer Ultra (Malvern Pananaytical). The RNA concentration was adjusted with PBS to a final concentration of 0.1 or 0.2 ug/ul. For formulations containing two RNA sequences the RNAs were either mixed before formulating in LNPs or after each RNA was formulated separately. For in vivo experiments, the final RNA formulated in LNPs were filtered through sterile 0.2 um regenerated cellulose filters.

### Example 17: Modulation of In Vivo Production of Gaussia Luciferase from Circular RNA in Mice Using Timed of Adjuvant Delivery

This example demonstrates the expression of proteins or immunogens from circular RNA in vivo whilst also delivering an adjuvant to stimulate an immune response.

In this example, circular RNA encoding GLuc was produced and purified according to the methods described herein. Circular RNAs were formulated as described in Example 16 to obtain circular RNA preparations (e.g., Trans-IT formulated, protamine formulated, PBS/unformulated). Mice were administered 50 μL injections of each circular RNA preparation via either a single intramuscular injection in a hind leg.. Another group of mice were administered a protamine formulated circular RNA preparation intradermally by single intradermal injection to the back.

To stimulate the immune response, Addavax™ adjuvant (Invivogen), which is a squalene-based oil-in-water nano-emulsion with a formulation similar to MF59© adjuvant, was injected into the mouse hind leg at 0 hours (simultaneous delivery with a circular RNA preparation) or at 24 hours. Addavax™ adjuvant was dosed at 50 μL according to the manufacturer's instructions.

Blood samples (˜25 μL) were collected from each mouse by submolar drawing. Blood was collected into EDTA tubes, at 0, 6, 24 and 48 hours post-dosing of the circular RNA. Plasma was isolated by centrifugation for 30 minutes at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read immediately after mixing in a luminometer instrument (Promega).

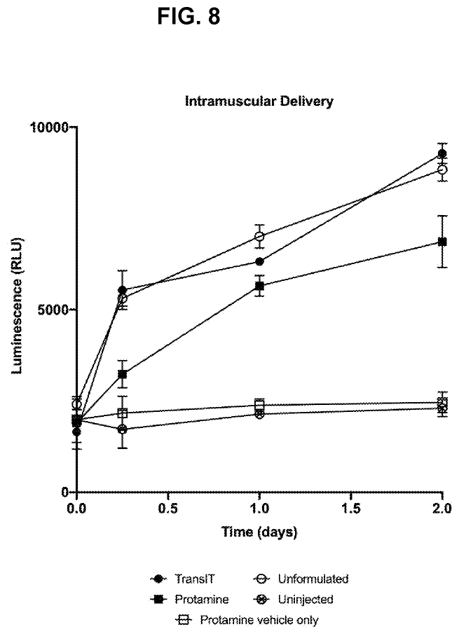

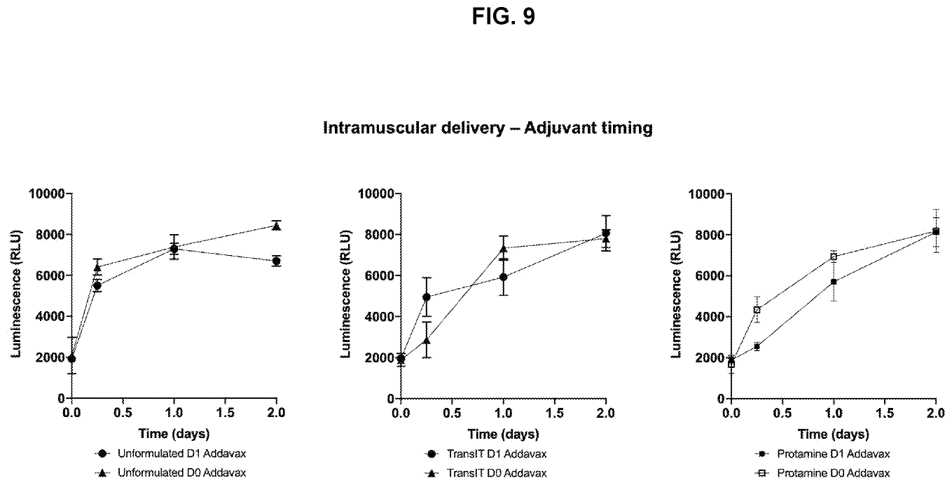

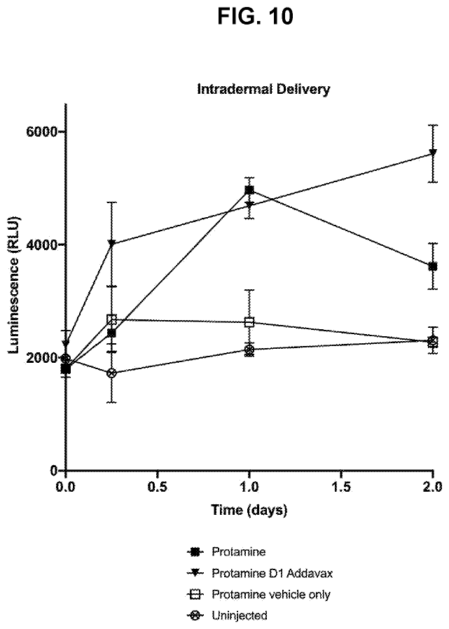

This example demonstrated successful protein expression from circular RNA in vivo for prolonged periods of time using: (a) intramuscular injection of TransIT formulated, protamine formulated and unformulated circular RNA preparations without adjuvant (FIG. 8), and with adjuvant delivered at 0 and 24 h (FIG. 9); and (b) intradermal injection of protamine formulated circular RNA preparation without adjuvant, and with adjuvant delivered at 24 h (FIG. 10).

### Example 18: Characterization of a Circular RNA Preparation by Assessing RNAse H-Produced Nucleic Acid Degradation Products

This example demonstrates that assessment of a circular RNA preparation for RNAse H-produced nucleic acid degradation products can detect linear and concatemerized versus circular products.

RNA, when incubated with a ligase, can either not react or form an intra- or intermolecular bond, generating a circular (no free ends) or a concatemeric RNA (linear), respectively. Treatment of each type of RNA with a complementary DNA primer and RNAse H, a nonspecific endonuclease that recognizes DNA/RNA duplexes, is expected to produce a unique number of degradation products of specific sizes depending on the starting RNA material.

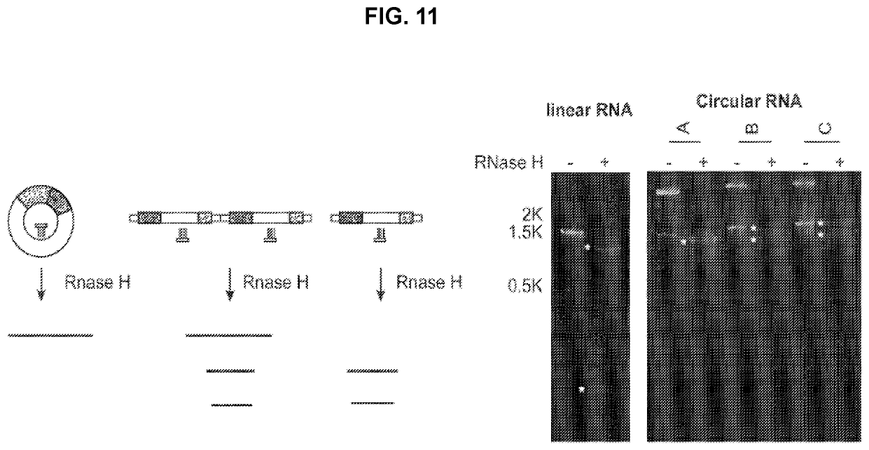

A ligated RNA may be shown to be circular RNA without concatemeric RNA contamination or circular RNA with concatemeric RNA contamination, based on the number and size of RNAs produced by RNAse H degradation. When the primer and RNase H are added to circular RNA, a single primer duplexes with the circular RNA and RNase H degrades the DNA/RNA duplex region to result in a single linear RNA product. When a primer and RNase H are added to a concatemer, at least two primers duplex with the concatemeric RNA and RNase H degrades the DNA/RNA duplexes to result in three products; one product is the RNA from the 5′ end to the first primer binding region, one product is the RNA between the first primer binding region and the next primer binding region which may include multiple RNAs depending on the number of concatemers ligated together, and a final product is the RNA from the last primer binding region to the 3′ end. When a primer and RNase H are added to linear RNA, a single primer duplexes with the linear RNA to result in one product for RNA from the 5′ end to the primer binding region and another product for the primer binding region to the 3′ end. The left side cartoon of FIG. 11 illustrates this strategy.

In this example, circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

To test circularization status of the RNA, 0.05 pmole/μl of linear or circular RNA preparation was incubated with 0.25 U/μl of RNAse H, an endoribonuclease that digests DNA/RNA duplexes, and 0.3 pmole/μl oligomer of 10 to 30 nucleic acids complementary to Nluc RNA at 37° C. for 30 min. After incubation, the reaction mixture was analyzed by 6% denaturing PAGE. The gel was stained with SYBR-green and visualized by E-gel Imager. The band intensity on the visualized gel was measured and analyzed by ImageJ.

The right side of FIG. 11 shows the actual cleavage products in this experiment. The number of bands in the linear RNA lane incubated with RNAse H endonuclease produced two bands as expected, whereas a single band was detected in the circular RNA lane in the case of lane A, indicating that the circular RNA was in fact circular and not concatemeric. In the case of lane B & lane C, bands from linear and concatemer contamination were visible after RNase H treatment due to the presence of multiple smaller fragment bands appearing in the RNAse H lanes.

### Example 19: Linear RNA in Circular RNA Preparations (Standard Curve)

This example demonstrates calculation of linear RNA in a circular RNA preparation.

In this example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

To purify the circular RNAs, ligation mixtures were resolved on 4% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol. Eluted circular RNA was analyzed by 6% denaturing PAGE. The gel was stained with SYBR-green and visualized by E-gel Imager.

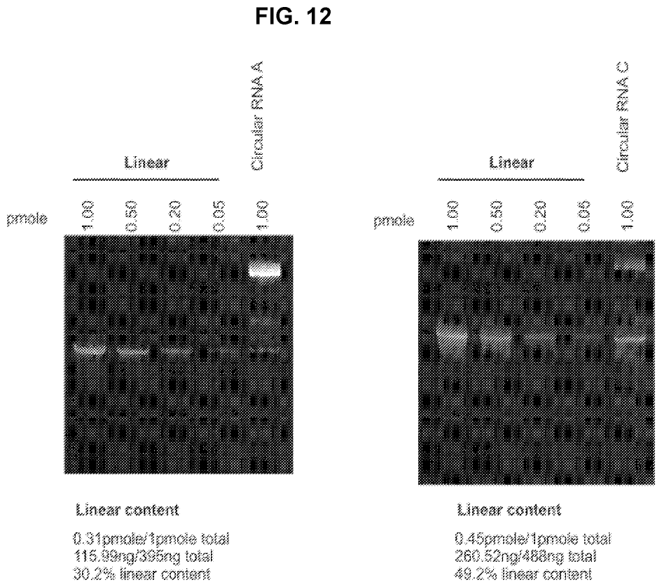

The amount of RNA on the gel was determined by comparing the band intensity of known amount and same size of RNA (standard RNA). A standard curve was generated to determine the amount of unknown sample on the gel (FIG. 12). To generate a standard curve 1, 0.5, 0.2 and 0.05 pmoles of linear counterpart of the circular RNA were loaded parallel with a circular RNA preparation on a 6% denaturing PAGE. The denaturing gel was stained with SYBR-green and visualized by E-gel Imager. Then each band intensity on the gel was measured and analyzed by ImageJ. The standard curve for linear RNA was generated through analysis of band intensity of the RNA loaded in each of the different lanes (R²>0.98 in all cases), and the amount of linear RNA in the circular RNA preparation was determined based on the linear RNA standard curve.

The amount of linear RNA in the different samples was as follows for circular RNA preparation A: linear RNA was calculated to be approximately 0.31 mole/mole, or 115.99 ng/395 ng, or 30.2%. For circular RNA preparation C: linear RNA was calculated to be approximately 0.45 mole/mole, or 260.52 ng/488 ng, or 49.2%.

### Example 20: Linear RNA in Circular RNA Preparations (Gel Excision and Extraction)

This example demonstrates purification and quantification of circular RNA in a preparation.

In this example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

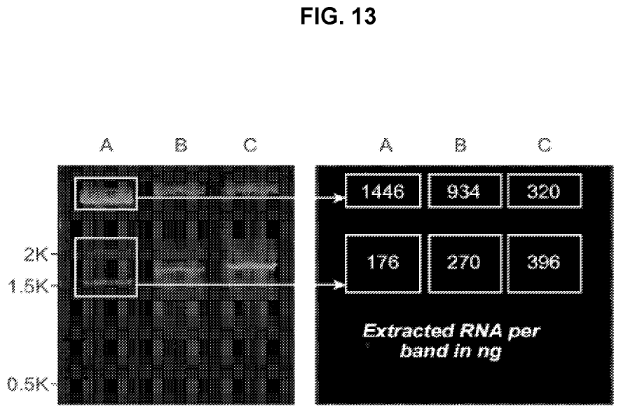

To purify the circular RNAs, ligation mixtures were resolved on 6% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol in the presence of 0.3M sodium acetate. Eluted circular RNA was analyzed by 6% denaturing PAGE. The gel was stained with SYBR-green and visualized by E-gel Imager (FIG. 13).

Visible bands were again excised and crushed using by gel breaker. To extract RNA, gel elution buffer (0.5M NaOAc, 1 mM EDTA, 0.1% SDS) was added to the crushed gel and incubated for 1 hr at 37° C.

Supernatant was harvested, and RNA was eluted once again by adding gel elution buffer to the crushed gel and incubated for an hour. Gel debris was removed by spin X column and extracted RNA was ethanol precipitated. Extracted RNA from individual band was measured by Qubit3 fluorometer.

The extracted RNA from the different samples were quantified as follows: (Preparation A) approximately 1446 ng circular RNA and 176 ng linear RNA (89.1% circular RNA); (Preparation B) approximately 934 ng circular RNA and 270 ng linear RNA (77.5% circular RNA); and (Preparation C) approximately 320 ng circular RNA and 396 ng linear RNA (44.6% circular RNA).

### Example 21: Linear RNA in Circular RNA Preparations

This example demonstrates production of 91% (w/w) pure circular RNA molecules relative to the total ribonucleotide molecules in the preparation and subsequent dosing in mice to generate a biological effect.

In this example, circular RNAs included an IRES, an ORF encoding Gaussia Luciferase (GLuc), and two spacer elements flanking the IRES-ORF.

In this example, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA between 10 and 40 nucleotides in length and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

Linear polyribonucleotides remained in the final circular RNA product preparation. The purity of circular RNA and percentage of remaining linear RNA in the final product preparation was quantified for each batch by running final product preparations on 6% TBE-urea gels and analyzing bands using ImageJ. Purity was assessed by calculating the intensity of circular RNA compared to the total RNA intensity and noted as a percentage. Here, circular RNA was of 91% (w/w) purity relative to the total RNA in the preparation.

Prior to administration, PBS and 10% TransIT carrier were added to achieve the desired final circular RNA concentration of 0.25 pmole in a 100 μL final volume. Mice received a single intravenous tail-vein injection of 0.25 pmole of the circular RNA encoding the Gaussia Luciferase ORF (100 μL).

Blood samples (˜25 μL) was collected from each mouse by submolar drawing. Blood was collected into EDTA tubes, at 0, 6 hours, 1, 2, 3, 7, 14, 21, 28 and 35 days post-dosing. Plasma was isolated by centrifugation for 30 minutes at 1300 g at 4° C. and the activity of Gaussia Luciferase, a secreted enzyme, was tested using a Gaussia Luciferase activity assay (Thermo Scientific Pierce). Briefly, 50 μL of 1× GLuc substrate was added to 5 μL of plasma to carry out the GLuc luciferase activity assay. Plates were read immediately after mixing in a luminometer instrument (Promega).

Gaussia Luciferase activity was detected in plasma at 6 hours and 1, 2, 3, 7, 14, and 21 days post-dosing of circular RNA. Highest expression of circular RNA was observed approximately 2 days post-injection and high levels of expression were maintained for prolonged periods of time and was still detectable at 21 days. At all timepoints, these levels of activity were greater than those observed for the negative control (PBS vehicle only).

This example demonstrates that circular RNA of 91% (w/w) purity relative to the total RNA in the preparation was successfully produced, successfully delivered via intravenous injection and was able to express protein detectable in blood for prolonged periods of time.

### Example 22: Quantification of Nicked Circular RNA in Gel-Purified Circular RNA

This example demonstrates that circular RNA by purified by gel extraction contains no more than 1.1% (w/w) nicked RNA relative to the total RNA molecules in the preparation.

In this example, RNAs included an IRES, an ORF encoding Gaussia Luciferase (GLuc), and two spacer elements flanking the IRES-ORF.

In this example, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH-treated linear RNA was circularized using a splint DNA between 10 and 40 nucleotides in length and T4 RNA ligase 2 (New England Biolabs, M0239). This results in circularization of the RNA at the ligation junction to generate circular RNA. Circular RNA was Urea-PAGE purified on a 4% PAGE gel, eluted in a buffer (0.5 M Sodium Acetate, 0.1% SDS, 1 mM EDTA), ethanol precipitated and resuspended in RNA storage solution (ThermoFisher Scientific, cat #AM7000). In this example, purified circular RNA was evaluated to have a purity of 80% (w/w) relative to the total RNA in the preparation.

In this example, the sequence of linear RNAs was assessed by next generation sequencing. The purified circular RNA preparation (80% purity) was prepared for the NGS pipeline using the library preparation method described in TruSeq Small RNA Workflow (Illumina, RS-200-0012). This method preserved 3′ end identity with high fidelity. Briefly, adapters were ligated to any RNA molecules with available 3′ or 5′ ends in the solution. Intact circular RNA will not undergo this ligation—as a result, this step selected for non-circular RNA only. These products are then reverse transcribed and amplified to generate cDNA libraries which were subsequently purified, quality-controlled and multiplexed. Libraries then underwent sequencing on an Illumina Miniseq machine.

In a similar manner to that described above, linear RNA product from in vitro transcription after RppH-treatment was processed for sequencing.

Sequencing results of both the linear RNA product of IVT and non-circular RNA in the gel-purified circular RNA preparation were compared by mapping reads back to the template sequence used to generate the circular RNA and evaluating the number of reads that map over the ligation junction.

In this analysis, the non-circular RNA that remains is assumed to be a mixture of nicked RNA and residual linear RNA product from IVT. In this example, if the non-circular RNA is assumed to comprise only nicked RNA, the percentage of fragments that map over the ligation junction is expected to be 50%.

If the non-circular RNA is assumed to comprise only residual linear RNA product from IVT, the percentage of fragments that maps over the ligation junction is expected to be 0%. Using these statistical assumptions and control experiments, a standard curve was generated that enabled quantification of nicked RNA. This yielded a calculation of 5.4% of non-circular RNA as nicked RNA.

This example demonstrates that 5.4% (w/w) of the non-circular RNA fraction of a purified circular RNA preparation (equivalent to 1.1% (w/w) of the total RNA) was nicked RNA.

### Example 23: Reduction of Linear RNA Present in Circular RNA Preparations Increased Expression of the Encoded Protein

This example demonstrates that reducing linear RNA present in a predominantly circular RNA composition increased expression of the encoded protein in cells.

For this example, circular RNAs included an IRES, an ORF encoding Gaussia Luciferase (GLuc), and two spacer elements flanking the IRES-ORF.

Two batches of circular RNA were generated. In each case, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA between 10 and 40 nucleotides in length and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated, and resuspended in RNA storage solution (ThermoFisher Scientific, cat #AM7000).

Linear RNA remained in the final circular RNA product. The purity of circular RNA and percentage of remaining linear RNA in the final product was quantified for each batch by running final products on 6% TBE-urea gels and analyzing bands using ImageJ. Purity of circRNA was assessed by calculating the intensity of circRNA compared to the total RNA intensity and noted as a percentage. Here, batches were of 71% purity and 84% purity.

0.2 pmoles of each RNA batch was used for cell transfections. RNA was combined with Optimem and Messenger Max according to the manufacturer's recommendations. A vehicle only control was similarly prepared but did not contain any RNA. At time=0, each preparation was added to BJ fibroblast cells.

The activity of Gaussia Luciferase was tested using a Gaussia Luciferase Activity assay (Thermo Scientific Pierce). Samples of 20 μL of the cell supernatant were added to a 96 well plate (Corning 3990). Samples were taken at 6, 24, 48, 72, 96 and 120 hours after transfection. In brief, 1× coelenterazine substrate was added to each well. Plates were read immediately after substrate addition and mixing in a luminometer instrument (Promega).

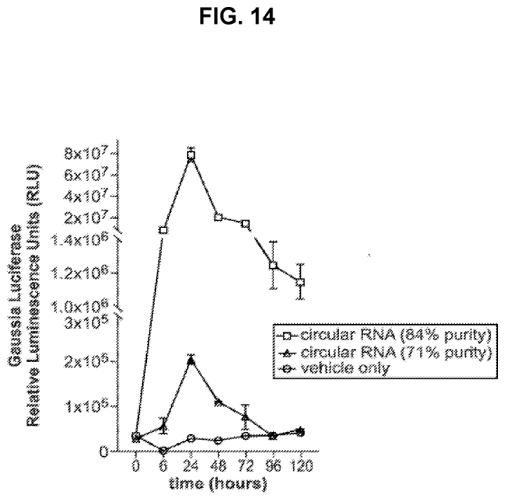

Gaussia Luciferase activity was detected in cells in experiments using circular RNA of 84% purity at 6, 24, 48, 72, 96, and 120 hours post-transfection (FIG. 12) and was higher than the expression afforded by the vehicle only control. Expression derived from circular RNA of 84% purity was significantly higher than the expression derived from circular RNA of 71% purity. At 24 hours post-transfection, GLuc activity was 397-fold greater when circular RNA of 84% purity was transfected compared to when circular RNA of 71% purity was transfected. GLuc activity was detected in cells in experiments using circular RNA of 71% purity at 6, 24, 48, and 72 hours post-transfection (FIG. 14) and was higher than the expression afforded by the vehicle only control.

This example demonstrated that circular RNA of greater purity (with reduced linear RNA) increases and prolongs expression of the encoded protein.

### Example 24: Linear RNA Present in Circular RNA Preparations Affected Innate Immune Response in a Dose Dependent Manner in Cells

This example demonstrates that linear RNA present in a circular RNA preparation negatively affected innate immune response in a dose dependent manner.

In this example, circular RNA and linear RNA were generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Gaussia luciferase (GLuc) and two spacer elements flanking the IRES-ORF.

Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

The impact of varying levels of linear RNA counterparts in preparations of gel purified circular RNA was determined by monitoring circular RNA levels in BJ fibroblast cells. Cells in a 96-well plate were suspension (reverse) transfected with either a gel purified circular RNA preparation, or an equal amount of gel purified circular RNA preparation but supplemented with varying levels of gel purified linear RNA using a lipid-based transfection reagent (ThermoFisher Scientific (LMRNA003). Immune genes levels were analyzed by Q-PCR at 6 hrs and 1-5 days post-transfection. In brief, cDNA was generated from cell lysates by random priming using the Power SYBR Green cells to C_{T} kit (ThermoFisher Scientific, cat #4402953) and following manufacturer's instructions. Q-PCR was performed using immune gene specific primers, and relative RNA levels were calculated using the Pfaffl method and B-Actin as the housekeeping gene.

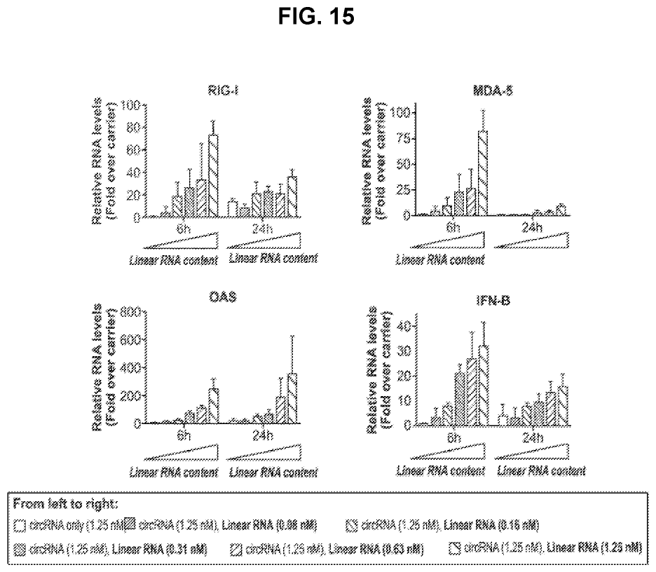

Circular RNA in cells transfected with the gel purified circular RNA preparation alone showed limited increased expression of innate immune genes. Conversely cells transfected with the combined circular and linear RNAs, demonstrated upregulation of innate immune genes in a dose dependent manner (FIG. 15).

### Example 25: Rolling Circle Translation of Synthetic Circular RNA Produced Discrete Protein Products in Cells

This example demonstrates discrete protein or immunogen products were translated via rolling circle translation from synthetic circular RNA lacking a termination element (stop codon), e.g., having a stagger element in lieu of a termination element (stop codon), in cells. Additionally, this example shows that circular RNA with a stagger element expressed more protein or immunogen product having the correct molecular weight than its linear counterpart.

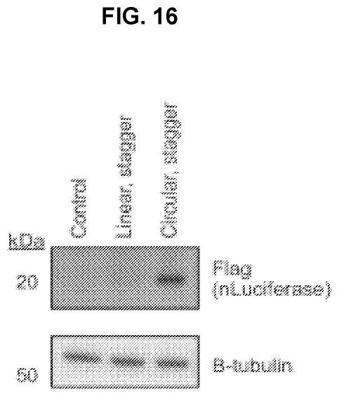

Circular RNAs were designed to include a nanoluciferase gene (nLUC) with a stagger element in place of a termination element (stop codon). Cells were transfected with vehicle: transfection reagent only; linear nLUC: EMCV IRES, stagger element (2A sequence), 3×FLAG tagged nLuc sequences, and a stagger element (2A sequence); or circular nLUC: EMCV IRES, stagger element (2A sequence), 3× FLAG tagged nLuc sequences, and a stagger element (2A sequence). As shown in the FIG. 16, circular RNA produced greater levels of protein having the correct molecular weight as compared to linear RNA.

After 24 hrs, cells were harvested by adding 100 μl of RIPA buffer. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit and western blot band intensity was measured by ImageJ.

As shown in FIG. 16, circular RNA translation product was detected in cells. In particular, circular RNA without a termination element (stop codon) produced higher levels of discrete protein product having the correct molecular weight than its linear RNA counterpart.

### Example 26: Preparation of Circular RNA with Regulatory Nucleic Acid Sites

This example demonstrates in vitro production of circular RNA with a regulatory RNA binding site.

Different cell types possess unique nucleic acid regulatory machinery to target specific RNA sequences. Encoding these specific sequences in a circular RNA could confer unique properties in different cell types. As shown in the following example, circular RNA was engineered to encode a microRNA binding site.

In this example, circular RNA included a sequence encoding a WT EMCV IRES, a mir692 microRNA binding site, and two spacer elements flanking the IRES-ORF.

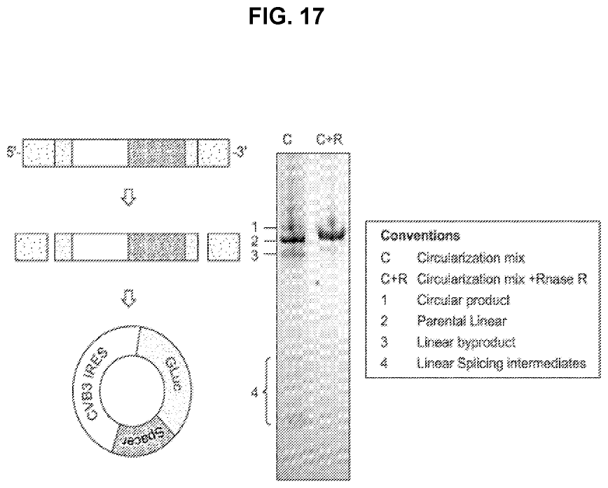

The circular RNA was generated in vitro. Unmodified linear RNA was in vitro transcribed from a DNA template including all the motifs listed above, in addition to the T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA of 10 to 40 nucleotides in length and T4 RNA ligase 2 (New England Biolabs, M0239). Circular RNA was Urea-PAGE purified (FIG. 17), eluted in a buffer (0.5M Sodium Acetate, 0.1% SDS, 1 mM EDTA), ethanol precipitated and resuspended in RNase free water.

As shown in FIG. 17, circular RNA was generated with a miRNA binding site.

### Example 27: Formulation of RNA for Topical Delivery

This example demonstrates formulation of RNA for topical delivery.

To determine topical effects of RNA, RNA was formulated for delivery to epithelial tissues.

As described herein, RNA was formulated with a cell-penetrating agent according to the following:
10 ng RNA linear or circular comprising an EGF ORF
5 μL 80% ethanol
35 μL PBS+glucose (4.5 g/L)

### Example 28: Topical Delivery of Circular RNA

This example demonstrates topical delivery of RNA.

To determine topical delivery effects, RNA was formulated and delivered to epithelial tissues. As described herein, RNA was formulated with a cell-penetrating agent and delivered topically to ear tissue.

Samples of circular RNA were formulated as in Example 27 (50 μL) and applied to both ears of a mouse. Ears were wiped with an isopropyl alcohol wipe prior to application of the samples to the ears. Samples were dried on the ears by exposing the ears briefly to a heat lamp and fan in a sterilized hood. Mice were placed back in cages under normal conditions.

At select timepoints (6 hrs, 1 day, 3 days or 12 days post application), ear tissues (through a single ear punch) were collected for each RNA sample.

### Example 29: Persistence of RNA after Topical Delivery

This example demonstrates RNA presence after topical delivery.

To determine RNA persistence, tissue samples were analyzed for delivered RNA. As described herein, ear punches were analyzed for persistence at varying timepoints after topical delivery of the RNA.

Ear punch samples from Example 28 and untreated ear punch samples were collected in an RNA stabilization reagent and RNA was extracted using a standard RNA tissue extraction kit (Maxwell RSC simply RNA).

A volume of 200 μl of 1-Thioglycerol/Homogenization Solution was added to each sample. A working solution was prepared by adding 20 μl of 1-Thioglycerol per milliliter of Homogenization Solution. Alternatively, 600 μl of 1-Thioglycerol was added to the 30 ml bottle of Homogenization Solution. Before use, the 1-Thioglycerol/Homogenization Solution was chilled on ice or at 2-10° C.

The tissue samples were homogenized in 200 μl of chilled 1-Thioglycerol/Homogenization Solution with a handheld homogenizer and sterile pestle until no visible tissue fragments remained. Each sample was homogenized an additional 15-30 seconds for complete homogenization.

To check for the presence of RNA at the different timepoints, the samples were checked for RNA via q-PCR. qPCR was used to measure the presence of both linear and circular RNA in the ear punches.

To detect linear and circular RNA, primers that amplified the Nluc ORF were used. To detect only circular RNA, primers that amplified the 5′-3′ junction allowed for detection of circular but not linear RNA constructs.

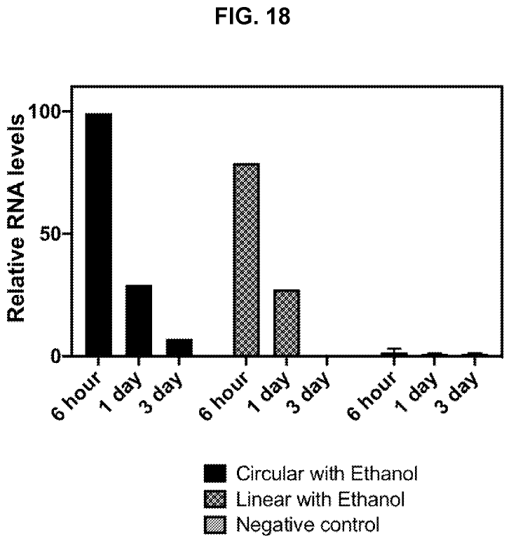

Linear and circular RNA was detected at 6 hrs, 24 hrs, and 72 hrs after topical delivery. Higher levels of circular RNA compared to linear RNA were detected in ears of mice at 3-days post-injection (FIG. 18).

As shown in this example, linear and circular RNA administered topically were detectable in vivo.

### Example 30: Protein Expression of mRNA after Topical Delivery

This example describes protein presence after topical delivery.

To determine if topical delivered RNA can be translated, tissue samples are analyzed for protein or immunogen expression at the different timepoints by western blot. Ear punches are analyzed for protein expression after topical delivery of the RNA.

In short, the ear punches are collected and stored in an RNA stabilization reagent (Invitrogen).

The tissue is homogenized in RIPA buffer with micro tube homogenizer (Fisher scientific) and protein is extracted. Each sample is centrifuged at 14k×g for 15 mins.

The supernatant is removed, and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min.

A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

It is expected that the GFP protein is visualized in ear punch samples and is detected in circular RNA and linear RNA.

### Example 31: Topical Administration of RNA Results in RNA Delivery to Tissue when Ethanol is Included in the RNA Solution

This example demonstrates the delivery of RNA to cells and tissues via topical administration in vivo when ethanol is included in the RNA solution.

In this example, circular RNA was designed with an EMCV IRES and ORF encoding Nanoluciferase (NLuc).

The circular RNA was generated in vitro. Unmodified linear RNA was transcribed in vitro from a DNA template. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA circularized using a splint DNA between 10 and 40 nucleotides in length and T4 RNA ligase 2 (New England Biolabs, M0239). Circular RNA was then Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000). In this example, circular RNA was also HPLC-purified.

In this example, linear mRNA was designed with an ORF encoding a Nano Luciferase (NLuc). In this example, modified linear mRNA was made in-house by in vitro transcription. In this example, linear RNA was fully substituted with Pseudo-Uridine and 5-Methyl-C, capped with CleanCap™ AG, included 5′ and 3′ human alpha-globin UTRs, and is polyadenylated.

RNA was then diluted in PBS/Glucose (4.5 g/L) and ethanol (10% v/v) such that total sample volume for each sample was 50 μL, and total RNA for each sample was 3.5 pmoles. All reagents are brought to room temperature prior to mixing and mixtures are prepared immediately prior to use.

At time=0, 50 μL dose of each sample was applied topically to the ear of a BALB/c mouse dropwise using a pipet tip. As a negative control, an untreated mouse was used. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 6 hours, 1, 3, and 12 days post-administration, a 2 mm ear punch was taken from each animal and stored in RNAlater solution (ThermoFisher Scientific, cat #AM7020). Total RNA was isolated from ear punches using trizol extraction. The aqueous phase was precipitated with isopropanol and the pellet was washed with 70% ETOH as per manufacturer's instructions. cDNA was synthesized from the total RNA and RT-PCR was performed on cDNA templates using primers specific to the NLuc ORF. All samples were assayed in triplicate on the Bio-Rad CFX384 Thermal Cycler. RNA levels were then relativized to actin and to the untreated negative control.

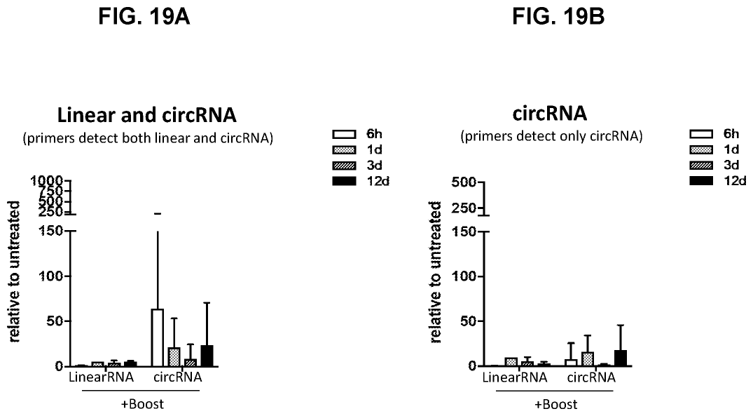

Circular RNA and linear RNA were detected in tissue samples at 6 hours and 1, 3 and 12 days after topical administration and showed greater signal than the vehicle only control (FIG. 19A & FIG. 19B).

This example demonstrates that circular RNA and linear RNA are successfully delivered via topical administration to the tissue when delivered with ethanol and persists in tissue over prolonged periods of time.

### Example 32: Topical Administration of Circular RNA Results in RNA Delivery to Tissue

This example demonstrates the delivery of circular RNA to cells and tissues via topical administration in vivo.

In this example, circular RNA was designed with an ORF encoding a protein or immunogen. In this case, the circular RNA encoded an erythropoietin protein (EPO).

The circular RNA was generated in vitro. Linear RNA was transcribed in vitro from a DNA template including all the motifs listed above, as well as a T7 RNA polymerase promoter to drive transcription. In this example, Cy5-UTP is used to generate Cy5-labeled RNA. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA between 10 and 40 nucleotides and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

Circular RNA was diluted in PBS/glucose (4.5 g/L) with 5% ethanol such that total sample volume for each sample was 25 μL, and total RNA for each sample was 12 picomoles. All reagents were brought to room temperature prior to mixing and mixtures were prepared immediately prior to use.

At time=0, the ear of the mouse was wiped with an isopropyl alcohol wipe, dried and a 25 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine circular RNA delivery to tissue, tissue samples were analyzed by fluorescence microscopy at varying timepoints after administration. At 6 hours, 1 day, and 3 days post administration, a 2 mm ear punch was taken from each animal and stored in ice-cold PBS. Tissue samples were then observed under an EVOS II fluorescent microscope. Images were then quantified for fluorescence using ImageJ.

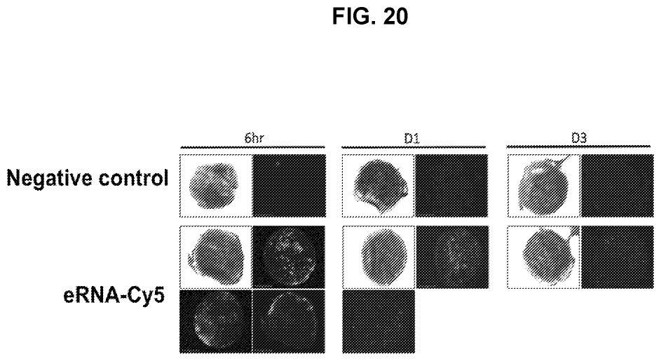

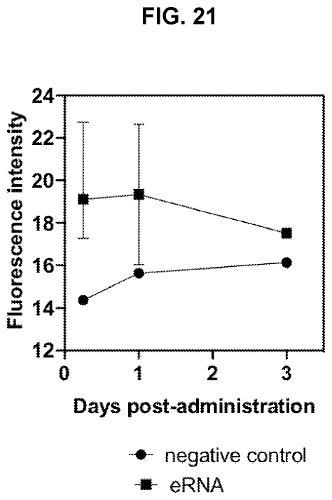

Cy5 signal was detected in tissue samples at 6 hours, 1 and 3 days after topical administration and showed greater signal than the negative control which did not show any fluorescence (FIG. 20 and FIG. 21). This indicates that circular RNA is successfully delivered to the tissue.

This example demonstrates that circular RNA is successfully delivered via topical administration when the skin and persists in tissue over prolonged periods of time.

### Example 33: Topical Administration of Unmodified RNA to the Nasal Mucosal Epithelium Results in Persistence of RNA in Tissue

This example describes the delivery of unmodified RNA via topical administration to the nasal mucosal epithelium and achieve uptake of linear and circular RNA via topical administration.

For this example, an IRES, an ORF encoding Nano Luciferase (NLuc), and two spacer elements flanking the IRES-ORF are included in the RNA.

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA is circularized using a splint DNA between 10 and 40 nucleotides in length and T4 RNA ligase 2 (New England Biolabs, M0239). Circular RNA is Urea-PAGE purified, eluted in a buffer (0.5 M Sodium Acetate, 0.1% SDS, 1 mM EDTA), ethanol precipitated and resuspended in RNaA storage solution (ThermoFisher Scientific, cat #AM7000). Linear RNA counterparts are generated and included the same NLuc ORF and coding components described above.

RNA is diluted to a concentration of 1 pmole/μL. 5 pmole of RNA in citrate buffer is combined with sterile PBS. Total sample volume to be used per application is 20 μL Vehicle only control samples are prepared similarly but without RNA. All reagents are brought to room temperature prior to mixing and mixtures are prepared immediately prior to use.

In a sterilized hood, mice are placed in a hanging position by their ears. At time=0, a 20 μL dose of each sample is gradually release into the nostrils of a BALB/c mouse (10 μL in each nostril) using a micropipette. Mouth and alternate nostril are closed during application to ensure uptake. Mice are kept in the hanging position until breathing rate returned to normal. Mice are placed back in cages under normal conditions.

At 6, 24 and 48 hours post administration, mice are sacrificed, and nasal tissue is taken from the mouse. Each tissue sample (˜2 mg) is placed in 200 μL of chilled 1-thioglycerol/Homogenization solution and homogenized using a handheld homogenized and sterile pestle until no visible tissue fragments remain. Each sample is homogenized for an additional 15-30 seconds for complete homogenization.

To determine RNA persistence in tissue, tissue samples are analyzed for RNA at varying timepoints after delivery using qPCR. qPCR is used to measure both linear and circular RNA in the extracted tissue. Primers that amplify the NLuc ORF are used. To detect circular RNA only, primers that amplify the 5′-3′ junction allow for detection of circular but not linear RNA constructs.

### Example 34: Topical Administration of Circular RNA Results in RNA Delivery to Tissue when Tissue is Wiped with an Ethanol Wipe Prior to Application

This example demonstrates the delivery of unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA having between 10 and 40 nucleotides in length and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in either PBS only, or PBS with 10% (v/v) ethanol, such that total sample volume for each sample was 35 μL, and total RNA for each sample was 20 pmoles. As negative controls, vehicle only controls were prepared as described above but without RNA. All reagents are brought to room temperature prior to mixing and mixtures are prepared immediately prior to use.

At time=0, the ear of the mouse was wiped with an ethanol wipe, dried and a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR®Green Supermix (Bio-Rad, catalog #1725124) and primers specific to the NLuc ORF. All samples were assayed in triplicate on the Bio-Rad CFX384 Thermal Cycler. RNA levels were then relativized to housekeeping gene (28s).

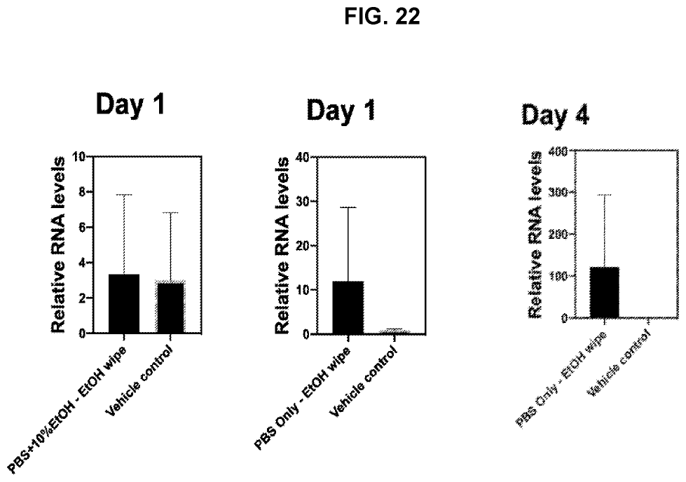

Circular RNA was detected in tissue samples at 1 and 4 days for circular RNA in PBS only, and at 1 days for circular RNA in PBS+10% EtOH after topical administration following wiping the skin with an ethanol wipe and showed greater signal than the relevant vehicle only control (FIG. 22).

This example demonstrates that circular RNA is successfully delivered via topical administration when the skin is wiped with an ethanol wipe prior to administration and persists in tissue over prolonged periods of time.

### Example 35: Topical Administration of Circular RNA Results in RNA Delivery to Tissue when Tissue is Wiped with an Isopropyl Alcohol Wipe Prior to Application

This example demonstrates the delivery of unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA circularized using a splint DNA between 10 and 40 nucleotides in length and T4 RNA ligase 2 (New England Biolabs, M0239). Circular RNA Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS only such that total sample volume for each sample was 35 μL, and total RNA for each sample was 20 pmoles. As negative controls, vehicle only controls were prepared as described above but without RNA. All reagents are brought to room temperature prior to mixing and mixtures are prepared immediately prior to use.

At time=0, the ear of the mouse was wiped with an isopropyl alcohol wipe (CVS, 297584), dried and a 35 μL dose of each sample was applied topically to the ear of a BALB/c mouse dropwise using a pipet tip. As a negative control, an isopropyl alcohol wipe alone was used. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 day and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR®Green Supermix (Bio-Rad, catalog #1725124) and primers specific to the NLuc ORF. All samples were assayed in triplicate on the Bio-Rad CFX384 Thermal Cycler. RNA levels were then relativized to housekeeping gene (28s).

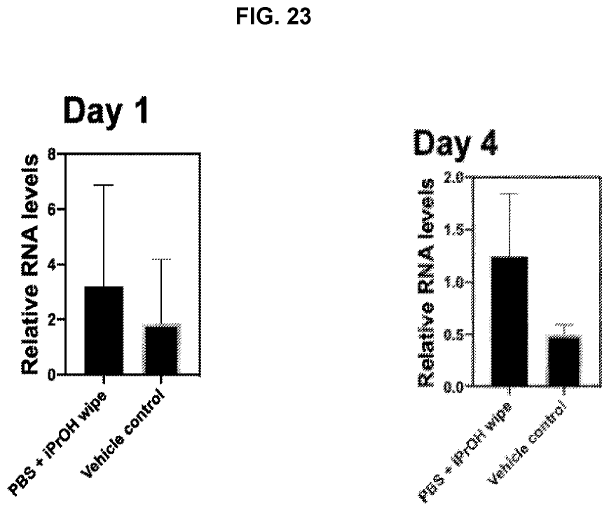

Circular RNA was detected in tissue samples at 1 day and 4 days after topical administration and showed greater signal than the vehicle only control (FIG. 23).

This example demonstrates that endless RNA is successfully delivered via topical administration to the tissue after wiping the skin with an isopropyl alcohol wipe and persists in tissue over prolonged periods of time.

### Example 36: Topical Administration of Circular RNA Results in Protein Expression in Tissue

This example demonstrates the delivery of unmodified circular RNA to cells and tissues via topical administration in vivo and achieve subsequent protein expression.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA between 10 and 40 nucleotides in length and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS with 10% (v/v) ethanol, such that total sample volume for each sample was 35 μL, and total RNA for each sample was 20 pmoles. As negative controls, vehicle only controls were prepared as described above but without RNA. All reagents were brought to room temperature prior to mixing and mixtures were prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 μL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 μL of furimazine substrate and 49 μL of PBS were added to each well and mixed. Plates were incubated for 10 min after substrate addition and mixing and then read in a luminometer instrument (Promega).

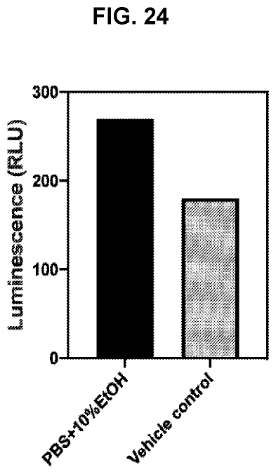

Nano Luciferase activity was detected in tissue samples at 4 days after topical administration for circular RNA in PBS with 10% ethanol (v/v) and was observed to be higher than the relevant vehicle only control (FIG. 24).

This example demonstrated that circular RNA was successfully delivered via topical administration and was able to express functional protein, detectable in tissue for prolonged periods of time.

### Example 37: Topical Administration of Circular RNA Results in RNA Delivery to Tissue when Tissue is Wiped with a Povidone Iodine Prior to Application

This example describes the delivery of unmodified circular RNA to cells and tissues via topical administration in vivo.

In this example, circular RNA is designed with an IRES and ORF encoding Nanoluciferase (NLuc).

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), is treated with RNA 5′phosphohydrolase (RppH) (New England Biolabs, M0356) following the manufacturer's instructions, and is purified again with an RNA purification column. RppH treated linear RNA is circularized using a splint DNA between 10 and 40 nucleotides in length and T4 RNA ligase 2 (New England Biolabs, M0239). Circular RNA is Urea-PAGE purified, eluted in a buffer (0.5 M Sodium Acetate, 0.1% SDS, 1 mM EDTA), is ethanol precipitated and is resuspended in RNA storage solution (ThermoFisher Scientific, cat #AM7000).

RNA is then diluted in PBS only such that total sample volume for each sample is 35 μL, and total RNA for each sample is 20 pmoles. As negative controls, vehicle only controls are prepared as described above but without RNA. All reagents are brought to room temperature prior to mixing and mixtures are prepared immediately prior to use.

At time=0, the ear of the mouse is wiped with commercial povidone iodine (10%), which is a sterilizing agent. Excess povidone iodine is removed with a sterile cotton swab and a 35 μL dose of each sample is applied topically to the ear of a BALB/c mouse dropwise using a pipet tip. Samples are dried by exposing the ears briefly to a heat lamp and fan in a sterilized hood. Mice are placed back in cages under normal conditions.

To determine RNA persistence in tissue, tissue samples are analyzed for RNA at varying timepoints after delivery using RT-qPCR. At 1 day and 4 days post-administration, a 2 mm ear punch is taken from each animal and stored in RNAlater solution (ThermoFisher Scientific, cat #AM7020). Total RNA is isolated from ear punches by snap-cooling and homogenizing the tissue in liquid nitrogen with a glass mortar and pestle followed by trizol extraction (ThermoFisher Scientific cat #15596026). The aqueous phase is precipitated with isopropanol and the pellet is washed with 70% ETOH as per manufacturer's instructions. cDNA is synthesized from the total RNA using Superscript IV (Thermo Scientific, cat #11766500). RT-PCR is performed on cDNA templates using iTaq™ Universal SYBR®Green Supermix (Bio-Rad, catalog #1725124) and primers specific to the NLuc ORF. All samples are assayed in triplicate on the Bio-Rad CFX384 Thermal Cycler. RNA levels are then be relativized to housekeeping gene (28s).

### Example 38: Topical Administration of Circular RNA Results in RNA Delivery to Tissue when Tissue is Sprayed with a Hydrogen Peroxide Spray Prior to Application

This example describes the delivery of unmodified circular RNA to cells and tissues via topical administration in vivo.

In this example, circular RNA is designed with an IRES and ORF encoding Nanoluciferase (NLuc).

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA will be circularized using a splint DNA between 10 and 40 nucleotides in length and T4 RNA ligase 2 (New England Biolabs, M0239). Circular RNA is Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA is then diluted in PBS only such that total sample volume for each sample is 35 μL, and total RNA for each sample is 20 pmoles. As negative controls, vehicle only controls are prepared as described above but without RNA. All reagents are brought to room temperature prior to mixing and mixtures are prepared immediately prior to use.

At time=0, the ear of the mouse is sprayed with commercial hydrogen peroxide (3%), which is a sterilizing agent, dried with a sterile cotton swab and a 35 μL dose of each sample is applied topically to the ear of a BALB/c mouse dropwise using a pipet tip. Samples are dried by exposing the ears briefly to a heat lamp and fan in a sterilized hood. Mice are placed back in cages under normal conditions.

To determine RNA persistence in tissue, tissue samples are analyzed for RNA at varying timepoints after delivery using RT-qPCR. At 1 day and 4 days post-administration, a 2 mm ear punch is taken from each animal and stored in RNAlater solution (ThermoFisher Scientific, cat #AM7020). Total RNA is isolated from ear punches by snap-cooling and homogenizing the tissue in liquid nitrogen with a glass mortar and pestle followed by trizol extraction (ThermoFisher Scientific cat #15596026). The aqueous phase is precipitated with isopropanol and the pellet is washed with 70% ETOH as per manufacturer's instructions. cDNA is synthesized from the total RNA using Superscript IV (Thermo Scientific, cat #11766500). RT-PCR is performed on cDNA templates using iTaq™ Universal SYBR®Green Supermix (Bio-Rad, catalog #1725124) and primers specific to the NLuc ORF. All samples are assayed in triplicate on the Bio-Rad CFX384 Thermal Cycler. RNA levels are then relativized to housekeeping gene (28s).

### Example 39: Detection of Secreted Immunogen in Blood

Blood samples (˜25 μL) are collected from each mouse for analysis by submolar drawing. Blood is collected into EDTA tubes, at 0, 6 hours, 24, 48 hours and 7 days post-dosing of the circular RNA.

Plasma is isolated by centrifugation for 30 minutes at 1300 g at 4° C. Expression of secreted immunogen is assessed using an ELISA or Western blot, e.g. for RBD immunogen, using methods as described in Example 14.

### Example 40: Detection of Antibodies to Immunogen

This example describes how to determine the presence of antibodies to immunogen.

An ELISA is used as described by Chen X et al. (medRxiv, doi: doi.org/10.1101/2020.04.06.20055475 (2020)). Briefly, SARS-CoV-2 protein in 100 μL PBS per well is coated on ELISA plates overnight at 4° C. ELISA plates are then blocked for 1 hour with blocking buffer (5% FBS plus 0.05% Tween 20). 10-fold diluted plasma is then added to each well in 100 μL blocking buffer over 1 hour. After washing with 1× phosphate-buffered saline with Tween® detergent (PBST), bound antibodies are incubated with anti-mouse IgG HRP detection antibody (Invitrogen) for 30 mins, followed by wash with PBST, then PBS, and addition of tetramethylbenzene. The ELISA plate is allowed to react for 5 min and then quenched using 1 M HCl Stop buffer. The optical density (OD) value is determined at 450 nm.

A. For antibodies to SARS-CoV-2 RBD immunogen, the SARS-CoV-2 protein used is SARS-CoV-2 RBD (Sino Biological, 40592-V08B).

B. For antibodies to SARS-CoV-2 spike immunogen, the SARS-CoV-2 protein used is SARS-CoV-2 spike protein (Sino Biological, 40591-V08H)

### Example 41: Increased Protein Expressed from Circular RNA

This example demonstrates synthetic circular RNA translation in cells. Additionally, this example shows that circular RNA produced more expression product of the correct molecular weight than its linear counterpart.

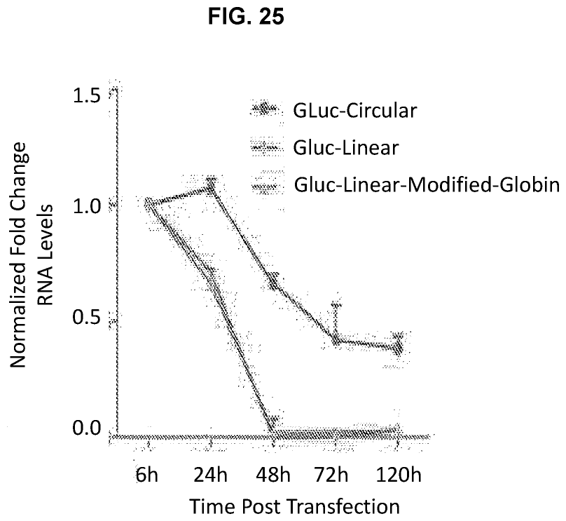

Linear and circular RNAs were designed to include a nanoluciferase gene (nLUC) with a termination element (stop codon). Cells were transfected with vehicle: transfection reagent only; linear nLUC: EMCV IRES, stagger element (2A sequence), 3×FLAG tagged nLuc sequences, a stagger element (2A sequence), and termination element (stop codon); or circular nLUC: EMCV IRES, stagger element (2A sequence), 3×FLAG tagged nLuc sequences, a stagger element (2A sequence), and a termination element (stop codon). As shown in the FIG. 25, circular RNA produced greater levels of protein having the correct molecular weight as compared to linear RNA.

After 24 hrs, cells were harvested by adding 100 μl of RIPA buffer. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit and western blot band intensity was measured by ImageJ.

As shown in FIG. 25, circular RNA was translated into protein in cells. In particular, circular RNA produced higher levels of protein having the correct molecular weight as compared to its linear RNA counterpart.

### Example 42: In Vivo Re-Dosing of Circular RNA

This example demonstrates the ability to drive expression from circular RNA in vivo using two doses of circular RNA.

For this example, circular RNAs included an EMCV IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with a Monarch RNA cleanup kit (New England Biolabs, T2050), treated with RNA 5′-phosphohydrolase (RppH) (New England Biolabs, M0356) following the manufacturer's instructions, and purified again with a Monarch RNA cleanup system. RppH treated RNA was circularized using a splint DNA between 10 and 40 nucleotides in length and T4 RNA ligase 2 (New England Biolabs, M0239). Circular RNA was Urea-PAGE purified, eluted in a buffer (0.5M Sodium Acetate, 0.1% SDS, 1 mM EDTA), ethanol precipitated and resuspended in RNA storage solution (ThermoFisher Scientific, cat #AM7000).

Mice received a single tail vein injection dose of 0.25 μg of circular RNA with the Gaussia Luciferase ORF, or linear RNA as a control, both formulated in a lipid-based transfection reagent (Mirus) as a carrier at day 0, a second dose was administered at day 56.

Blood samples (50 μl) were collected from the tail-vein of each mouse into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1× GLuc substrate was added to 5 μl of plasma to carry out the GLuc luciferase activity assay. Plates were read right after mixing in a luminometer instrument (Promega).

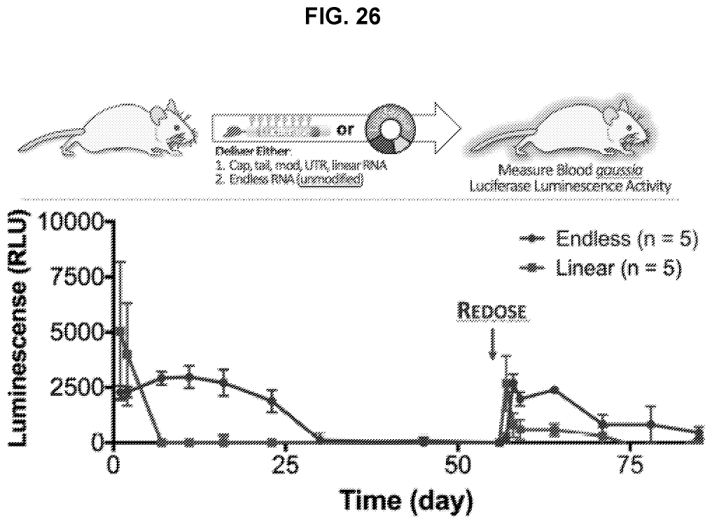

Gaussia Luciferase activity was detected in plasma at 1, 2, 7, 11, 16, and 23 days post-dosing of the first dose of circular RNA (FIG. 26).

In contrast, Gaussia Luciferase activity was only detected in plasma at 1, and 2 days post-dosing of modified linear RNA (FIG. 26).

Gaussia Luciferase activity was detected again in plasma at 2, 3, 8, and 15 days post-dosing of the second dose of circular RNA (FIG. 26).

In contrast, Gaussia Luciferase activity was only detected in plasma at 1, 2, 3 days post-dosing of modified linear RNA.

This example demonstrated that circular RNA expressed protein in vivo for prolonged periods of time, with levels of protein activity in the plasma at multiple days post injection. Additionally, it demonstrates re-dosing of circular RNA results in a similar expression profile.

### Example 43: In Vivo Staggered Dosing of Circular RNA

This example demonstrates the ability to drive higher expression of a protein or immunogen from circular RNA in vivo using continuous staggered doses of circular RNA.

For this example, circular RNAs included an EMCV IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA between 10 and 40 nucleotides in length and T4 RNA ligase 2 (New England Biolabs, M0239). Circular RNA was Urea-PAGE purified, eluted in a buffer (0.5M Sodium Acetate, 0.1% SDS, 1 mM EDTA), ethanol precipitated and resuspended in RNase free water.

Mice received a tail vein injection dose of 0.25 pmol of circular RNA with the Gaussia Luciferase ORF, or linear RNA as a control, both formulated in a lipid-based transfection reagent (Mirus) as a carrier at day 0, day 2 and day 5.

Blood samples (50 μl) were collected from the tail-vein of each mouse into EDTA tubes, at 6 hours, 1, 2, 3, 5, 7, 14, 21, 28, 35, 42 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1× GLuc substrate was added to 5 μl of plasma to carry out the GLuc luciferase activity assay. Plates were read right after mixing in a luminometer instrument (Promega).

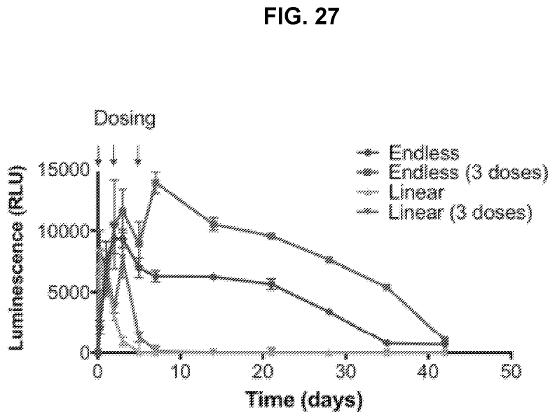

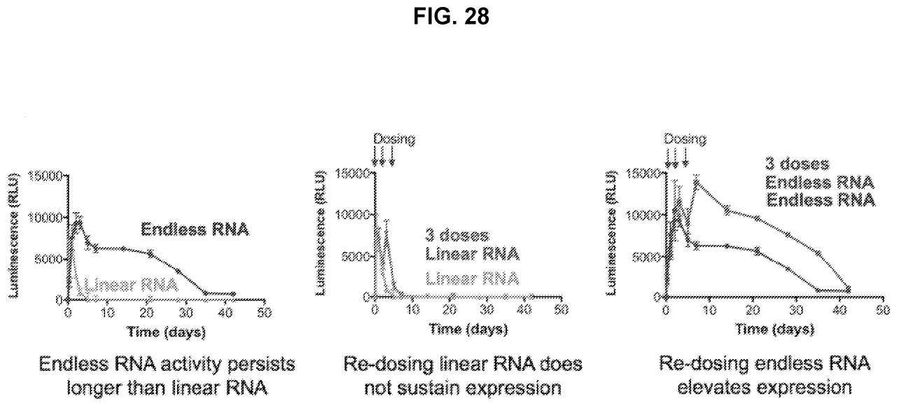

Gaussia Luciferase activity was detected in plasma at 6 hours, 1, 2, 3, 5, 7, 14, 21, 28 days post-dosing of a single dose of circular RNA (FIG. 27 and FIG. 28). Gaussia Luciferase activity was detected in plasma at 6 hours, 1, 2, 3, 5, 7, 14, 21, 28, 35 days post-dosing of the first dose of circular RNA when dosed with 3 doses (FIG. 27 and FIG. 28).

In contrast, Gaussia Luciferase activity was only detected in plasma at 6 hours, 1, 2, 3 days post-dosing of modified linear RNA and expression levels never increased beyond its initial dose. Enzyme activity from linear RNA derived protein was not detected above background levels at day x or beyond even though additional linear RNA was dosed (FIG. 27 and FIG. 28).

This example demonstrated that circular RNA expressed protein in vivo for prolonged periods of time, with increased levels of protein activity in the plasma after multiple injections. Additionally, it demonstrates repeated dosing of circular RNA but not linear RNA results in expression.

### Example 44: Naked Dose and Redose of Circular RNA Via Intramuscular Injection

This example demonstrates the ability to drive expression of a protein or immunogen from circular RNA in vivo using two doses of circular administered intramuscularly.

For this example, circular RNAs included an EMCV IRES, an ORF encoding Gaussia Luciferase (GLuc), and two spacer elements flanking the IRES-ORF.

The circular RNA and mRNA were generated according to the methods described herein.

To generate unformulated RNA, circular RNA and mRNA were then diluted to a final concentration of 2.5 picomoles in 100 μL of PBS.

Mice received a single intramuscular injection to the hind leg of dose of 2.5 picomoles of circular RNA with the Gaussia Luciferase ORF. Injections were performed at day 0, and a second dose was administered at day 49. Vehicle only was used as control.

Blood samples (50 μL) were collected by submental puncture into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μl of plasma to carry out the GLuc luciferase activity assay. Plates were read right after mixing in a luminometer instrument (Promega).

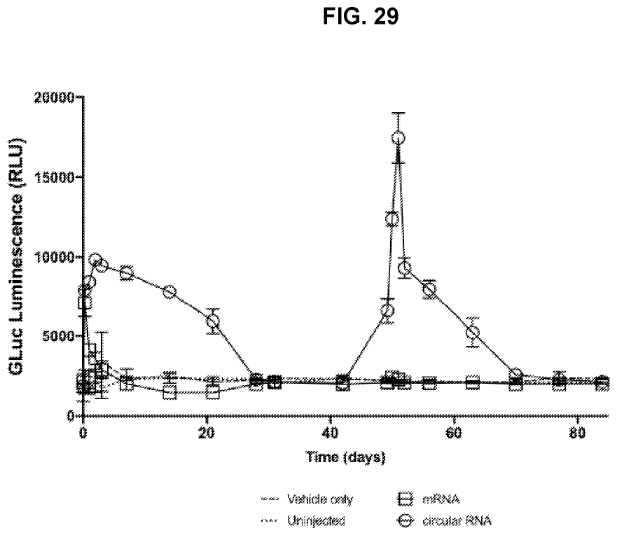

Gaussia Luciferase activity was detected in plasma at 1, 2, 7, 11, 16, and 23 days post-dosing of the first dose of unformulated circular RNA. (FIG. 29)

In contrast, Gaussia Luciferase activity was only detected in plasma at 1, and 2 days post-dosing of unformulated mRNA. (FIG. 29)

Gaussia Luciferase activity was detected again in plasma at 2, 3, 8, and 15 days post-dosing of the second dose of unformulated circular RNA. (FIG. 29)

In contrast, Gaussia Luciferase activity was only detected in plasma at 1, 2, 3 days post-dosing of unformulated modified mRNA. (FIG. 29)

In each case, Gaussia Luciferase activity was greater than the vehicle only control.

This example demonstrated that circular RNA administered intramuscularly, without a carrier, expressed protein in vivo for prolonged periods of time, with levels of protein activity in the plasma at multiple days post injection. Additionally, it demonstrates re-dosing of circular RNA results in a similar expression profile.

### Example 45: Carrier Redose of Circular RNA Via Intravenous Injection Repeated Five Times, Results in Expression of Functional Protein

This example demonstrates the ability to drive expression from circular RNA in vivo using five doses of circular RNA administered intravenously.

For this example, circular RNAs included an EMCV IRES, an ORF encoding Gaussia Luciferase (GLuc), and two spacer elements flanking the IRES-ORF.

The circular RNA and mRNA were generated according to the methods described herein.

Circular RNA and mRNA were formulated using a cationic lipid carrier. In this example, 10% TransIT (Mirus Bio) and 5% Boost were complexed with the RNA according to the manufacturer's instructions.

Mice received a single tail vein injection dose of 0.25 picomoles of circular RNA including the Gaussia Luciferase ORF. Injections were performed at: day 0, day 71, day 120, day 196, and day 359. Vehicle only was used as control.

Blood samples (50 μL) were collected submental puncture into EDTA tubes, at 0.25, 1, 2, 3, 7, 14, 21, 28, and 35 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read right after mixing in a luminometer instrument (Promega).

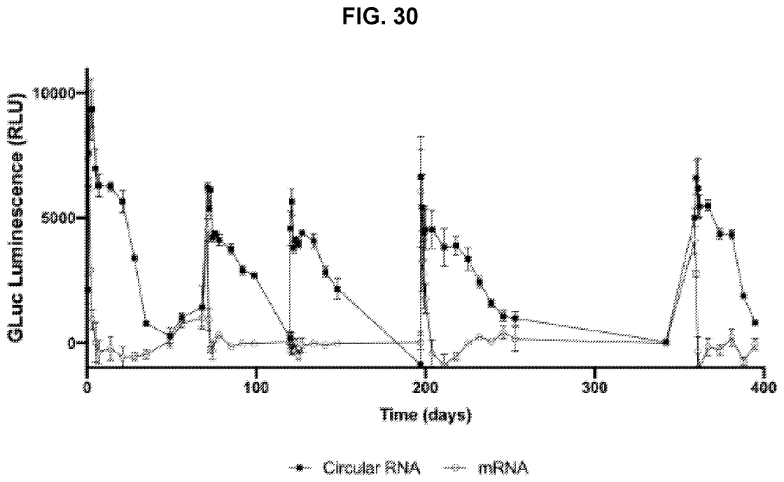

When dosed with Trans-IT formulated circular RNA, Gaussia Luciferase activity was detected in plasma at: days 1, 2, 3, 7, 14, 21 and 28 post-doing of the first dose; days 1, 2, 3, 7, 14 and 21 post-dosing of the second dose; 1, 2, 3, 7, 14 and 21 post-doing of the third dose; days 1, 2, 3, 7, 14, 21 and 28 post-doing of the fourth dose; and, days 1, 2, 3, 7, 14 and 21 post-doing of the fifth dose. (FIG. 30)

In contrast, when dosed with Trans-IT formulated modified mRNA, Gaussia Luciferase activity was detected in plasma at: days 0.25, 1 and 2 post-doing of the first dose; days 0.25, 1 and 2 post-dosing of the second dose; days 0.25, 1 and 2 post-doing of the third dose; days 0.25, 1 and 2 post-doing of the fourth dose; and, days 0.25, 1 and 2 post-doing of the fifth dose. (FIG. 30)

In each case, Gaussia Luciferase activity and thus expression was greater for circular RNA than for the mRNA.

This example demonstrated that circular RNA administered intravenously, expressed protein in vivo for prolonged periods of time, with levels of protein activity in the plasma at multiple days post injection and could be redosed at least 5 times. Additionally, it demonstrates extended re-dosing of circular RNA results in a similar expression profile.

### Example 46: Expression of Multiple Immunogens from Circular RNAs in Mammalian Cells

This example demonstrates expression of multiple immunogens from circular RNAs in mammalian cells.

### Experiment 1

A first circular RNA encoding a SARS-CoV-2 RBD immunogen including an N-terminal Gluc signal sequence (Nucleic acid SEQ ID NO: 33; Amino acid SEQ ID NO: 32) and a second circular RNA encoding SARS-CoV-2 Spike immunogen (Nucleic acid SEQ ID NO: 31; Amino acid SEQ ID NO: 30) were designed and purified according to the methods described herein. The first circular RNA and the second circular RNA were mixed together to obtain a mixture. The mixture (1 picomole of each of the circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD immunogen expression was measured at 24 hours using a SARS-CoV-2 RBD immunogen-specific ELISA. Spike immunogen expression was measured at 24 hours by flow cytometry.

From the transfection with the mixture, SARS-Co-V-2 RBD immunogen was detected in the HeLa cell supernatant and SARS-CoV-2 Spike immunogen was detected on the cell surface of the HeLa cells. From the transfection with the first circular RNA, SARS-CoV-2 RBD immunogen was detected, but SARS-CoV-2 Spike immunogen was not detected. From the transfection with the second circular RNA, SARS-CoV-2 Spike immunogen was detected, but SARS-CoV-2 RBD immunogen was not detected. This demonstrates that both SAR-CoV-2 RBD and SARS-CoV-2 Spike immunogens were expressed in mammalian cells from a combination mixture of circular RNAs.

### Experiment 2

A first circular RNA encoding a SARS-CoV-2 RBD immunogen including an N-terminal Gaussia Luciferase (Gluc) signal sequence (Nucleic acid SEQ ID NO: 33; Amino acid SEQ ID NO: 32) and a second circular RNA encoding a GLuc polypeptide (Nucleic acid SEQ ID NO: 37; Amino acid SEQ ID NO: 36) were designed and produced according to the methods described herein. The first circular RNA and the second circular RNA were separately complexed with Lipofectamine MessengerMax (ThermoFisher, LMRNA015), and then mixed together to obtain a mixture. The mixture (0.1 picomoles of each circular RNAs) was transfected into HeLa cells (20,000 cells per well in a 96 well plate). As controls, the first circular RNA and the second circular RNA were also separately transfected into HeLa cells using MessengerMax.

RBD immunogen expression was measured at 24 hours using a SARS-CoV-2 RBD immunogen-specific ELISA. GLuc activity was measured at 24 hours using a Gaussia Luciferase activity assay (Thermo Scientific Pierce).

From the transfection with the mixture, SARS-CoV-2 RBD immunogen and GLuc activity were detected in the HeLa cell supernatant at 24 hrs. From the transfection with the first circular RNA, SARS-CoV-2 RBD immunogen was detected, but GLuc activity was not detected. From the transfection with the second circular RNA, GLuc activity was detected, but SARS-CoV-2 RBD immunogen was not detected. This demonstrates that both SAR-CoV-2 RBD and GLuc immunogens were expressed in mammalian cells from a combination mixture of circular RNAs.

### Experiment 3

A first circular RNA encoding a SARS-CoV-2 RBD immunogen including an N-terminal Gluc signal sequence (Nucleic acid SEQ ID NO: 33; Amino acid SEQ ID NO: 32) and a second circular RNA encoding hemagglutinin (HA) immunogen from Influenza A H1N1, A/California/07/2009 (Nucleic acid SEQ ID NO: 35; Amino acid SEQ ID NO: 34), were designed and produced according to the methods described herein. The first circular RNA and the second circular RNA were mixed together to obtain a mixture. The mixture (1 picomoles of each circular RNA) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD immunogen expression was measured at 24 hours using a SARS-CoV-2 RBD immunogen-specific ELISA. HA immunogen expression was measured at 24 hours using immunoblot. Briefly, for immunoblot, 24 hours after transfection, cells were lysed and Western blot was performed to detect the HA immunogen using Influenza A H1N1 HA (A/California/07/2009) monoclonal antibody (MA5-29920 (Thermo Fisher)) as the primary antibody and goat anti-mouse IgG H&L (HRP) as the secondary antibody (Abcam, ab 97023). For loading control alpha tubulin was used with alpha tubulin (DM1A) mouse antibody as the primary antibody (Cell Signaling Technology, CST #3873) and goat anti-mouse IgG H&L (HRP) as the secondary antibody (Abcam, ab 97023).

From the transfection with the mixture, both SARS-CoV-2 RBD and Influenza HA immunogens were detected. From the transfection with the first circular RNA, SARS-CoV-2 RBD was detected, but Influenza HA immunogen was not detected. From the transfection with the second circular RNA, Influenza HA immunogen was detected, but SARS-CoV-2 RBD immunogen was not detected. This demonstrates that both SAR-CoV-2 RBD and Influenza HA immunogens were expressed in mammalian cells from a combination mixture of circular RNAs.

### Experiment 4

A first circular RNA encoding a SARS-CoV-2 Spike immunogen (Nucleic acid SEQ ID NO: 31; Amino acid SEQ ID NO: 30) and a second circular RNA encoding hemagglutinin (HA) from Influenza A H1N1, A/California/07/2009 (Nucleic acid SEQ ID NO: 35; Amino acid SEQ ID NO: 34), were designed and produced according to the methods described herein. The first circular RNA and the second circular RNA were mixed together to obtain a mixture. The mixture (1 picomoles of each circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

Spike immunogen expression was measured at 24 hours by flow cytometry. HA immunogen expression was measured at 24 hours by immunoblot as described above in Experiment 3.

From the transfection with the mixture, both SARS-CoV-2 Spike immunogen and Influenza HA immunogen were detected. From the transfection with the first circular RNA, SARS-CoV-2 Spike immunogen was detected, but Influenza HA immunogen was not detected. From the transfection with the second circular RNA, Influenza HA immunogen was detected, but SARS-CoV-2 Spike immunogen was not detected. This demonstrates that both SAR-CoV-2 Spike and Influenza HA immunogens were expressed in mammalian cells from a combination mixture of circular RNAs.

This example shows that multiple immunogens were expressed in mammalian cells from circular RNA preparations comprising different combinations of circular RNAs.

### Example 47: Multi-Immunogen Expression from Circular RNA

This example demonstrates expression of multiple immunogens from a circular RNA in mammalian cells.

### Experiment 1

In this example, a circular RNA was designed to include an IRES followed by an ORF encoding a GLuc polypeptide, a stop codon, a spacer, an IRES, an ORF encoding a SARS-CoV-2 RBD immunogen, and a stop codon. The circular RNA was produced and purified according to the methods described herein.

As controls, the following circular RNAs were produced as described above: (i) a circular RNA with an IRES and ORF encoding a SARS-CoV-2 RBD immunogen; (ii) a circular RNA with an IRES and ORF encoding GLuc polypeptide.

The circular RNAs (0.1 picomoles) were transfected into HeLa cells (10,000 cells per well in a 96 well plate) using Lipofectamine MessengerMax (ThermoFisher, LMRNA015).

RBD immunogen expression was measured at 24 hours using a SARS-CoV-2 RBD immunogen-specific ELISA. GLuc activity was measured at 24 hours using a Gaussia Luciferase activity assay (Thermo Scientific Pierce).

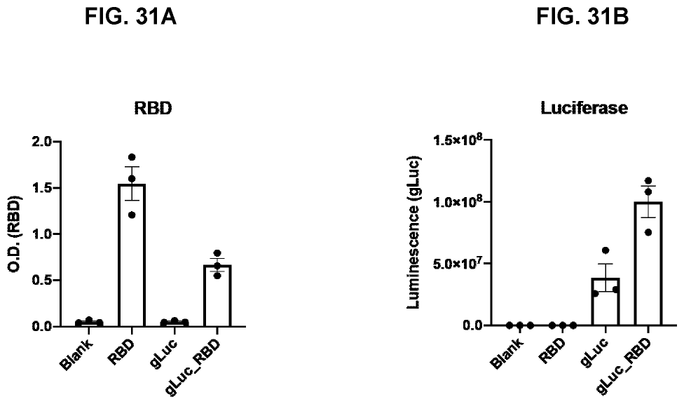

RBD immunogen expression was detected from circular RNAs encoding a SARSs-CoV-2 RBD immunogen and GLuc polypeptide (FIG. 31A). GLuc activity was detected from circular RNAs encoding a SARSs-CoV-2 RBD immunogen and GLuc (FIG. 31B). This demonstrates that both SAR-CoV-2 RBD and GLuc immunogens were expressed in mammalian cells from a circular RNA encoding both SARS-CoV-2 RBD and GLuc immunogens.

### Experiment 2

In this example, a circular RNA designed to include an IRES followed by an ORF encoding a SARS-CoV-2 RBD immunogen, a stop codon, a spacer, an IRES, an ORF encoding a Middle Eastern Respiratory Syndrome (MERS) RBD immunogen, and a stop codon. The circular RNA is produced and purified according to the methods described herein.

The circular RNAs are transfected at various concentrations into HeLa cells (10,000 cells per well in a 96 well plate) using Lipofectamine MessengerMax (ThermoFisher, LMRNA015).

SARS-CoV-2 RBD immunogen expression is measured at 24 hours using a SARS-CoV-2 RBD immunogen-specific ELISA. MERS RBD immunogen expression is measured at 24 hours using a MERS RBD immunogen specific antibody capable of detection.

### Example 48: Immunogenicity of Multiple Immunogens from Circular RNAs in Mouse Model

This example describes expression of multiple immunogens in a subject by administrating multiple circular RNA molecules.

### Experiment 1

The immunogenicity of a circular RNA preparation comprising (a) a circular RNA encoding a SARS-CoV-2 RBD immunogen and (b) a circular RNA encoding GLuc polypeptide as a model immunogen, formulated in lipid nanoparticles, was evaluated in a mouse model. Production of antibodies to the SARS-CoV-2 RBD immunogen and GLuc activity were also evaluated in the mouse model.

A first circular RNA encoding a SARS-CoV-2 RBD immunogen including an N-terminal Gluc signal sequence (Nucleic acid SEQ ID NO: 33; Amino acid SEQ ID NO: 32) and a second circular RNA encoding GLuc polypeptide (Nucleic acid SEQ ID NO: 37; Amino acid SEQ ID NO: 36) were designed and purified according to the methods described herein. The first circular RNA and the second circular RNA were mixed together to obtain a mixture. This mixture was then formulated with lipid nanoparticles as described in Example 16 to obtain a first circular RNA preparation. The first circular RNA and the second circular RNA were also separately formulated with lipid nanoparticles as described in Example 16, and then mixed together to obtain a second circular RNA preparation.

Three mice were vaccinated intramuscularly with the first circular RNA preparation (for a total dose of 10 ug RBD+10 ug GLuc) at day 0 and with the second circular RNA preparation (for a total dose of 10 ug RBD+10 ug GLuc) at day 12. Additional mice (3 or 4 per group) were also vaccinated intramuscularly at day 0 and day 12 with: (i) a 10 ug dose of the first circular RNA formulated with lipid nanoparticles; (ii) a 10 ug dose of the second circular RNA formulated with lipid nanoparticles; or (iii) PBS.

Blood collection from each mouse was by submandibular drawing. Blood was collected into dry-anticoagulant free-tubes, at 2 and 17, days post-priming with the first circular RNA preparation. Serum was separated from whole blood by centrifugation at 1200 g for 30 minutes at 4° C. Individual serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 uL of 1× coating buffer (Biolegend, 421701). The plates were then blocked for 1 hour with blocking buffer (TBS with 2% BSA and 0.05% Tween 20). Serum dilutions (1:500, 1:1500, 1:4500, and 1:13,500) were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Abcam, ab97023) for 1 hour followed by three washes with TBS-T, then addition of tetramethylbenzene (Biolegend, 421101). The ELISA plate was allowed to react for 10-20 minutes and then quenched using 0.2N sulfuric acid. The optical density (O.D.) value was determined at 450 nm.

The optical density of each serum sample was divided by that of the background (plates coated with RBD, incubated only with secondary antibody). The fold over background of each sample was plotted.

The activity of GLuc was tested using a Gaussia Luciferase activity assay (Thermo Scientific Pierce). 50 uL of 1× GLuc substrate was added to 10 uL of serum to carry out the GLuc luciferase activity assay. Plates were read immediately after mixing in a luminometer instrument (Promega).

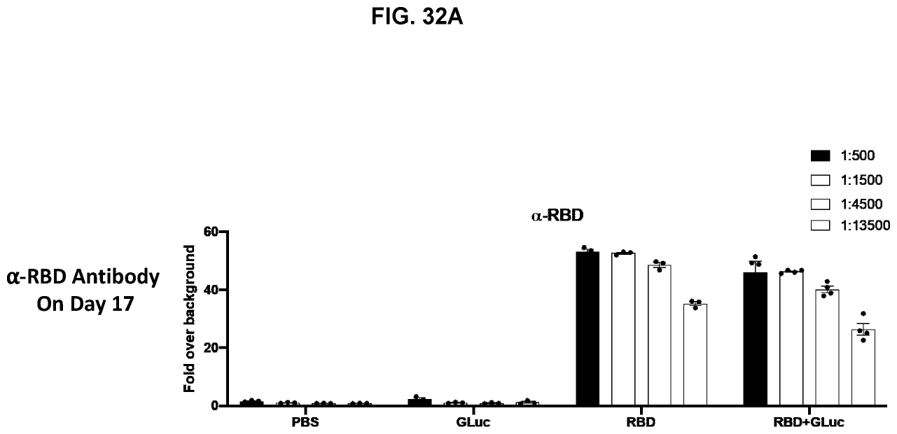

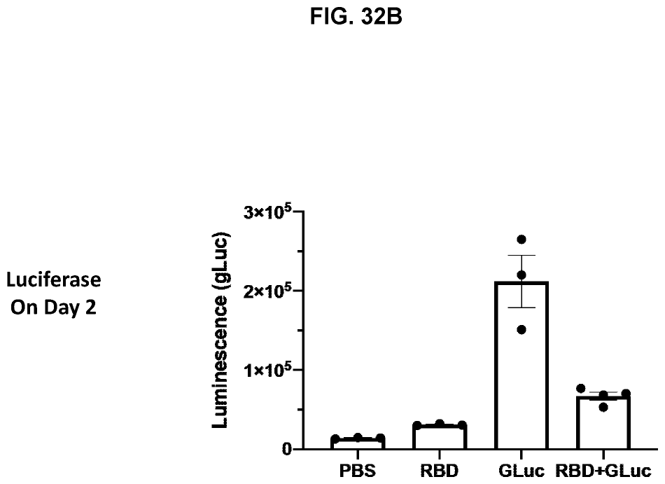

The results showed that anti-RBD antibodies were obtained at 17 days post prime (i.e., 17 days after injection with the first circular RNA preparation) (FIG. 32A) and GLuc activity was detected at 2 days post prime (i.e. 2 days after injection with the first circular RNA preparation) (FIG. 32B).

These results showed that circular RNA preparations comprising two circular RNAs encoding different immunogens induced immunogen-specific immune responses.

### Experiment 2

The immunogenicity of a circular RNA preparation comprising (a) a circular RNA encoding a SARS-CoV-2 RBD immunogen and (b) a circular RNA encoding an Influenza hemagglutinin (HA) immunogen, formulated in lipid nanoparticles, was evaluated in a mouse model. Production of antibodies to the SARS-CoV-2 RBD and Influenza HA immunogens were also evaluated in the mouse model.

A first circular RNA encoding a SARS-CoV-2 RBD immunogen including an N-terminal Gluc signal sequence (Nucleic acid SEQ ID NO: 33; Amino acid SEQ ID NO: 32) and a second circular RNA encoding hemagglutinin (HA) from Influenza A H1N1, A/California/07/2009 (Nucleic acid SEQ ID NO: 35; Amino acid SEQ ID NO: 34), were designed and produced according to the methods described herein. The first circular RNA and the second circular RNA were mixed together to obtain a mixture. This mixture was then formulated with lipid nanoparticles as described in Example 16 to obtain a first circular RNA preparation. The first circular RNA and the second circular RNA were also separately formulated with lipid nanoparticles as described in Example 16, and then mixed together to obtain a second circular RNA preparation.

Three mice were vaccinated intramuscularly with the first circular RNA preparation (for a total dose of 10 ug RBD+10 ug HA) at day 0 and with the second circular RNA preparation (for a total dose of 10 ug RBD+10 ug HA) at day 12. Additional mice (3 or 4 per group) were also vaccinated intramuscularly at day 0 and day 12 with: (i) a 10 ug dose of the first circular RNA formulated with lipid nanoparticles; (ii) a 10 ug dose of the second circular RNA formulated with lipid nanoparticles; or (iii) PBS.

Blood collection was as described in Experiment 1. The presence of RBD-specific IgG by ELISA was determined as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

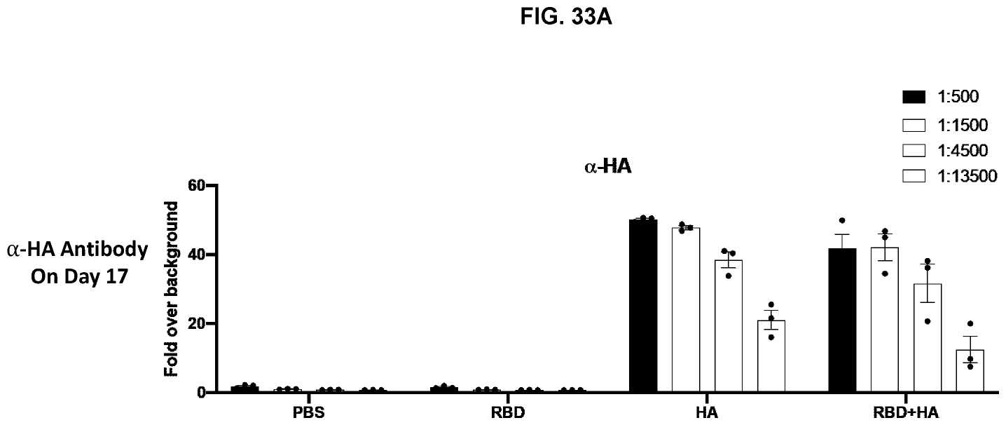

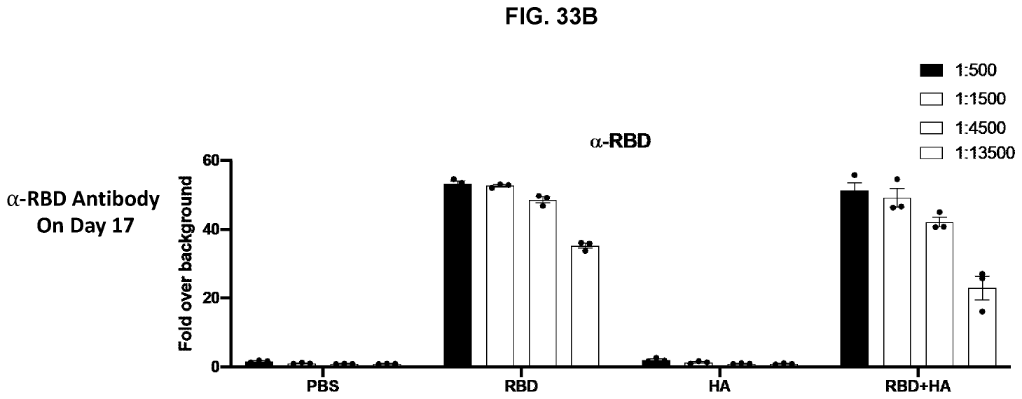

The results showed that anti-RBD and anti-HA antibodies were obtained at 17 days post prime (i.e., 17 days after injection with the first circular RNA preparation (FIG. 33A and FIG. 33B).

The results also showed that circular RNA preparations comprising two circular RNAs encoding different immunogens were expressed in vivo and induced immunogen-specific immune responses.

### Experiment 3

The immunogenicity of a circular RNA preparation comprising (a) a circular RNA encoding a SARS-CoV-2 Spike immunogen and (b) a circular RNA encoding an Influenza hemagglutinin (HA) immunogen, formulated in lipid nanoparticles, was evaluated in a mouse model. Production of antibodies to the SARS-CoV-2 Spike and Influenza HA immunogens were also evaluated in the mouse model.

A first circular RNA encoding a SARS-CoV-2 Spike immunogen (Nucleic acid SEQ ID NO: 31; Amino acid SEQ ID NO: 30) and a second circular RNA encoding hemagglutinin (HA) from Influenza A H1N1, A/California/07/2009 (Nucleic acid SEQ ID NO: 35; Amino acid SEQ ID NO: 34), were designed and produced according to the methods described herein. The first circular RNA and the second circular RNA were mixed together to obtain a mixture. This mixture was then formulated with lipid nanoparticles as described in Example 16 to obtain a first circular RNA preparation. The first circular RNA and the second circular RNA were also separately formulated with lipid nanoparticles as described in Example 16, and then mixed together to obtain a second circular RNA preparation.

Three mice were vaccinated intramuscularly with the first circular RNA preparation (for a total dose of 10 ug Spike+10 ug HA) at day 0 and with the second circular RNA preparation (for a total dose of 10 ug Spike+10 ug HA) at day 12. Additional mice (3 or 4 per group) were also vaccinated intramuscularly at day 0 and day 12 with: (i) a 10 ug dose of the first circular RNA formulated with lipid nanoparticules; (ii) a 10 ug dose of the second circular RNA formulated with lipid nanoparticles; or (iii) PBS.

Blood collection was as described in Experiment 1. Serum was separated from whole blood by centrifugation at 1200 g for 30 minutes at 40C. Individual serum samples were assayed for the presence of RBD (i.e., RBD of Spike)-specific IgG by ELISA as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

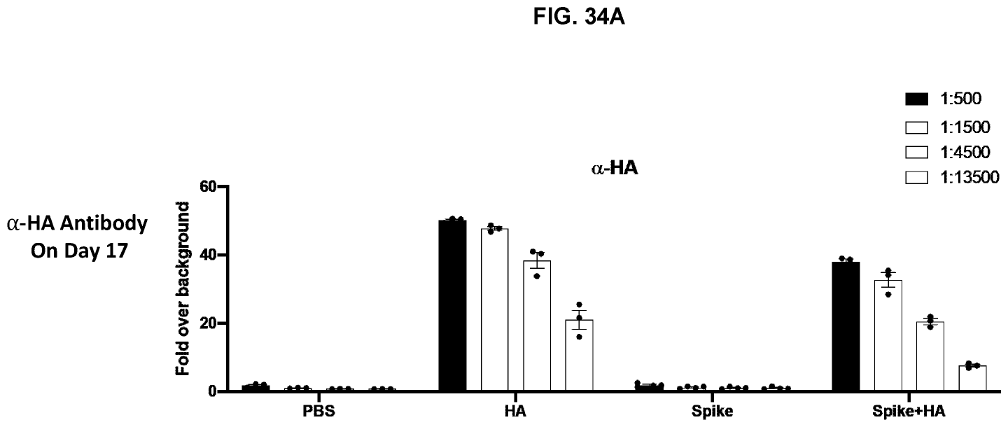

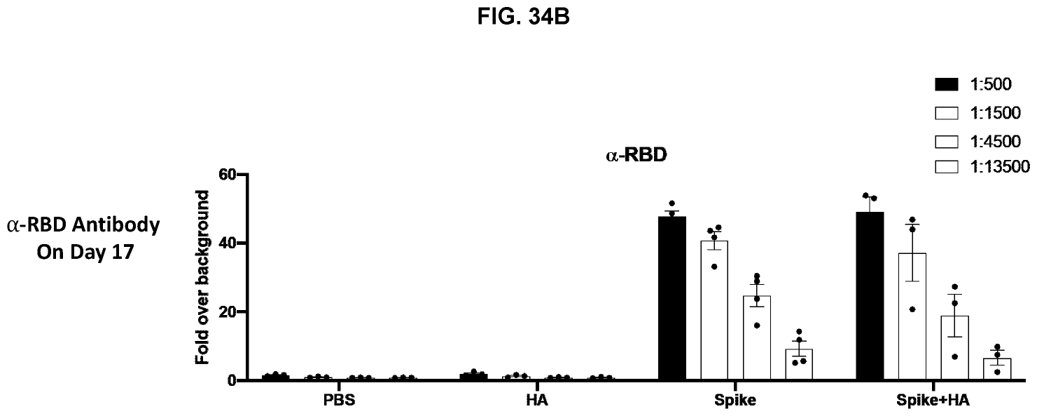

The results showed that anti-RBD antibodies and anti-HA antibodies were obtained at 17 days post prime (i.e., 17 days after injection with the first circular RNA preparation (FIG. 34A and FIG. 34B).

The results also showed that circular RNA preparations comprising two circular RNAs encoding different immunogens induced immunogen-specific immune responses in mice.

### Example 49: Immunogenicity of a Circular RNA Comprising Multiple Immunogens in a Mouse Model

This example describes the immunogenicity of a circular RNA comprising multiples immunogens. This example also describes production of antibodies in a mouse model to multiple immunogens encoded by a single circular RNA.

### Experiment 1

This example describes the immunogenicity of a circular RNA comprising multiple immunogens. This example also describes production of antibodies in a mouse model to multiple immunogens encoded by a single circular RNA.

In this experiment, a first circular RNA was designed to include an IRES followed by an ORF encoding Gluc polypeptide, a stop codon, a spacer, an IRES, an ORF encoding SARS-CoV-2 RBD immunogen, and a stop codon, produced and purified as described in Example 47. The following circular RNAs were also produced as described above: (i) a second circular RNA with an IRES and ORF encoding Gluc polypeptide; and (ii) a third circular RNA with an IRES and ORF encoding a SARS-CoV-2 RBD immunogen.

The first circular RNA and the second circular RNA were each formulated with lipid nanoparticles as described in Example 16 to obtain a first circular RNA preparation and a second circular RNA preparation, respectively. The second circular RNA and the third circular RNA were also separately formulated with lipid nanoparticles, and then mixed together to obtain a third circular RNA preparation.

Three mice per group were vaccinated intramuscularly at day 0 and day 28 with: (i) the first circular RNA preparation (40 ug dose per injection); or (ii) the second circular RNA preparation (10 ug dose per injection); or (iii) the third circular RNA preparation (10 ug Gluc+10 ug RBD dose per injection).

Blood collection from each mouse was by submandibular drawing. Blood was collected from each mouse into dry-anticoagulant free-tubes at day 2, 14, 28, 35, 43, 49, 61, 62 and 63 post-priming of the circular RNAs. Serum was separated from whole blood by centrifugation at 1200 g for 30 minutes at 4° C. Individual serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 uL of 1× coating buffer (Biolegend, 421701). The plates were then blocked for 1 hour with blocking buffer (TBS with 2% BSA and 0.05% Tween 20). Serum was serially diluted (4-fold, 8 times: 1:500 to 1:8192000) and then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Abcam, ab97023) for 1 hour followed by three washes with TBS-T, then addition of TMB ELISA substrate (Pierce, 34021). The ELISA plate was allowed to react for 10-20 minutes and then stopped using 2N sulfuric acid. The optical density (O.D.) value was determined at 450 nm. Serum titer was defined as the reciprocal of the highest serum dilution that gives an O.D. value 5-fold above background.

The activity of Gluc was tested using a Gaussia Luciferase activity assay (ThermoScientific, 16159). 50 uL of 1× Gluc substrate was added to 10 uL of serum to carry out the Gluc luciferase activity assay. Plates were read immediately after mixing in a luminometer instrument (Promega).

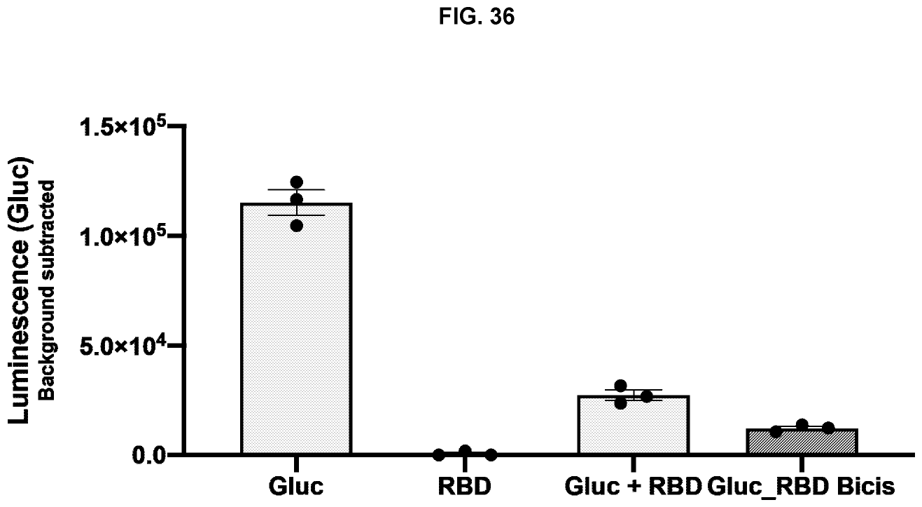

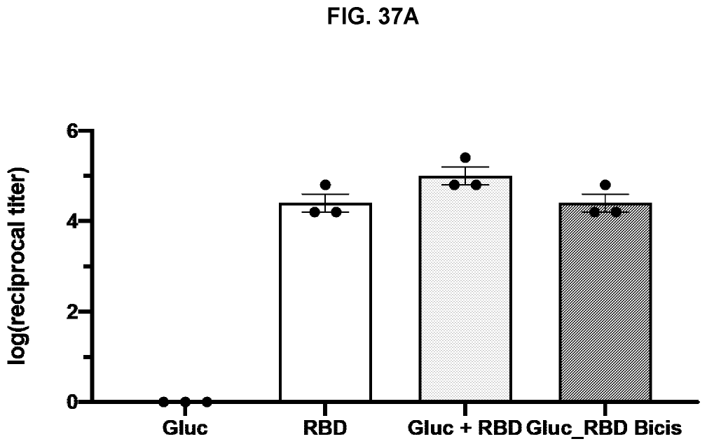

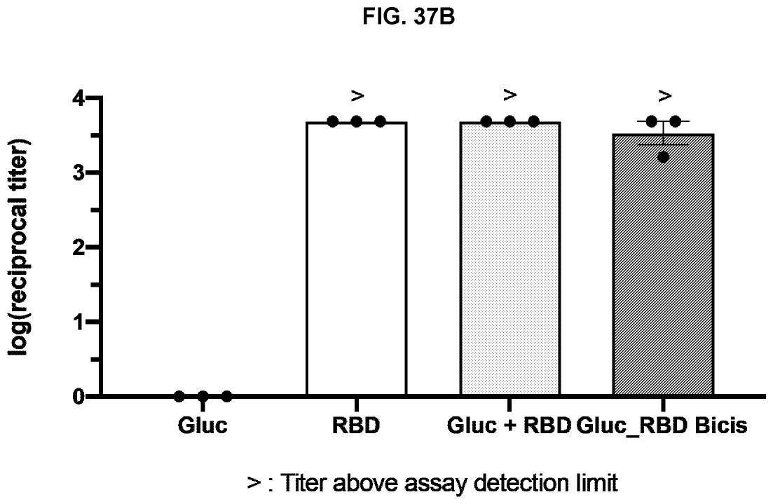

The results showed that Gluc activity was detected at 2 days post prime (i.e., 2 days after injection with the first, second or third circular RNA preparation) (FIG. 36), and that anti-RBD antibodies were obtained at 14 days post prime (i.e., 14 days after injection with the first, second or third circular RNA preparation) (FIG. 37A). Anti-RBD antibodies were also obtained at 28, 35, 43, 49, 61, 62 and 63 days post-priming of the circular RNA (data not shown).

Serum antibodies at day 63 post-dosing were characterized using an assay to measure relative IgG1 vs IgG2a isotypes, and the ability of serum antibodies to neutralize the virus was characterized using a Plaque Reduction Neutralization Test assay. The results showed that neutralizing antibody was obtained in mice dosed with the first circular RNA preparation and the third circular RNA preparation (FIG. 37B).

These results show that a single circular RNA encoding two immunogens can prime an immunogen-specific immune response. In addition, the results show that the circular RNAs comprising an ORF encoding a SARS-CoV-2 RBD immunogen had neutralizing ability.

### Experiment 2

The immunogenicity of a circular RNA preparation comprising a circular RNA designed to include an IRES followed by an ORF encoding a SARS-CoV-2 RBD immunogen, a stop codon, a spacer, an IRES, an ORF encoding a MERS RBD immunogen, and a stop codon, formulated in lipid nanoparticles, is evaluated in a mouse model. Production of antibodies to the SARS-CoV-2 RBD and MERS RBD immunogens are also evaluated in the mouse model.

This circular RNA is then formulated with lipid nanoparticles as described in Example 16 to obtain a circular RNA preparation.

Mice are vaccinated intramuscularly or intradermally with the circular RNA preparation with amounts of 5 μg, 10 μg, 20 μg, or 50 μg at day 0 and again at least one day after the initial administration.

Blood collection is as described in Experiment 1. The presence of SARS-CoV-2 RBD-specific and MERS RBD-specific IgGs by ELISA is determined as described in Experiment 1.

Individual serum samples are assayed for the presence of anti-SARS-CoV-2 RBD binding antibodies, anti-MERS RBD binding antibodies, neutralizing antibodies against the SARS-CoV-2 RBD immunogen, neutralizing antibodies against the MERS RBD immunogen, a cellular response to the SARS-CoV-2 immunogen, and a cellular response to the MERS RBD immunogen.

### Example 50: Evaluation of T Cell Responses

### Experiment 1

An ELISpot assay is used to detect the presence of SARS-CoV-2 Spike or RBD-specific T cells or Influenza HA-specific T cells. This assay is performed on the following groups of mice from Example 48:
1. RBD
2. GLuc
3. HA
4. Spike
5. RBD+HA
6. Spike+HA
7. PBS

Mice spleens are harvested on day 30 post boost (i.e., 30 days after injection with the first circular RNA preparation), and processed into a single cell suspension. Splenocytes are plated at 0.5M cells per well on IFN-gamma or IL-4 ELISpot plates (ImmunoSpot). Splenocytes are either left unstimulated or stimulated with SARS CoV-2 and HA peptide pools (JPT, PM-WCPV-SRB and PM-IFNA_HACal).

ELISPOT plates are processed according to manufacturer's protocol.

### Experiment 2

An ELISpot assay was used to detect the presence of SARS-CoV-2 RBD-specific T cells. This assay was performed on the groups of mice from Example 49.

Mice spleens were harvested on day 63 post prime (i.e., 63 days after injection with the first, second or third circular RNA preparation), and processed into a single cell suspension. Splenocytes were plated at 0.5M cells per well on IFN-γ (Mabtech). Splenocytes were either left unstimulated or stimulated with SARS CoV-2 RBD peptide pool (JPT, PM-WCPV-SRBD). ELISPOT plates were processed according to manufacturer's protocol.

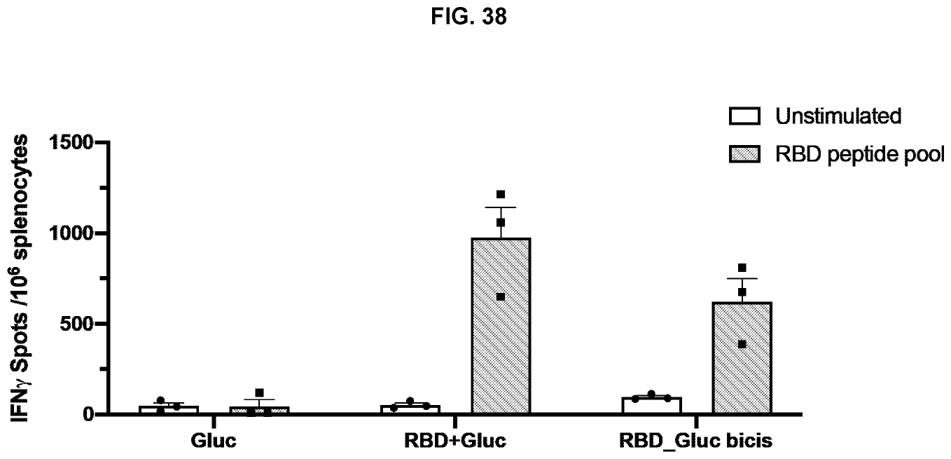

The results showed that a preparation of circular RNA comprising of a single circular RNA encoding different two immunogens induced immunogen specific T cell responses (FIG. 38)

### Example 51: Evaluation of Antibody Response in Mice Administered Circular RNA Encoding Multiple Immunogens

This example demonstrates an antibody response resulting from administration of a circular RNA encoding the expression of the multiple immunogens.

A hemagglutination inhibition assay (HAI) was used to measure anti-Influenza HA antibodies that prevent hemagglutination in serum from mice. Mice were administered a preparation of circular RNA each of which was designed and produced the methods described herein, and which encode for the expression of: a SARS-CoV-2 RBD immunogen, a SARS-CoV-2 Spike immunogen, an Influenza HA immunogen, a SARS-CoV-2 RBD immunogen and an Influenza HA immunogen, a SARS-CoV-2 RBD immunogen and a GLuc polypeptide, or a SARS-CoV-2 RBD immunogen and a SARS-CoV-2 Spike immunogen. Blood collection was as described in Example 47, Experiment 1 and was performed on day 2 and day 17 after injection.

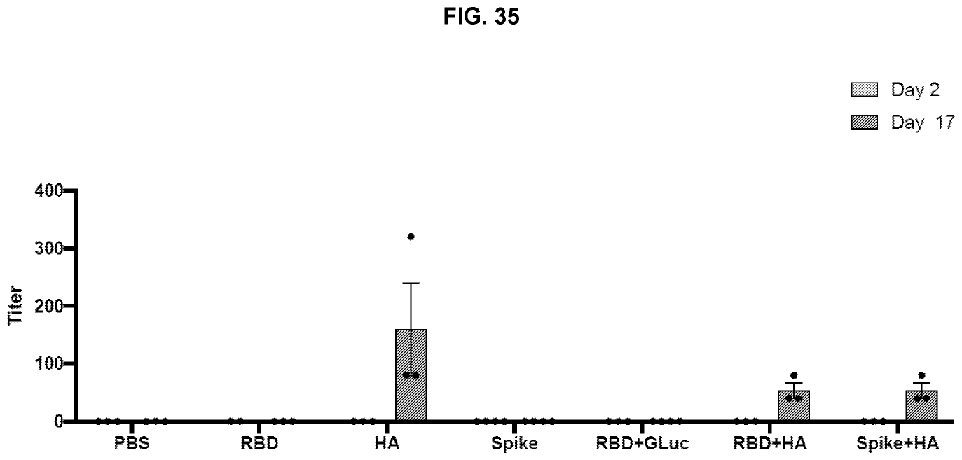

Two-fold serial dilutions of the collected sample from mice on day 2 and day 17 were prepared. A fixed amount of influenza virus with known hemagglutinin (HA) titer was added to every well of a 96-well plate, to a concentration equivalent to 4 hemagglutinin units, with the exception of the serum control wells, where no virus was added. The plate was allowed to stand at room temperature for 60 minutes, after which the red blood cell samples were added and allowed to incubate at 4° C. for 30 minutes. The highest serum dilution that prevented hemagglutination was determined to be the HAI titer of the serum. The sample collected on day 17 showed HAI titer in samples that were administered circular RNA preparations encoding the Influenza HA immunogen when it was administered alone or when administered in combination with SARS-CoV-2 immunogens e.g. RBD or Spike (FIG. 35). HAI titers on day 17 were not seen from samples where HA immunogen had not been administered e.g. the SARS-CoV-2 RBD immunogen alone or SARS-CoV-2 Spike immunogen alone.

### Example 52: Expression of an Adjuvant from Circular RNA in Mammalian Cells

This example demonstrates expression of a polypeptide adjuvant from circular RNA in mammalian cells.

In this example, a circular RNA was designed to include an IRES, an ORF encoding the adjuvant IL-12 (Nucleic acid SEQ ID NO: 38; Amino acid SEQ ID NO: 39), and two spacer elements flanking the IRES-ORF. As a control, a circular RNA including an IRES, an ORF encoding a SARS-CoV-2 RBD immunogen, and two spacer elements flanking the IRES-ORF was used. The circular RNAs were produced and purified according to the methods described herein.

Purified circular RNAs (0.1 and 1 picomoles) were transfected into HeLa cells (10,000 cells per well) using Lipofectamine MessengerMax (Invitrogen LMRNA001) according to the manufacturer's instructions.

IL-12 expression was measured using an IL-12 specific ELISA (ThermoFisher, BMS6004) in cell culture supernatant. Data are shown as Mean and SEM values of two replicates.

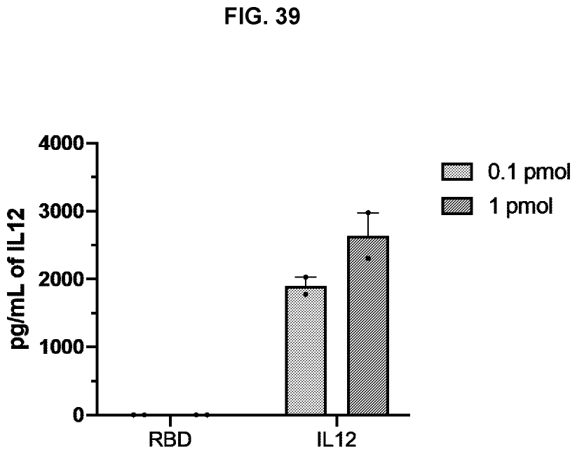

The results showed that IL-12 encoded by circular RNA was expressed by HeLa cells and not in the control (FIG. 39). This example shows that the IL-12 adjuvant was expressed from circular RNA in mammalian cells.

### Example 53: In Vivo Expression of an Adjuvant from Circular RNA in Mouse Model

This example demonstrates in vivo expression of a polypeptide adjuvant from a circular RNA.

In this example, the following circular RNAs were produced and purified according to the methods described herein: (i) a first circular RNA with an IRES and an ORF encoding an IL-12 adjuvant (Nucleic acid SEQ ID NO: 38; Amino acid SEQ ID NO: 39); and (ii) a second circular RNA with an IRES and ORF encoding a SARS-CoV-2 RBD immunogen with an N-terminal Gluc signal sequence (Nucleic acid SEQ ID NO: 33; Amino acid SEQ ID NO: 32). The first circular RNA and the second circular RNA were each separately formulated with lipid nanoparticles as described in Example 16 and then mixed together to obtain a first circular RNA preparation.

Three mice were vaccinated intramuscularly at day 0 with the first circular RNA preparation (10 ug dose). As controls, three mice per group were vaccinated intramuscularly with: (i) the second circular RNA formulated with lipid nanoparticles (10 ug dose) (i.e., the second circular RNA preparation); or (ii) PBS. Blood collection from each mouse was by submandibular drawing. Blood was collected from each mouse into dry-anticoagulant free-tubes at 2 days after administration of circular RNA or PBS. Serum was separated from whole blood by centrifugation at 1200 g for 30 minutes at 4° C. Individual serum samples were assayed for the presence of IL12 using a cytokine bead array (Biolegend, 749622). Data are shown as Mean and SEM values of three replicates.

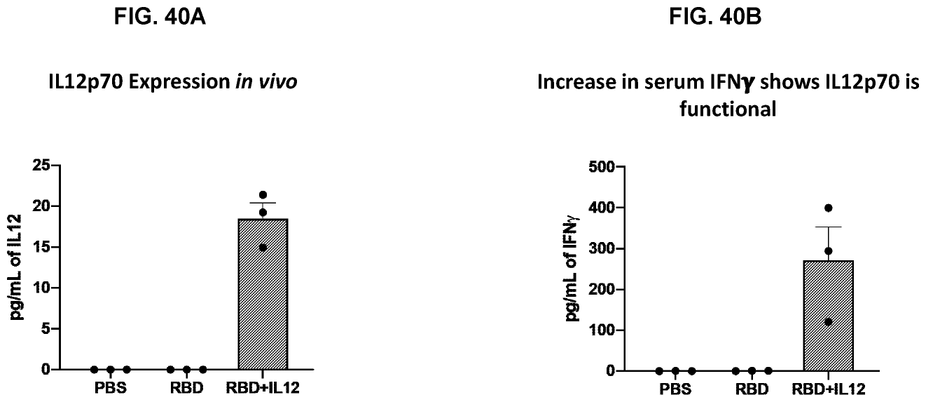

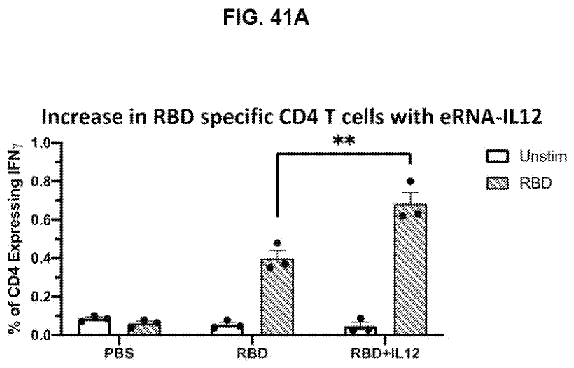

The results showed that IL-12 expression was detected in serum at 2 days after injection with the first circular RNA preparation but was not detected after injection with either of the controls (FIG. 40A).

To determine if the expressed IL12 is functional, IFN-γ (directly downstream of IL12 signaling) production in the serum was measured 2 days after injection with the circular RNA preparations. IFN-γ production was detected in serum in the same cytokine bead array assay described herein (Biolegend, 749622). Data are shown as Mean and SEM values of 3 replicates.

The results showed an increase in serum IFN-γ, indicating that circular RNA expressed IL12 is functional (FIG. 40B).

### Example 54: Induction of Immunogenicity in a Mouse Model by Co-Administration of an Immunogen and an Adjuvant Encoded by a Plurality of Circular RNAs

This example demonstrates the immunogenicity induced by administration of a plurality of circular RNAs to a subject. One circular RNA administered encodes an immunogen. Another circular RNA administered encodes a polypeptide adjuvant.

In this example, the following circular RNAs were produced and purified according to the methods described herein: (i) a first circular RNA with an IRES and an ORF encoding an IL-12 adjuvant (Nucleic acid SEQ ID NO: 38; Amino acid SEQ ID NO: 39); and (ii) a second circular RNA with an IRES and ORF encoding a SARS-CoV-2 RBD immunogen with an N-terminal Gluc signal sequence (Nucleic acid SEQ ID NO: 33; Amino acid SEQ ID NO: 32). The first circular RNA and the second circular RNA were each separately formulated with lipid nanoparticles as described in Example 16 and then mixed together to obtain a first circular RNA preparation.

Three mice were vaccinated intramuscularly at day 0 and day 14 with the first circular RNA preparation (2.5 ug dose per circular RNA per injection). As controls, three mice per group were vaccinated intramuscularly with: (i) the second circular RNA formulated with lipid nanoparticles (2.5 ug dose per injection) (i.e., the second circular RNA preparation); or (ii) PBS. Mice were euthanized 22 days post the first dose and splenocytes processed into a single cell suspension. Splenocytes were either left unstimulated or stimulated with RBD peptide pool (JPT, PM-WCPV-S-RBD-2) for 1 hour. Protein transport inhibitors (Monensin, BD 554724) and Brefeldin A, BD 555029)) were then added to the media followed by 5 more hours of culture. The cells were stained using the BD fixation and permeabilization kit (BD, 554714) according to the manufacturer's protocol. Antibodies used: viability dye (ThermoFisher, L1011), CD8 (ThermoFisher, MA5-16759) and CD44 (Biolegend, 103026). Stained cells were analyzed by flow cytometry.

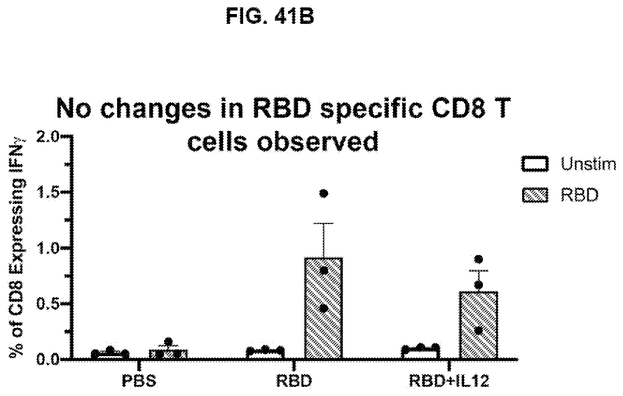

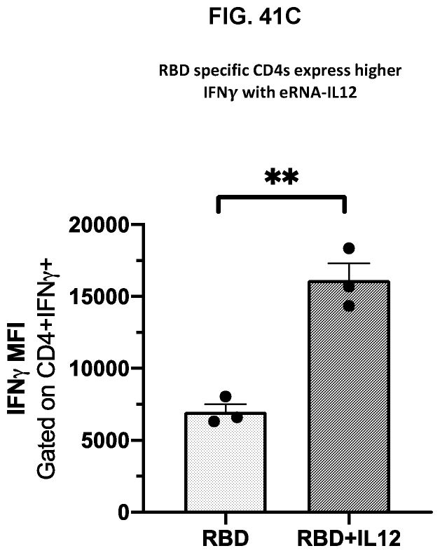

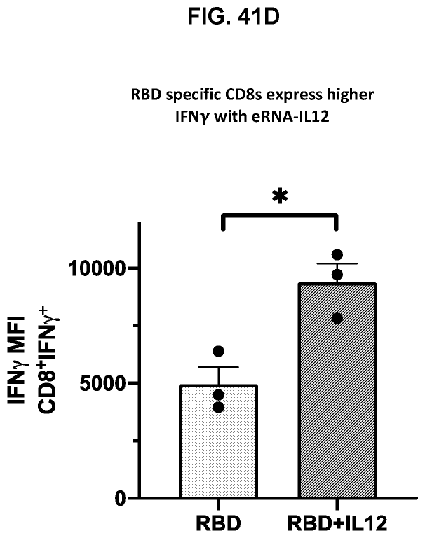

The data shows that the first circular RNA preparation increased the number of RBD specific CD4 T cells relative to the control, i.e., the second circular RNA preparation (FIG. 41A) and that no changes in RBD specific CD8 T cells were observed (FIG. 41B). In addition, the first circular RNA preparation increased the amount of IFN-γ production by both CD4 and CD8 T cells (FIGS. 41C, 41D). This shows that circular RNA expressing IL12 is acting as an adjuvant boosting cellular immune response elicited by circular RNA expressing an immunogen. Data shown as mean and SEM of 3 replicates. FIG. 41A: Asterisks denotes statistical significance as determined by a two-way RM ANOVA protected Tukey's post hoc test. FIGS. 41C, 41D: Asterisks denotes statistical significance as determined by unpaired t-test.

### Example 55: Evaluation of T Cell Responses after Co-Administration of an Immunogen and an Adjuvant Encoded by a Plurality of Circular RNAs

In this experiment, the following circular RNAs were produced and purified according to the methods described herein: (i) a first circular RNA including an IRES, an ORF encoding Ovalbumin (OVA) (Nucleic acid SEQ ID NO: 40; Amino acid SEQ ID NO: 41), and two spacer elements flanking the IRES-ORF; and (ii) a second circular RNA including an IRES, an ORF encoding the adjuvant IL-12 (Nucleic acid SEQ ID NO: 38; Amino acid SEQ ID NO: 39), and two spacer elements flanking the IRES-ORF. The first circular RNA and the second circular RNA were mixed together and complexed with Lipofectamine MessengerMax (Invitrogen LMRNA001) to obtain a mixture. As a control, a third circular RNA including an IRES, an ORF encoding a Gluc polypeptide, and two spacer elements flanking the IRES-ORF was also produced.

The following were transfected into EL4 cells (10,000 cells per well; EL4 cells were obtained from ATCC) using Lipofectamine MessengerMax according to the manufacturer's instructions:
1) Mixture of the first circular RNA and second circular RNA complexed with Lipofectamine MessengerMax (0.1 picomoles eRNA OVA+0.1 picomoles eRNA IL12);
2) first circular RNA (0.1 picomoles, eRNA OVA);
3) second circular RNA (0.1 picomoles, eRNA IL12);
4) third circular RNA (0.1 picomoles, eRNA Gluc);
5) transfection reagent alone (Blank transfection).

The transfected EL4 cells were cocultured with OT-1 CD8 T cells (OVA specific T cells; isolated from OT-1 mice from The Jackson Laboratory) for 2 days. The activation status of the T cells was monitored by the production of IFN-γ. Supernatant was harvested after 2 days and IFN-γ detected using a cytokine bead array (Biolegend, 741044). Data shown as mean and SEM of 3 replicates. Asterisks denotes statistical significance as determined by a one-way ANOVA protected Tukey's post hoc test.

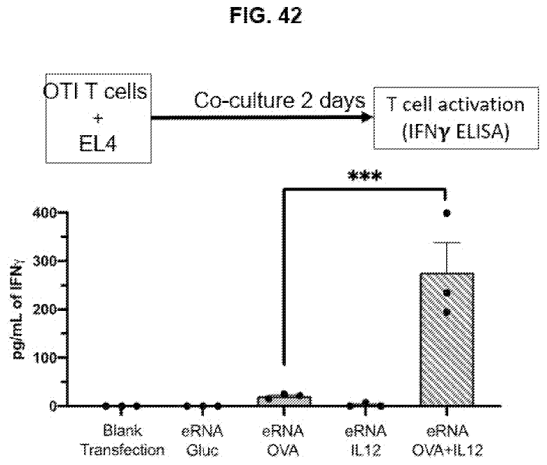

FIG. 42 shows that circular RNA expressing OVA combined with circular RNA expressing IL12 induces a stronger CD8 T cell response when compared to the control (circular RNA expressing OVA only). This demonstrates that circular RNA expressing IL12 acts as an adjuvant.

### SEQUENCES REFERENCED IN THE EXAMPLES

SEQ ID NO: 1
acgttactggccgaagccgcttggaataaggccggtgtgcgtttgtctatatgttattttccaccatattgccgtcttttggcaatgtgagggccc
ggaaacctggccctgtcttcttgacgagcattcctaggggtctttcccctctcgccaaaggaatgcaaggtctgttgaatgtcgtgaaggaagcag
ttcctctggaagcttcttgaagacaaacaacgtctgtagcgaccctttgcaggcagcggaaccccccacctggcgacaggtgcctctgcggccaaa
agccacgtgtataagatacacctgcaaaggcggcacaaccccagtgccacgttgtgagttggatagttgtggaaagagtcaaatggctctcctcaa
gcgtattcaacaaggggctgaaggatgcccagaaggtaccccattgtatgggatctgatctggggcctcggtgcacatgctttacatgtgtttagt
cgaggttaaaaaacgtctaggccccccgaaccacggggacgtggttttcctttgaaaaacacgatgataata
SEQ ID NO: 2
atgaaagcgattctggtggtgctgctgtatacctttgcgaccgcgaacgcggataccctgtgcattggctatcatgcgaacaacagcaccgataccg
tggataccgtgctggaaaaaaacgtgaccgtgacccatagcgtgaacctgctggaagataaacataacggcaaactgtgcaaactgcgcggcgt
ggcgccgctgcatctgggcaaatgcaacattgcgggctggattctgggcaacccggaatgcgaaagcctgagcaccgcgagcagctggagctat
attgtggaaaccccgagcagcgataacggcacctgctatccgggcgattttattgattatgaagaactgcgcgaacagctgagcagcgtgagcag
ctttgaacgctttgaaatttttccgaaaaccagcagctggccgaaccatgatagcaacaaaggcgtgaccgcggcgtgcccgcatgcgggcgcga
aaagcttttataaaaacctgatttggctggtgaaaaaaggcaacagctatccgaaactgagcaaaagctatattaacgataaaggcaaagaagtg
ctggtgctgtggggcattcatcatccgagcaccagcgcggatcagcagagcctgtatcagaacgcggatgcgtatgtgtttgtgggcagcagccgct
atagcaaaaaatttaaaccggaaattgcgattcgcccgaaagtgcgcgatcaggaaggccgcatgaactattattggaccctggtggaaccgggc
gataaaattacctttgaagcgaccggcaacctggtggtgccgcgctatgcgtttgcgatggaacgcaacgcgggcagcggcattattattagcgata
ccccggtgcatgattgcaacaccacctgccagaccccgaaaggcgcgattaacaccagcctgccgtttcagaacattcatccgattaccattggca
aatgcccgaaatatgtgaaaagcaccaaactgcgcctggcgaccggcctgcgcaacattccgagcattcagagccgcggcctgtttggcgcgatt
gcgggctttattgaaggcggctggaccggcatggtggatggctggtatggctatcatcatcagaacgaacagggcagcggctatgcggcggatctg
aaaagcacccagaacgcgattgatgaaattaccaacaaagtgaacagcgtgattgaaaaaatgaacacccagtttaccgcgggggcaaagaa
tttaaccatctggaaaaacgcattgaaaacctgaacaaaaaagtggatgatggctttctggatatttggacctataacgcggaactgctggtgctgc
tggaaaacgaacgcaccctggattatcatgatagcaacgtgaaaaacctgtatgaaaaagtgcgcagccagctgaaaaacaacgcgaaagaaa
ttggcaacggctgctttgaattttatcataaatgcgataacacctgcatggaaagcgtgaaaaacggcacctatgattatccgaaatatagcgaaga
agcgaaactgaaccgcgaagaaattgatggcgtgaaactggaaagcacccgcatttatcagattctggcgatttatagcaccgtggcgagcagcc
tggtgctggtggtgagcctgggcgcgattagcttttggatgtgcagcaacggcagcctgcagtgccgcatttgcatt
SEQ ID NO: 3
atgaaagcgaacctgctggtgctgctgtgcgcgctggcggcggcggatgcggataccatttgcattggctatcatgcgaacaacagcaccgatacc
gtggataccgtgctggaaaaaaacgtgaccgtgacccatagcgtgaacctgctggaagatagccataacggcaaactgtgccgcctgaaaggc
attgcgccgctgcagctgggcaaatgcaacattgcgggctggctgctgggcaacccggaatgcgatccgctgctgccggtgcgcagctggagcta
tattgtggaaaccccgaacagcgaaaacggcatttgctatccgggcgattttattgattatgaagaactgcgcgaacagctgagcagcgtgagcag
ctttgaacgctttgaaatttttccgaaagaaagcagctggccgaaccataacaccaacggcgtgaccgcggcgtgcagccatgaaggcaaaagc
agcttttatcgcaacctgctgtggctgaccgaaaaagaaggcagctatccgaaactgaaaaacagctatgtgaacaaaaaaggcaaagaagtgc
tggtgctgtggggcattcatcatccgccgaacagcaaagaacagcagaacctgtatcagaacgaaaacgcgtatgtgagcgtggtgaccagcaa
ctataaccgccgctttaccccggaaattgcggaacgcccgaaagtgcgcgatcaggcgggccgcatgaactattattggaccctgctgaaaccgg
gcgataccattatttttgaagcgaacggcaacctgattgcgccgatgtatgcgtttgcgctgagccgcggctttggcagcggcattattaccagca
acgcgagcatgcatgaatgcaacaccaaatgccagaccccgctgggcgcgattaacagcagcctgccgtatcagaacattcatccggtgaccattgg
cgaatgcccgaaatatgtgcgcagcgcgaaactgcgcatggtgaccggcctgcgcaacattccgagcattcagagccgcggcctgtttggcgcgat
tgcgggctttattgaaggcggctggaccggcatgattgatggctggtatggctatcatcatcagaacgaacagggcagcggctatgcggcggatca
gaaaagcacccagaacgcgattaacggcattaccaacaaagtgaacaccgtgattgaaaaaatgaacattcagtttaccgcggtgggcaaaga
atttaacaaactggaaaaacgcatggaaaacctgaacaaaaaagtggatgatggctttctggatatttggacctataacgcggaactgctggtgctg
ctggaaaacgaacgcaccctggattttcatgatagcaacgtgaaaaacctgtatgaaaaagtgaaaagccagctgaaaaacaacgcgaaaga
aattggcaacggctgctttgaattttatcataaatgcgataacgaatgcatggaaagcgtgcgcaacggcacctatgattatccgaaatatagcgaa
gaaagcaaactgaaccgcgaaaaagtggatggcgtgaaactggaaagcatgggcatttatcagattctggcgatttatagcaccgtggcgagca
gcctggtgctgctggtgagcctgggcgcgattagcttttggatgtgcagcaacggcagcctgcagtgccgcatttgcatt
SEQ ID NO: 4
aaaaaacaaaaaacaaaacggctattaatagccgaaaaacaaaaaacaaaaaaaacaaaaaaaaaaccaaaaaaaaaaacaca
SEQ ID NO: 30
MFVFLVLLPLVSSQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFNATRFASVYAWNRK
RISNCVADYSVLYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGFNCYFPLQSYGF
QPTNGVGYQPYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKGYHLMSFPQSAPHGVVFLHVT
YVPAQEKNFTTAPAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNESLIDLQELGKYEQY
IKWPWYIWLGFIAGLIAIVMVTIMLCCMTSCCSCLKGCCSCGSCCKFDEDDSEPVLKGVKLHYT
SEQ ID NO: 31
atgtttgtttttcttgttttattgccactagtctctagtcagtgtgttaatcttacaaccagaactcaattaccccctgcatacactaattctttc
acacgtggtgtttattaccctgacaaagttttcagatcctcagttttacattcaactcaggatttgttcttacctttcttttccaatgttacttgg
ttccatgctatacatgtctctgggaccaatggtactaagaggtttgataaccctgtcctaccatttaatgatggtgtttattttgcttccactgag
aagtctaacataataagaggctggatctttggtactactttagattcgaagacccagtccctacttattgttaataacgctactaatgttgttatt
aaagtctgtgaatttcaattttgtaatgatccatttttgggtgtttattaccacaaaaacaacaaaagttggatggaaagtgagttcagagttta
ttctagtgcgaataattgcacttttgaatatgtctctcagccttttcttatggaccttgaaggaaaacagggtaatttcaaaaatcttagggaa
tttgtgtttaagaatattgatggttattttaaaatatattctaagcacacgcctattaatttagtgcgtgatctccctcagggtttttcggcttt
agaaccattggtagatttgccaataggtattaacatcactaggtttcaaactttacttgctttacatagaagttatttgactcctggtgattctt
cttcaggttggacagctggtgctgcagcttattatgtgggttatcttcaacctaggacttttctattaaaatataatgaaaatggaaccattacag
atgctgtagactgtgcacttgaccctctctcagaaacaaagtgtacgttgaaatccttcactgtagaaaaaggaatctatcaaacttctaacttta
gagtccaaccaacagaatctattgttagatttcctaatattacaaacttgtgcccttttggtgaagtttttaacgccaccagatttgcatccg
tgtatgcttggaacaggaagagaatcagcaactgtgttgctgattattctgtcctatataattccgcatcattttccacttttaagtgttatggag
tgtctcctactaaattaaatgatctctgctttactaatgtctatgcagattcatttgtaattagaggtgatgaagtcagacaaatcgctccagggc
aaactggaaagattgctgattataattataaattaccagatgattttacaggctgcgttatagcttggaattctaacaatcttgattctaaggttg
gtggtaattataattacctgtatagattgtttaggaagtctaatctcaaaccttttgagagagatatttcaactgaaatctatcaggccggtagca
caccttgtaatggtgttgaaggttttaattgttactttcctttacaatcatatggtttccaacccactaatggtgttggttaccaaccatacagag
tagtagtactttcttttgaacttctacatgcaccagcaactgtttgtggacctaaaaagtctactaatttggttaaaaacaaatgtgtcaatttca
acttcaatggtttaacaggcacaggtgttcttactgagtctaacaaaaagtttctgcctttccaacaatttggcagagacattgctgacactactg
atgctgtccgtgatccacagacacttgagattcttgacattacaccatgttcttttggtggtgtcagtgttataacaccaggaacaaatacttcta
accaggttgctgttctttatcaggatgttaactgcacagaagtccctgttgctattcatgcagatcaacttactcctacttggcgtgtttattcta
caggttctaatgtttttcaaacacgtgcaggctgtttaataggggctgaacatgtcaacaactcatatgagtgtgacatacccattggtgcaggta
tatgcgctagttatcagactcagactaattctcctcgggggcacgtagtgtagctagtcaatccatcattgcctacactatgtcacttggtgcaga
aaattcagttgcttactctaataactctattgccatacccacaaattttactattagtgttaccacagaaattctaccagtgtctatgaccaagac
atcagtagattgtacaatgtacatttgtggtgattcaactgaatgcagcaatcttttgttgcaatatggcagtttttgtacacaattaaaccgtgc
tttaactgggatagctgttgaacaagacaaaaacacccaagaagtttttgcacaagtcaaacaaatttacaaaacaccaccaattaaagattttgg
tggttttaatttttcacaaatattaccagatccatcaaaaccaagcaagaggtcatttattgaagatctacttttcaacaaagtgacacttgcag
atgctggcttcatcaaacaatatggtgattgccttggtgatattgctgctagggacctcatttgtgcacaaaagtttaacggccttactgttttgc
cacctttgctcacagatgaaatgattgctcaatacacttctgcactgttagcgggtacaatcacttctggttggacctttggtgcaggtgctgcat
tacaaataccatttgctatgcaaatggcttataggtttaatggtattggagttacacagaatgttctctatgagaaccaaaaattgattgccaacc
aatttaatagtgccattggcaaaattcaagactcactttcttccacagcaagtgcacttggaaaacttcaagatgtggtcaaccaaaatgcacaa
gctttaaacacgcttgttaaacaacttagctccaattttggtgcaatttcaagtgttttaaatgatatcctttcacgtcttgaccctcccgaggct
gaagtgcaaattgataggttgatcacaggcagacttcaaagtttgcagacatatgtgactcaacaattaattagagctgcagaaatcagagcttct
gctaatcttgctgctactaaaatgtcagagtgtgtacttggacaatcaaaaagagttgatttttgtggaaagggctatcatcttatgtccttccct
cagtcagcacctcatggtgtagtcttcttgcatgtgacttatgtccctgcacaagaaaagaacttcacaactgctcctgccatttgtcatgatgga
aaagcacactttcctcgtgaaggtgtctttgtttcaaatggcacacactggtttgtaacacaaaggaatttttatgaaccacaaatcattactaca
gacaacacatttgtgtctggtaactgtgatgttgtaataggaattgtcaacaacacagtttatgatcctttgcaacctgaattagactcattcaag
gaggagttagataaatattttaagaatcatacatcaccagatgttgatttaggtgacatctctggcattaatgcttcagttgtaaacattcaaaaa
gaaattgaccgcctcaatgaggttgccaagaatttaaatgaatctctcatcgatctccaagaacttggaaagtatgagcagtatataaaatggcc
atggtacatttggctaggttttatagctggcttgattgccatagtaatggtgacaattatgctttgctgtatgaccagttgctgtagttgtctcaa
gggctgttgttcttgtggatcctgctgcaaatttgatgaagacgactctgagccagtgctcaaaggagtcaaattacattacaca
SEQ ID NO: 32
MGVKVLFALICIAVAEAKRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFPLQSYGFQPTNGVGYQPYRV
VVLSFELLHAPATVCGPKKSTNLVKNKCVNF
SEQ ID NO: 33
atgggagtcaaagttctgtttgccctgatctgcattgctgtggccgaggccaagagagtccaaccaacagaatctattgttagatttcctaatatt
acaaacttgtgcccttttggtgaagtttttaacgccaccagatttgcatccgtgtatgcttggaacaggaagagaatcagcaactgtgttgctga
ttattctgtcctatataattccgcatcattttccacttttaagtgttatggagtgtctcctactaaattaaatgatctctgctttactaatgtcta
tgcagattcatttgtaattagaggtgatgaagtcagacaaatcgctccagggcaaactggaaagattgctgattataattataaattaccagatga
ttttacaggctgcgttatagcttggaattctaacaatcttgattctaaggttggtggtaattataattacctgtatagattgtttaggaagtcta
atctcaaaccttttgagagagatatttcaactgaaatctatcaggccggtagcacaccttgtaatggtgttgaaggttttaattgttactttcct
ttacaatcatatggtttccaacccactaatggtgttggttaccaaccatacagagtagtagtactttcttttgaacttctacatgcaccagcaac
tgtttgtggacctaaaaagtctactaatttggttaaaaacaaatgtgtcaatttc
SEQ ID NO: 34
MKAILVVLLYTFATANADTLCIGYHANNSTDTVDTVLEKNVTVTHSVNLLEDKHNGKLCKLRGVAPLHLGK
CNIAGWILGNPECESLSTASSWSYIVETPSSDNGTCYPGDFIDYEELREQLSSVSSFERFEIFPKTSSWPN
HDSNKGVTAACPHAGAKSFYKNLIWLVKKGNSYPKLSKSYINDKGKEVLVLWGIHHPSTSADQQSLYQN
ADAYVFVGSSRYSKKFKPEIAIRPKVRDQEGRMNYYWTLVEPGDKITFEATGNLVVPRYAFAMERNAGS
GIIISDTPVHDCNTTCQTPKGAINTSLPFQNIHPITIGKCPKYVKSTKLRLATGLRNIPSIQSRGLFGAIAGFIE
GGWTGMVDGWYGYHHQNEQGSGYAADLKSTQNAIDEITNKVNSVIEKMNTQFTAVGKEFNHLEKRIENL
NKKVDDGFLDIWTYNAELLVLLENERTLDYHDSNVKNLYEKVRSQLKNNAKEIGNGCFEFYHKCDNTCM
ESVKNGTYDYPKYSEEAKLNREEIDGVKLESTRIYQILAIYSTVASSLVLVVSLGAISFWMCSNGSLQCRICI
SEQ ID NO: 35
ATGAAAGCAATACTAGTAGTTCTTCTATATACATTTGCAACCGCTAACGCTGATACATTGTGTATAGGA
TATCACGCGAACAACTCCACCGATACAGTAGATACAGTACTAGAGAAGAACGTAACAGTAACACATT
CTGTTAATCTTCTAGAAGACAAGCATAACGGCAAACTGTGCAAACTAAGAGGTGTAGCCCCATTGCA
TCTAGGAAAGTGTAATATAGCTGGCTGGATTTTGGGAAATCCAGAGTGTGAATCATTAAGTACAGCAA
GCTCCTGGTCCTATATAGTGGAAACACCTAGTAGTGATAACGGAACGTGTTACCCAGGAGATTTTATA
GATTACGAGGAGCTAAGAGAGCAGCTGTCGTCAGTATCATCATTTGAAAGGTTTGAAATTTTCCCGAA
AACATCCTCCTGGCCCAATCACGATAGTAACAAAGGAGTAACAGCAGCCTGTCCTCACGCTGGAGCA
AAAAGCTTCTATAAAAATTTAATCTGGCTAGTGAAGAAGGGAAATTCATATCCAAAGCTAAGTAAAAGT
TATATAAACGATAAGGGCAAGGAAGTACTCGTACTGTGGGGCATTCATCATCCATCTACTAGTGCTG
ATCAACAAAGTTTATATCAAAACGCAGACGCATACGTTTTTGTGGGGTCAAGTAGATATAGCAAGAAA
TTTAAACCAGAAATAGCAATAAGACCTAAAGTAAGGGATCAAGAAGGCAGAATGAACTATTATTGGAC
ACTAGTAGAACCGGGAGATAAAATAACTTTTGAAGCAACAGGAAATCTAGTGGTTCCCAGGTACGCA
TTTGCAATGGAAAGAAACGCTGGATCAGGCATCATTATATCTGATACACCAGTCCACGATTGTAATAC
AACTTGTCAAACACCTAAAGGAGCTATAAACACCAGCTTACCATTTCAAAATATTCATCCTATCACAAT
TGGAAAGTGTCCAAAATACGTAAAAAGTACAAAATTGAGATTGGCCACAGGATTACGAAATATTCCAT
CAATTCAATCTAGAGGACTTTTTGGTGCAATTGCAGGTTTCATAGAAGGAGGCTGGACTGGGATGGT
AGACGGCTGGTACGGTTATCATCATCAAAACGAACAGGGAAGTGGATACGCAGCTGATCTTAAAAGT
ACACAAAACGCAATTGACGAGATTACTAATAAAGTAAATTCTGTAATTGAAAAAATGAATACTCAGTTT
ACAGCAGTAGGGAAAGAGTTTAACCACCTGGAAAAAAGAATAGAAAATTTAAATAAAAAAGTAGACGA
CGGATTTCTTGACATTTGGACTTATAACGCCGAACTATTGGTATTACTAGAAAACGAAAGAACTCTAG
ATTATCACGATTCAAACGTAAAAAATTTATACGAAAAAGTAAGAAGCCAACTTAAAAATAACGCAAAAG
AAATAGGAAACGGCTGTTTTGAATTTTATCACAAGTGTGATAATACCTGCATGGAAAGTGTTAAAAAC
GGGACATACGATTATCCAAAATACTCAGAAGAAGCAAAATTAAATAGAGAAGAAATAGACGGCGTAA
AATTAGAATCAACAAGGATATATCAAATATTAGCAATATATTCAACTGTCGCTTCTTCATTGGTACTGG
TAGTTTCTCTAGGTGCAATATCATTTTGGATGTGCTCTAACGGCTCCCTACAGTGTAGAATTTGTATA
SEQ ID NO: 36
MGVKVLFALICIAVAEAKPTENNEDFNIVAVASNFATTDLDADRGKLPGKKLPLEVLKEMEANARKAGCTR
GCLICLSHIKCTPKMKKFIPGRCHTYEGDKESAQGGIGEAIVDIPEIPGFKDLEPMEQFIAQVDLCVDCTTG
CLKGLANVQCSDLLKKWLPQRCATFASKIQGQVDKIKGAGGD
SEQ ID NO: 37
ATGGGAGTCAAAGTTCTGTTTGCCCTGATCTGCATCGCTGTGGCCGAGGCCAAGCCCACCGAGAAC
AACGAAGACTTCAACATCGTGGCCGTGGCCAGCAACTTCGCGACCACGGATCTCGATGCTGACCGC
GGGAAGTTGCCCGGCAAGAAGCTGCCGCTGGAGGTGCTCAAAGAGATGGAAGCCAATGCCCGGAA
AGCTGGCTGCACCAGGGGCTGTCTGATCTGCCTGTCCCACATCAAGTGCACGCCCAAGATGAAGAA
GTTCATCCCAGGACGCTGCCACACCTACGAAGGCGACAAAGAGTCCGCACAGGGGGGCATAGGCG
AGGCGATCGTCGACATTCCTGAGATTCCTGGGTTCAAGGACTTGGAGCCCATGGAGCAGTTCATCG
CACAGGTCGATCTGTGTGTGGACTGCACAACTGGCTGCCTCAAAGGGCTTGCCAACGTGCAGTGTT
CTGACCTGCTCAAGAAGTGGCTGCCGCAACGCTGTGCGACCTTTGCCAGCAAGATCCAGGGCCAGG
TGGACAAGATCAAGGGGGCCGGTGGTGACTAA
SEQ ID NO: 38
atggctccccaaaagctgaccatcagctggttcgccatcgtgctgctcgtgagccccctgatggccatgtgggagctggagaaggacgtgtacgtg
gtcgaggtggactggacccccgacgcccccggcgaaacagtgaacctgacctgcgacacccccgaggaagacgatatcacctggacaagcg
accagcggcacggcgtgatcggcagcggcaagaccctgaccatcaccgtgaaggagttcctggacgccggccagtacacctgccacaagggc
ggagaaacactgagccacagccacctgctgttgcacaagaaagagaacggcatctggagcaccgagatcctcaagaatttcaagaacaagac
cttcctgaagtgcgaggcccccaactacagcggccggttcacctgcagctggctggtgcagcggaacatggacctgaagttcaacatcaagagct
ccagctctagccccgacagccgggccgtgacctgcggcatggccagcctgagcgccgagaaggtgaccctggaccagcgggactacgagaa
gtacagcgtgagctgccaggaggacgtgacctgccctaccgctgaggaaacactgcccatcgagctggccctggaggcccggcagcaaaaca
agtacgagaactacagcaccagcttctttatccgggacatcattaagcccgacccccctaagaacctgcagatgaagcccctgaagaacagcca
ggtggaggtgagctgggagtaccccgacagctggagcaccccccacagctacttcagcctgaagttcttcgtgcggatccagcggaagaaggag
aagatgaaggaaacagaggagggctgcaaccagaagggcgccttcctggtggaaaagacaagcacagaggtgcagtgcaagggcggcaa
cgtgtgcgtgcaggcccaggaccggtactataacagcagctgcagcaagtgggcctgcgtgccctgccgggtgcggagtgtgcctggcgtcggc
gtgcccggcgtgggccgggtgatccccgtgagcggccccgcccggtgcctgagccagagccggaacctgctgaagaccacagacgacatggt
gaagaccgcccgggagaagctgaagcactacagctgcaccgccgaggacatcgaccacgaggacatcacccgggaccagaccagcaccct
gaagacctgcctccccctggagctgcacaagaacgagagctgcctggccacccgggaaacaagctctaccacacggggcagctgcctgcctcc
tcagaagaccagcctgatgatgaccctgtgcctgggcagcatctacgaggacctgaagatgtaccagaccgagttccaggccatcaacgccgctc
tgcagaaccacaaccaccagcagatcatcctggacaagggcatgctggtggccatcgacgagctgatgcagagcctgaaccacaacggcgaa
acactgcggcagaagcccccagtgggcgaggccgacccctaccgggtgaagatgaagctgtgcatcctgctgcacgccttcagcacccgggtg
gtgaccatcaaccgggtgatgggctacctgagcagcgcc
SEQ ID NO: 39
MCPQKLTISWFAIVLLVSPLMAMWELEKDVYVVEVDWTPDAPGETVNLTCDTPEEDDITWTSDQRHGVI
GSGKTLTITVKEFLDAGQYTCHKGGETLSHSHLLLHKKENGIWSTEILKNFKNKTFLKCEAPNYSGRFTCS
WLVQRNMDLKFNIKSSSSSPDSRAVTCGMASLSAEKVTLDQRDYEKYSVSCQEDVTCPTAEETLPIELAL
EARQQNKYENYSTSFFIRDIIKPDPPKNLQMKPLKNSQVEVSWEYPDSWSTPHSYFSLKFFVRIQRKKEK
MKETEEGCNQKGAFLVEKTSTEVQCKGGNVCVQAQDRYYNSSCSKWACVPCRVRSVPGVGVPGVGR
VIPVSGPARCLSQSRNLLKTTDDMVKTAREKLKHYSCTAEDIDHEDITRDQTSTLKTCLPLELHKNESCLA
TRETSSTTRGSCLPPQKTSLMMTLCLGSIYEDLKMYQTEFQAINAALQNHNHQQIILDKGMLVAIDELMQS
LNHNGETLRQKPPVGEADPYRVKMKLCILLHAFSTRVVTINRVMGYLSSA
SEQ ID NO: 40
atgggcagcatcggcgccgctagcatggagttctgctttgacgtcttcaaagagctgaaggtgcaccacgccaacgagaacatcttctactgccct
attgccatcatgagcgccctggccatggtgtacctgggcgccaaggacagcacccggacccagatcaacaaggtggtccggttcgacaagctgcc
cggcttcggcgacagcatcgaggcccagtgcggcaccagcgtgaacgtgcacagctccctgcgggacatcctgaaccagatcaccaagcccaa
cgacgtgtacagcttcagcctggccagccggctgtacgccgaggaacggtaccccatcctgcccgagtacctgcagtgcgtgaaggagctgtacc
ggggcggactggagcccatcaacttccagaccgccgccgaccaggcccgggagctgatcaacagctgggtggagagccagaccaacggcat
cattcggaacgtgctgcagcccagcagcgtggacagccagaccgccatggtgctggtgaacgccatcgtgttcaagggcctgtgggagaaggcc
ttcaaggacgaggacacccaggccatgcccttccgggtgaccgagcaggagagcaagcccgtgcagatgatgtaccagatcggcctgttccgg
gtggccagcatggccagcgagaagatgaagatcctggagctgcccttcgccagcggcaccatgagcatgctggtgctgctccccgacgaggtga
gcggcctggagcagctggagagcatcatcaacttcgagaagctgaccgagtggaccagctctaacgtgatggaggagcggaagatcaaggtgt
acctgccccggatgaagatggaggagaagtacaacctgaccagcgtgctgatggccatgggcatcaccgacgtgttcagcagcagcgccaacc
tgagcggcatcagctccgccgagagcctgaagatcagccaggccgtgcacgccgcacacgccgagatcaacgaggccggccgggaggtggt
gggcagcgccgaggccggcgtggacgccgccagcgtgagcgaggagttccgggccgaccaccccttcctgttctgcatcaagcatattgccacc
aacgccgtgctgttctttggccggtgcgtgagcccc
SEQ ID NO: 41
MGSIGAASMEFCFDVFKELKVHHANENIFYCPIAIMSALAMVYLGAKDSTRTQINKVVRFDKLPGFGDSIE
AQCGTSVNVHSSLRDILNQITKPNDVYSFSLASRLYAEERYPILPEYLQCVKELYRGGLEPINFQTAADQA
RELINSWVESQTNGIIRNVLQPSSVDSQTAMVLVNAIVFKGLWEKAFKDEDTQAMPFRVTEQESKPVQM
MYQIGLFRVASMASEKMKILELPFASGTMSMLVLLPDEVSGLEQLESIINFEKLTEWTSSNVMEERKIKVYL
PRMKMEEKYNLTSVLMAMGITDVFSSSANLSGISSAESLKISQAVHAAHAEINEAGREVVGSAEAGVDAA
SVSEEFRADHPFLFCIKHIATNAVLFFGRCVSP

1. A circular polyribonucleotide comprising (a) a sequence encoding a polypeptide immunogen and (b) a sequence encoding an adjuvant or a sequence that is an innate immune system stimulator.

2. The circular polyribonucleotide of claim 1, wherein the sequence encoding the polypeptide immunogen is operably linked to an IRES.

3. The circular polyribonucleotide of claim 1 or 2, wherein the sequence encoding the adjuvant is operably linked to an IRES.

4. The circular polyribonucleotide of any one of claims 1-3, wherein the adjuvant is a polypeptide.

5. The circular polyribonucleotide of claim 4, wherein the polypeptide immunogen and the adjuvant polypeptide are encoded by a single open-reading frame operably linked to the single IRES, wherein the expression of the open reading frame produces a polypeptide comprising the amino acid sequence of each of the polypeptide immunogen and the adjuvant polypeptide.

6. The circular polyribonucleotide of claim 5, wherein the polypeptide immunogen and the adjuvant polypeptide are each separated by a polypeptide linker.

7. The circular polyribonucleotide of claim 5, wherein the polypeptide immunogen and the adjuvant polypeptide are each separated by a cleavage domain.

8. The circular polyribonucleotide of claim 6, wherein the cleavage domain is a 2A self-cleaving peptide.

9. The circular polyribonucleotide of claim 4, wherein the circular polyribonucleotide comprises a plurality of IRESs.

10. The circular polyribonucleotide of claim 9, wherein a first IRES is operably linked to the sequence encoding the polypeptide immunogen and a second IRES is operably linked to the sequence encoding the adjuvant polypeptide.

11. The circular polyribonucleotide of any one of claims 1-10, wherein the expression of the sequence encoding the polypeptide immunogen is at least 2-fold greater than the expression of the sequence encoding the adjuvant.

12. The circular polyribonucleotide of claim 11, wherein the expression of the sequence encoding the polypeptide immunogen is at least 5-fold, 10-fold, or 100-fold greater than the expression of the sequence encoding the adjuvant.

13. An immunogenic composition comprising a plurality of circular polyribonucleotides, wherein the composition comprises (a) at least a first circular polyribonucleotide comprising a sequence encoding a polypeptide immunogen and (b) at least a second circular polyribonucleotide comprising a sequence encoding an adjuvant or a sequence that is an innate immune system stimulator.

14. The immunogenic composition of claim 13, wherein the adjuvant is a polypeptide.

15. The immunogenic composition of claims 13 or 14, wherein the polypeptide immunogen and the adjuvant are each operably linked to an IRES.

16. The circular polyribonucleotide or immunogenic composition of any one of claims 1-15, wherein the adjuvant is a cytokine, a chemokine, a costimulatory molecule, an innate immune stimulator, a signaling molecule, a transcriptional activator, a cytokine receptor, a bacterial component, or a component of the innate immune system.

17. The circular polyribonucleotide or immunogenic composition of claim 16, wherein the cytokine is a pro-inflammatory cytokine.

18. The circular polyribonucleotide or immunogenic composition of claim 17, wherein the pro-inflammatory cytokine is selected from GM-CSF, IL-1 alpha, IL-1 beta, TGF-beta, TNF-alpha, and TNF-beta.

19. The circular polyribonucleotide or immunogenic composition of claim 16, wherein the cytokine is a Th-1 inducing cytokine.

20. The circular polyribonucleotide or immunogenic composition of claim 19, wherein the Th-1 inducing cytokine is selected from IFN-gamma, IL-2, IL-12, IL-15, and IL-18.

21. The circular polyribonucleotide or immunogenic composition of claim 16, wherein the cytokine is a Th-2 inducing cytokine.

22. The circular polyribonucleotide or immunogenic composition of claim 21, wherein the Th-2 inducing cytokine is selected from IFN-gamma, IL-4, IL-5, IL-6, IL-10, and IL-13.

23. The circular polyribonucleotide or immunogenic composition of claim 16, wherein the chemokine is selected from MCP-1, MIP-1alpha, MIP-1 beta, Rantes, and TCA-3.

24. The circular polyribonucleotide or immunogenic composition of claim 16, wherein the costimulatory molecule is selected from CD80, CD86, CD40-L, CD70, and CD27.

25. The circular polyribonucleotide or immunogenic composition of claim 16, wherein the innate immune stimulator is selected from STING, TLR3, TLR4, TLR9, TLR7, TLR8, TLR7, RIG-I/DDX58, and MDA-5/IFIH1.

26. The circular polyribonucleotide or immunogenic composition of claim 25, wherein the innate immune stimulator is a constitutively active mutant.

27. The circular polyribonucleotide or immunogenic composition of claim 16, wherein the signaling molecule is selected from STING, TRIF, TRAM, MyD88, IPS1, ASC, MAVS, MAPKs, IKK-alpha, IKK complex, TBK1, B-catenin, and caspase 1.

28. The circular polyribonucleotide or immunogenic composition of claim 16, wherein the transcriptional activator is selected from AP1, NF-kappa B, IRF3, IRF7, IRF1, and IRFS.

29. The circular polyribonucleotide or immunogenic composition of claim 16, wherein the cytokine receptor is selected from IL-2betaR, IFN-gammaR, and IL-6R.

30. The circular polyribonucleotide or immunogenic composition of claim 16, wherein the bacterial component is selected from flagellin and MBL.

31. The circular polyribonucleotide or immunogenic composition of any one of claims 1-30, wherein the sequence encoding the adjuvant further encodes a signal sequence.

32. The circular polyribonucleotide or immunogenic composition of any one of claims 1-31, wherein the sequence encoding the polypeptide immunogen further encodes a signal sequence.

33. The circular polyribonucleotide or immunogenic composition of any one of claims 1-31, wherein the sequence encoding the polypeptide immunogen does not encode a signal sequence.

34. The circular polyribonucleotide or immunogenic composition of claim 1 or 13, wherein the sequence that is an innate immune system stimulator is selected from a GU-rich motif, an AU-rich motif, a structured region comprising dsRNA, or an aptamer.

35. A circular polyribonucleotide comprising a plurality of sequences, each sequence encoding a polypeptide immunogen, wherein at least two of the polypeptide immunogens have at least 90% amino acid sequence identity.

36. The circular polyribonucleotide of claim 35, wherein the circular polyribonucleotide comprises at least three, at least four, at least five, at least six, at least seven, at least eight, or at least nine sequences, each sequence encoding a polypeptide immunogen.

37. The circular polyribonucleotide of claims 35 or 36, wherein each of the sequences encoding each of the polypeptide immunogens is operably linked to an internal ribosomal entry site (IRES).

38. The circular polyribonucleotide of claim 37, wherein the circular polyribonucleotide comprises a single IRES.

39. The circular polyribonucleotide of claim 38, wherein each of the polypeptide immunogens is encoded by a single open-reading frame operably linked to the single IRES, wherein the expression of the open reading frame produces a polypeptide comprising the amino acid sequence of each the polypeptide immunogens.

40. The circular polyribonucleotide of claim 39, wherein the polypeptide immunogens are each separated by a polypeptide linker.

41. The circular polyribonucleotide of claim 39, wherein the polypeptide immunogens are each separated by a cleavage domain.

42. The circular polyribonucleotide of claim 41, wherein the cleavage domain is a 2A self-cleaving peptide.

43. The circular polyribonucleotide of claim 37, wherein the circular polyribonucleotide comprises a plurality of IRESs.

44. The circular polyribonucleotide of claim 43, wherein each IRES is operably linked to an open reading frame comprising a sequence encoding a polypeptide immunogen.

45. The circular polyribonucleotide of any one of claims 35-44, wherein the circular polyribonucleotide further comprises at least one sequence encoding an adjuvant.

46. The circular polyribonucleotide of claim 45, wherein the at least one sequence encoding the adjuvant is operably linked to an IRES.

47. The circular polyribonucleotide of claim 45 or 46, wherein the adjuvant is a polypeptide.

48. The circular polyribonucleotide of claim 47, wherein the adjuvant is a cytokine, a chemokine, a costimulatory molecule, an innate immune stimulator, a signaling molecule, a transcriptional activator, a cytokine receptor, a bacterial component, or a component of the innate immune system.

49. The circular polyribonucleotide of any one of claims 35-48, wherein the circular polyribonucleotide further comprises a sequence that is an innate immune system stimulator.

50. The circular polyribonucleotide of claim 49, wherein the sequence that is an innate immune system stimulator is a GU-rich motif, an AU-rich motif, a structured region comprising dsRNA, or an aptamer.

51. An immunogenic composition comprising (a) at least a first circular polyribonucleotide comprising a sequence encoding a first polypeptide immunogen and (b) at least a second circular polyribonucleotide comprising a sequence encoding a second polypeptide immunogen, wherein the first and second polypeptide immunogens have at least 90% amino acid sequence identity.

52. The immunogenic composition of claim 51, wherein each polypeptide immunogen is operably linked to an IRES.

53. The circular polyribonucleotide or immunogenic composition of any one of claims 35-52, wherein each of the polypeptide immunogens have less than 100% amino acid sequence identity.

54. The circular polyribonucleotide of any one of claims 35-53, wherein each of the polypeptide immunogens comprises one or more epitopes that identifies a target.

55. The circular polyribonucleotide of claim 54, wherein the target is a pathogen.

56. The circular polyribonucleotide of claim 55, wherein the pathogen is a virus, a bacterium, a fungus, or a parasite.

57. The circular polyribonucleotide of claim 54, wherein the target is a cancer cell.

58. The circular polyribonucleotide of claim 55, wherein the target is a toxin or an allergen.

59. A pharmaceutical composition comprising the circular polyribonucleotide or immunogenic composition of any one of claims 1-58, and a pharmaceutically acceptable excipient.

60. A method of inducing an immune response in a subject, the method comprising administering to the subject the circular polyribonucleotide or immunogenic composition, or pharmaceutical composition of any one of claims 1-59.
